(19) United States
(12) Patent Application Publication
(10) Pub. No.: US 2022/0263030 A1
(43) Pub. Date: Aug. 18, 2022

(54) ORGANIC ELECTROLUMINESCENT ELEMENT AND ELECTRONIC DEVICE

(71) Applicants: IDEMITSU KOSAN CO.,LTD., Chiyoda-ku, Tokyo (JP); TORAY INDUSTRIES, INC., Chuo-ku, Tokyo (JP)

(72) Inventors: Hiromi NAKANO, Sodegaura-shi (JP); Hisato MATSUMOTO, Sodegaura-shi (JP); Takushi SHIOMI, Sodegaura-shi (JP); Toshinari OGIWARA, Sodegaura-shi (JP); Keiji OKINAKA, Sodegaura-shi (JP); Kazunari KAWAMOTO, Otsu-shi (JP); Takashi TOKUDA, Otsu-shi (JP); Kazumasa NAGAO, Otsu-shi (JP)

(73) Assignees: IDEMITSU KOSAN CO.,LTD., Chiyoda-ku, Tokyo (JP); TORAY INDUSTRIES, INC., Chuo-ku, Tokyo (JP)

(21) Appl. No.: 17/629,289

(22) PCT Filed: Jul. 20, 2020
(86) PCT No.: PCT/JP2020/028145

§ 371 Date: Jan. 21, 2022

(30) Foreign Application Priority Data
Jul. 24, 2019 (JP) 2019-136109

(51) Int. Cl. H01L 51/00; C07D 491/048; C09K 11/06; C07D 495/04; C07F 5/02

(52) U.S. Cl. CPC H01L 51/0071; C07D 491/048; C09K 11/06; H01L 51/5012; C07D 495/04; C07F 5/027; H01L 51/008; H01L 51/0072

(57) ABSTRACT

An organic-EL device includes an emitting-layer, first-layer adjacent to the emitting-layer near the anode, and second-layer adjacent to the emitting-layer near the cathode. The emitting-layer contains first, second and third compounds. The first-layer contains a compound of formula-(A). The second-layer contains a compound of formula-(B). The first, second and third compounds are a fluorescent compound of formula-(1), delayed-fluorescent compound of formula-(2), and compound of formula-(3), respectively. Singlet energies S₁ of the first to third compounds satisfy S₁(M3)>S₁(M2)>S₁(M1). In the formula-(A), Ra₁-Ra₅ etc. each represent a substituent etc. In the formula-(B), X₁-X₃ each represent N atom etc, Ar₁ and Ar₂ each represent a group of formula-(1B) etc. A represents the group of the formula-(1B). In the formula-(1B), HAr is of formula-(2B), a is 1 to 5, and L₁ is a linking group etc. In the formula-(2B), X₁₁-X₁₈ each represent N atom etc, and Y₁ represent O, S, or N atom etc.

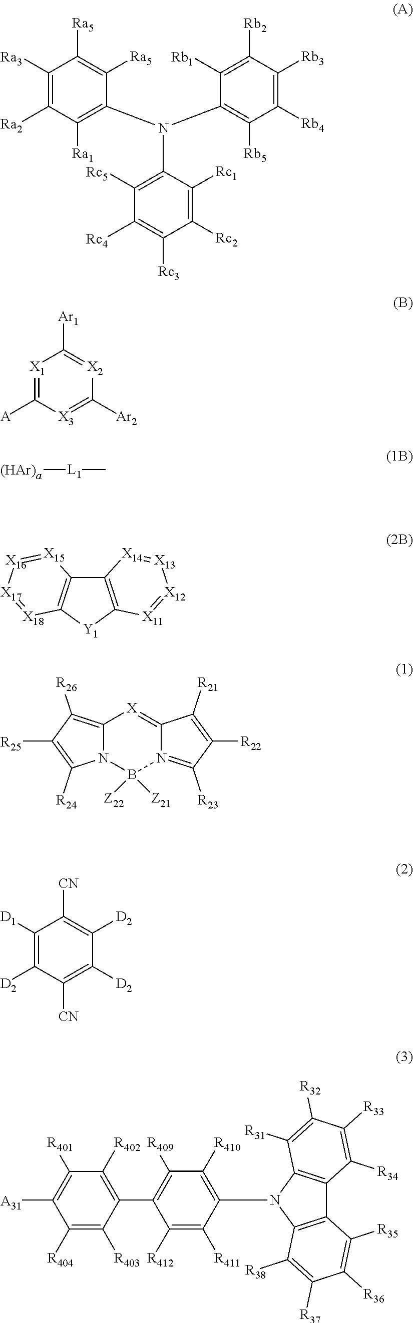

### TECHNICAL FIELD

The present invention relates to an organic electroluminescence device and an electronic device.

### BACKGROUND ART

When a voltage is applied to an organic electroluminescence device (hereinafter, occasionally referred to as an organic EL device), holes are injected from an anode and electrons are injected from a cathode into an emitting layer. The injected electrons and holes are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Accordingly, studies has been made to improve a performance of the organic EL device.

For instance, it is expected to further efficiently emit the organic EL device using triplet excitons in addition to singlet excitons. In view of the above, a highly efficient fluorescent organic EL device using thermally activated delayed fluorescence (hereinafter, sometimes simply referred to as “delayed fluorescence”) has been proposed and studied.

For instance, a TADF (Thermally Activated Delayed Fluorescence) mechanism has been studied. This TADF mechanism uses such a phenomenon in which inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI, Chihaya, published by Kodansha, issued on Apr. 1, 2012, on pages 261-268).

Patent Literatures 1 and 2 each disclose an organic EL device including a hole transporting layer, an emitting layer containing a TADF compound, and an electron transporting layer. The hole transporting layer described in Patent Literatures 1 and 2 contains an amine compound. The electron transporting layer described in Patent Literatures 1 and 2 contains a compound in which a heteroaryl group is bonded directly or with a linking group to an azine ring having an aryl group.

### CITATION LIST

### Patent Literature(s)

Patent Literature 1: International Publication No. WO2019/013063
Patent Literature 2: International Publication No. WO2016/056559

### SUMMARY OF THE INVENTION

### Problems to be Solved by the Invention

Further improvement in performance have been required for an organic EL device using a TADF mechanism.

An object of the invention is to provide a high-performance organic electroluminescence device and an electronic device.

### Means for Solving the Problem(s)

According to an aspect of the invention, an organic electroluminescence device includes:

an anode;

a cathode;

an emitting layer provided between the anode and the cathode;

a first layer provided between the anode and the emitting layer and adjacent to the emitting layer, and

a second layer provided between the cathode and the emitting layer and adjacent to the emitting layer, in which

the emitting layer contains a first compound, a second compound, and a third compound,

the first layer contains a compound represented by a formula (A) below,

the second layer contains a compound represented by a formula (B) below,

the first compound is a fluorescent compound and is represented by a formula (1) below,

the second compound is a delayed fluorescent compound and is represented by a formula (2) below.

the third compound is represented by a formula (3) below, and

a singlet energy S₁(M1) of the first compound, a singlet energy S₁(M2) of the second compound, and a singlet energy S₁(M3) of the third compound satisfy a relationship of a numerical formula (Numerical Formula 1) below.

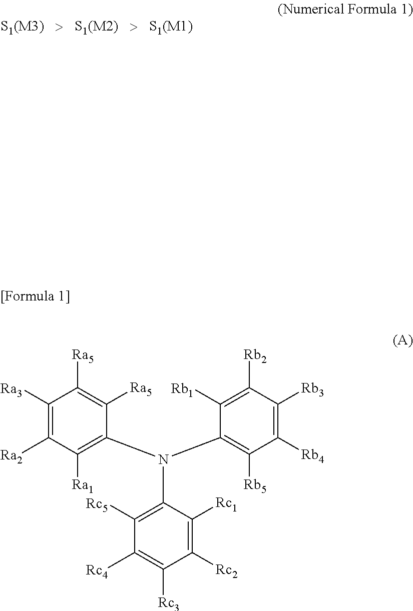

In the formula (A):

Ra₁ to Ra₅, Rb₁ to Rb₅, and Rc₃ to Rc₅ are each independently a hydrogen atom or a substituent; Ra₁ to Ra₅, Rb₁ to Rb₅, and Rc₃ to Rc₅ serving as the substituent are each independently a halogen atom, a cyano group, a substituted or unsubstituted aryl group having 6 to 30 ring carbon atoms, or a substituted or unsubstituted heteroaryl group having 5 to 30 ring atoms;

Rc₁ is a hydrogen atom or a substituent, or a pair of Rc₁ and Rc₂ are mutually bonded to form a ring; Rc₁ serving as the substituent is a substituted or unsubstituted aryl group having 6 to 12 ring carbon atoms;

Rc₂ is a hydrogen atom or a substituent, or a pair of Rc₁ and Rc₂ are mutually bonded to form a ring;

when a pair of Rc₁ and Rc₂ are mutually bonded to form a ring, the ring at least includes a five-membered ring, the five-membered ring including at least one of a carbon atom, an oxygen atom, a sulfur atom, or a nitrogen atom;

Rc₁ and Rc₂ are not hydrogen atoms at the same time; and

Rc₂ serving as the substituent is a substituted or unsubstituted aryl group having 6 to 30 ring carbon atoms, a substituted or unsubstituted heteroaryl group having 5 to 30 ring atoms, a substituted or unsubstituted aryloxy group having 6 to 30 ring carbon atoms, a substituted or unsubstituted arylthio group having 6 to 30 ring carbon atoms, or a substituted or unsubstituted amino group.

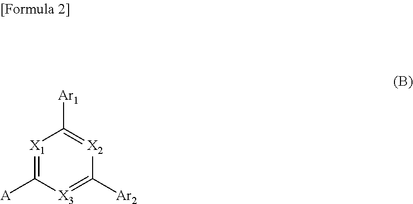

In the formula (B):

X₁ to X₃ are each independently a nitrogen atom or CR₁, at least one of X₁ to X₃ is a nitrogen atom;

R₁ is a hydrogen atom or a substituent;

R₁ serving as the substituent is each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms;

Ar₁ and Ar₂ are each independently represented by a formula (1B) below, or are a substituted or unsubstituted aryl group having 6 to 30 ring carbon atoms, or a substituted or unsubstituted heteroaryl group having 5 to 30 ring atoms; and

A is represented by the formula (1B) below.

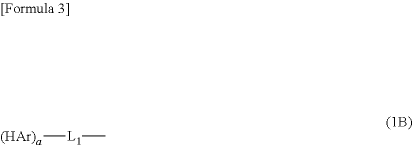

In the formula (1B):

HAr is represented by a formula (2B) below;

a is 1, 2, 3, 4 or 5;

when a is 1, L₁ is a single bond or a divalent linking group;

when a is 2, 3, 4 or 5, L₁ is a trivalent to hexavalent linking group;

a plurality of HAr are mutually the same or different;

the linking group is a group derived from a substituted or unsubstituted aryl group having 6 to 30 ring carbon atoms, a group derived from a substituted or unsubstituted heteroaryl group having 5 to 30 ring atoms, a group derived from a group formed by mutually bonding two groups selected from the group consisting of a substituted or unsubstituted aryl group having 6 to 30 ring carbon atoms and a substituted or unsubstituted heteroaryl group having 5 to 30 ring atoms, or a group derived from a group formed by mutually bonding three groups selected from the group consisting of a substituted or unsubstituted aryl group having 6 to 30 ring carbon atoms and a substituted or unsubstituted heteroaryl group having 5 to 30 ring atoms; and

the mutually bonded groups are mutually the same or different.

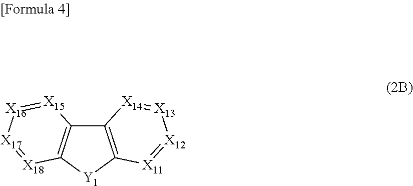

In the formula (2B):

X₁₁ to X₁₈ are each independently a nitrogen atom, CR₁₃, or a carbon atom bonded to L₁;

a plurality of R₁₃ are mutually the same or different;

Y₁ is an oxygen atom, a sulfur atom, NR₁₈, SiR₁₁R₁₂, CR₁₄R₁₅, a nitrogen atom bonded to L₁, a silicon atom bonded to each of R₁₆ and L₁, or a carbon atom bonded to each of R₁₇ and L₁;

among carbon atoms in X₁₁ to X₁₈, R₁₁ to R₁₂, and R₁₄ to R₁₅ as well as a nitrogen atom, a silicon atom, and a carbon atom in Y₁, one atom is bonded to L₁;

R₁₁ and R₁₂ are mutually the same or different, R₁₄ and R₁₅ are mutually the same or different;

R₁₁ to R₁₈ are each independently a hydrogen atom or a substituent, or at least one pair of a pair of adjacent ones of R₁₃, a pair of R₁₁ and R₁₂, or a pair of R₁₄ and R₁₅ are bonded to each other to form a ring; and

R₁₁ to R₁₈ serving as the substituent are each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms.

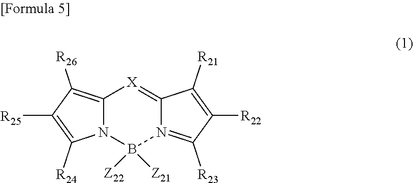

In the formula (1): X is a nitrogen atom, or a carbon atom bonded to Y;

Y is a hydrogen atom or a substituent;

R₂₁ to R₂₆ are each independently a hydrogen atom or a substituent, or at least one of a pair of R₂₁ and R₂₂, a pair of R₂₂ and R₂₃, a pair of R₂₄ and R₂₅, or a pair of R₂₅ and R₂₆ are mutually bonded to form a ring;

Y and R₂₁ to R₂₆ serving as the substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;

Z₂₁ and Z₂₂ are each independently a substituent, or are mutually bonded to form a ring; and

Z₂₁ and Z₂₂ serving as the substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

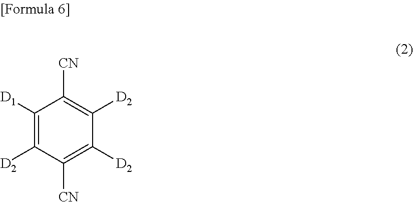

In the formula (2), D₁ is a group represented by a formula (2-1) below, D₂ is a group represented by a formula (2-2), and a plurality of D₂ are mutually the same group.

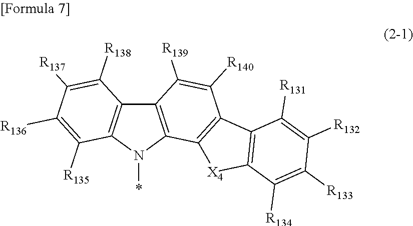

In the formula (2-1):

X₄ is an oxygen atom or a sulfur atom, and R₁₃₁ to R₁₄₀ are each independently a hydrogen atom or a substituent;

R₁₃₁ to R₁₄₀ serving as the substituent are each independently a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkylsilyl group having 3 to 6 carbon atoms, a substituted or unsubstituted alkoxy group having 1 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and

* represents a bonding position to a benzene ring in the formula (2).

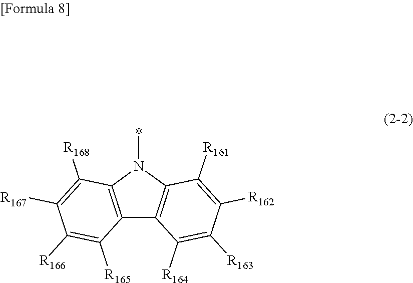

In the formula (2-2):

R₁₆₁ to R₁₆₈ are each independently a hydrogen atom or a substituent;

R₁₆₁ to R₁₆₈ serving as the substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted alkylsilyl group having 3 to 6 carbon atoms, a substituted or unsubstituted alkoxy group having 1 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and

* each independently represents a bonding position to a benzene ring in the formula (2).

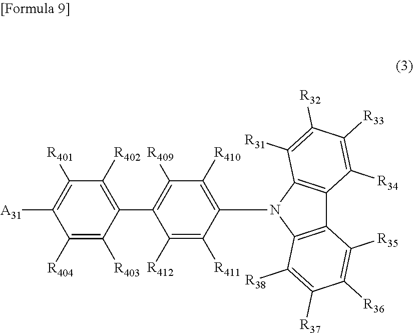

In the formula (3): A₃₁ is a group represented by a formula (31a), formula (31b), formula (31c), formula (31d), formula (31e) or formula (31f);

R₃₁ to R₃₈ are each independently a hydrogen atom or a substituent;

R₄₀₁ to R₄₀₄ and R₄₀₉ to R₄₁₂ are each independently a hydrogen atom or a substituent; and

R₃₁ to R₃₈ serving as the substituent and R₄₀₁ to R₄₀₄ and R₄₀₉ to R₄₁₂ serving as the substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms.

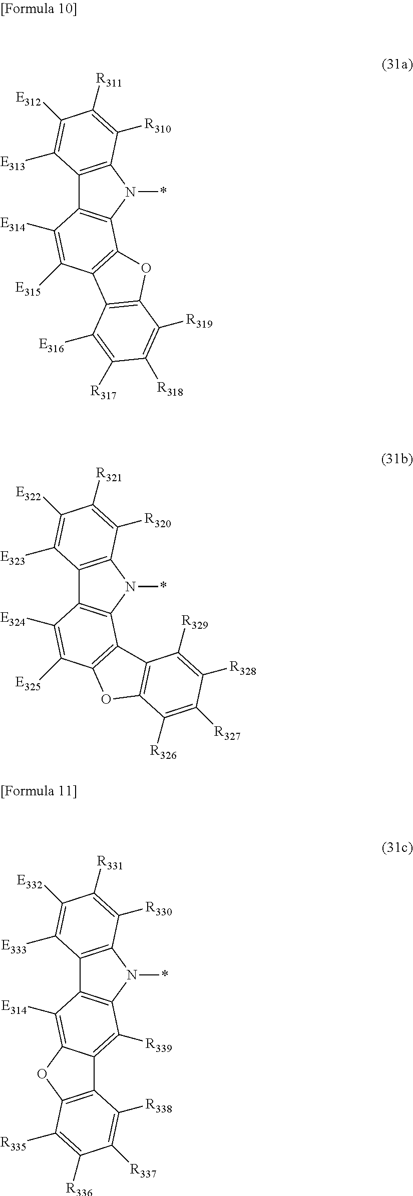

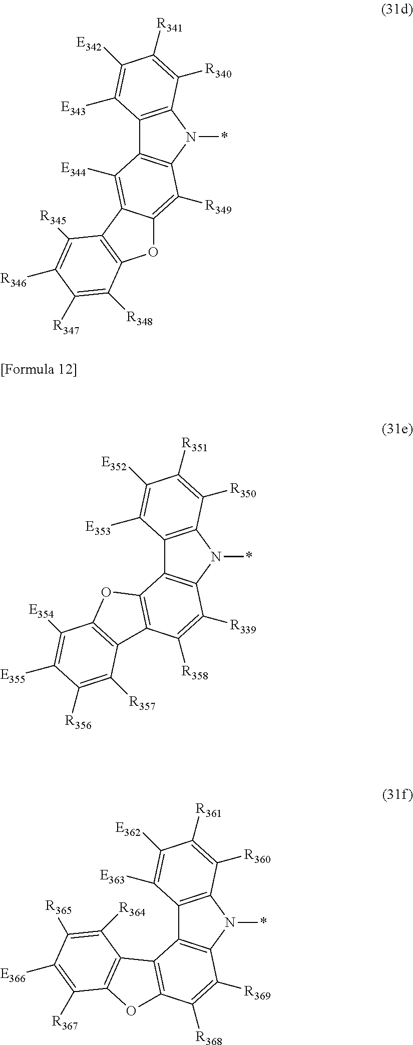

In the formula (31a), formula (31b), formula (31c), formula (31d), formula (31e) and formula (31f):

R₃₁₀ to R₃₁₉ are each independently a hydrogen atom or a substituent;

Ra₃₂₀ to R₃₂₉ are each independently a hydrogen atom or a substituent;

R₃₃₀ to R₃₃₉ are each independently a hydrogen atom or a substituent;

R₃₄₀ to R₃₄₉ are each independently a hydrogen atom or a substituent;

R₃₅₀ to R₃₅₉ are each independently a hydrogen atom or a substituent;

R₃₆₀ to R₃₆₉ are each independently a hydrogen atom or a substituent;

R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ serving as the substituent each independently represent the same as R₃₁ to R₃₈ serving as the substituent and R₄₀₁ to R₄₀₄ and R₄₀₉ to R₄₁₂ serving as the substituent in the formula (3); and

* each independently represents a bonding position to a benzene ring having R₄₀₁ to R₄₀₄ in the formula (3).

According to another aspect of the invention, an electronic device including the organic electroluminescence device according to the above aspect of the invention is provided.

According to the above aspects of the invention, a high-performance organic electroluminescence device and electronic device can be provided.

### BRIEF EXPLANATION OF DRAWINGS

FIG. 1 schematically shows an exemplary arrangement of an organic EL device according to a first exemplary embodiment.

FIG. 2 schematically shows a device that measures transient PL.

FIG. 3 shows an example of a decay curve of the transient PL.

FIG. 4 shows a relationship in energy level and energy transfer between a first compound, a second compound, and a third compound in an emitting layer of an exemplary organic EL device according to the first exemplary embodiment.

### DESCRIPTION OF EMBODIMENT(S)

### First Exemplary Embodiment

Arrangement(s) of an organic EL device according to a first exemplary embodiment of the invention will be described below.

The organic EL device includes an anode, a cathode, and an organic layer between the anode and the cathode. This organic layer includes a plurality of layers formed of an organic compound(s). The organic layer may further contain an inorganic compound. At least one of the layers forming the organic layer is an emitting layer.

In the first exemplary embodiment, the organic layer includes the emitting layer provided between the anode and the cathode, a first layer provided between the anode and the emitting layer and adjacent to the emitting layer, and a second layer provided between the cathode and the emitting layer and adjacent to the emitting layer.

The emitting layer contains a first compound represented by a formula (1), a second compound represented by a formula (2), and a third compound represented by a formula (3). The first compound is a fluorescent compound and the second compound is a delayed fluorescent compound.

The first layer contains a compound represented by a formula (A). The first layer is not particularly limited, but is at least one layer selected from the group consisting of a hole injecting layer, hole transporting layer, and electron blocking layer. The first layer is preferably the electron blocking layer.

The second layer contains a compound represented by a formula (B). The second layer is not particularly limited, but is at least one layer selected from the group consisting of an electron injecting layer, electron transporting layer, and hole blocking layer. The second layer is preferably the hole blocking layer.

Specifically, the organic layer of the organic EL device in the exemplary embodiment preferably has a layer arrangement below.
electron blocking layer/emitting layer/hole blocking layer
hole injecting layer/electron blocking layer/emitting layer/hole blocking layer
hole transporting layer/electron blocking layer/emitting layer/hole blocking layer
hole injecting layer/hole transporting layer/electron blocking layer/emitting layer/hole blocking layer
electron blocking layer/emitting layer/hole blocking layer/electron injecting layer
electron blocking layer/emitting layer/hole blocking layer/electron transporting layer
electron blocking layer/emitting layer/hole blocking layer/electron transporting layer/electron injecting layer
hole injecting layer/electron blocking layer/emitting layer/hole blocking layer/electron injecting layer
hole injecting layer/electron blocking layer/emitting layer/hole blocking layer/electron transporting layer
hole injecting layer/electron blocking layer/emitting layer/hole blocking layer/electron transporting layer/electron injecting layer
hole transporting layer/electron blocking layer/emitting layer/hole blocking layer/electron injecting layer
hole transporting layer/electron blocking layer/emitting layer/hole blocking layer/electron transporting layer
hole transporting layer/electron blocking layer/emitting layer/hole blocking layer/electron transporting layer/electron injecting layer
hole injecting layer/hole transporting layer/electron blocking layer/emitting layer/hole blocking layer/electron injecting layer
hole injecting layer/hole transporting layer/electron blocking layer/emitting layer/hole blocking layer/electron transporting layer
hole injecting layer/hole transporting layer/electron blocking layer/emitting layer/hole blocking layer/electron transporting layer/electron injecting layer

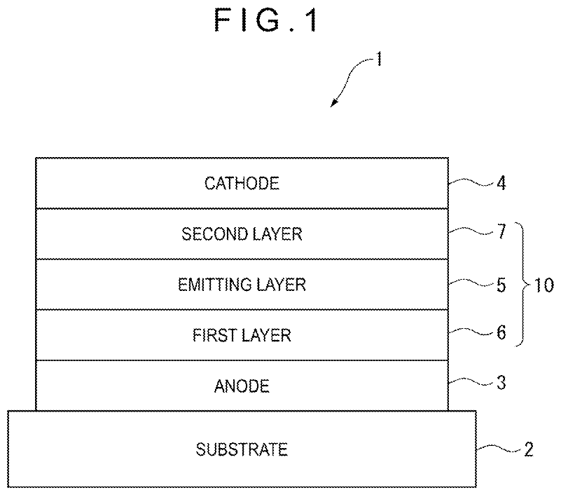

FIG. 1 schematically shows an exemplary arrangement of the organic EL device of the exemplary embodiment.

An organic EL device 1 includes a light-transmissive substrate 2, an anode 3, a cathode 4, and an organic layer 10 provided between the anode 3 and the cathode 4. The organic layer 10 includes a first layer 6, an emitting layer 5, and a second layer 7, which are sequentially laminated on the anode 3. The first layer 6 is adjacent to a side of the emitting layer 5 close to the anode 3. The second layer 7 is adjacent to a side of the emitting layer 5 close to the cathode 4.

The emitting layer 5 may contain a metal complex.

It is preferable that the emitting layer 5 does not contain a phosphorescent material (dopant material).

It is preferable that the emitting layer 5 does not contain a heavy-metal complex and a phosphorescent rare-earth metal complex. Examples of the heavy-metal complex herein include iridium complex, osmium complex, and platinum complex.

It is also preferable that the emitting layer 5 does not contain a metal complex.

The first compound is preferably a dopant material (occasionally referred to as a guest material, emitter or luminescent material).

The second compound is preferably a host material (occasionally referred to as a matrix material).

It is preferable that the third compound is a host material. Occasionally, one of the second compound and the third compound is referred to as a first host material and the other thereof is referred to as a second host material. The third compound may be a delayed fluorescent compound and a compound that does not exhibit delayed fluorescence.

A typical organic EL device including the emitting layer containing three compounds of a fluorescent compound, a TADF compound, and the third compound has been known. In order to realize a high-performance organic EL device configured to emit light at a lower voltage or higher efficiency or having a longer lifetime than a typical organic EL device, it is necessary to improve hole injectability into the emitting layer. In addition, it is also necessary to trap holes, which have been injected into the emitting layer, in the emitting layer for a longer time and generate excitons efficiently. However, a known combination of the emitting layer and neighboring layers (e.g., an electron blocking layer and a hole blocking layer) makes it insufficient to improve hole injectability into the emitting layer and generate excitons efficiently in the emitting layer.

The inventors have found a high-performance organic EL device achievable by providing the first layer containing the compound represented by the formula (A) adjacent to a side of the emitting layer close to the anode, providing the second layer containing the compound represented by the formula (B) adjacent to a side of the emitting layer close to the cathode, and further containing the fluorescent first compound (compound represented by the formula (1)), the delayed fluorescent second compound (compound represented by the formula (2)), and the third compound (compound represented by the formula (3)) in the emitting layer.

An arrangement of the organic EL device according to the first exemplary embodiment will be described below.

### First Layer

A first layer 6 contains a compound represented by the formula (A) below.

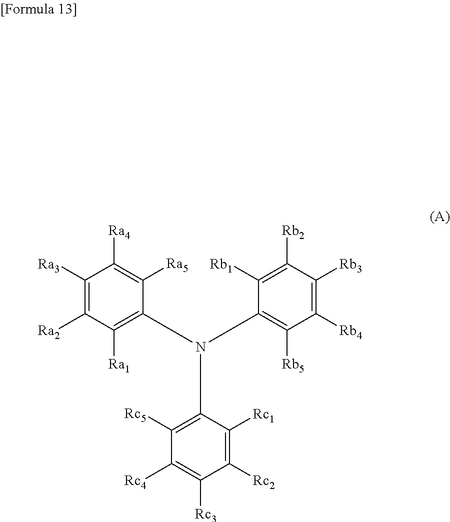

In the formula (A):

Ra₁ to Ra₅, Rb₁ to Rb₅, and Rc₃ to Rc₅ are each independently a hydrogen atom or a substituent. Ra₁ to Ra₅, Rb₁ to Rb₅, and Rc₃ to Rc₅ serving as the substituent are each independently a halogen atom, a cyano group, a substituted or unsubstituted aryl group having 6 to 30 ring carbon atoms, or a substituted or unsubstituted heteroaryl group having 5 to 30 ring atoms;

Rc₁ is a hydrogen atom or a substituent, or is bonded to Rc₂ to form a ring, and Rc₁ serving as the substituent is a substituted or unsubstituted aryl group having 6 to 12 ring carbon atoms;

Rc₂ is a hydrogen atom or a substituent, or a pair of Rc₁ and Rc₂ are mutually bonded to form a ring;

when a pair of Rc₁ and Rc₂ are mutually bonded to form a ring, the ring at least comprises a five-membered ring, the five-membered ring comprising at least one of a carbon atom, an oxygen atom, a sulfur atom, or a nitrogen atom;

Rc₁ and Rc₂ are not hydrogen atoms at the same time; and

Rc₂ serving as the substituent is a substituted or unsubstituted aryl group having 6 to 30 ring carbon atoms, a substituted or unsubstituted heteroaryl group having 5 to 30 ring atoms, a substituted or unsubstituted aryloxy group having 6 to 30 ring carbon atoms, a substituted or unsubstituted arylthio group having 6 to 30 ring carbon atoms, or a substituted or unsubstituted amino group.

In the formula (A), it is preferable that none of Rb₁ to Rb₅ and Rc₂ to Rc₅ is an unsubstituted dibenzofuranyl group when at least one of Ra₁ to Ra₅ is an unsubstituted dibenzofuranyl group, none of Ra₁ to Ra₅ and Rc₂ to Rc₅ is an unsubstituted dibenzofuranyl group when at least one of Rb₁ to Rb₅ is an unsubstituted dibenzofuranyl group, and none of Ra₁ to Ra₅ and Rb₁ to Rb₅ is an unsubstituted dibenzofuranyl group when at least one of Rc₂ to Rc₅ is an unsubstituted dibenzofuranyl group.

In the formula (A), it is more preferable that none of Rb₁ to Rb₅ and Rc₂ to Rc₅ is a substituted or unsubstituted dibenzofuranyl group when at least one of Ra₁ to Ra₅ is a substituted or unsubstituted dibenzofuranyl group, none of Ra₁ to Ra₅ and Rc₂ to Rc₅ is a substituted or unsubstituted dibenzofuranyl group when at least one of Rb₁ to Rb₅ is a substituted or unsubstituted dibenzofuranyl group, and none of Ra₁ to Ra₅ and Rb₁ to Rb₅ is a substituted or unsubstituted dibenzofuranyl group when at least one of Rc₂ to Rc₅ is a substituted or unsubstituted dibenzofuranyl group.

In the formula (A), it is preferable that a pair of Rc₁ and Rc₂ are bonded to each other to form a ring.

In the formula (A), it is also preferable that Rc₁ is a hydrogen atom or a substituent and Rc₂ is a hydrogen atom or a substituent. It should be noted that Rc₁ and Rc₂ are not hydrogen atoms at the same time.

Here, significance that a pair of Rc₁ and Rc₂ are bonded to each other to form a ring and that at least one of Rc₁ or Rc₂ is a specific substituent will be described using a formula (1A) below.

The formula (1A) is a partial structure of the compound represented by the formula (A).

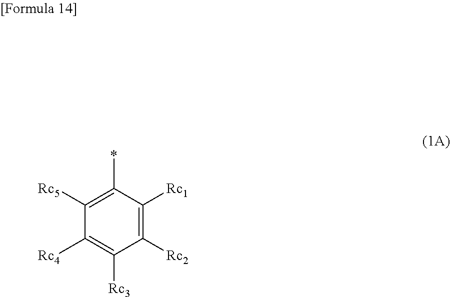

In the formula (1A), Rc₁ represents the same as Rc₁ in the formula (A), Rc₂ represents the same as Rc₂ in the formula (A), Rc₃ to Rc₅ each independently represent the same as Rc₃ to Rc₅ in the formula (A), and * represents a bonding position to a nitrogen atom in the compound represented by the formula (A).

In the formula (1A), that a pair of Rc₁ and Rc₂ are bonded to each other to form a ring means that Rc₁ and Rc₂ form, for instance, a ring Z_{11A} represented by a formula (11A) below.

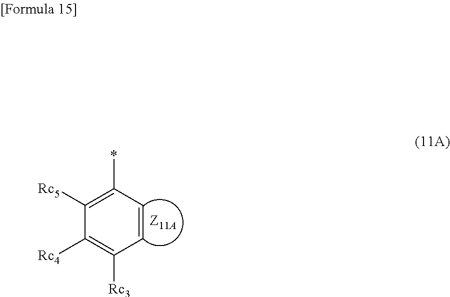

In the formula (1A), when Rc₂ and Rc₃ form a ring Z_{11B} represented by a formula (11B) below and when Rc₃ and Rc₄ form a ring Z_{11C} represented by a formula (11C) below, the formulae (11B) and (11C) do not satisfy the formula (1A).

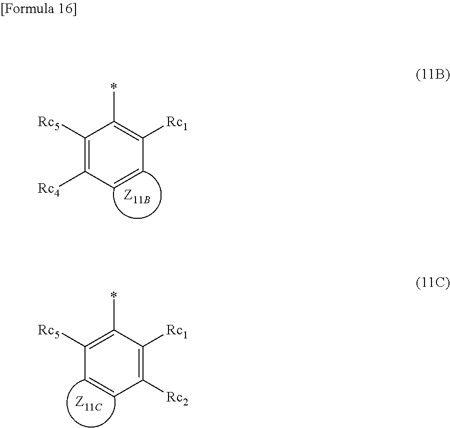

In the compound represented by the formula (A), Rc₁ and Rc₂ located close to a nitrogen atom are bonded to each other to form the ring Z_{11A}, or at least one of Rc₁ or Rc₂ has a specific substituent. Accordingly, the compound represented by the formula (A) has a bulkier structure around the nitrogen atom than those of, for instance, a compound having the ring Z_{11B} formed by bonding Rc₂ and Rc₃ to each other, a compound having the ring Z_{11C} formed by bonding Rc₃ and Rc₄ to each other, and a compound with substituted Rc₃. Consequently, it is considered that the compound represented by the formula (A) has a narrow HOMO (highest occupied molecular orbital) and a deep ionization potential Ip (large absolute value).

It is thus considered that the organic EL device 1 according to the exemplary embodiment improves hole injectability into the emitting layer 5 and an efficiency of generating excitons in the emitting layer since the first layer adjacent to the side of the emitting layer 5 close to the anode 3 contains the compound represented by the formula (A), resulting in improving performance of the organic EL device.

In the formula (A), a moiety represented by the formula (1A) is preferably a group represented by one of formulae (1A-1) to (1A-10).

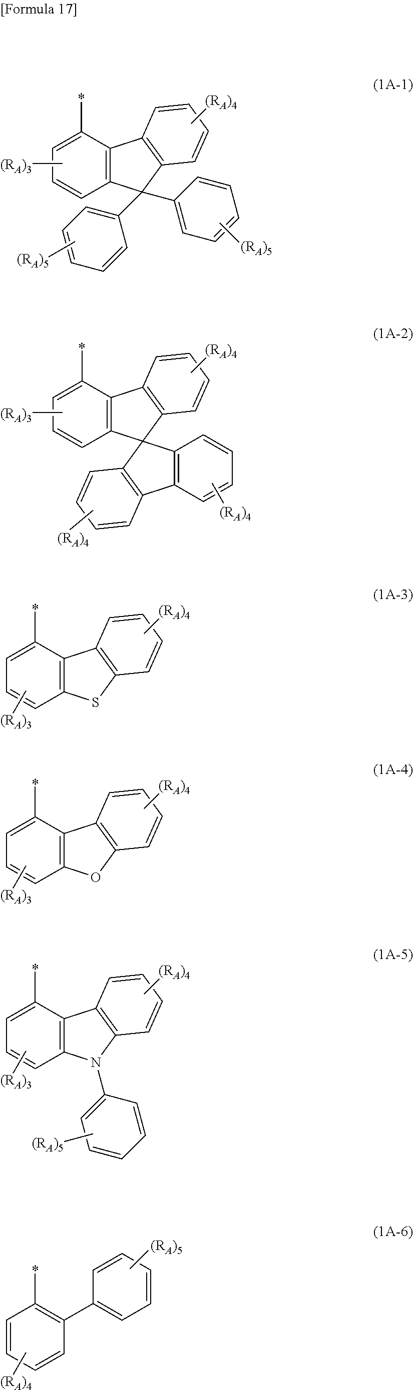

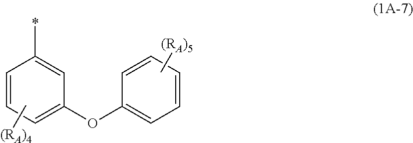

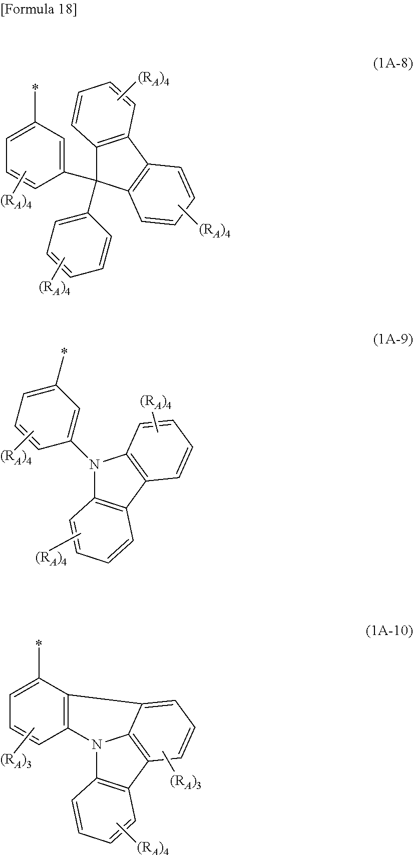

In the formulae (1A-1) to (1A-10): R_{A} is a hydrogen atom or a substituent;

R_{A} as the substituent is each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms;

when a plurality of R_{A} are present, the plurality of R_{A} are mutually the same or different; and

* represents a bonding position to a nitrogen atom in the compound represented by the formula (A).

The group represented by the formula (1A) is preferably the group represented by one of the formulae (1A-1) to (1A-5) and (1A-10), more preferably the group represented by the formula (1A-1) or (1A-4).

The group represented by the formula (1A) is also preferably the group represented by one of the formulae (1A-6) to (1A-9), more preferably the group represented by the formula (1A-9).

The group represented by the formula (1A) is more preferably the group represented by the formula (1A-1), (1A-4) or (1A-9).

In the formulae (1A-1) to (1A-10), R_{A} is preferably a hydrogen atom.

In the formula (A), Ra₁ to Ra₅ and Rb₁ to Rb₅ are preferably each independently a hydrogen atom or a substituted or unsubstituted aryl group having 6 to 30 ring carbon atoms.

In the formula (A), it is also preferable that Ra₁ to Ra₅ are each independently a hydrogen atom or a substituted or unsubstituted aryl group having 6 to 30 ring carbon atoms and Rb₁ to Rb₅ are each independently a hydrogen atom or a substituted or unsubstituted heteroaryl group having 5 to 30 ring atoms.

In the formula (A), it is also preferable that Ra₁ to Ra₅ are each independently a hydrogen atom or an aryl group having 6 to 30 ring carbon atoms and substituted by a heteroaryl group having 5 to 30 ring atoms and Rb₁ to Rb₅ are each independently a hydrogen atom or a substituted or unsubstituted heteroaryl group having 5 to 30 ring atoms.

In the formula (A), it is also preferable that Ra₁ to Ra₅ and Rb₁ to Rb₅ are each independently a hydrogen atom or a substituted or unsubstituted heteroaryl group having 5 to 30 ring atoms.

In the formula (A), it is preferable that one of Ra₁ to Ra₅ is a substituent and Ra₁ to Ra₅ that are not the substituent are hydrogen atoms, one of Rb₁ to Rb₅ is a substituent and Rb₁ to Rb₅ that are not the substituent are hydrogen atoms, and Rc₃ to Rc₅ are hydrogen atoms.

In the formula (A), Ra₁ to Ra₅, Rb₁ to Rb₅ and Rc₃ to Rc₅ serving as the substituent are preferably each independently a halogen atom, cyano group, unsubstituted aryl group having 6 to 30 ring carbon atoms, or unsubstituted heteroaryl group having 5 to 30 ring atoms.

In the formula (A), it is preferable that one of Ra₁ to Ra₅ is a substituent and Ra₁ to Ra₅ that are not the substituent are hydrogen atoms, one of Rb₁ to Rb₅ is a substituent and Rb₁ to Rb₅ that are not the substituent are hydrogen atoms, Rc₃ to Rc₅ are hydrogen atoms, and Ra₁ to Ra₅ and Rb₁ to Rb₅ serving as the substituent are preferably each independently a halogen atom, cyano group, unsubstituted aryl group having 6 to 30 ring carbon atoms, or unsubstituted heteroaryl group having 5 to 30 ring atoms.

In the formula (A), it is preferable that at least one of Ra₁ to Ra₅ is each independently a group represented by one of formulae (1B-1) to (1B-10) and at least one of Rb₁ to Rb₅ is each independently a group represented by one of the formulae (1B-1) to (1B-10).

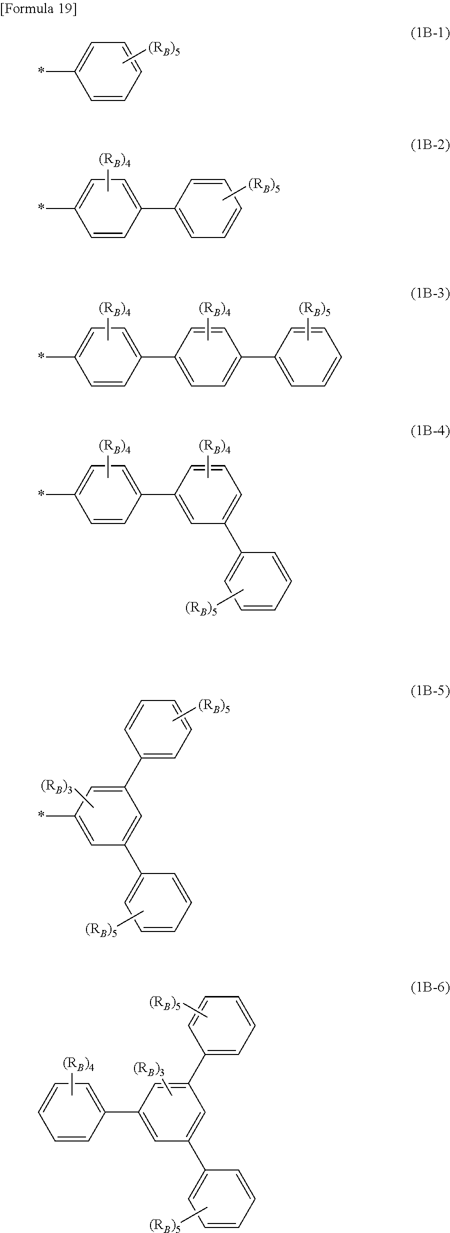

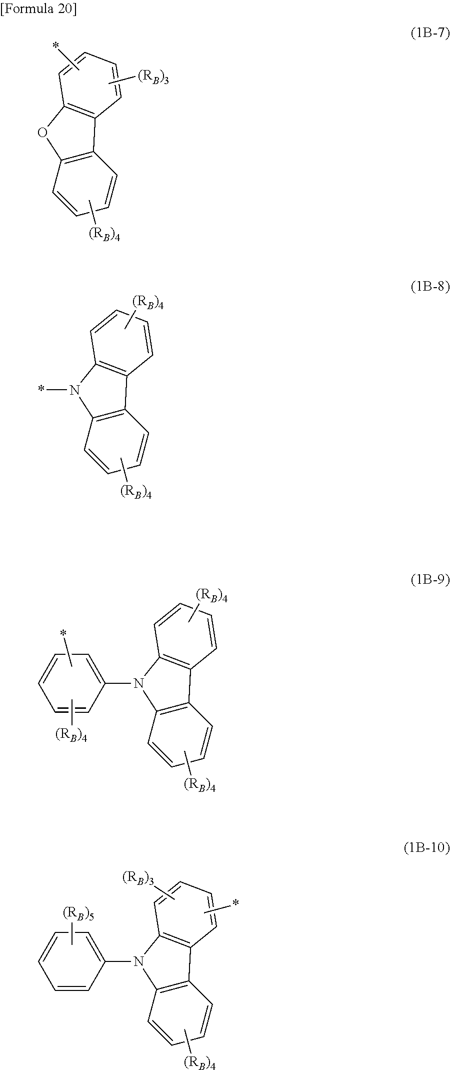

In the formulae (18-1) to (18-10): R_{B} is a hydrogen atom or a substituent;

R_{B} as the substituent is each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms;

when a plurality of R_{B} are present, the plurality of R_{B} are mutually the same or different; and * represents a bonding position to each of a benzene ring to which Ra₁ to Ra₅ are bonded and a benzene ring to which Rb₁ to Rb₅ are bonded in the compound represented by the formula (A).

In the formulae (18-1) to (1B-10), R_{B} is preferably a hydrogen atom.

The compound represented by the formula (A) is preferably a compound represented by a formula (1X), a formula (1Y) or a formula (1Z) below, more preferably the compound represented by the formula (1X).

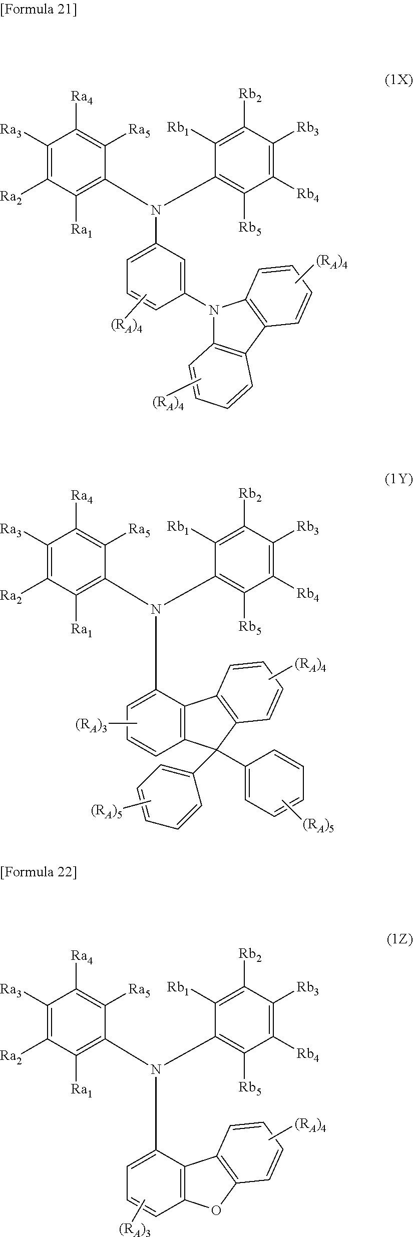

In the formula (1X), the formula (1Y) and the formula (1Z), Ra₁ to Ra₅ and Rb₁ to Rb₅ each represent the same as Ra₁ to Ra₅ and Rb₁ to Rb₅ in the formula (A) and R_{A} represents the same as R_{A} in the formulae (1A-1) to (1A-10).

In the formula (1X), the formula (1Y) and the formula (1Z), it is preferable that at least one of Ra₁ to Ra₅ is each independently a group represented by one of formulae (1B-1) to (11-10) and at least one of Rb₁ to Rb₅ is each independently a group represented by one of the formulae (18-1) to (16-10).

In the formula (1X), the formula (1Y) and the formula (1Z), it is more preferable that one of Ra₁ to Ra₅ is a group represented by one of formulae (1B-1) to (1B-10) and one of Rb₁ to Rb₅ is a group represented by one of the formulae (18-1) to (1B-10).

In the formulae (1X), (1Y) and (1Z), R_{A} is preferably a hydrogen atom.

In the formulae (18-1) to (1B-10), R_{B} is preferably a hydrogen atom.

An ionization potential Ip of the compound represented by the formula (A) is preferably 5.78 eV or more, more preferably 5.80 eV or more, further preferably 5.85 eV or more in order to improve hole injectability into the emitting layer and generate excitons efficiently in the emitting layer.

A measurement method of the ionization potential Ip of the compound represented by the formula (A) is the same as described in Examples described later.

### Manufacturing Method of Compound Represented by Formula (A)

The compound represented by the formula (A) can be manufactured by a publicly known method.

Specific examples of the compound represented by the formula (A) are shown below. It should be noted that the compound represented by the formula (A) is not limited to the specific examples.

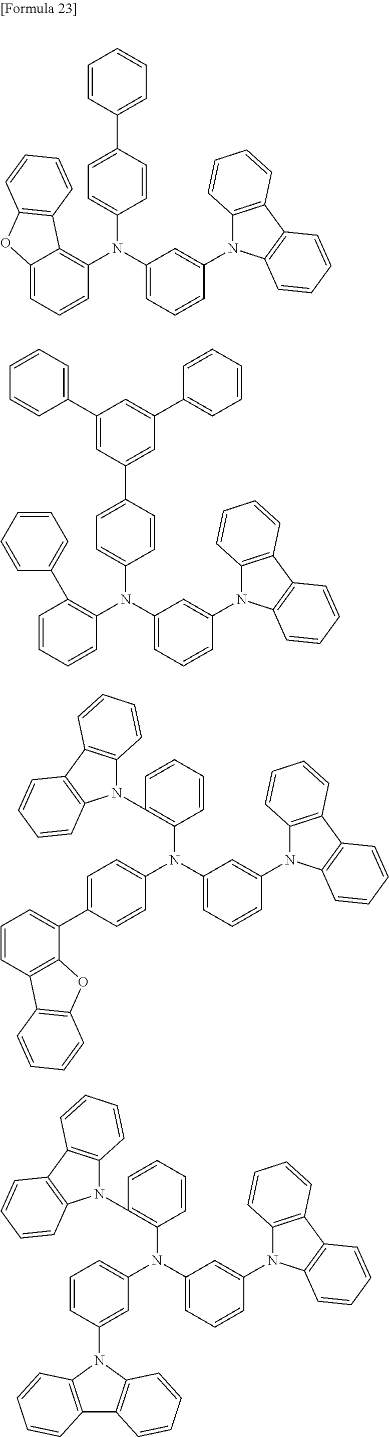

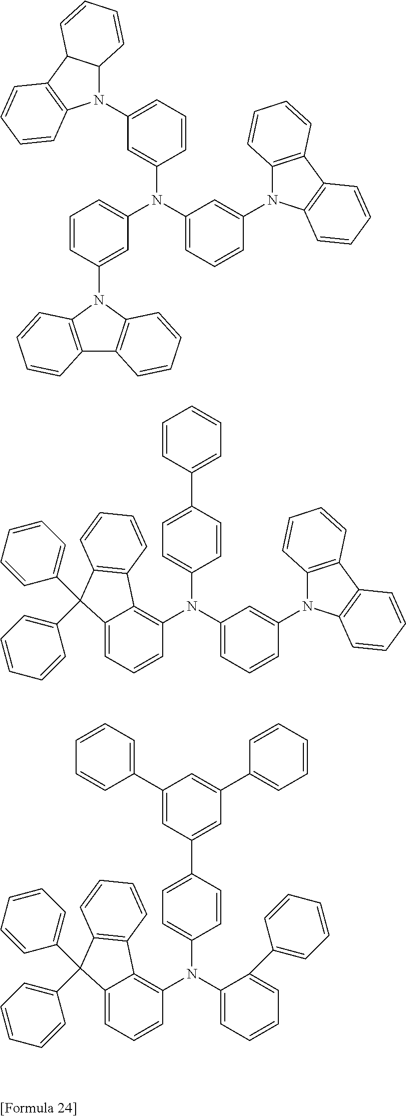

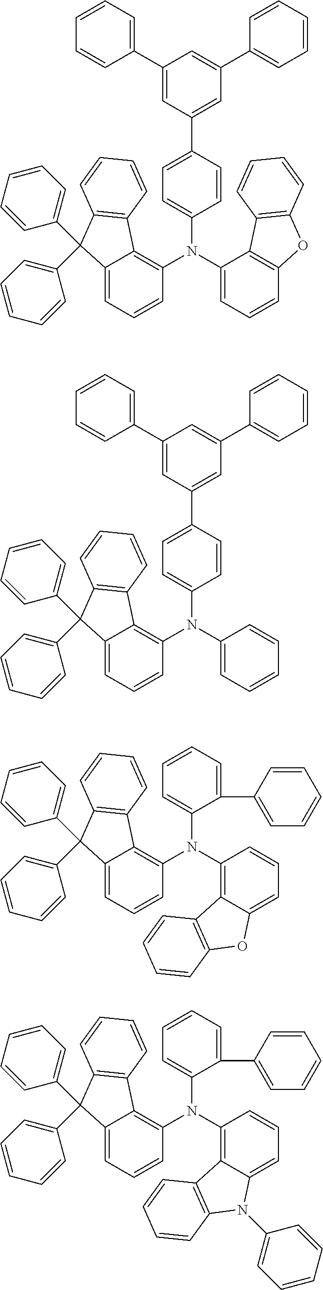

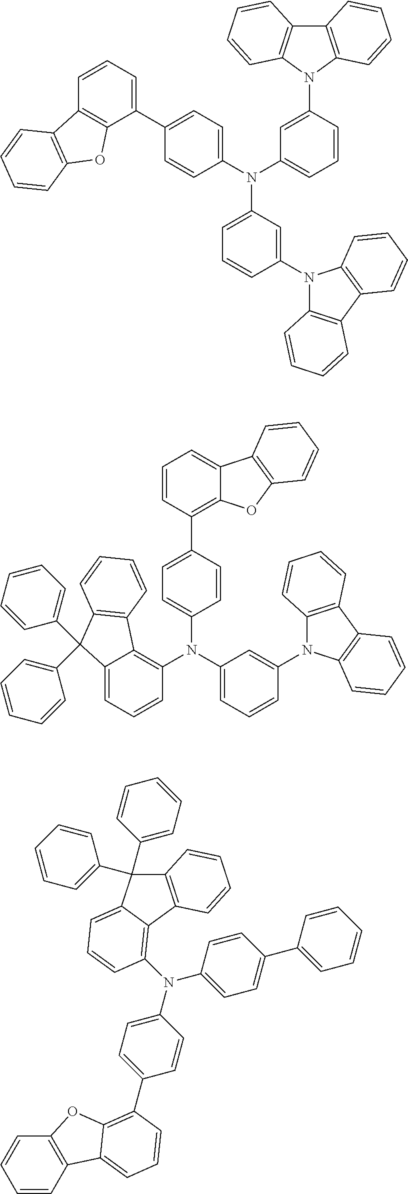 [SMILES: c1cc(N(c2ccc(-c3cccc4c3oc3ccccc34)cc2)c2cccc(-n3c4ccccc4c4ccccc43)c2)cc(-n2c3ccccc3c3ccccc32)c1 ; c1ccc(C2(c3ccccc3)c3ccccc3-c3c(N(c4ccc(-c5cccc6c5oc5ccccc56)cc4)c4cccc(-n5c6ccccc6c6ccccc65)c4)cccc32)cc1 ; c1ccc(-c2ccc(N(c3ccc(-c4cccc5c4oc4ccccc45)cc3)c3cccc4c3-c3ccccc3C4(c3ccccc3)c3ccccc3)cc2)cc1]

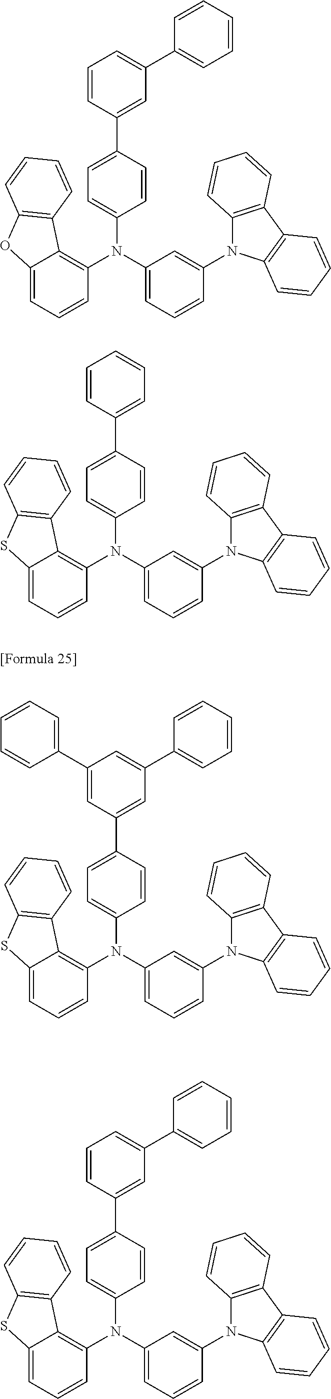 [SMILES: c1ccc(-c2cccc(-c3ccc(N(c4cccc(-n5c6ccccc6c6ccccc65)c4)c4cccc5oc6ccccc6c45)cc3)c2)cc1 ; c1ccc(-c2ccc(N(c3cccc(-n4c5ccccc5c5ccccc54)c3)c3cccc4sc5ccccc5c34)cc2)cc1 ; c1ccc(-c2cc(-c3ccccc3)cc(-c3ccc(N(c4cccc(-n5c6ccccc6c6ccccc65)c4)c4cccc5sc6ccccc6c45)cc3)c2)cc1 ; c1ccc(-c2cccc(-c3ccc(N(c4cccc(-n5c6ccccc6c6ccccc65)c4)c4cccc5sc6ccccc6c45)cc3)c2)cc1]

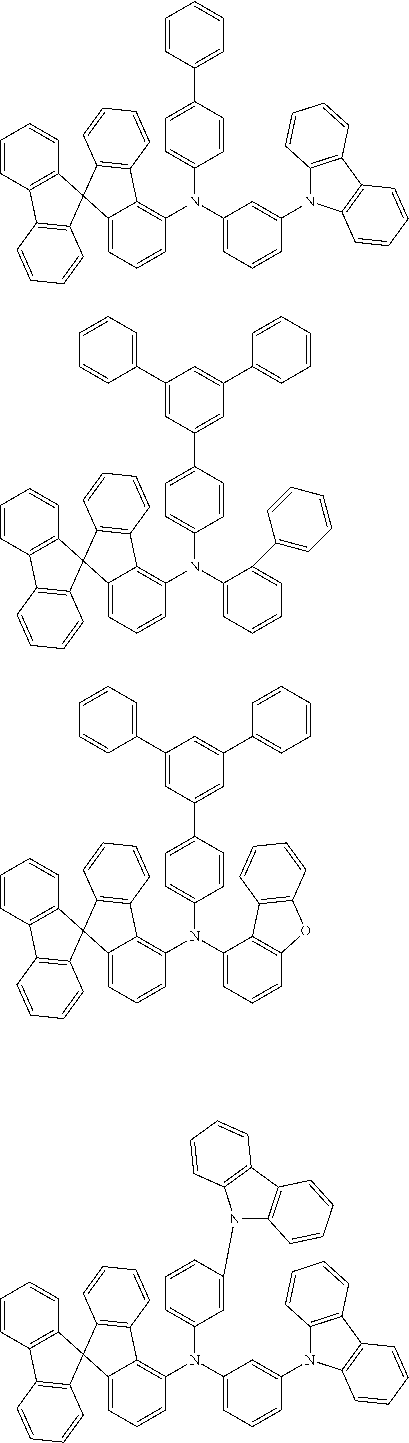 [SMILES: c1ccc(-c2ccc(N(c3cccc(-n4c5ccccc5c5ccccc54)c3)c3cccc4c3-c3ccccc3C43c4ccccc4-c4ccccc43)cc2)cc1 ; c1ccc(-c2cc(-c3ccccc3)cc(-c3ccc(N(c4ccccc4-c4ccccc4)c4cccc5c4-c4ccccc4C54c5ccccc5-c5ccccc54)cc3)c2)cc1 ; c1ccc(-c2cc(-c3ccccc3)cc(-c3ccc(N(c4cccc5c4-c4ccccc4C54c5ccccc5-c5ccccc54)c4cccc5oc6ccccc6c45)cc3)c2)cc1 ; c1cc(N(c2cccc(-n3c4ccccc4c4ccccc43)c2)c2cccc3c2-c2ccccc2C32c3ccccc3-c3ccccc32)cc(-n2c3ccccc3c3ccccc32)c1]

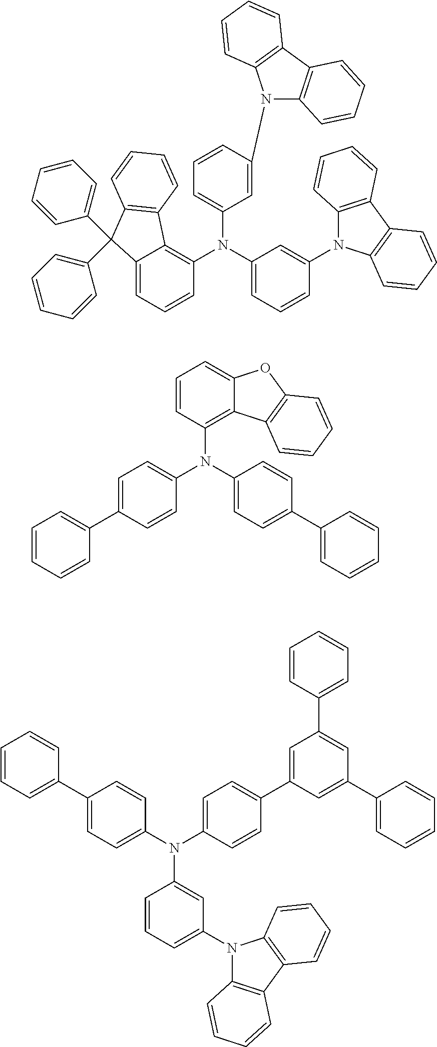 [SMILES: c1ccc(C2(c3ccccc3)c3ccccc3-c3c(N(c4cccc(-n5c6ccccc6c6ccccc65)c4)c4cccc(-n5c6ccccc6c6ccccc65)c4)cccc32)cc1 ; c1ccc(-c2ccc(N(c3ccc(-c4ccccc4)cc3)c3cccc4oc5ccccc5c34)cc2)cc1 ; c1ccc(-c2ccc(N(c3ccc(-c4cc(-c5ccccc5)cc(-c5ccccc5)c4)cc3)c3cccc(-n4c5ccccc5c5ccccc54)c3)cc2)cc1]

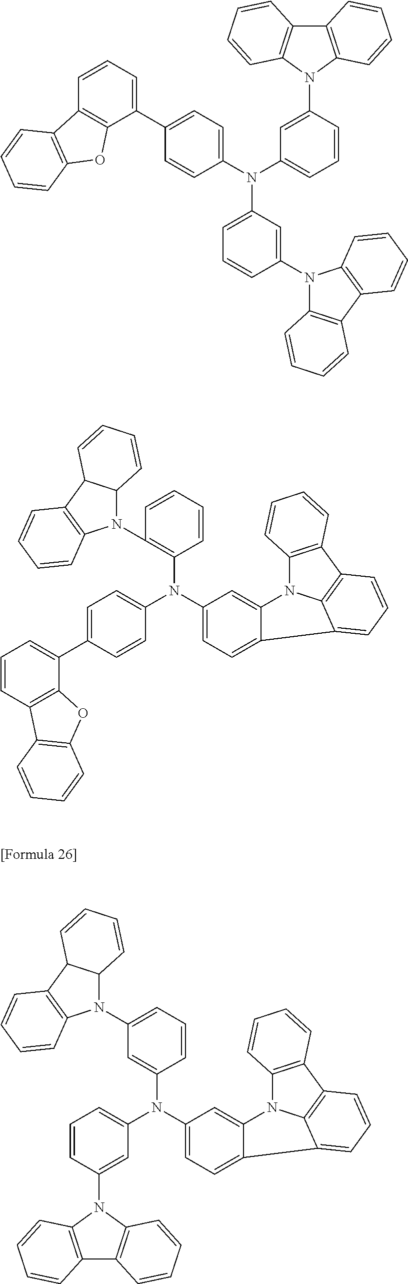 [SMILES: c1cc(N(c2ccc(-c3cccc4c3oc3ccccc34)cc2)c2cccc(-n3c4ccccc4c4ccccc43)c2)cc(-n2c3ccccc3c3ccccc32)c1 ; C1=CC2c3ccccc3N(c3ccccc3N(c3ccc(-c4cccc5c4oc4ccccc45)cc3)c3ccc4c5cccc6c7ccccc7n(c4c3)c65)C2C=C1 ; C1=CC2c3ccccc3N(c3cccc(N(c4cccc(-n5c6ccccc6c6ccccc65)c4)c4ccc5c6cccc7c8ccccc8n(c5c4)c76)c3)C2C=C1]

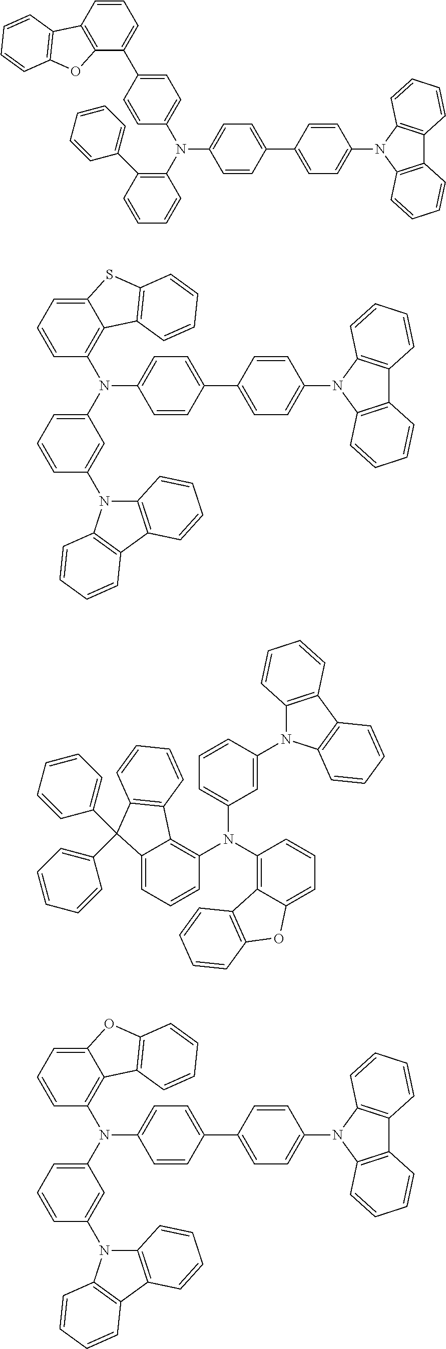 [SMILES: c1ccc(-c2ccccc2N(c2ccc(-c3ccc(-n4c5ccccc5c5ccccc54)cc3)cc2)c2ccc(-c3cccc4c3oc3ccccc34)cc2)cc1 ; c1cc(N(c2ccc(-c3ccc(-n4c5ccccc5c5ccccc54)cc3)cc2)c2cccc3sc4ccccc4c23)cc(-n2c3ccccc3c3ccccc32)c1 ; c1ccc(C2(c3ccccc3)c3ccccc3-c3c(N(c4cccc(-n5c6ccccc6c6ccccc65)c4)c4cccc5oc6ccccc6c45)cccc32)cc1 ; c1cc(N(c2ccc(-c3ccc(-n4c5ccccc5c5ccccc54)cc3)cc2)c2cccc3oc4ccccc4c23)cc(-n2c3ccccc3c3ccccc32)c1]

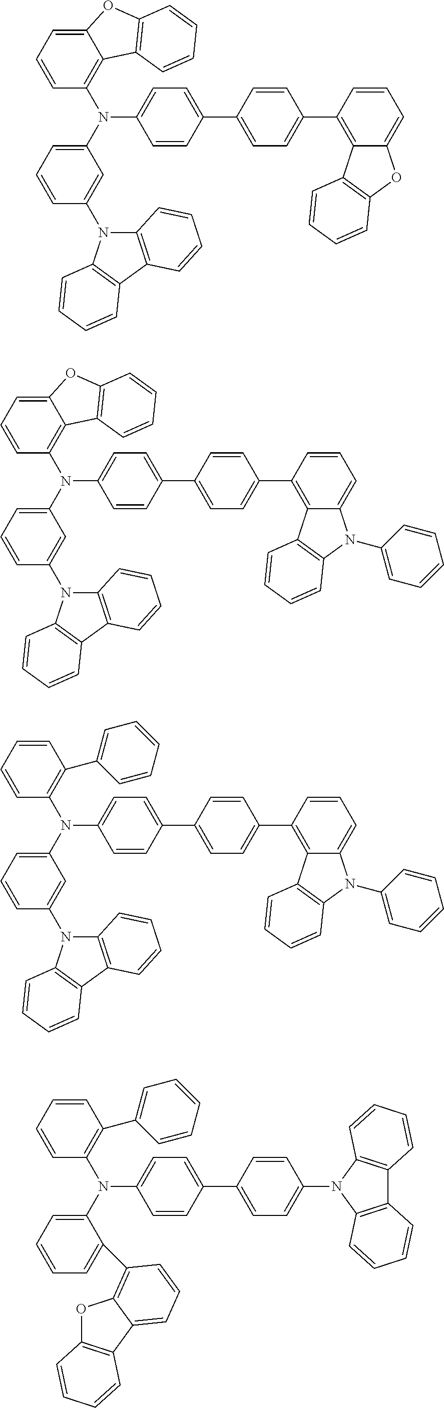

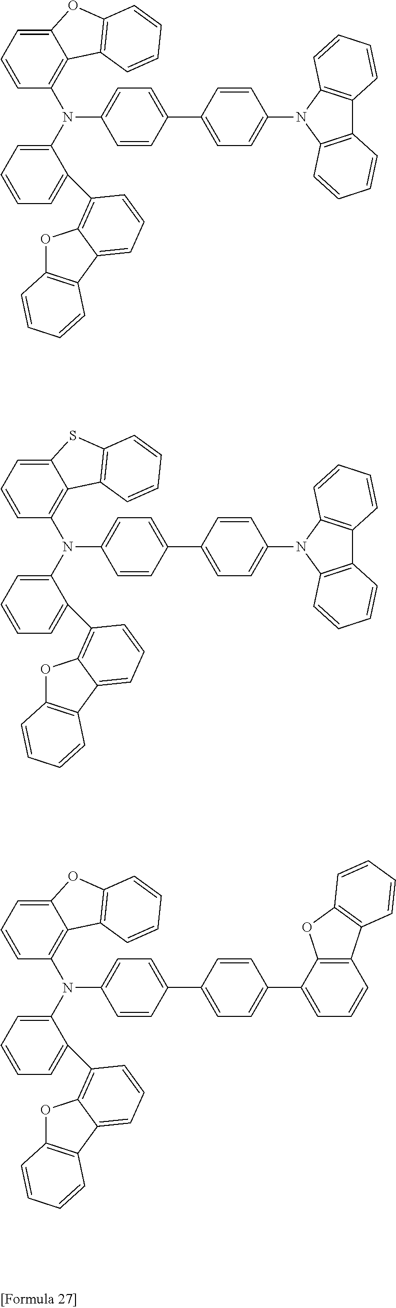 [SMILES: c1ccc(N(c2ccc(-c3ccc(-n4c5ccccc5c5ccccc54)cc3)cc2)c2cccc3oc4ccccc4c23)c(-c2cccc3c2oc2ccccc23)c1 ; c1ccc(N(c2ccc(-c3ccc(-n4c5ccccc5c5ccccc54)cc3)cc2)c2cccc3sc4ccccc4c23)c(-c2cccc3c2oc2ccccc23)c1 ; c1ccc(N(c2ccc(-c3ccc(-c4cccc5c4oc4ccccc45)cc3)cc2)c2cccc3oc4ccccc4c23)c(-c2cccc3c2oc2ccccc23)c1]

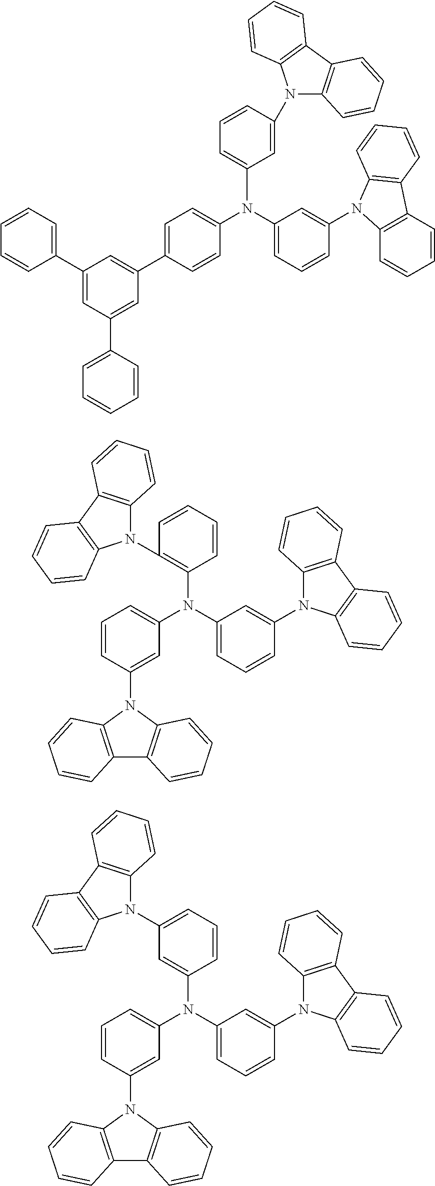

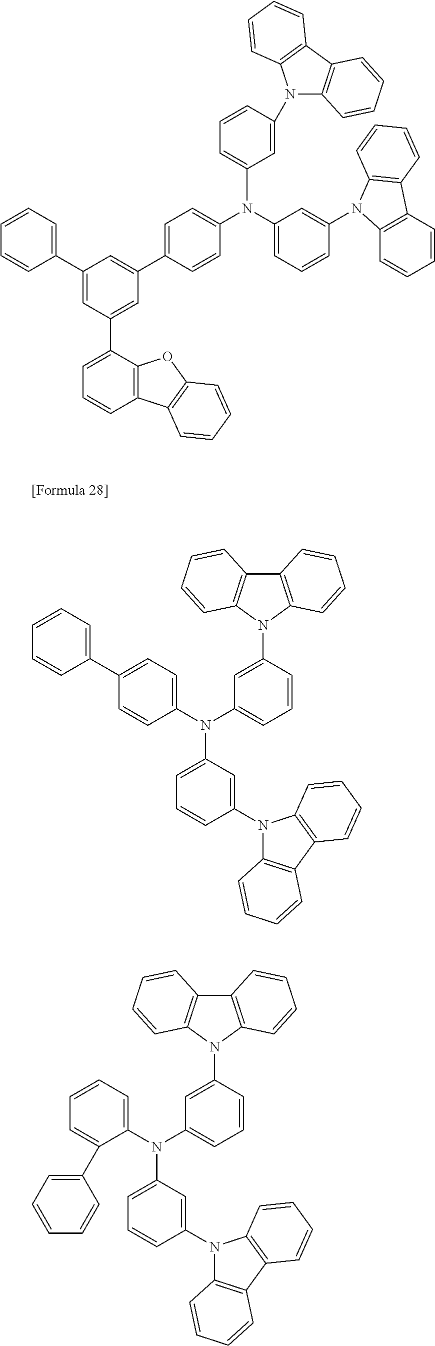 [SMILES: c1ccc(-c2cc(-c3ccc(N(c4cccc(-n5c6ccccc6c6ccccc65)c4)c4cccc(-n5c6ccccc6c6ccccc65)c4)cc3)cc(-c3cccc4c3oc3ccccc34)c2)cc1 ; c1ccc(-c2ccc(N(c3cccc(-n4c5ccccc5c5ccccc54)c3)c3cccc(-n4c5ccccc5c5ccccc54)c3)cc2)cc1 ; c1ccc(-c2ccccc2N(c2cccc(-n3c4ccccc4c4ccccc43)c2)c2cccc(-n3c4ccccc4c4ccccc43)c2)cc1]

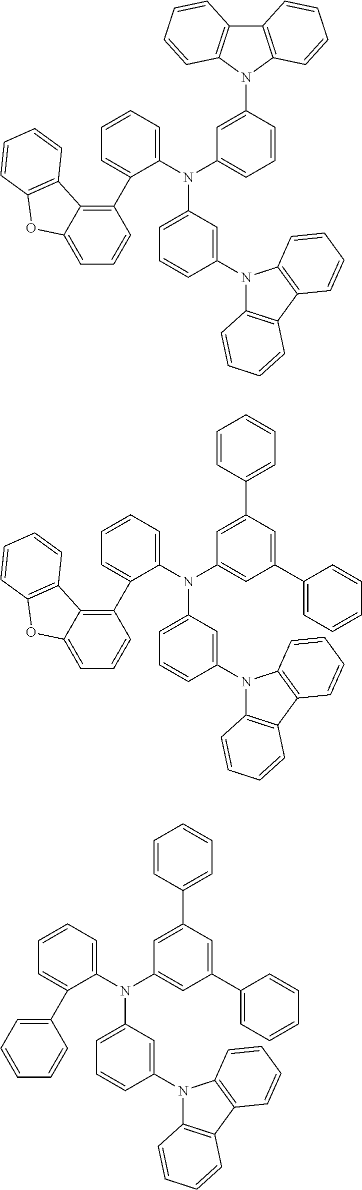

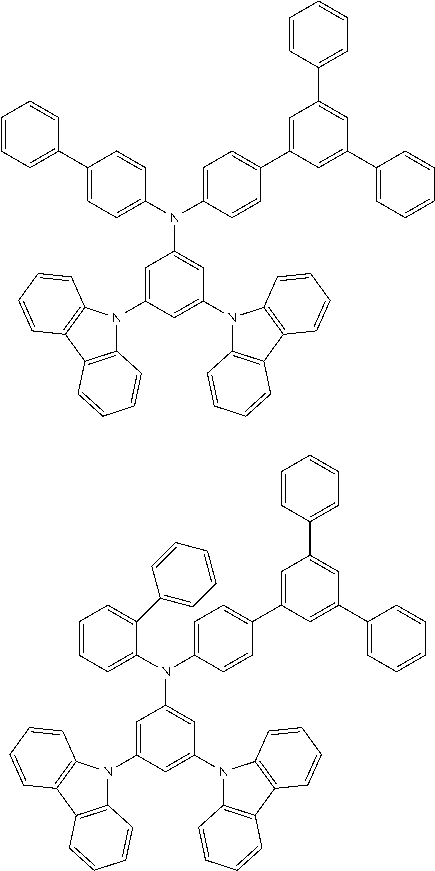 [SMILES: c1ccc(-c2ccc(N(c3ccc(-c4cc(-c5ccccc5)cc(-c5ccccc5)c4)cc3)c3cc(-n4c5ccccc5c5ccccc54)cc(-n4c5ccccc5c5ccccc54)c3)cc2)cc1 ; c1ccc(-c2cc(-c3ccccc3)cc(-c3ccc(N(c4cc(-n5c6ccccc6c6ccccc65)cc(-n5c6ccccc6c6ccccc65)c4)c4ccccc4-c4ccccc4)cc3)c2)cc1]

### Second Layer

A second layer 7 contains a compound represented by the formula (B) below.

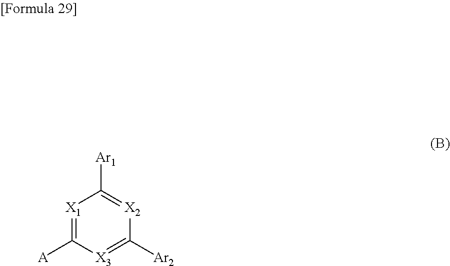

In the formula (B):

X₁ to X₃ are each independently a nitrogen atom or CR₁, at least one of X₁ to X₃ is a nitrogen atom;

R₁ is a hydrogen atom or a substituent;

R₁ serving as the substituent is each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms;

Ar₁ and Ar₂ are each independently represented by a formula (1B) below, or are a substituted or unsubstituted aryl group having 6 to 30 ring carbon atoms, or a substituted or unsubstituted heteroaryl group having 5 to 30 ring atoms; and

A is represented by a formula (1B) below.

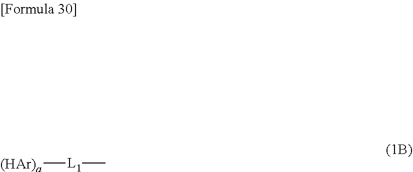

In the formula (1B):

HAr is represented by a formula (2B) below;

a is 1, 2, 3, 4 or 5;

when a is 1, L₁ is a single bond or a divalent linking group;

when a is 2, 3, 4 or 5, L₁ is a trivalent to hexavalent linking group;

a plurality of HAr are mutually the same or different;

the linking group is a group derived from a substituted or unsubstituted aryl group having 6 to 30 ring carbon atoms, a group derived from a substituted or unsubstituted heteroaryl group having 5 to 30 ring atoms, a group derived from a group formed by mutually bonding two groups selected from the group consisting of a substituted or unsubstituted aryl group having 6 to 30 ring carbon atoms and a substituted or unsubstituted heteroaryl group having 5 to 30 ring atoms, or a group derived from a group formed by mutually bonding three groups selected from the group consisting of a substituted or unsubstituted aryl group having 6 to 30 ring carbon atoms and a substituted or unsubstituted heteroaryl group having 5 to 30 ring atoms; and

the mutually bonded groups are mutually the same or different.

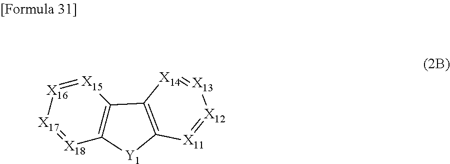

In the formula (2B):

X₁₁ to X₁₈ are each independently a nitrogen atom, CR₁₃, or a carbon atom bonded to L₁;

a plurality of R₁₃ are mutually the same or different;

Y₁ is an oxygen atom, a sulfur atom, NR₁₈, SiR₁₁R₁₂, CR₁₄R₁₅, a nitrogen atom bonded to L₁, a silicon atom bonded to each of R₁₆ and L₁, or a carbon atom bonded to each of R₁₇ and L₁;

among carbon atoms in X₁₁ to X₁₈, R₁₁ to R₁₂, and R₁₄ to R₁₅ as well as a nitrogen atom, a silicon atom, and a carbon atom in Y₁, one atom is bonded to L₁;

R₁₁ and R₁₂ are mutually the same or different, R₁₄ and R₁₅ are mutually the same or different;

R₁₁ to R₁₈ are each independently a hydrogen atom or a substituent, or at least one pair of a pair of adjacent ones of R₁₃, a pair of R₁₁ and R₁₂, or a pair of R₁₄ and R₁₅ are bonded to each other to form a ring; and

R₁₁ to R₁₈ serving as the substituent are each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms.

When Y₁ is a silicon atom bonded to both R₁₆ and L₁, the formula (2B) is represented by a formula (2B-1). In the formula (2B-1), X₁₁ to X₁₈ represent the same as X₁₁ to X₁₈ in the formula (2B).

When Y₁ is a carbon atom bonded to each of R₁₇ and L₁, the formula (2B) is represented by a formula (28-2). In the formula (2B-2), X₁₁ to X₁₈ represent the same as X₁₁ to X₁₈ in the formula (2B).

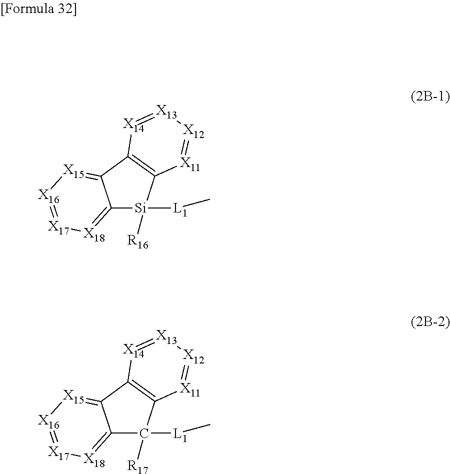

In the formula (1B), L₁ as a linking group is also preferably a divalent to hexavalent residue derived from a substituted or unsubstituted aryl group having 6 to 30 ring carbon atoms.

In the formula (1B), L₁ as a linking group is also preferably a trivalent to hexavalent residue derived from a substituted or unsubstituted aryl group having 6 to 30 ring carbon atoms.

In the formula (1B), a is preferably 1, 2 or 3, more preferably 1 or 2.

When a is 1, L₁ is a divalent linking group and the formula (1B) is represented by a formula (11B-1).

When a is 2, 3, 4 or 5, L₁ is a trivalent to hexavalent linking group. When a is 2, L₁ is a trivalent linking group and the formula (1B) is represented by a formula (11B-2). At this time, HAr are the same or different.

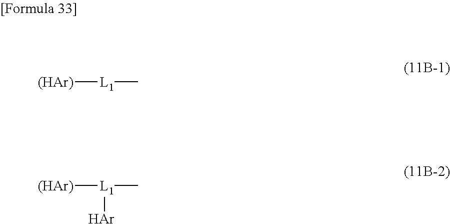

In the formulae (11B-1) and (11B-2), L₁ is a divalent or trivalent linking group. The linking group is a group derived from a substituted or unsubstituted aryl group having 6 to 30 ring carbon atoms, a group derived from a substituted or unsubstituted heteroaryl group having 5 to 30 ring atoms, a group derived from a group formed by mutually bonding two groups selected from the group consisting of a substituted or unsubstituted aryl group having 6 to 30 ring carbon atoms and a substituted or unsubstituted heteroaryl group having 5 to 30 ring atoms, or a group derived from a group formed by mutually bonding three groups selected from the group consisting of a substituted or unsubstituted aryl group having 6 to 30 ring carbon atoms and a substituted or unsubstituted heteroaryl group having 5 to 30 ring atoms.

As for L₁ in the formulae (1B), (11B-1) and (11B-2), the group formed by mutually bonding the two or three groups is a group formed by mutually bonding, with a single bond, two or three of divalent or trivalent residues derived from the aryl group having 6 to 30 ring carbon atoms and the heteroaryl group having 5 to 30 ring atoms. The mutually bonded groups as the linking group are the same or different.

In the formulae (1B), (11B-1) and (11B-2), L₁ as the linking group is preferably a divalent or trivalent residue derived from a substituted or unsubstituted aryl group having 6 to 30 ring carbon atoms or a divalent or trivalent residue derived from a substituted or unsubstituted heteroaryl group having 5 to 30 ring atoms.

In the formulae (1B), (11B-1) and (11B-2), L₁ as the linking group is also preferably a divalent or trivalent residue derived from one of benzene, biphenyl, terphenyl, naphthalene, and phenanthrene.

In the formula (1B), it is also preferable that a is 1 or 2 and L₁ is a divalent or trivalent linking group.

In the formula (1B), it is also preferable that a is 1, L₁ is a linking group, L₁ as the linking group is a divalent residue derived from a substituted or unsubstituted aryl group having 6 to 30 ring carbon atoms or a divalent residue derived from a substituted or unsubstituted heteroaryl group having 5 to 30 ring atoms.

In the formula (1B), it is also preferable that a is 2, L₁ is a linking group, L₁ as the linking group is a trivalent residue derived from a substituted or unsubstituted aryl group having 6 to 30 ring carbon atoms or a trivalent residue derived from a substituted or unsubstituted heteroaryl group having 5 to 30 ring atoms.

In the formula (1B), it is also preferable that L₁ is a single bond.

In the formula (2B), it is also preferable that X₁₃ or X₁₆ is a carbon atom bonded to L₁.

In the formula (2B), Y₁ is preferably NR₁₈, an oxygen atom, a sulfur atom, CR₁₄R₁₅, or a nitrogen atom bonded to L₁.

In the formula (2B), Y₁ is also preferably CR₁₄R₁₅.

When Y₁ is CR₁₄R₁₅, it is preferable that one of X₁₁ to X₁₈ is a carbon atom bonded to L₁ and the rest of X₁₁ to X₁₈ are a nitrogen atom or CR₁₃.

In the formula (2B), it is also preferable that Y₁ is NR₁₈ or a nitrogen atom bonded to L₁. When Y₁ is NR₁₈, it is preferable that one of X₁₁ to X₁₈ is a carbon atom bonded to L₁ and the rest of X₁₁ to X₁₈ are each a nitrogen atom or CR₁₃. When Y₁ is a nitrogen atom bonded to L₁, it is preferable that X₁₁ to X₁₈ are each independently a nitrogen atom or CR₁₃.

In the formula (2B), Y₁ is preferably an oxygen atom or a sulfur atom, more preferably an oxygen atom.

In the formula (2B), it is also preferable that Y₁ is an oxygen atom or a sulfur atom, one of X₁₁ to X₁₈ is a carbon atom bonded to L₁ and the rest of X₁₁ to X₁₈ are each CR₁₃.

In the formula (2B), it is more preferable that Y₁ is an oxygen atom, X₁₁ and X₁₈ are CR₁₃, one of X₁₂ to X₁₇ is a carbon atom bonded to L₁, and the rest of X₁₂ to X₁₇ are CR₁₃.

In the formula (1B), two or three of X₁ to X₃ are preferably nitrogen atoms.

When two of X₁ to X₃ are nitrogen atoms, it is preferable that X₁ and X₂ are nitrogen atoms and X₃ is CR₁.

In the formula (B), it is more preferable that X₁ and X₂ are nitrogen atoms, X₃ is CR₁, and R₁ is a hydrogen atom. In this case, a third compound is represented by a formula (21) below.

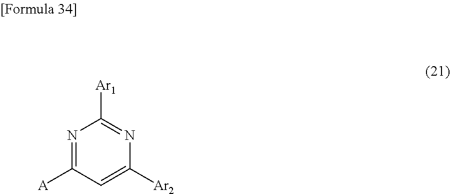

In the formula (21), A, Ar₁, and Ar₂ represent the same as A, Ar₁, and Ar₂ in the formula (B).

### Manufacturing Method of Compound Represented by Formula (B)

The compound represented by the formula (B) can be manufactured by a publicly known method.

Specific examples of the compound represented by the formula (B) are shown below. It should be noted that the compound represented by the formula (B) is not limited to the specific examples.

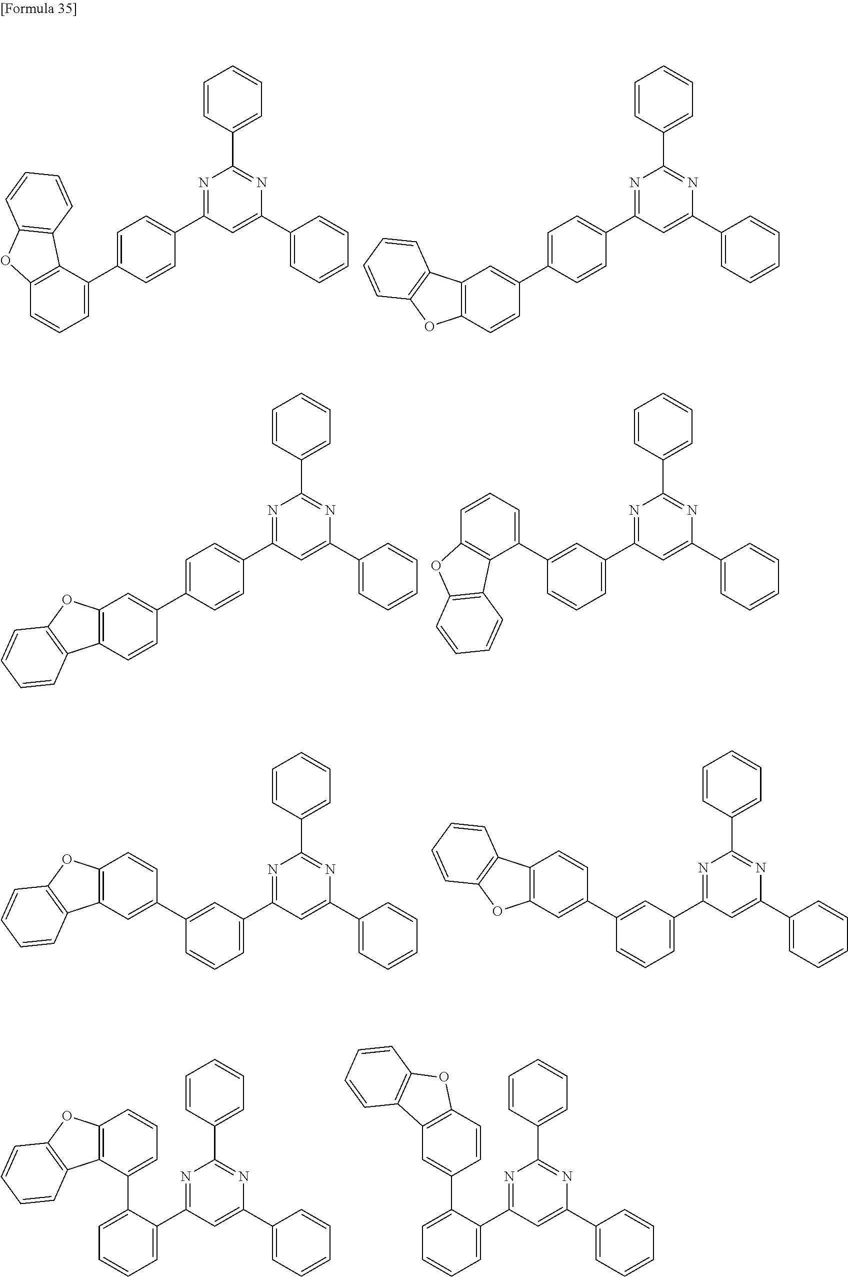

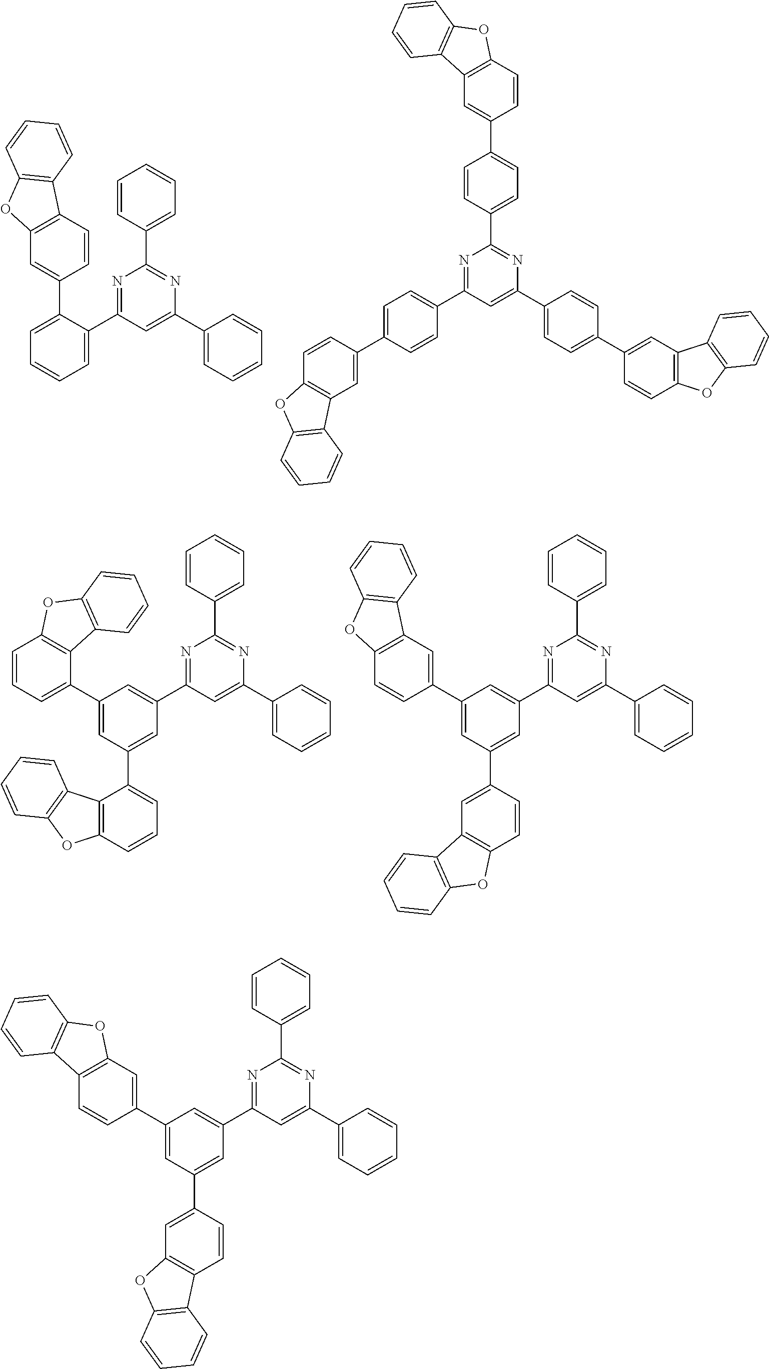

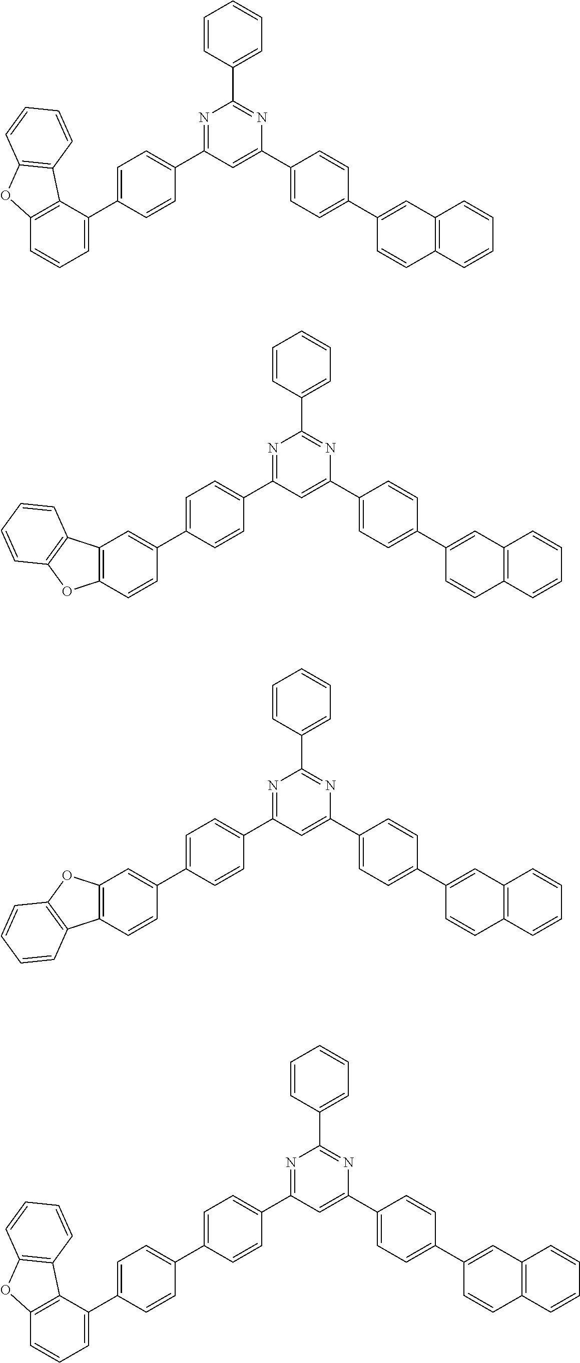 [SMILES: c1ccc(-c2nc(-c3ccc(-c4ccc5ccccc5c4)cc3)cc(-c3ccc(-c4cccc5oc6ccccc6c45)cc3)n2)cc1 ; c1ccc(-c2nc(-c3ccc(-c4ccc5ccccc5c4)cc3)cc(-c3ccc(-c4ccc5oc6ccccc6c5c4)cc3)n2)cc1 ; c1ccc(-c2nc(-c3ccc(-c4ccc5ccccc5c4)cc3)cc(-c3ccc(-c4ccc5c(c4)oc4ccccc45)cc3)n2)cc1 ; c1ccc(-c2nc(-c3ccc(-c4ccc(-c5cccc6oc7ccccc7c56)cc4)cc3)cc(-c3ccc(-c4ccc5ccccc5c4)cc3)n2)cc1]

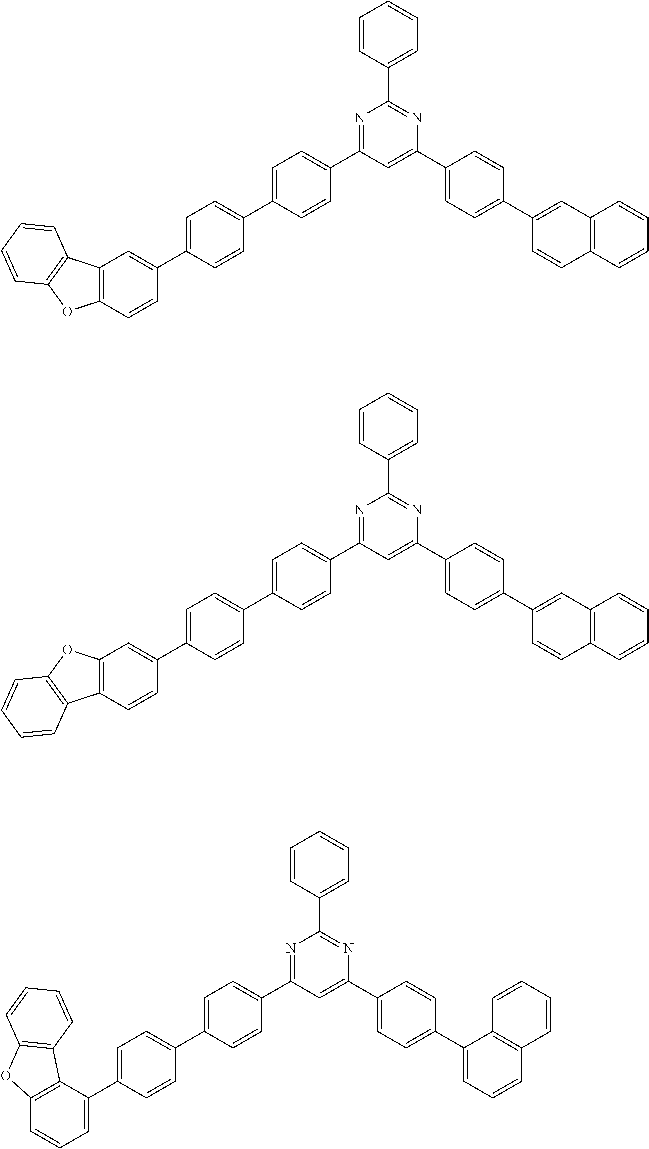 [SMILES: c1ccc(-c2nc(-c3ccc(-c4ccc(-c5ccc6oc7ccccc7c6c5)cc4)cc3)cc(-c3ccc(-c4ccc5ccccc5c4)cc3)n2)cc1 ; c1ccc(-c2nc(-c3ccc(-c4ccc(-c5ccc6c(c5)oc5ccccc56)cc4)cc3)cc(-c3ccc(-c4ccc5ccccc5c4)cc3)n2)cc1 ; c1ccc(-c2nc(-c3ccc(-c4ccc(-c5cccc6oc7ccccc7c56)cc4)cc3)cc(-c3ccc(-c4cccc5ccccc45)cc3)n2)cc1]

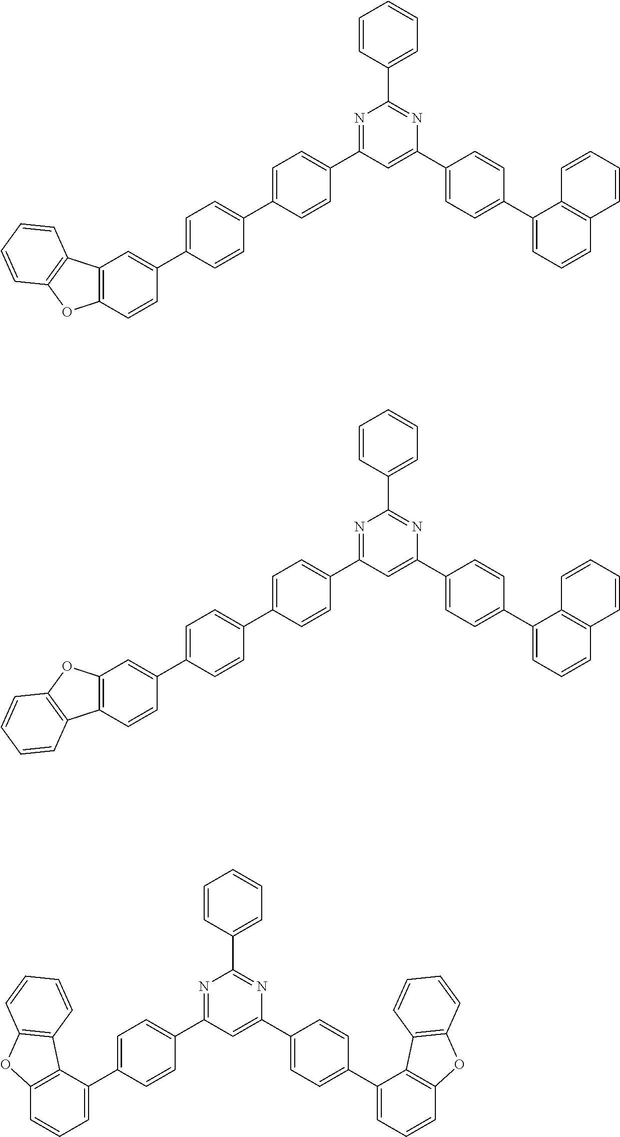 [SMILES: c1ccc(-c2nc(-c3ccc(-c4ccc(-c5ccc6oc7ccccc7c6c5)cc4)cc3)cc(-c3ccc(-c4cccc5ccccc45)cc3)n2)cc1 ; c1ccc(-c2nc(-c3ccc(-c4ccc(-c5ccc6c(c5)oc5ccccc56)cc4)cc3)cc(-c3ccc(-c4cccc5ccccc45)cc3)n2)cc1 ; c1ccc(-c2nc(-c3ccc(-c4cccc5oc6ccccc6c45)cc3)cc(-c3ccc(-c4cccc5oc6ccccc6c45)cc3)n2)cc1]

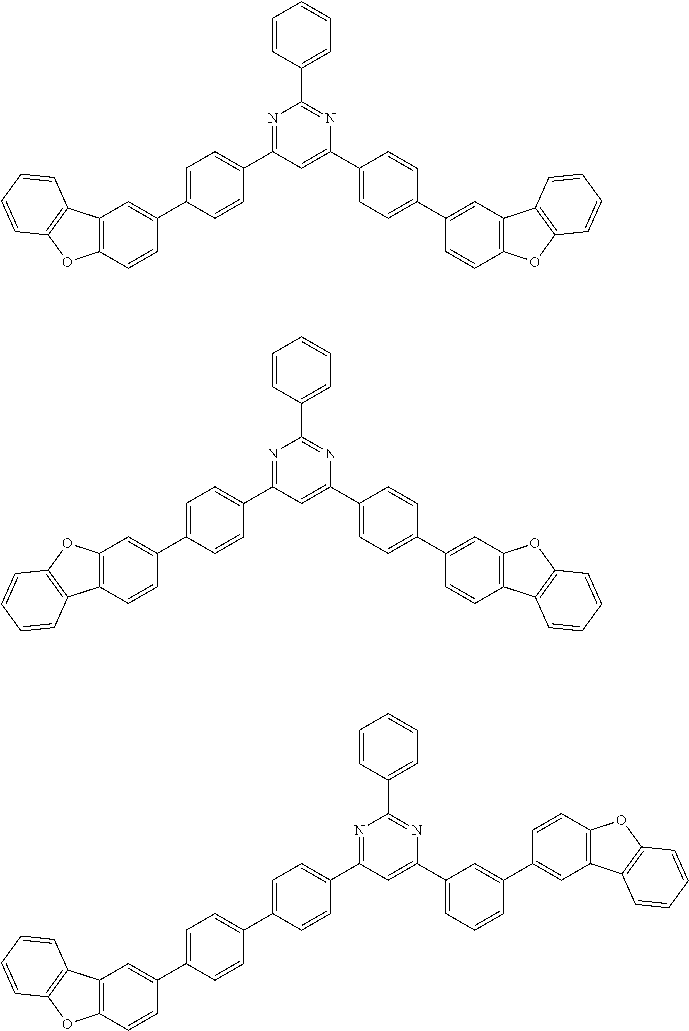 [SMILES: c1ccc(-c2nc(-c3ccc(-c4ccc5oc6ccccc6c5c4)cc3)cc(-c3ccc(-c4ccc5oc6ccccc6c5c4)cc3)n2)cc1 ; c1ccc(-c2nc(-c3ccc(-c4ccc5c(c4)oc4ccccc45)cc3)cc(-c3ccc(-c4ccc5c(c4)oc4ccccc45)cc3)n2)cc1 ; c1ccc(-c2nc(-c3ccc(-c4ccc(-c5ccc6oc7ccccc7c6c5)cc4)cc3)cc(-c3cccc(-c4ccc5oc6ccccc6c5c4)c3)n2)cc1]

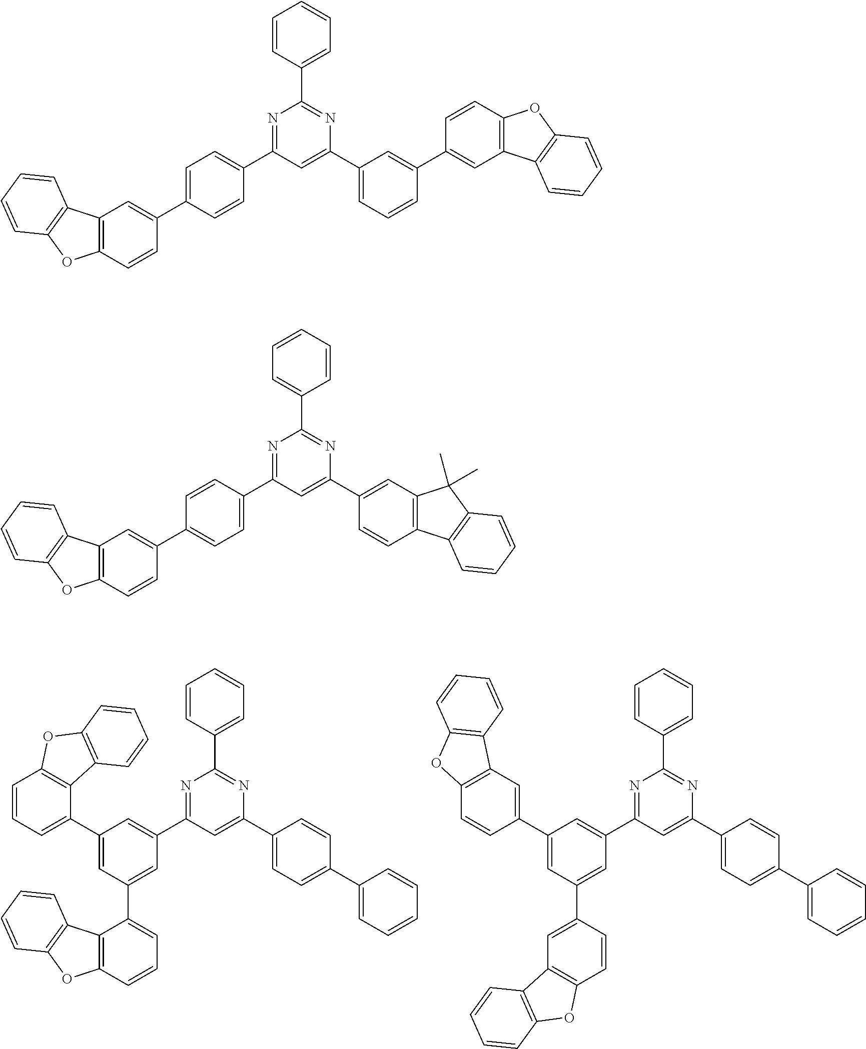 [SMILES: c1ccc(-c2nc(-c3ccc(-c4ccc5oc6ccccc6c5c4)cc3)cc(-c3cccc(-c4ccc5oc6ccccc6c5c4)c3)n2)cc1 ; CC1(C)c2ccccc2-c2ccc(-c3cc(-c4ccc(-c5ccc6oc7ccccc7c6c5)cc4)nc(-c4ccccc4)n3)cc21 ; c1ccc(-c2ccc(-c3cc(-c4cc(-c5cccc6oc7ccccc7c56)cc(-c5cccc6oc7ccccc7c56)c4)nc(-c4ccccc4)n3)cc2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

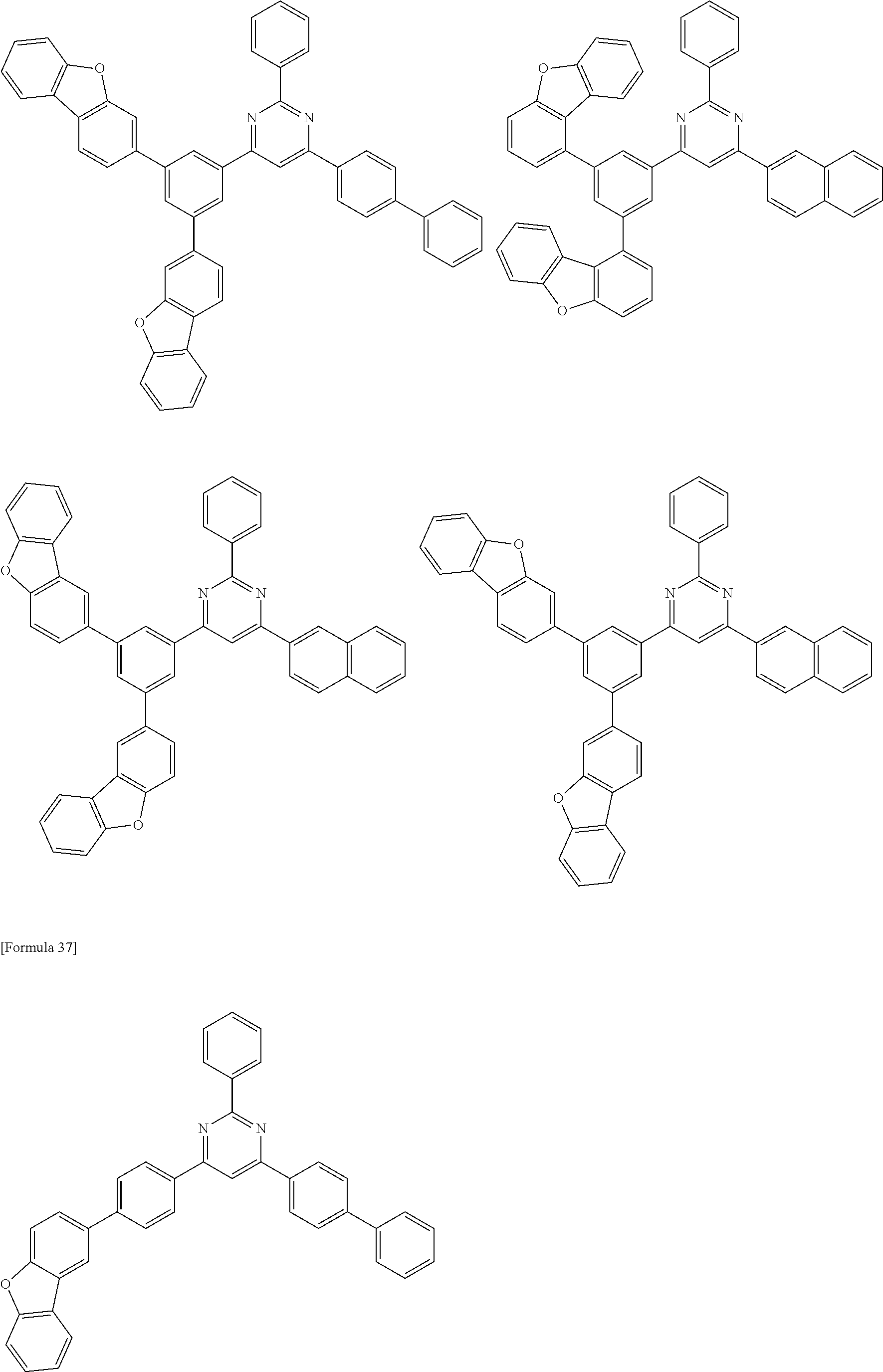

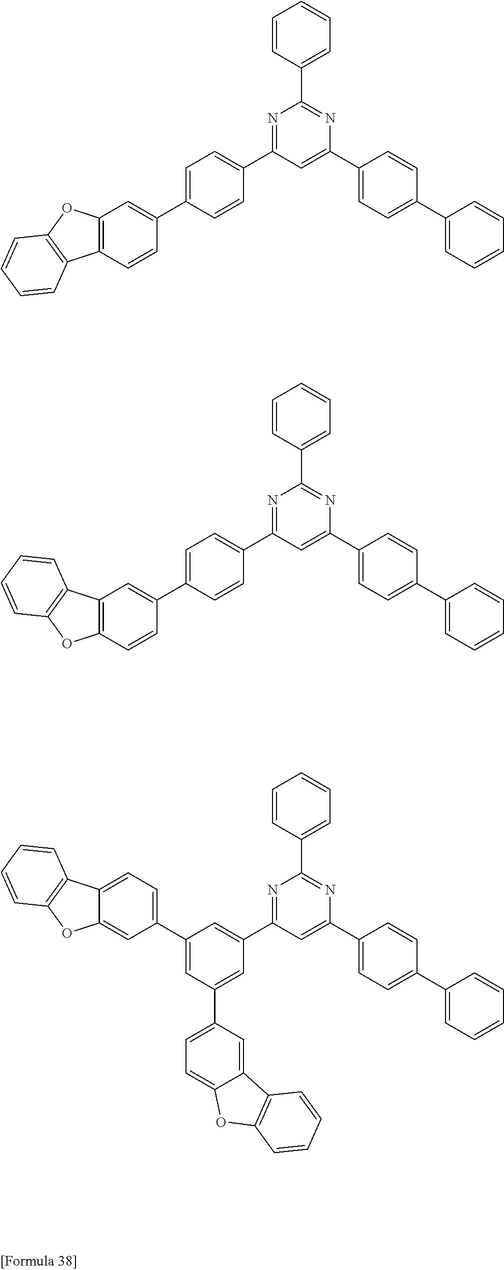 [SMILES: c1ccc(-c2ccc(-c3cc(-c4ccc(-c5ccc6c(c5)oc5ccccc56)cc4)nc(-c4ccccc4)n3)cc2)cc1 ; c1ccc(-c2ccc(-c3cc(-c4ccc(-c5ccc6oc7ccccc7c6c5)cc4)nc(-c4ccccc4)n3)cc2)cc1 ; c1ccc(-c2ccc(-c3cc(-c4cc(-c5ccc6c(c5)oc5ccccc56)cc(-c5ccc6oc7ccccc7c6c5)c4)nc(-c4ccccc4)n3)cc2)cc1]

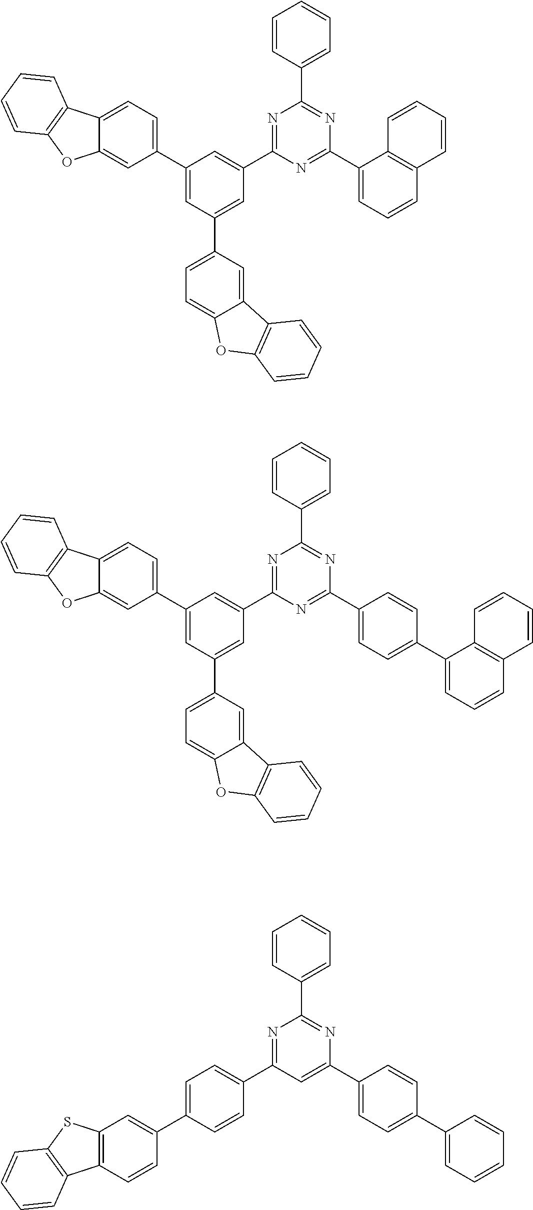 [SMILES: c1ccc(-c2nc(-c3cc(-c4ccc5c(c4)oc4ccccc45)cc(-c4ccc5oc6ccccc6c5c4)c3)nc(-c3cccc4ccccc34)n2)cc1 ; c1ccc(-c2nc(-c3ccc(-c4cccc5ccccc45)cc3)nc(-c3cc(-c4ccc5c(c4)oc4ccccc45)cc(-c4ccc5oc6ccccc6c5c4)c3)n2)cc1 ; c1ccc(-c2ccc(-c3cc(-c4ccc(-c5ccc6c(c5)sc5ccccc56)cc4)nc(-c4ccccc4)n3)cc2)cc1]

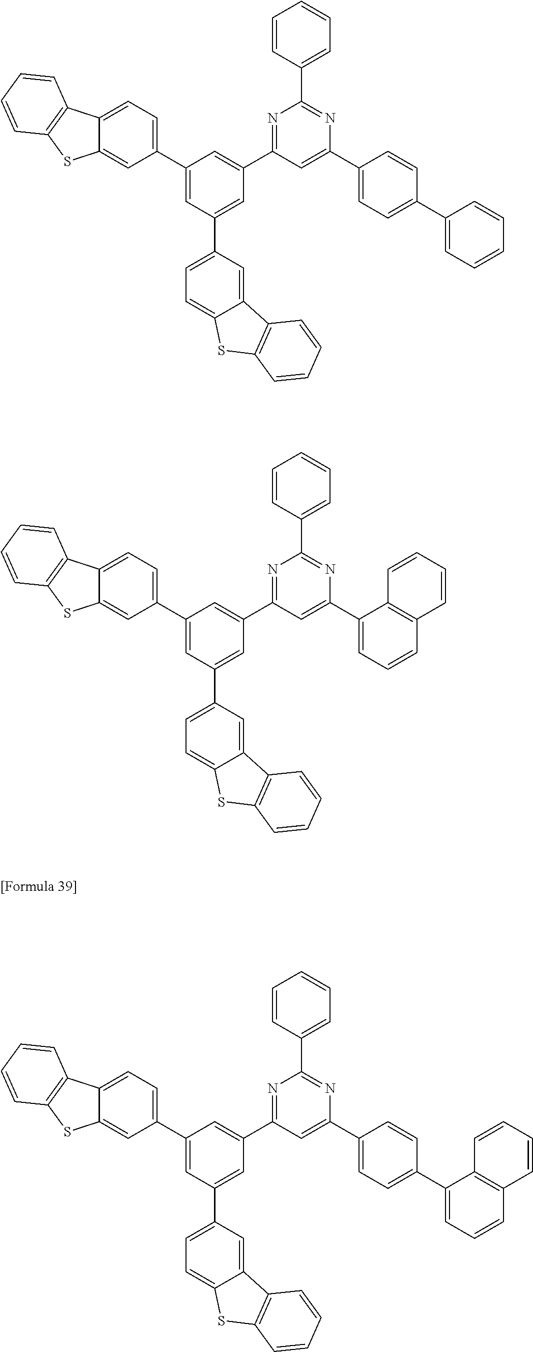 [SMILES: c1ccc(-c2ccc(-c3cc(-c4cc(-c5ccc6c(c5)sc5ccccc56)cc(-c5ccc6sc7ccccc7c6c5)c4)nc(-c4ccccc4)n3)cc2)cc1 ; c1ccc(-c2nc(-c3cc(-c4ccc5c(c4)sc4ccccc45)cc(-c4ccc5sc6ccccc6c5c4)c3)cc(-c3cccc4ccccc34)n2)cc1 ; c1ccc(-c2nc(-c3ccc(-c4cccc5ccccc45)cc3)cc(-c3cc(-c4ccc5c(c4)sc4ccccc45)cc(-c4ccc5sc6ccccc6c5c4)c3)n2)cc1]

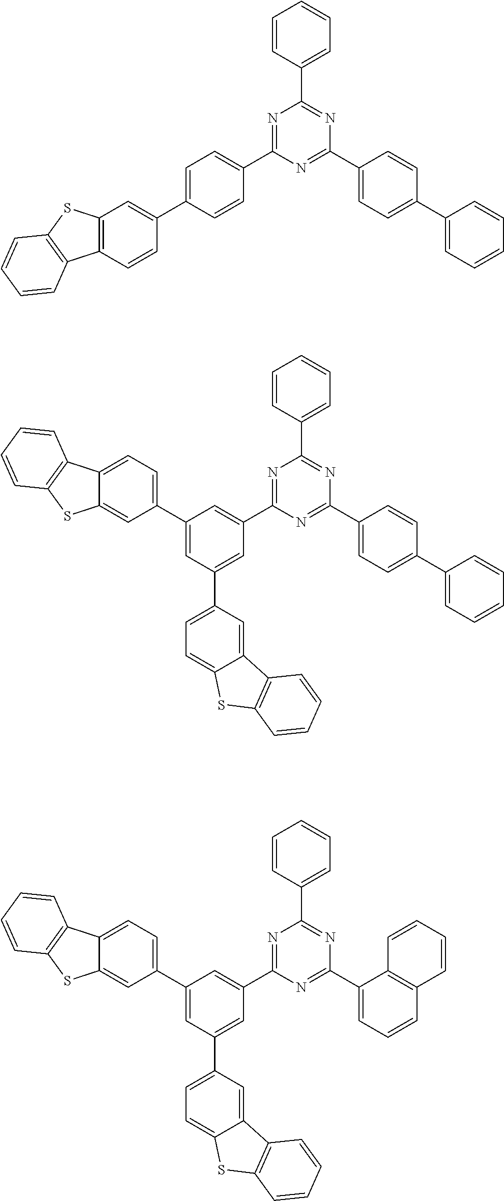 [SMILES: c1ccc(-c2ccc(-c3nc(-c4ccccc4)nc(-c4ccc(-c5ccc6c(c5)sc5ccccc56)cc4)n3)cc2)cc1 ; c1ccc(-c2ccc(-c3nc(-c4ccccc4)nc(-c4cc(-c5ccc6c(c5)sc5ccccc56)cc(-c5ccc6sc7ccccc7c6c5)c4)n3)cc2)cc1 ; c1ccc(-c2nc(-c3cc(-c4ccc5c(c4)sc4ccccc45)cc(-c4ccc5sc6ccccc6c5c4)c3)nc(-c3cccc4ccccc34)n2)cc1]

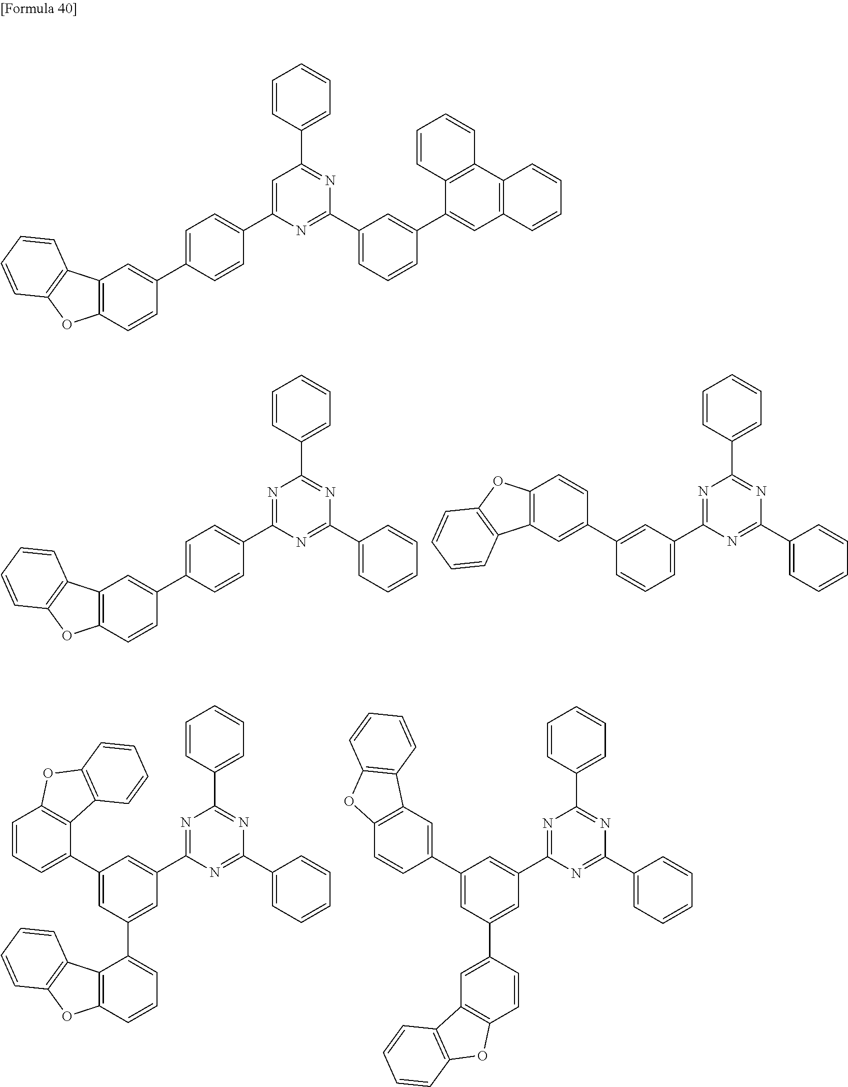

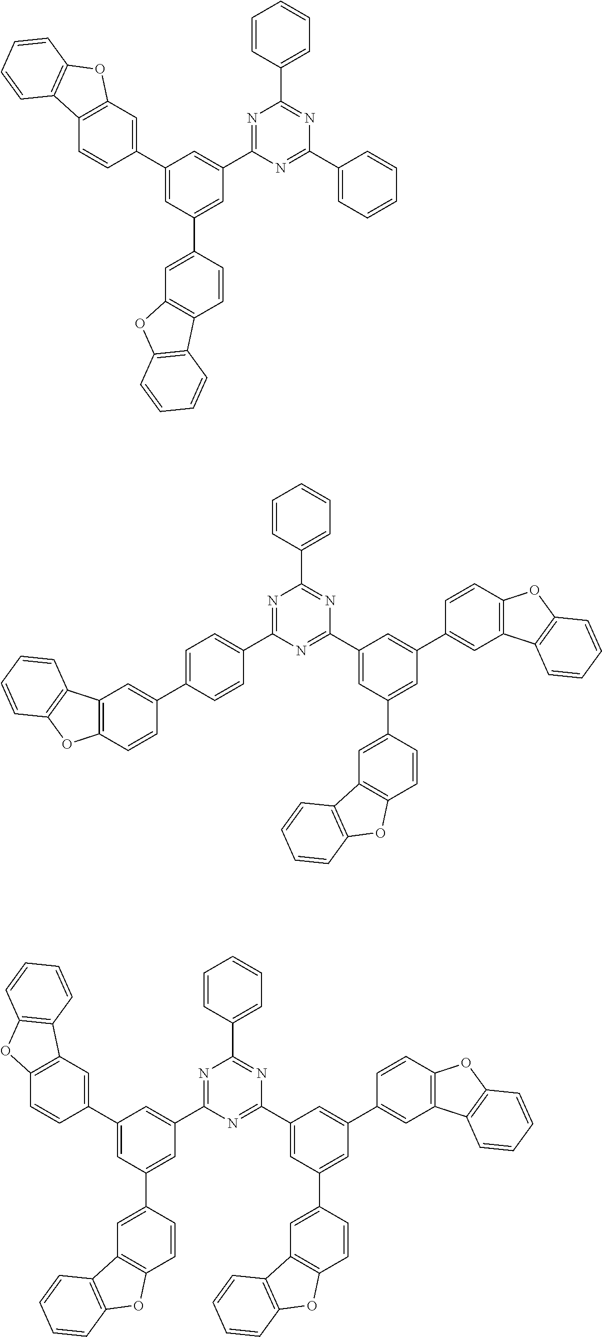 [SMILES: c1ccc(-c2nc(-c3ccccc3)nc(-c3cc(-c4ccc5c(c4)oc4ccccc45)cc(-c4ccc5c(c4)oc4ccccc45)c3)n2)cc1 ; c1ccc(-c2nc(-c3ccc(-c4ccc5oc6ccccc6c5c4)cc3)nc(-c3cc(-c4ccc5oc6ccccc6c5c4)cc(-c4ccc5oc6ccccc6c5c4)c3)n2)cc1 ; c1ccc(-c2nc(-c3cc(-c4ccc5oc6ccccc6c5c4)cc(-c4ccc5oc6ccccc6c5c4)c3)nc(-c3cc(-c4ccc5oc6ccccc6c5c4)cc(-c4ccc5oc6ccccc6c5c4)c3)n2)cc1]

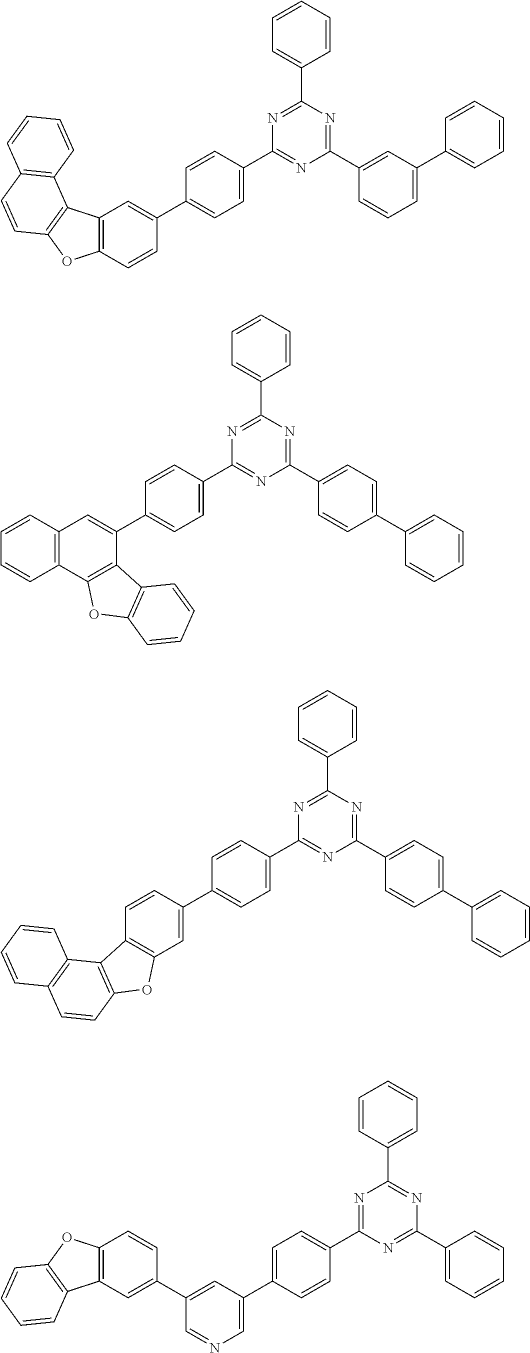 [SMILES: c1ccc(-c2cccc(-c3nc(-c4ccccc4)nc(-c4ccc(-c5ccc6oc7ccc8ccccc8c7c6c5)cc4)n3)c2)cc1 ; c1ccc(-c2ccc(-c3nc(-c4ccccc4)nc(-c4ccc(-c5cc6ccccc6c6oc7ccccc7c56)cc4)n3)cc2)cc1 ; c1ccc(-c2ccc(-c3nc(-c4ccccc4)nc(-c4ccc(-c5ccc6c(c5)oc5ccc7ccccc7c56)cc4)n3)cc2)cc1 ; c1ccc(-c2nc(-c3ccccc3)nc(-c3ccc(-c4cncc(-c5ccc6oc7ccccc7c6c5)c4)cc3)n2)cc1]

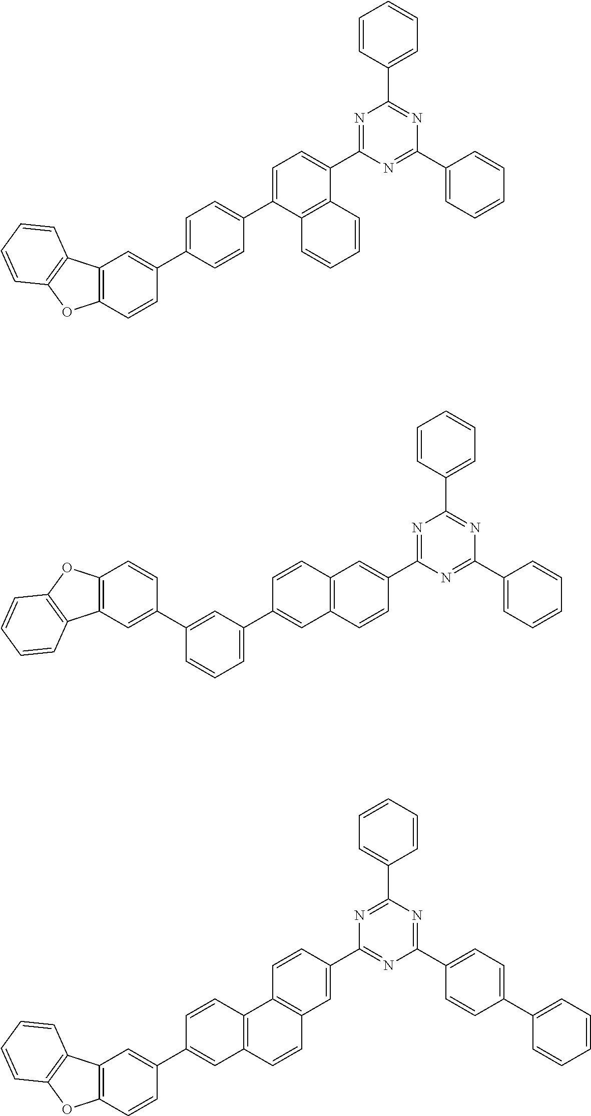 [SMILES: c1ccc(-c2nc(-c3ccccc3)nc(-c3ccc(-c4ccc(-c5ccc6oc7ccccc7c6c5)cc4)c4ccccc34)n2)cc1 ; c1ccc(-c2nc(-c3ccccc3)nc(-c3ccc4cc(-c5cccc(-c6ccc7oc8ccccc8c7c6)c5)ccc4c3)n2)cc1 ; c1ccc(-c2ccc(-c3nc(-c4ccccc4)nc(-c4ccc5c(ccc6cc(-c7ccc8oc9ccccc9c8c7)ccc65)c4)n3)cc2)cc1]

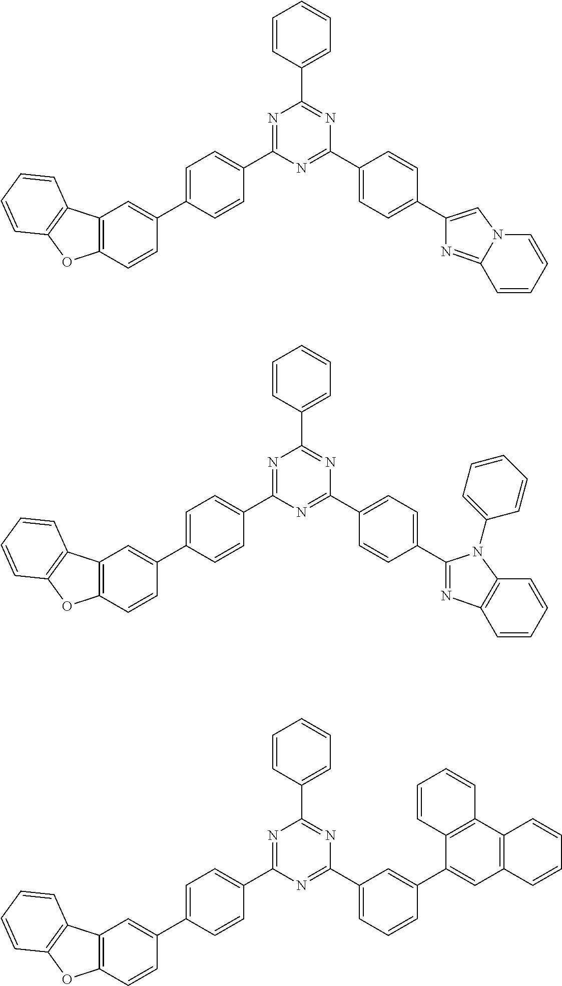 [SMILES: c1ccc(-c2nc(-c3ccc(-c4ccc5oc6ccccc6c5c4)cc3)nc(-c3ccc(-c4cn5ccccc5n4)cc3)n2)cc1 ; c1ccc(-c2nc(-c3ccc(-c4ccc5oc6ccccc6c5c4)cc3)nc(-c3ccc(-c4nc5ccccc5n4-c4ccccc4)cc3)n2)cc1 ; c1ccc(-c2nc(-c3ccc(-c4ccc5oc6ccccc6c5c4)cc3)nc(-c3cccc(-c4cc5ccccc5c5ccccc45)c3)n2)cc1]

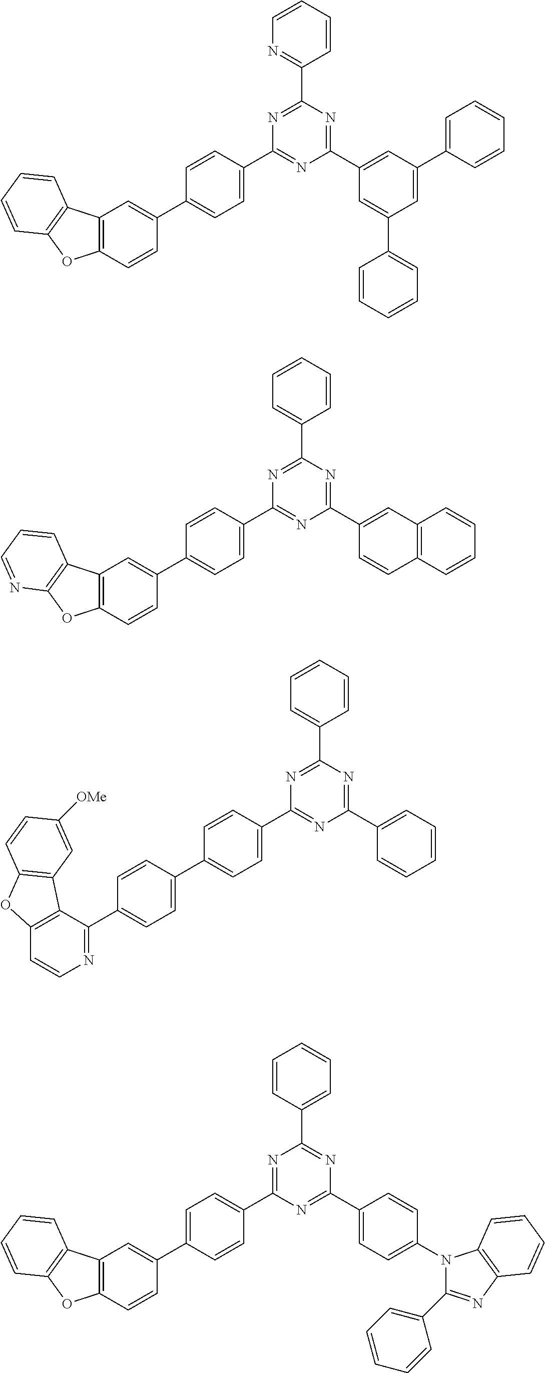 [SMILES: c1ccc(-c2cc(-c3ccccc3)cc(-c3nc(-c4ccc(-c5ccc6oc7ccccc7c6c5)cc4)nc(-c4ccccn4)n3)c2)cc1 ; c1ccc(-c2nc(-c3ccc(-c4ccc5oc6ncccc6c5c4)cc3)nc(-c3ccc4ccccc4c3)n2)cc1 ; COc1ccc2oc3ccnc(-c4ccc(-c5ccc(-c6nc(-c7ccccc7)nc(-c7ccccc7)n6)cc5)cc4)c3c2c1 ; c1ccc(-c2nc(-c3ccc(-c4ccc5oc6ccccc6c5c4)cc3)nc(-c3ccc(-n4c(-c5ccccc5)nc5ccccc54)cc3)n2)cc1]

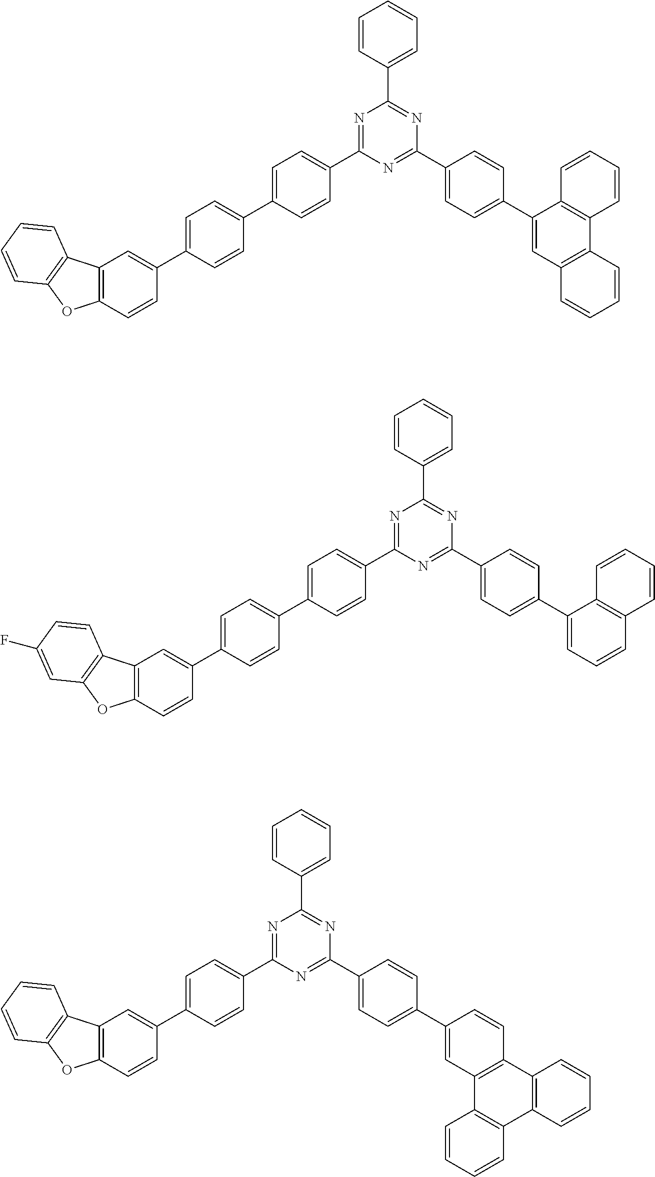 [SMILES: c1ccc(-c2nc(-c3ccc(-c4ccc(-c5ccc6oc7ccccc7c6c5)cc4)cc3)nc(-c3ccc(-c4cc5ccccc5c5ccccc45)cc3)n2)cc1 ; Fc1ccc2c(c1)oc1ccc(-c3ccc(-c4ccc(-c5nc(-c6ccccc6)nc(-c6ccc(-c7cccc8ccccc78)cc6)n5)cc4)cc3)cc12 ; c1ccc(-c2nc(-c3ccc(-c4ccc5oc6ccccc6c5c4)cc3)nc(-c3ccc(-c4ccc5c6ccccc6c6ccccc6c5c4)cc3)n2)cc1]

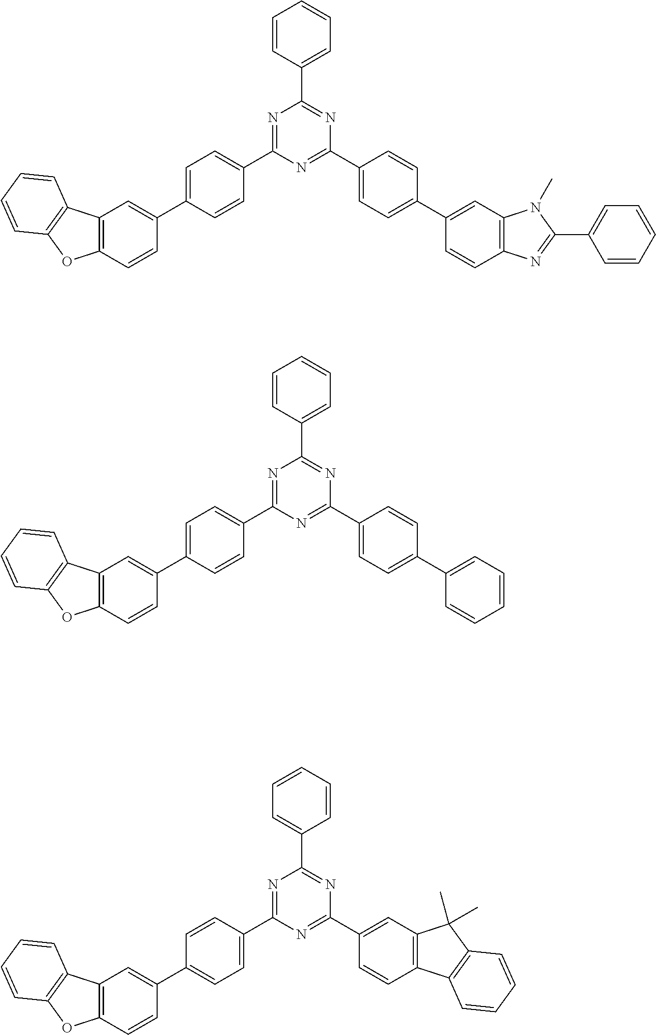 [SMILES: Cn1c(-c2ccccc2)nc2ccc(-c3ccc(-c4nc(-c5ccccc5)nc(-c5ccc(-c6ccc7oc8ccccc8c7c6)cc5)n4)cc3)cc21 ; c1ccc(-c2ccc(-c3nc(-c4ccccc4)nc(-c4ccc(-c5ccc6oc7ccccc7c6c5)cc4)n3)cc2)cc1 ; CC1(C)c2ccccc2-c2ccc(-c3nc(-c4ccccc4)nc(-c4ccc(-c5ccc6oc7ccccc7c6c5)cc4)n3)cc21]

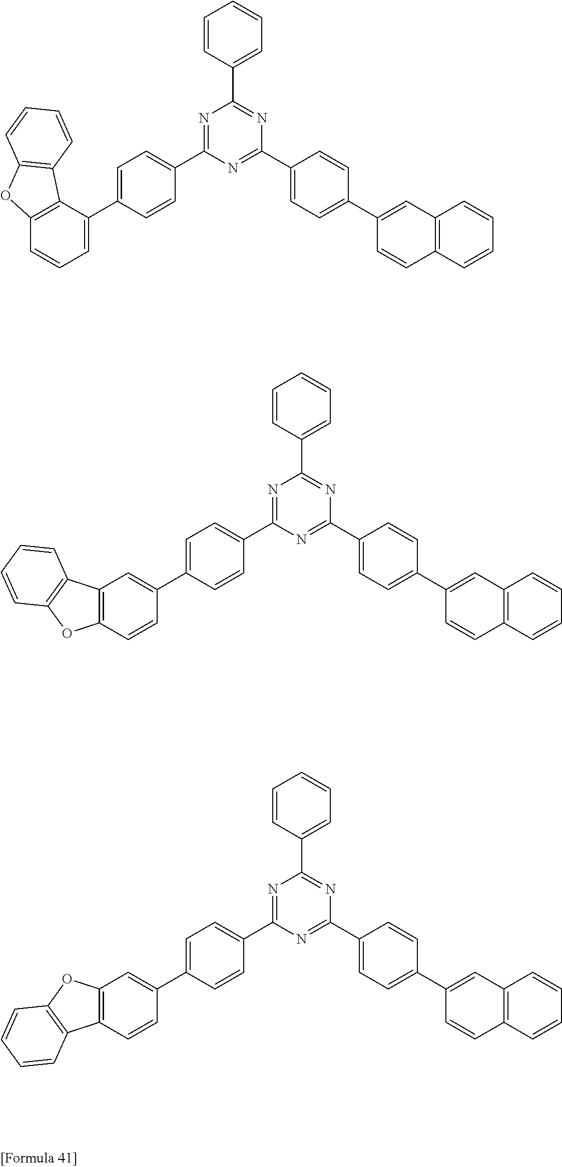 [SMILES: c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccc(-c4ccc5ccccc5c4)cc3)nc(-c3ccc(-c4ccc5oc6ccccc6c5c4)cc3)n2)cc1 ; c1ccc(-c2nc(-c3ccc(-c4ccc5ccccc5c4)cc3)nc(-c3ccc(-c4ccc5c(c4)oc4ccccc45)cc3)n2)cc1]

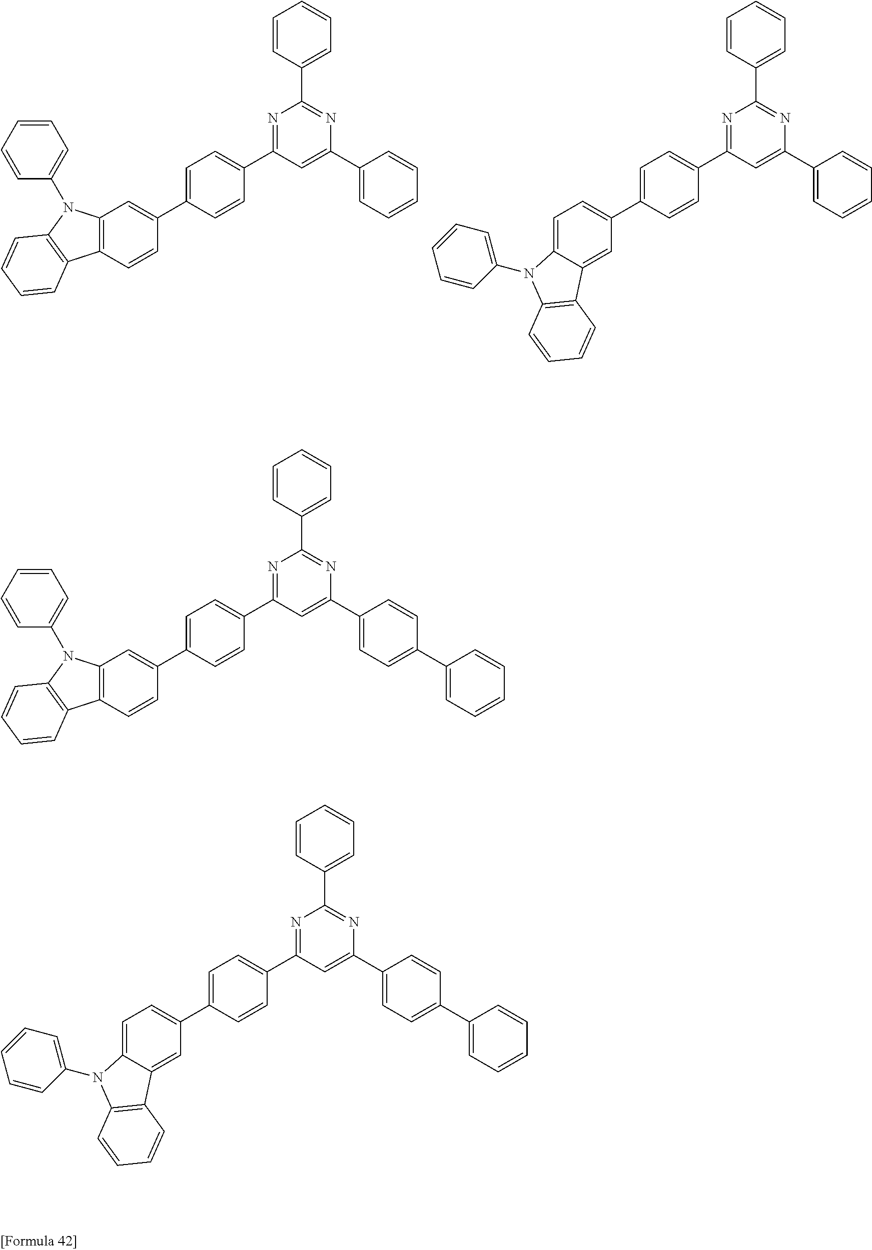

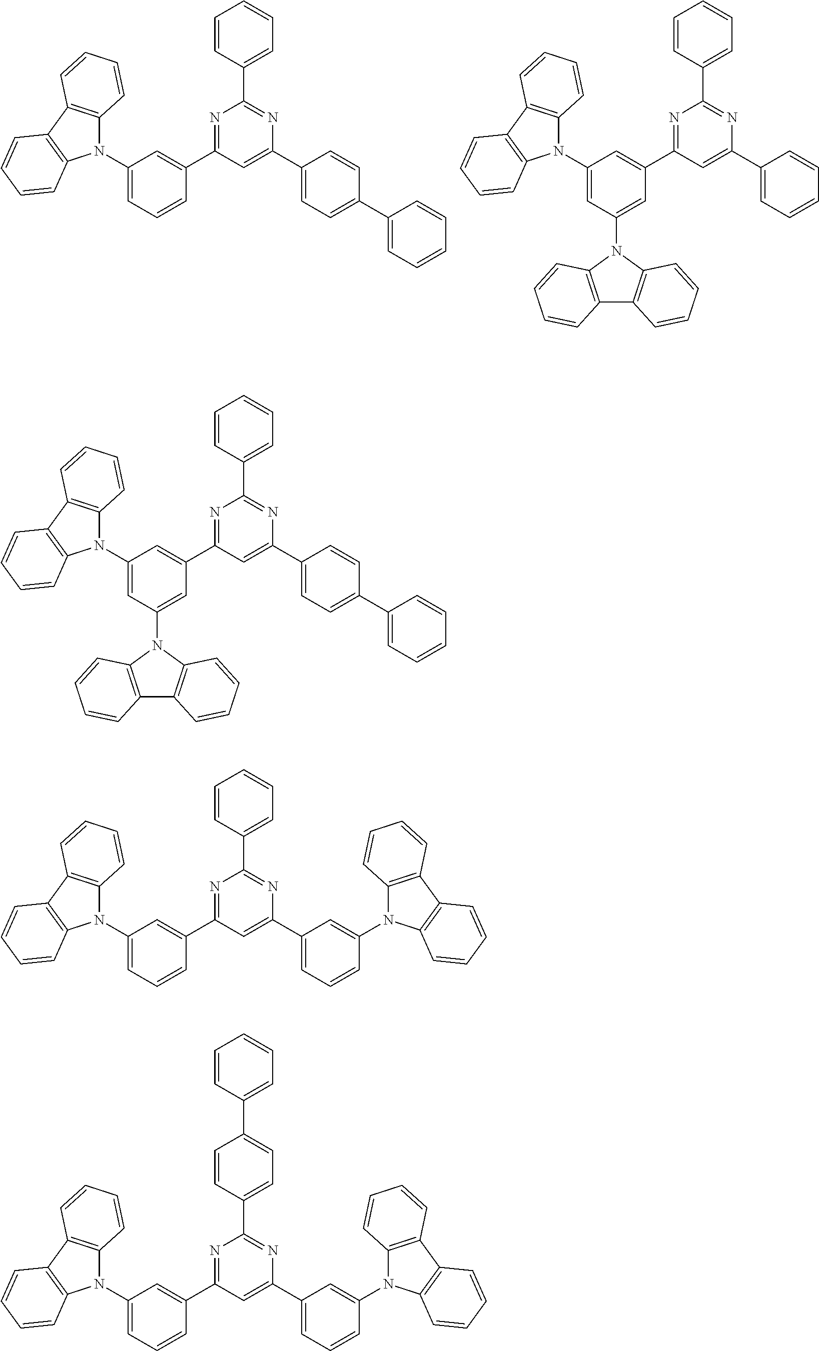

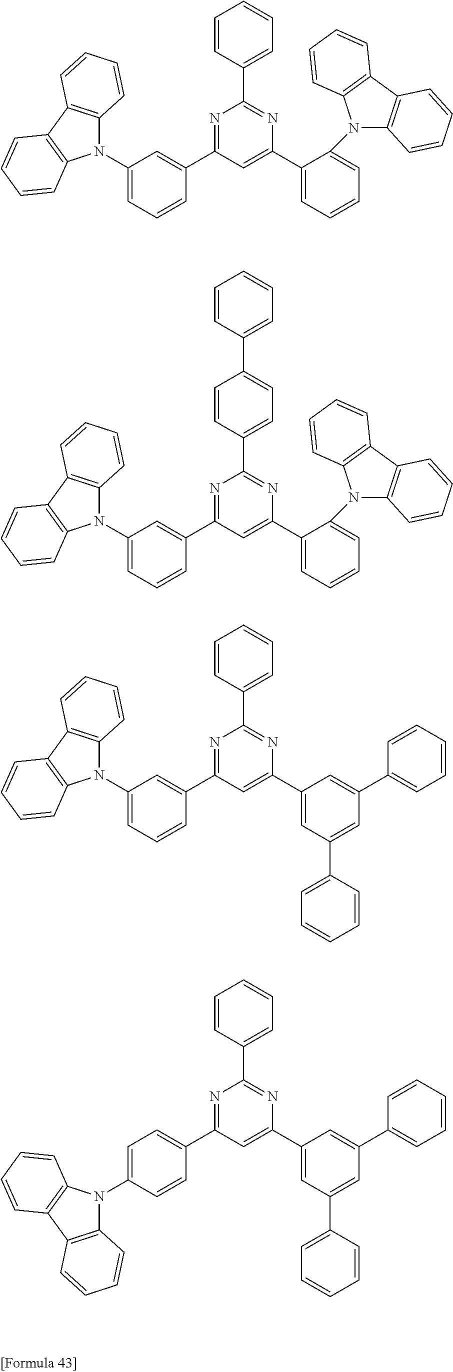 [SMILES: c1ccc(-c2nc(-c3cccc(-n4c5ccccc5c5ccccc54)c3)cc(-c3ccccc3-n3c4ccccc4c4ccccc43)n2)cc1 ; c1ccc(-c2ccc(-c3nc(-c4cccc(-n5c6ccccc6c6ccccc65)c4)cc(-c4ccccc4-n4c5ccccc5c5ccccc54)n3)cc2)cc1 ; c1ccc(-c2cc(-c3ccccc3)cc(-c3cc(-c4cccc(-n5c6ccccc6c6ccccc65)c4)nc(-c4ccccc4)n3)c2)cc1 ; c1ccc(-c2cc(-c3ccccc3)cc(-c3cc(-c4ccc(-n5c6ccccc6c6ccccc65)cc4)nc(-c4ccccc4)n3)c2)cc1]

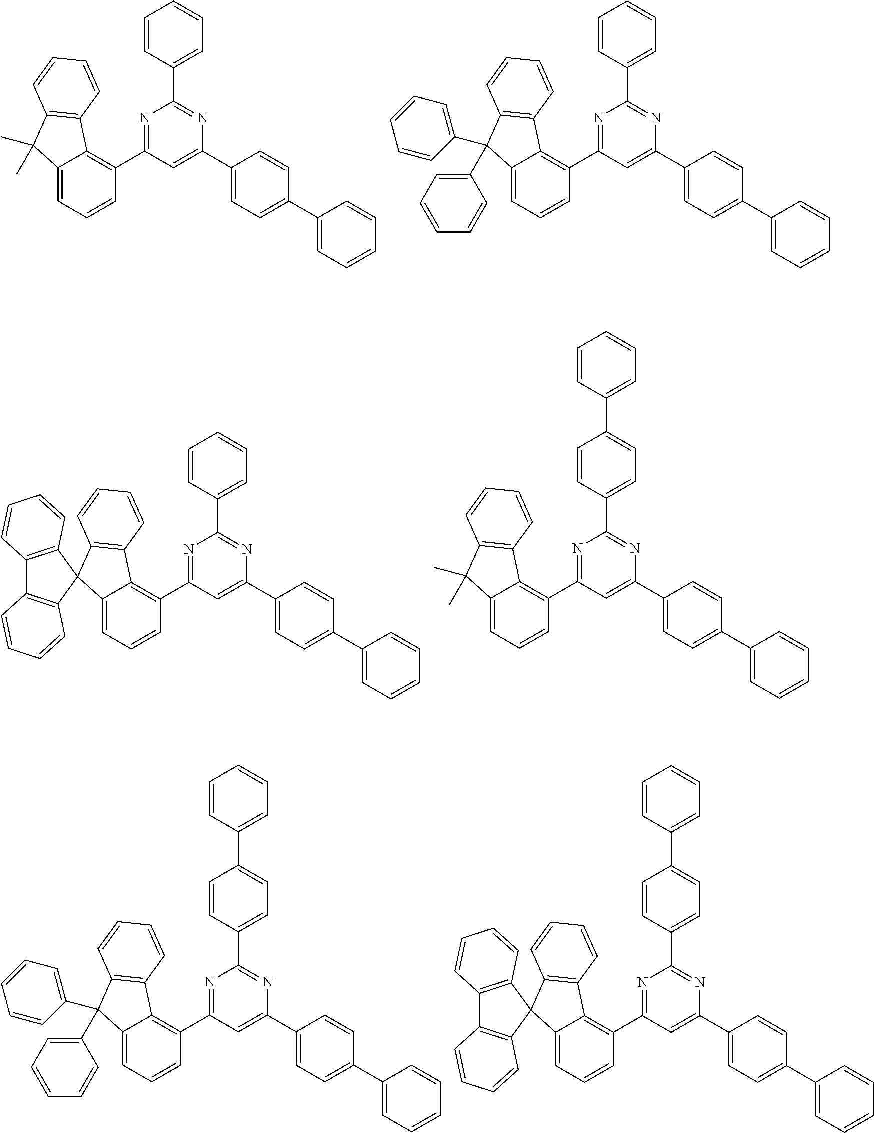

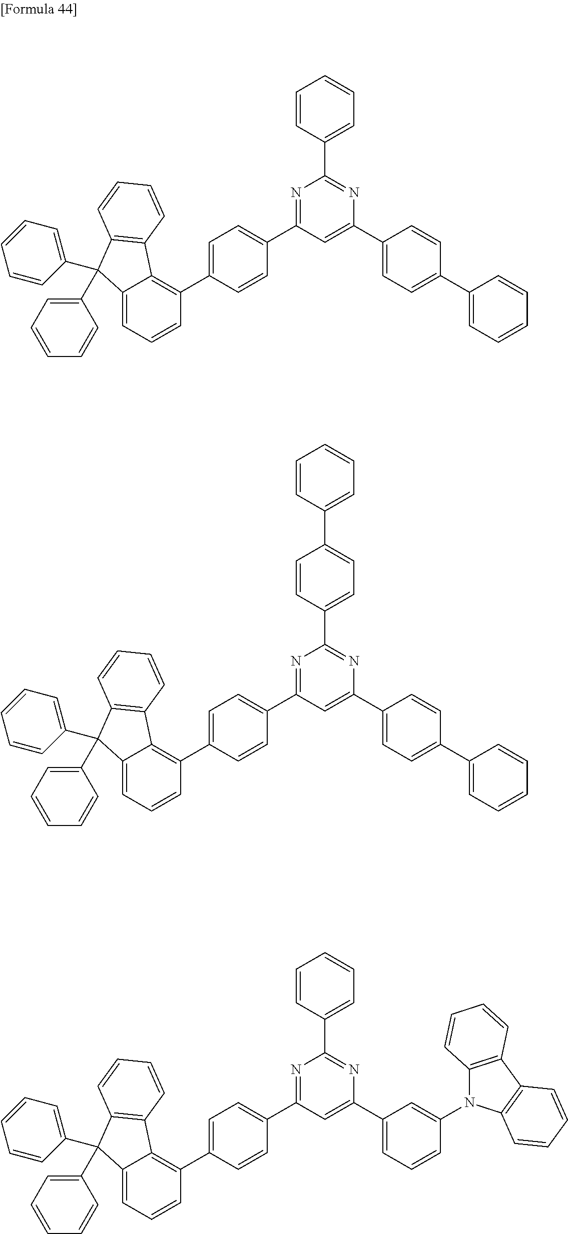 [SMILES: c1ccc(-c2ccc(-c3cc(-c4ccc(-c5cccc6c5-c5ccccc5C6(c5ccccc5)c5ccccc5)cc4)nc(-c4ccccc4)n3)cc2)cc1 ; c1ccc(-c2ccc(-c3cc(-c4ccc(-c5cccc6c5-c5ccccc5C6(c5ccccc5)c5ccccc5)cc4)nc(-c4ccc(-c5ccccc5)cc4)n3)cc2)cc1 ; c1ccc(-c2nc(-c3ccc(-c4cccc5c4-c4ccccc4C5(c4ccccc4)c4ccccc4)cc3)cc(-c3cccc(-n4c5ccccc5c5ccccc54)c3)n2)cc1]

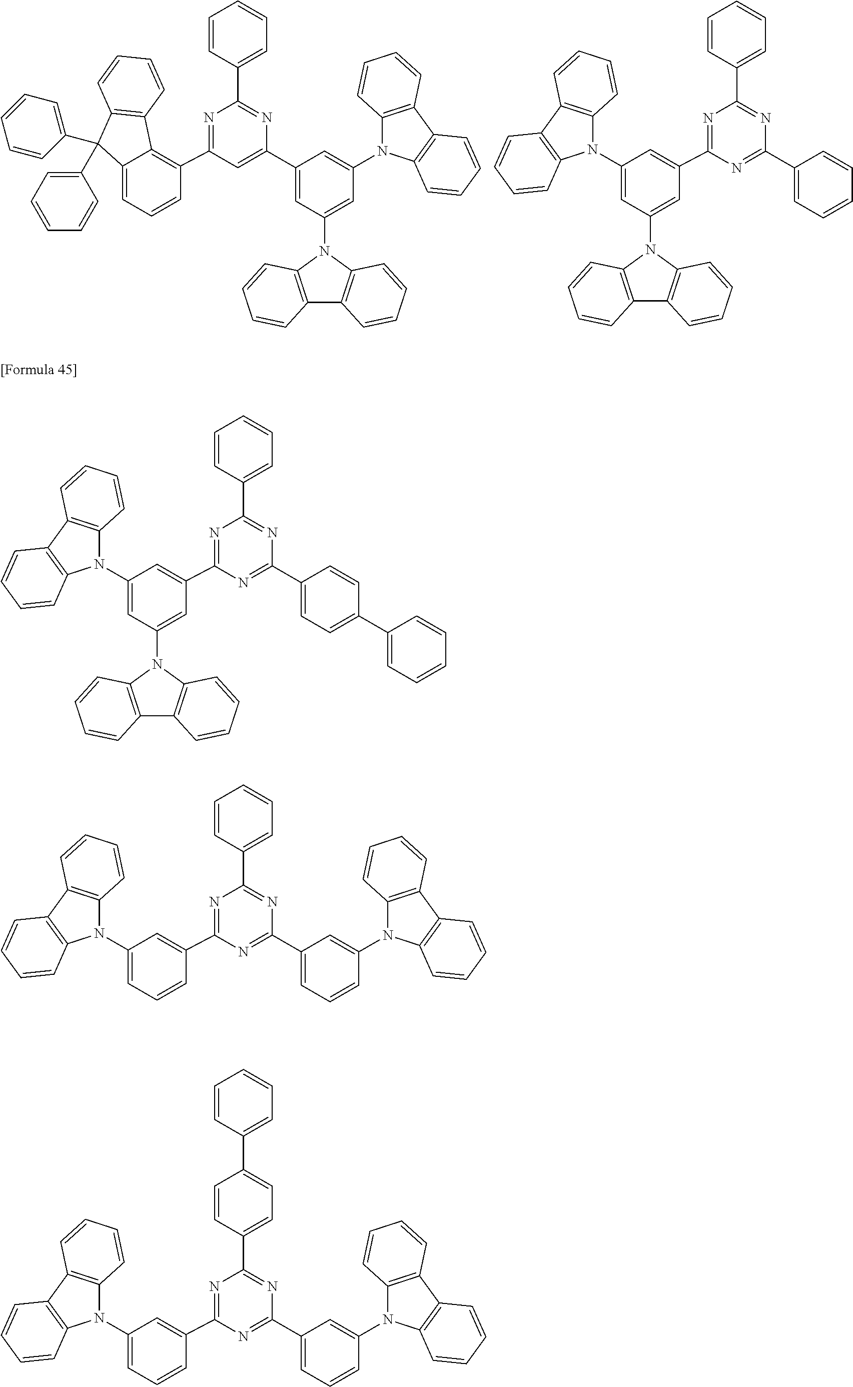

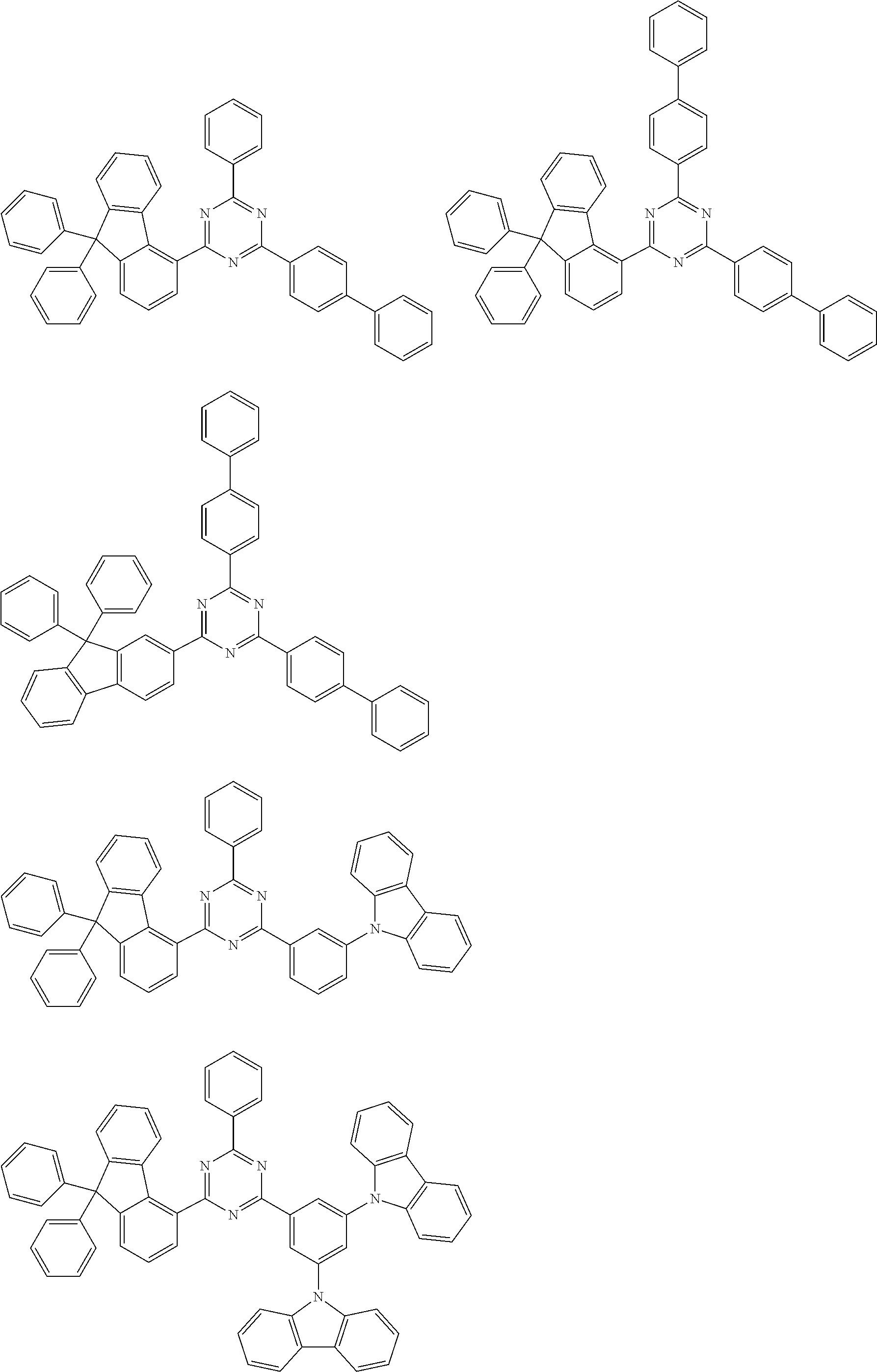 [SMILES: c1ccc(-c2ccc(-c3nc(-c4ccccc4)nc(-c4cccc5c4-c4ccccc4C5(c4ccccc4)c4ccccc4)n3)cc2)cc1 ; c1ccc(-c2ccc(-c3nc(-c4ccc(-c5ccccc5)cc4)nc(-c4cccc5c4-c4ccccc4C5(c4ccccc4)c4ccccc4)n3)cc2)cc1 ; c1ccc(-c2ccc(-c3nc(-c4ccc(-c5ccccc5)cc4)nc(-c4ccc5c(c4)C(c4ccccc4)(c4ccccc4)c4ccccc4-5)n3)cc2)cc1 ; c1ccc(-c2nc(-c3cccc(-n4c5ccccc5c5ccccc54)c3)nc(-c3cccc4c3-c3ccccc3C4(c3ccccc3)c3ccccc3)n2)cc1 ; c1ccc(-c2nc(-c3cc(-n4c5ccccc5c5ccccc54)cc(-n4c5ccccc5c5ccccc54)c3)nc(-c3cccc4c3-c3ccccc3C4(c3ccccc3)c3ccccc3)n2)cc1]

### Emitting Layer

The emitting layer 5 contains the first compound, the second compound, and the third compound.

### First Compound

The first compound is a fluorescent compound. The first compound may be a delayed fluorescent compound and a compound that does not exhibit delayed fluorescence.

In the exemplary embodiment, the first compound is a compound represented by a formula (1) below.

### Compound Represented by Formula (1)

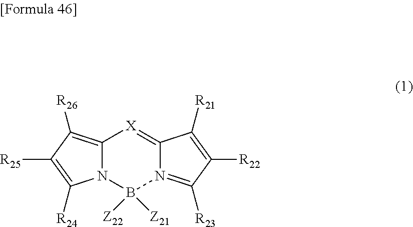

In the formula (1): X is a nitrogen atom, or a carbon atom bonded to Y;

Y is a hydrogen atom or a substituent;

R₂₁ to R₂₆ are each independently a hydrogen atom or a substituent, or at least one of a pair of R₂₁ and R₂₂, a pair of R₂₂ and R₂₃, a pair of R₂₄ and R₂₅, or a pair of R₂₅ and R₂₆ are mutually bonded to form a ring;

Y and R₂₁ to R₂₆ serving as the substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;

Z₂₁ and Z₂₂ are each independently a substituent, or are mutually bonded to form a ring; and

Z₂₁ and Z₂₂ serving as the substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

In the formula (1), for instance, when a pair of R₂₅ and R₂₆ are bonded to each other to form a ring, the first compound is represented by a formula (11) below.

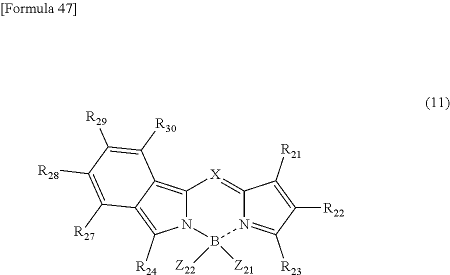

In the formula (11), X, Y, R₂₁ to R₂₄, Z₂₁, and Z₂₂ respectively represent the same as X, Y, R₂₁ to R₂₄, Z₂₁, and Z₂₂ in the formula (1), R₂₇ to R₃₀ are each independently a hydrogen atom or a substituent. When R₂₇ to R₃₀ are a substituent, the substituent represents the same as the examples of the substituent for R₂₁ to R₂₄.

In the formula (1), when Z₂₁ and Z₂₂ are bonded to each other to form a ring, the first compound is represented by, for instance, a formula (10A) or a formula (10B) below. It should be noted that a structure of the first compound is not limited to structures as follows.

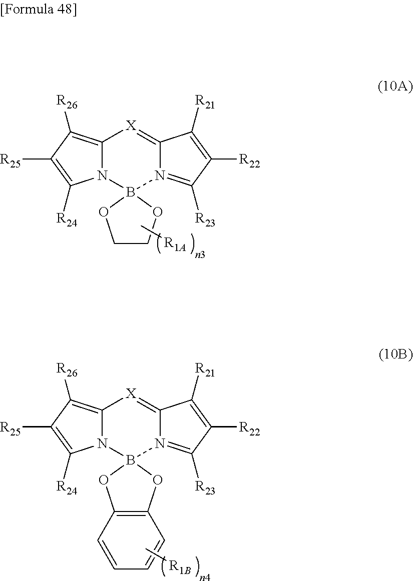

In the formula (10A), X, Y, and R₂₁ to R₂₆ respectively represent the same as X, Y, and R₂₁ to R₂₆ in the formula (1), R_{1A} is each independently a hydrogen atom or a substituent. When R_{1A} is a substituent, the substituent represents the same as the examples of the substituent for R₂₁ to R₂₆. n3 is 4.

In the formula (10B), X, Y, and R₂₁ to R₂₆ respectively represent the same as X, Y, and R₂₁ to R₂₆ in the formula (1), R_{1B} is each independently a hydrogen atom or a substituent. When R_{1B} is a substituent, the substituent represents the same as the examples of the substituent for R₂₁ to R₂₆. n4 is 4.

At least one of Z₂₁ or Z₂₂ (preferably Z₂₁ and Z₂₂) is preferably a group selected from the group consisting of a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

At least one of Z₂₁ or Z₂₂ is more preferably a group selected from the group consisting of an alkoxy group having 1 to 30 carbon atoms and substituted by a fluorine atom, an aryloxy group having 6 to 30 ring carbon atoms and substituted by a fluorine atom, and an aryloxy group having 6 to 30 ring carbon atoms and substituted by a fluoroalkyl group having 1 to 30 carbon atoms.

At least one of Z₂₁ or Z₂₂ is further preferably an alkoxy group having 1 to 30 carbon atoms and substituted by a fluorine atom. Z₂₁ and Z₂₂ are each still further preferably an alkoxy group having 1 to 30 carbon atoms and substituted by a fluorine atom.

It is also preferable that Z₂₁ and Z₂₂ are the same.

It is also preferable that at least one of Z₂₁ or Z₂₂ is a fluorine atom, and more preferable that Z₂₁ and Z₂₂ are fluorine atoms.

At least one of Z₂₁ or Z₂₂ is also preferably a group represented by a formula (10a).

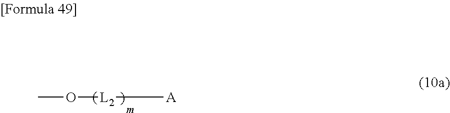

In the formula (10a), A is a substituted or unsubstituted alkyl group having 1 to 6 carbon atoms, a substituted or unsubstituted alkyl halide group having 1 to 6 carbon atoms, or a substituted or unsubstituted aryl group having 6 to 12 ring carbon atoms, L₂ is a substituted or unsubstituted alkylene group having 1 to 6 carbon atoms or a substituted or unsubstituted arylene group having 6 to 12 ring carbon atoms, m is 0, 1, 2, 3, 4, 5, 6, or 7. When m is 2, 3, 4, 5, 6, or 7, a plurality of L₂ are mutually the same or different. m is preferably 0, 1, or 2. When m is 0, A is directly bonded to O (an oxygen atom).

In the formula (1), when Z₂₁ and Z₂₂ are each the group represented by the formula (10a), the first compound is a compound represented by a formula (12) below.

The first compound is also preferably a compound represented by the formula (12) below.

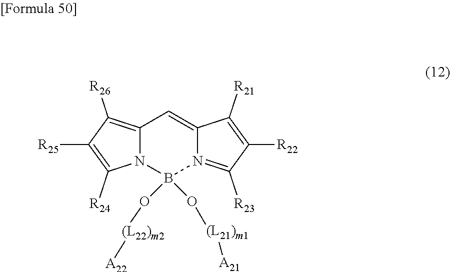

In the formula (12), X, Y bonded to X being a carbon atom, and R₂₁ to R₂₆ respectively represent the same as X, Y, and R₂₁ to R₂₆ in the formula (1). A₂₁ and A₂₂ represent the same as A in the formula (10a) and may be mutually the same or different. L₂₁ and L₂₂ represent the same as L₂ in the formula (10a) and may be mutually the same or different. m1 and m2 are each independently 0, 1, 2, 3, 4, 5, 6, or 7, preferably 0, 1, or 2. When m1 is 2, 3, 4, 5, 6, or 7, a plurality of L₂₁ are mutually the same or different. When m2 is 2, 3, 4, 5, 6, or 7, a plurality of L₂₂ are mutually the same or different. When m1 is 0, A₂₁ is directly bonded to O (an oxygen atom). When m2 is 0, A₂₂ is directly bonded to O (an oxygen atom).

At least one of A or L₂ in the formula (10a) is preferably substituted by a halogen atom, more preferably substituted by a fluorine atom.

A in the formula (10a) is more preferably a perfluoroalkyl group having 1 to 6 carbon atoms or a perfluoroaryl group having 6 to 12 ring carbon atoms, further preferably a perfluoroalkyl group having 1 to 6 carbon atoms.

L₂ in the formula (10a) is more preferably a perfluoroalkylene group having 1 to 6 carbon atoms or a perfluoroarylene group having 6 to 12 ring carbon atoms, further preferably a perfluoroalkylene group having 1 to 6 carbon atoms.

Specifically, the first compound is also preferably a compound represented by a formula (12a) below.

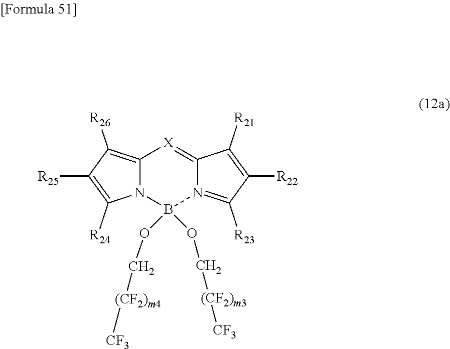

In the formula (12a):

X represents the same as X in the formula (1); Y bonded to X being a carbon atom represents the same as Y in the formula (1);

R₂₁ to R₂₆ each independently represent the same as R₂₁ to R₂₆ in the formula (1);

m3 is in a range from 0 to 4;

m4 is in a range from 0 to 4; and

m3 and m4 are mutually the same or different.

In the formulae (1), (11), (12), and (12a):

X is a carbon atom bonded to Y;

Y is a hydrogen atom or a substituent; and

Y serving as the substituent is preferably a substituent selected from the group consisting of a substituted or unsubstituted alkyl group having 1 to 30 carbon atoms, a substituted or unsubstituted alkyl halide group having 1 to 30 carbon atoms, and a substituted or unsubstituted aryl group having 6 to 30 ring carbon atoms, more preferably a substituted or unsubstituted aryl group having 6 to 30 ring carbon atoms.

In the formulae (1), (11), (12), and (12a):

it is more preferable that X is a carbon atom bonded to Y;

Y is a hydrogen atom or a substituent;

Y serving as the substituent is a substituted or unsubstituted aryl group having 6 to 30 ring carbon atoms; and

when Y as the substituent is an aryl group having 6 to 30 ring carbon atoms and having a substituent, the substituent is a substituted or unsubstituted alkyl group having 1 to 30 carbon atoms, a substituted or unsubstituted alkyl halide group having 1 to 30 carbon atoms, a substituted or unsubstituted alkoxy group having 1 to 30 carbon atoms, a substituted or unsubstituted alkoxy halide group having 1 to 30 carbon atoms, or an aryl group having 6 to 30 ring carbon atoms and substituted by an alkyl group having 1 to 30 carbon atoms.

In the first compound, although Z₂₁ and Z₂₂ may be bonded to each other to form a ring, it is preferable that Z₂₁ and Z₂₂ are not bonded to form no ring.

In the formulae (1), (12), and (12a), at least one of R₂₁, R₂₃, R₂₄, or R₂₆ is preferably a substituted or unsubstituted alkyl group having 1 to 30 carbon atoms or a substituted or unsubstituted alkyl halide group having 1 to 30 carbon atoms.

In the formulae (1), (12), and (12a), R₂₁, R₂₃, R₂₄, and R₂₆ are more preferably a substituted or unsubstituted alkyl group having 1 to 30 carbon atoms or a substituted or unsubstituted alkyl halide group having 1 to 30 carbon atoms. In this case, R₂₂ and R₂₅ are preferably hydrogen atoms.

In the formulae (1), (12), and (12a), at least one of R₂₁, R₂₃, R₂₄, or R₂₆ is preferably a substituted or unsubstituted aryl group having 6 to 30 ring carbon atoms.

In the formulae (1), (12), and (12a), R₂₁, R₂₃, R₂₄, and R₂₆ are more preferably a substituted or unsubstituted aryl group having 6 to 30 ring carbon atoms. In this case, R₂₂ and R₂₅ are preferably hydrogen atoms.

In the formulae (1), (12), and (12a):

it is more preferable that R₂₁, R₂₃, R₂₄, and R₂₆ are each independently a substituted or unsubstituted alkyl group having 1 to 30 carbon atoms (preferably 1 to 6 carbon atoms), a substituted or unsubstituted alkyl halide group having 1 to 30 carbon atoms (preferably 1 to 6 carbon atoms), or an aryl group having 6 to 30 ring carbon atoms (preferably 6 to 12 ring carbon atoms) substituted by an alkyl group having 1 to 30 carbon atoms; and

R₂₂ and R₂₅ are hydrogen atoms.

In the formula (11), at least one of R₂₁, R₂₃, or R₂₄ is preferably a substituted or unsubstituted alkyl group having 1 to 30 carbon atoms or a substituted or unsubstituted alkyl halide group having 1 to 30 carbon atoms.

In the formula (11), R₂₁, R₂₃, and R₂₄ are more preferably a substituted or unsubstituted alkyl group having 1 to 30 carbon atoms or a substituted or unsubstituted alkyl halide group having 1 to 30 carbon atoms. In this case, R₂₂ is preferably a hydrogen atom.

In the formula (11), at least one of R₂₁, R₂₃, or R₂₄ is preferably a substituted or unsubstituted aryl group having 6 to 30 ring carbon atoms.

In the formula (11), R₂₁, R₂₃, and R₂₄ are more preferably a substituted or unsubstituted aryl group having 6 to 30 ring carbon atoms. In this case, R₂₂ is preferably a hydrogen atom.

In the formula (11):

it is more preferable that R₂₁, R₂₃, and R₂₄ are each independently a substituted or unsubstituted alkyl group having 1 to 30 carbon atoms (preferably 1 to 6 carbon atoms), a substituted or unsubstituted alkyl halide group having 1 to 30 carbon atoms (preferably 1 to 6 carbon atoms), or an aryl group having 6 to 30 ring carbon atoms (preferably 6 to 12 ring carbon atoms) substituted by an alkyl group having 1 to 30 carbon atoms; and

R₂₂ is a hydrogen atom.

In an exemplary embodiment, the compound represented by the formula (1) is preferably a compound represented by a formula (n) below.

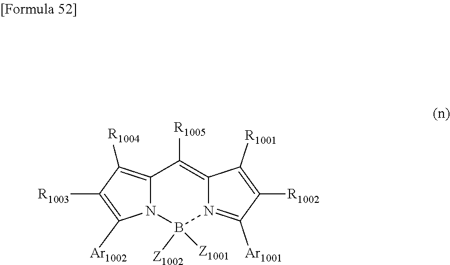

In the formula (n):

Ar₁₀₀₁ and Ar₁₀₀₂ are each independently selected from the group consisting of a substituted or unsubstituted aryl group having 6 to 30 ring carbon atoms, and a substituted or unsubstituted heteroaryl group having 5 to 30 ring atoms;

R₁₀₀₁ to R₁₀₀₅ are each independently a hydrogen atom or a substituent, or at least one pair of a pair of R₁₀₀₁ and R₁₀₀₂, a pair of R₁₀₀₂ and Ar₁₀₀₁, a pair of Ar₁₀₀₂ and R₁₀₀₃, or a pair of R₁₀₀₃ and R₁₀₀₄ are bonded to each other to form a ring;

R₁₀₀₁ to R₁₀₀₅ serving as the substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group; and

Z₁₀₀₁ and Z₁₀₀₂ are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

In the formula (n), Ar₁₀₀₁ and Ar₁₀₀₂ are preferably each independently a substituted or unsubstituted aryl group having 6 to 30 ring carbon atoms. Ar₁₀₀₁ and Ar₁₀₀₂ may be each independently a monocyclic ring or a fused ring. Examples of Ar₁₀₀₁ and Ar₁₀₀₂ include a substituted or unsubstituted phenyl group and a substituted or unsubstituted naphthyl group.

In the formula (n), it is preferable that at least one of R₁₀₀₁ or R₁₀₀₄ is a substituted or unsubstituted aryl group having 6 to 30 ring carbon atoms or a substituted or unsubstituted heteroaryl group having 5 to 30 ring atoms, and it is more preferable that both of R₁₀₀₁ and R₁₀₀₄ are each independently a substituted or unsubstituted aryl group having 6 to 30 ring carbon atoms or a substituted or unsubstituted heteroaryl group having 5 to 30 ring atom.

In the formula (n), R₁₀₀₂ and R₁₀₀₃ are preferably a hydrogen atom, a substituted or unsubstituted alkyl group having 1 to 30 carbon atoms, a substituted or unsubstituted cycloalkyl group having 3 to 30 ring carbon atoms, a substituted or unsubstituted aryl group having 6 to 30 ring carbon atoms, or a substituted or unsubstituted heteroaryl group having 5 to 30 ring atoms, more preferably a hydrogen atom or a substituted or unsubstituted alkyl group having 1 to 30 carbon atoms.

In the formula (n), R₁₀₀₅ is preferably a substituted or unsubstituted aryl group having 6 to 30 ring carbon atoms or a substituted or unsubstituted heteroaryl group having 5 to 30 ring atoms. Examples of R₁₀₀₅ include a substituted or unsubstituted phenyl group, a substituted or unsubstituted naphthyl group, a substituted or unsubstituted phenanthryl group, a substituted or unsubstituted anthryl group, and a substituted or unsubstituted dibenzofuranyl group.

In the formula (n), Z₁₀₀₁ and Z₁₀₀₂ are preferably each independently a halogen atom, a substituted or unsubstituted alkyl group having 1 to 30 carbon atoms, a substituted or unsubstituted aryl group having 6 to 30 ring carbon atoms, a substituted or unsubstituted alkoxy group having 1 to 30 carbon atoms, or a substituted or unsubstituted alkoxy halide group having 1 to 30 carbon atoms.

In the formula (n), when at least one of a pair of R₁₀₀₂ and Ar₁₀₀₁ or a pair of A₁₀₀₂ and R₁₀₀₃ is bonded to each other to form a ring, the first compound is preferably a compound represented by, for instance, a formula (n+1A) or a formula (n+1B) below.

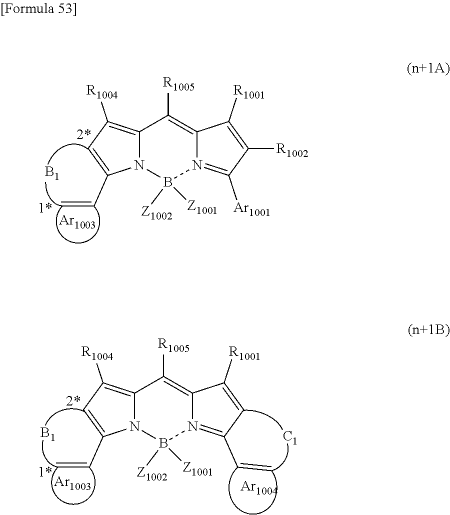

In the formula (n+1A), R₁₀₀₁, R₁₀₀₂, R₁₀₀₄, R₁₀₀₅, Ar₁₀₀₁, Z₁₀₀₁ and Z₁₀₀₂ each independently represent the same as R₁₀₀₁, R₁₀₀₂, R₁₀₀₄, R₁₀₀₅, Ar₁₀₀₁, Z₁₀₀₁ and Z₁₀₀₂ in the formula (n).

In the formula (n+1B), R₁₀₀₁, R₁₀₀₄, R₁₀₀₅, Z₁₀₀₁ and Z₁₀₀₂ each independently represent the same as R₁₀₀₁, R₁₀₀₄, R₁₀₀₅, Z₁₀₀₁ and Z₁₀₀₂ in the formula (n).

Ar₁₀₀₃ and Ar₁₀₀₄ are each independently selected from the group consisting of a substituted or unsubstituted aromatic hydrocarbon ring having 6 to 30 ring carbon atoms, and a substituted or unsubstituted aromatic heterocyclic ring having 5 to 30 ring atoms.

B¹ is a bridging structure in which three or more atoms are bonded in series, the atoms being selected from the group consisting of a substituted or unsubstituted carbon atom, a substituted or unsubstituted silicon atom, a substituted or unsubstituted nitrogen atom, a substituted or unsubstituted phosphorus atom, an oxygen atom, and a sulfur atom.

C¹ is a bridging structure in which one or more atoms are bonded in series, the atoms being selected from the group consisting of a substituted or unsubstituted carbon atom, a substituted or unsubstituted silicon atom, a substituted or unsubstituted nitrogen atom, a substituted or unsubstituted phosphorus atom, an oxygen atom, and a sulfur atom.

When B¹ is a trimethylene group, R₁₀₀₄ is neither a hydrogen atom nor a halogen atom.

Here, a double bond shown as a part of Ar₁₀₀₃ in the formula (n+1A) and the formula (n+1B) shows a part of an aromatic hydrocarbon ring or an aromatic heterocyclic ring to show that a carbon atom directly bonded to a pyrromethene skeleton is adjacent to a carbon atom bonded to the bridging structure B¹.

Similarly, a double bond shown as a part of Ar₁₀₀₄ in the formula (n+1A) and the formula (n+1B) shows a part of an aromatic hydrocarbon ring or an aromatic heterocyclic ring to show that a carbon atom directly bonded to a pyrromethene skeleton is adjacent to a carbon atom bonded to the bridging structure C¹.

The number of atoms forming a ring formed by mutually bonding a pair of R₁₀₀₂ and Ar₁₀₀₁ and the number of atoms forming a ring formed by mutually bonding a pair of Ar₁₀₀₂ and R₁₀₀₃ are preferably 30 or less.

Specifically, in the formula (n+1A) and the formula (n+11B), a total of the number of atoms in the bridging structure B¹ (i.e., the number of atoms bonded in series), the number of atoms forming a ring in Ar₁₀₀₃, and the number of carbon atoms (i.e., two carbon atoms) forming a pyrromethene skeleton is preferably 30 or less.

In the formula (n+1B), a total of the number of atoms in the bridging structure C¹ (i.e., the number of atoms bonded in series), the number of atoms forming a ring in Ar₁₀₀₄, and the number of carbon atoms (i.e., two carbon atoms) forming a pyrromethene skeleton is preferably 30 or less.

In the formula (n+1A) and the formula (n+1B), B¹ is preferably a bridging structure represented by a formula (n+2A) or a formula (n+2B).

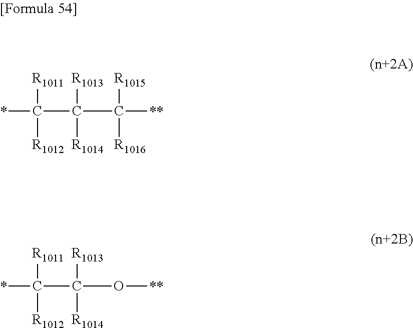

In the formula (n+2A), R₁₀₁₁ to R₁₀₁₆ are each independently a hydrogen atom or a substituent, or at least one pair of pairs of adjacent two or more of R₁₀₁₁ to R₁₀₁₆ are bonded to each other to form a ring.

In the formula (n+2B), R₁₀₁₁ to R₁₀₁₄ are each independently a hydrogen atom or a substituent, or at least one pair of pairs of adjacent two or more of R₁₀₁₁ to R₁₀₁₄ are bonded to each other to form a ring.

R₁₀₁₁ to R₁₀₁₆ serving as the substituent are each independently a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted heteroaryl group having 5 to 30 ring atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted alkynyl group having 2 to 30 carbon atoms, a halogen atom, a carboxy group, a substituted or unsubstituted amino group, a nitro group, a cyano group, a substituted or unsubstituted silyl group, a hydroxy group, an ester group, a siloxanyl group, or a carbamoyl group.

* represents a bonding moiety to a pyrrole ring and ** represents a bonding moiety to Ar₁₀₀₃ in the formula (n+1A) and the formula (n+1B).

* representing the bonding moiety to the pyrrole ring corresponds to 2* in the formula (n+1A) and the formula (n+1B). ** representing the bonding moiety to Ar₁₀₀₃ corresponds to 1* in the formula (n+1A) and the formula (n+1B).

In the formula (n+2A) and the formula (n+2B), R₁₀₁₁ to R₁₀₁₆ are preferably each independently a hydrogen atom or a substituted or unsubstituted alkyl group having 1 to 30 carbon atoms.

B¹ (a bridging structure in which three or more atoms are bonded in series)

In the formula (n+1A) and the formula (n+1B), B¹ is preferably a bridging structure in which three atoms are bonded in series.

C¹ (a Bridging Structure in which One or More Atoms are Bonded in Series)

In the formula (n+1B), C¹ is preferably a bridging structure in which one to three atoms are bonded in series.

Atoms forming C¹ are preferably selected from a substituted or unsubstituted carbon atom, an oxygen atom, and a sulfur atom, more preferably a substituted or unsubstituted carbon atom.

In the formula (n), R₁₀₀₅ is preferably a group represented by a formula (n+3).

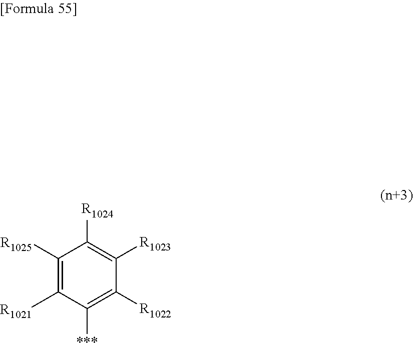

In the formula (n+3):

R₁₀₂₁ and R₁₀₂₂ are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, and a substituted or unsubstituted heteroaryl group having 5 to 30 ring atoms;

R₁₀₂₃ to R₁₀₂₅ are each independently a hydrogen atom or a substituent, or at least one pair of a pair of R₁₀₂₃ and R₁₀₂₄, or a pair of R₁₀₂₄ and R₁₀₂₅ are bonded to each other to form a ring; and

R₁₀₂₃ to R₁₀₂₅ serving as the substituent are each independently a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted heteroaryl group having 5 to 30 ring atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsulfonyl group having 1 to 30 carbon atoms, a substituted or unsubstituted arylcarbonyl group having 6 to 30 ring carbon atoms, a substituted or unsubstituted acyl group having 1 to 30 carbon atoms, a halogen atom, a carboxy group, a substituted or unsubstituted amino group, a nitro group, a cyano group, a substituted or unsubstituted silyl group, a hydroxy group, an ester group, a siloxanyl group, or a carbamoyl group.

In the formula (n+3), *** represents a bonding position to a carbon atom bonded to R₁₀₀₅ in the formula (n).

In the formula (n+3), R₁₀₂₁ and R₁₀₂₂ are preferably each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atom. When R₁₀₂₁ and R₁₀₂₂ are each an alkyl group, R₁₀₂₁ and R₁₀₂₂ are more preferably a substituted or unsubstituted alkyl group having 1 to 4 carbon atoms, further preferably a methyl group. When R₁₀₂₁ and R₁₀₂₂ are each an aryl group, R₁₀₂₁ and R₁₀₂₂ are more preferably a substituted or unsubstituted phenyl group. When R₁₀₂₁ and R₁₀₂₂ are each a heteroaryl group, R₁₀₂₁ and R₁₀₂₂ are more preferably a substituted or unsubstituted monocyclic heteroaryl group having 5 to 6 ring atoms.

In the formula (n+3), R₁₀₂₃ to R₁₀₂₅ are more preferably each independently a hydrogen atom, a substituted or unsubstituted alkyl group having 1 to 30 carbon atoms, a substituted or unsubstituted alkyl halide group having 1 to 30 carbon atoms, a substituted or unsubstituted alkoxy group having 1 to 30 carbon atoms, a substituted or unsubstituted aryl group having 6 to 30 ring carbon atoms, a halogen atom, a substituted or unsubstituted amino group, or a cyano group.

In the formula (n), the formula (n+1A), the formula (n+1B), and the formula (n+3), a substituent “for the substituted or unsubstituted” group in each of the formulae is preferably a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted heteroaryl group having 5 to 30 ring atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted alkynyl group having 2 to 30 carbon atoms, a substituted or unsubstituted aralkyl group having 7 to 30 carbon atoms, a substituted or unsubstituted alkylsulfonyl group having 1 to 30 carbon atoms, a substituted or unsubstituted arylcarbonyl group having 6 to 30 ring carbon atoms, a substituted or unsubstituted acyl group having 1 to 30 carbon atoms, a halogen atom, a carboxy group, a substituted or unsubstituted amino group, a nitro group, cyano group, a substituted or unsubstituted silyl group, a substituted phosphoryl group, a hydroxy group, a substituted phosphino group, an ester group, a siloxanyl group, or a carbamoyl group.

In the formula (n), the formula (n+1A), the formula (n+1B), and the formula (n+3), a substituent “for the substituted or unsubstituted” group in each of the formulae is more preferably an unsubstituted alkyl group having 1 to 30 carbon atoms, an unsubstituted alkyl halide group having 1 to 30 carbon atoms, an unsubstituted cycloalkyl group having 3 to 30 ring carbon atoms, an unsubstituted aryl group having 6 to 30 ring carbon atoms, an unsubstituted heteroaryl group having 5 to 30 ring atoms, an unsubstituted alkoxy group having 1 to 30 carbon atoms, an unsubstituted alkoxy halide group having 1 to 30 carbon atoms, an unsubstituted alkylthio group having 1 to 30 carbon atoms, an unsubstituted aryloxy group having 6 to 30 ring carbon atoms, an unsubstituted arylthio group having 6 to 30 ring carbon atoms, an unsubstituted alkenyl group having 2 to 30 carbon atoms, an unsubstituted alkynyl group having 2 to 30 carbon atoms, an unsubstituted aralkyl group having 7 to 30 carbon atoms, an unsubstituted alkylsulfonyl group having 1 to 30 carbon atoms, an unsubstituted arylcarbonyl group having 6 to 30 ring carbon atoms, an unsubstituted acyl group having 1 to 30 carbon atoms, a halogen atom, a carboxy group, a substituted or unsubstituted amino group, a nitro group, cyano group, a substituted or unsubstituted silyl group, a substituted phosphoryl group, a hydroxy group, a substituted phosphino group, an ester group, a siloxanyl group, or a carbamoyl group.

In the first compound, examples of an alkoxy group substituted by a fluorine atom include 2,2,2-trifluoroethoxy group, 2,2-difluoroethoxy group, 2,2,3,3,3-pentafluoro1-propoxy group, 2,2,3,3-tetrafluoro1-propoxy group, 1,1,1,3,3,3-hexafluoro2-propoxy group, 2,2,3,3,4,4,4-heptafluoro1-butyloxy group, 2,2,3,3,4,4-hexafluoro1-butyloxy group, nonafluoro-tertiary-butyloxy group, 2,2,3,3,4,4,5,5,5-nonafluoropentanoxy group, 2,2,3,3,4,4,5,5,6,6,6-undecafluorohexanoxy group, 2,3-bis(trifluoromethyl)-2,3-butanedioxy group, 1,1,2,2-tetra(trifluoromethyl)ethylene glycoxy group, 4,4,5,5,6,6,6-heptafluorohexane-1,2-dioxy group, and 4,4,5,5,6,6,7,7,8,8,9,9,9-tridecafluorononane-1,2-dioxy group.

In the first compound, examples of an aryloxy group substituted by an fluorine atom or an aryloxy group substituted by a fluoroalkyl group include pentafluoro phenoxy group, 3,4,5-trifluorophenoxy group, 4-trifluoromethylphenoxy group, 3,5-bistrifluoromethylphenoxy group, 3-fluoro-4-trifluoromethylphenoxy group, 2,3,5,6-tetrafluoro-4-trifluoromethylphenoxy group, 4-fluorocatecholato group, 4-trifluoromethylcatecholato group, and 3,5-bistrifluoromethylcatecholato group.

When the first compound is a fluorescent compound, the first compound preferably emits light whose main peak wavelength is in a range from 400 nm to 700 nm.

Herein, the main peak wavelength means a peak wavelength of a fluorescence spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 manufactured by Hitachi High-Tech Science Corporation) is used as a measurement device.

The first compound preferably emits red light or green light.

Herein, the red light emission refers to light emission whose main peak wavelength of fluorescence spectrum is in a range from 600 nm to 660 nm.

When the first compound is a red fluorescent compound, the main peak wavelength of the first compound is preferably in a range from 600 nm to 660 nm, more preferably in a range from 600 nm to 640 nm, further preferably in a range from 610 nm to 630 nm.

Herein, the green light emission refers to light emission whose main peak wavelength of fluorescence spectrum is in a range from 500 nm to 560 nm.

When the first compound is a green fluorescent compound, the main peak wavelength of the first compound is preferably in a range from 500 nm to 560 nm, more preferably in a range from 500 nm to 540 nm, further preferably in a range from 510 nm to 530 nm.

Herein, the blue light emission refers to a light emission in which a main peak wavelength of fluorescence spectrum is in a range from 430 nm to 480 nm.

When the first compound is a blue fluorescent compound, the main peak wavelength of the first compound is preferably in a range from 430 nm to 480 nm, more preferably in a range from 445 nm to 480 nm.

### Method of Preparing First Compound

The first compound can be prepared by any known method.

Specific examples of the first compound (the compound represented by the formula (1)) according to the exemplary embodiment are shown below. It should be noted that the first compound of the invention is not limited to the specific examples.

A coordinate bond between a boron atom and a nitrogen atom in a pyrromethene skeleton is shown by various means such as a solid line, a broken line, an arrow, and omission. Herein, the coordinate bond is shown by a solid line or a broken line, or the description of the coordinate bond is omitted. Me represents a methyl group.

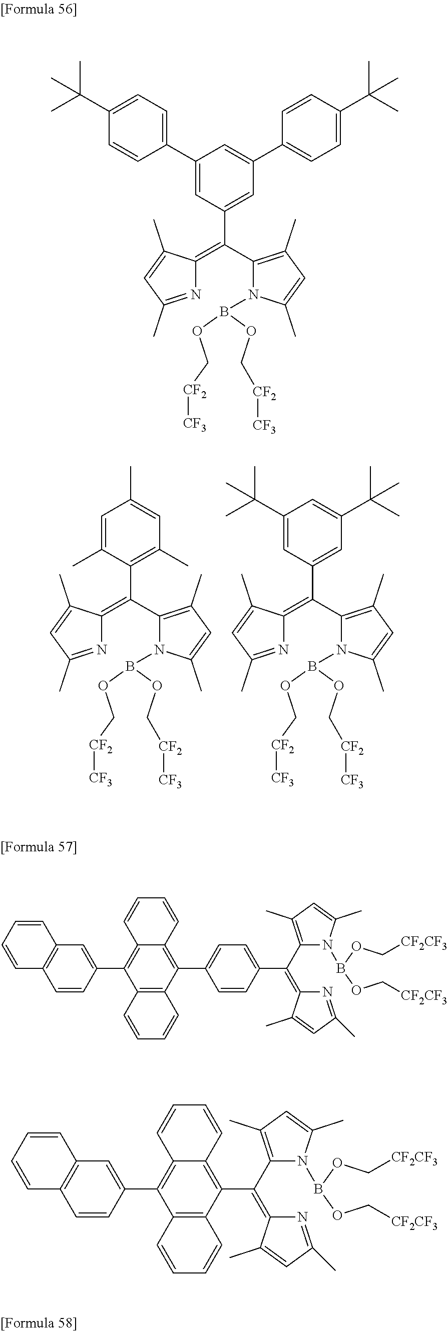

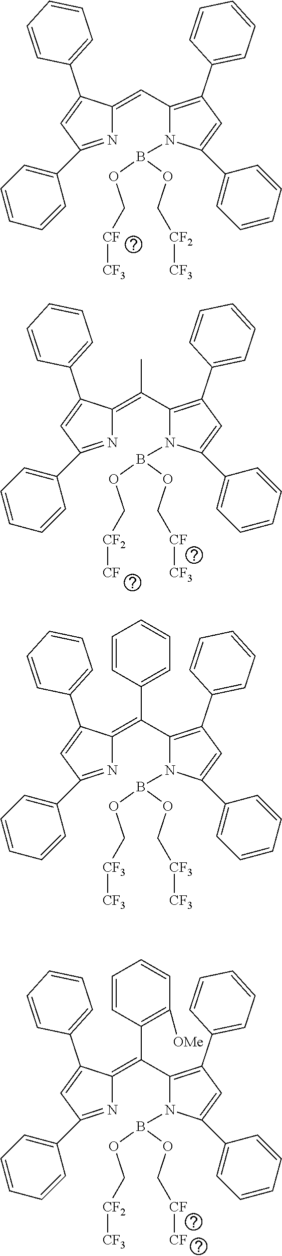

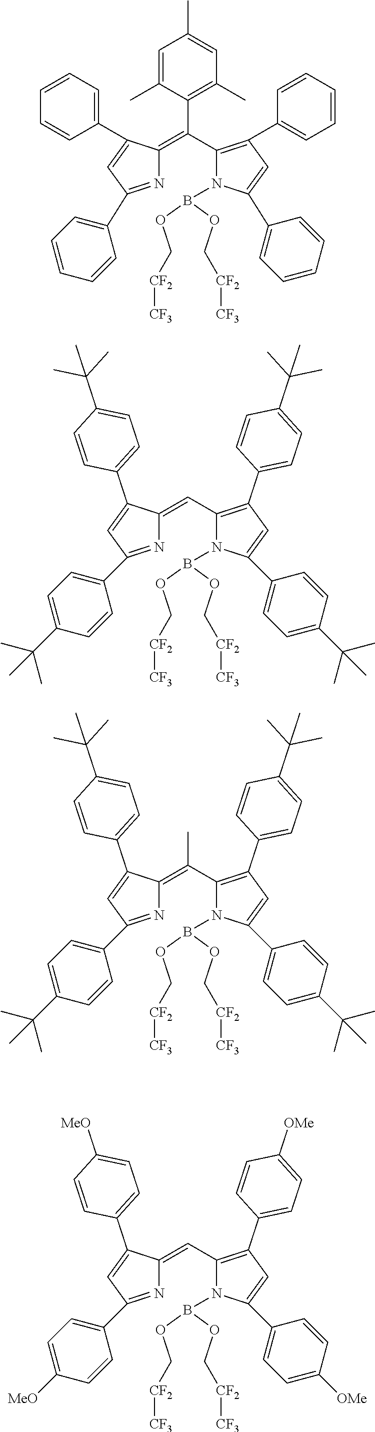

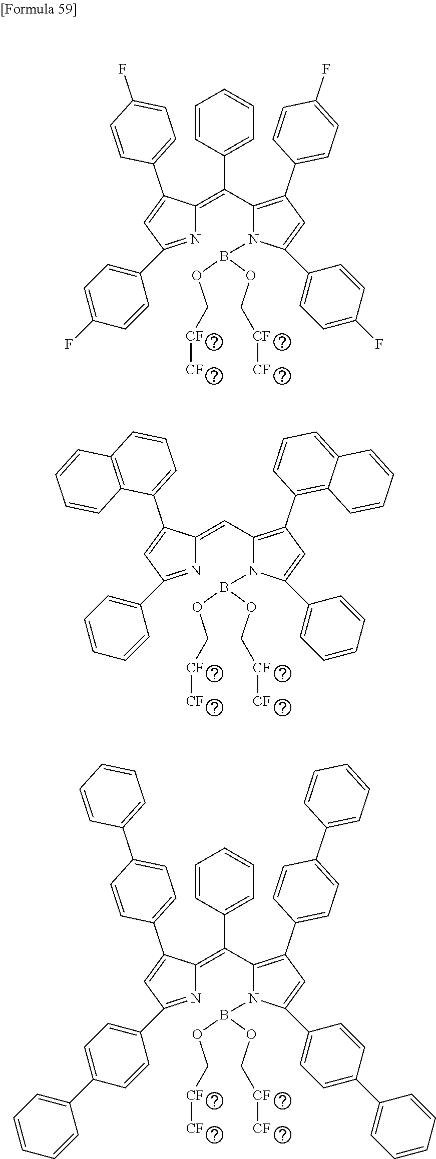

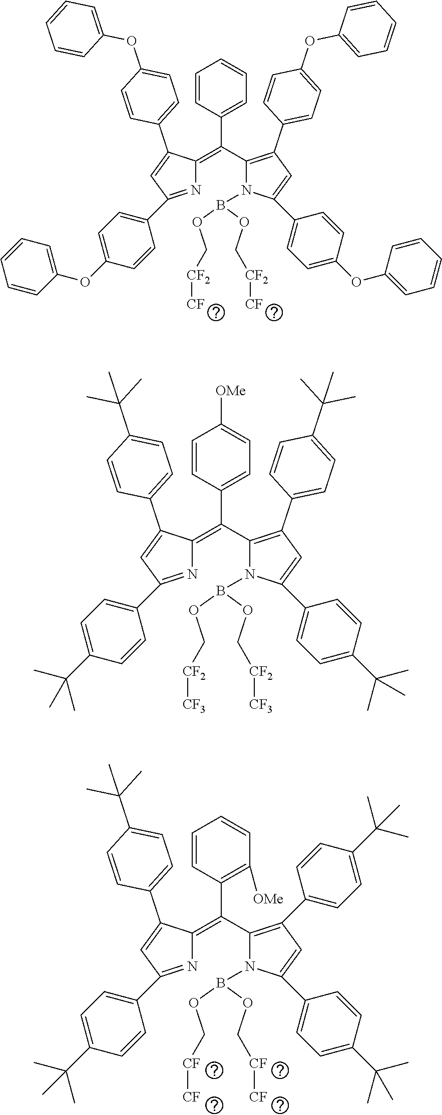

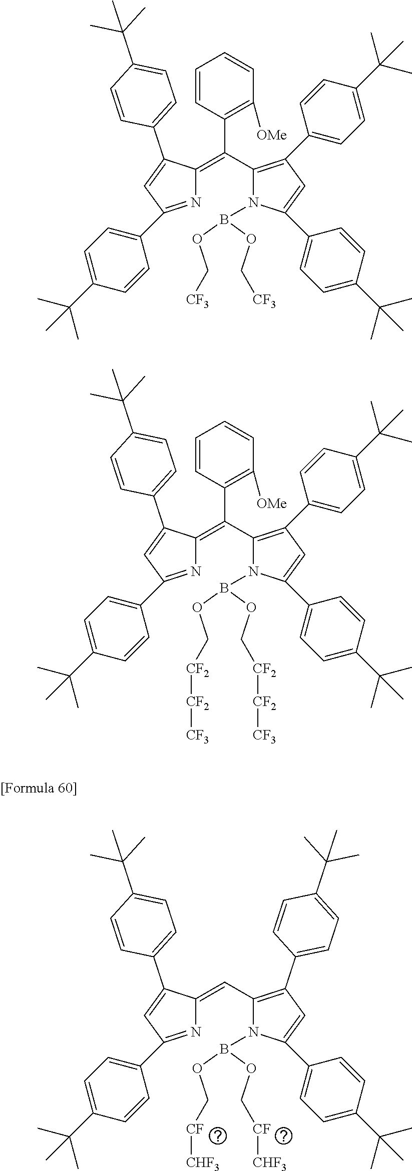

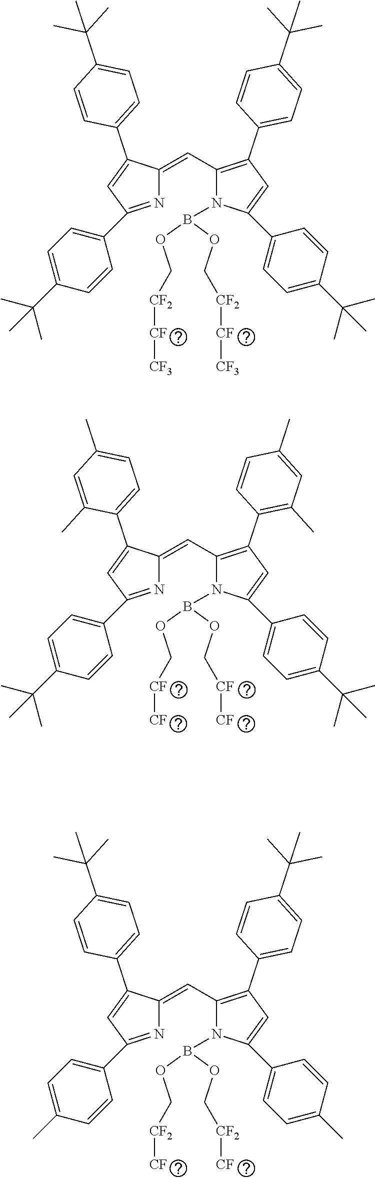

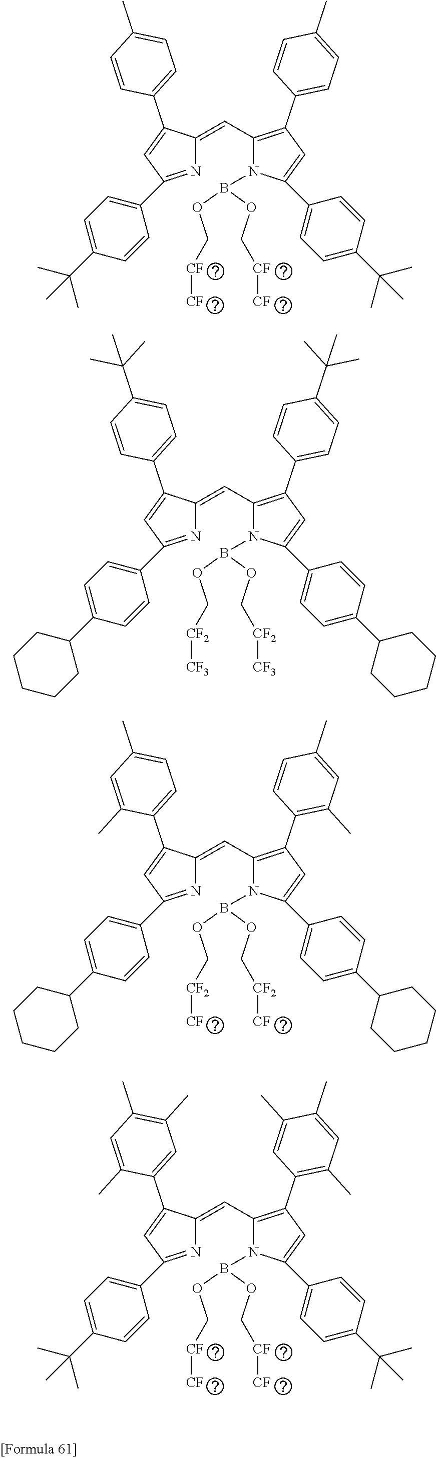

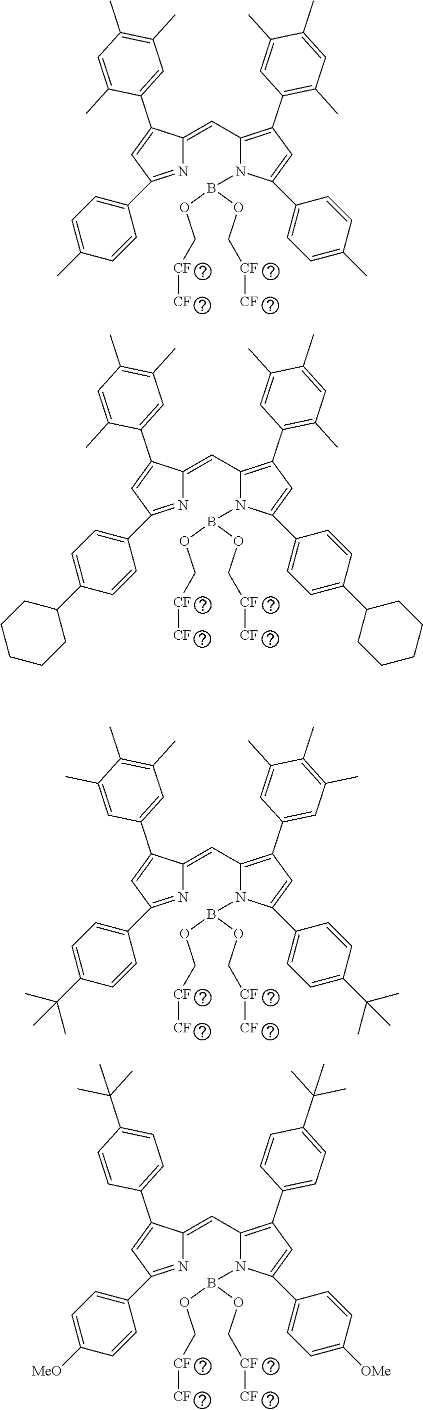

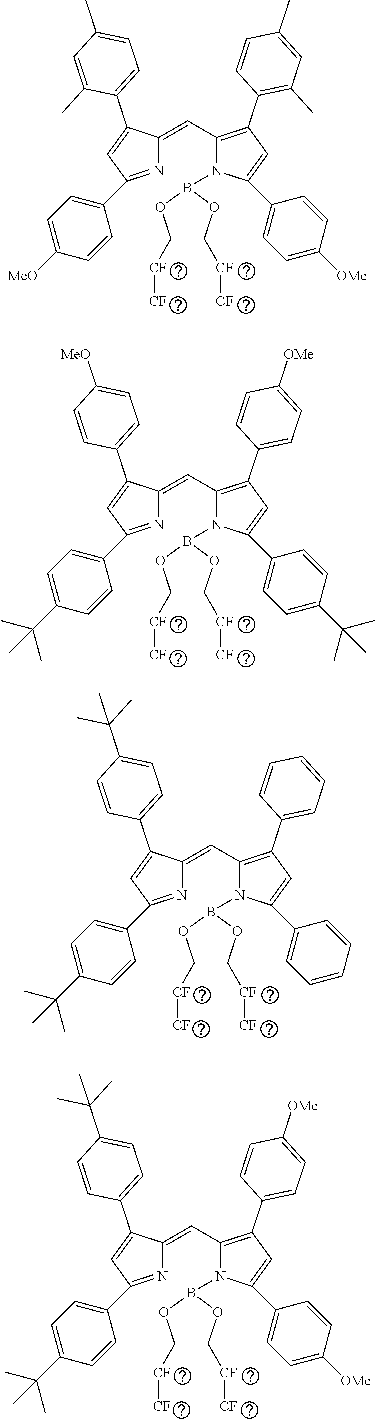

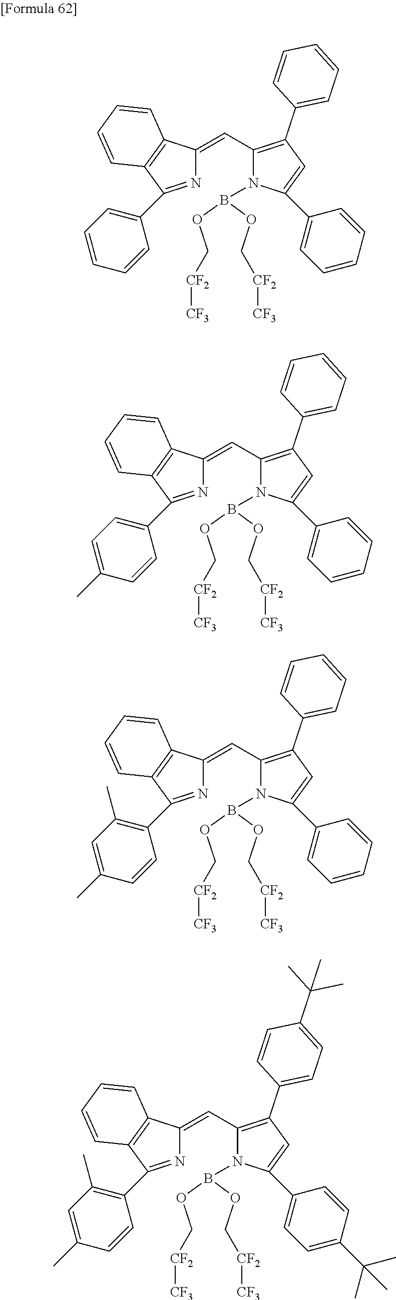 [SMILES: FC(F)(F)C(F)(F)COB(OCC(F)(F)C(F)(F)F)n1c(-c2ccccc2)cc(-c2ccccc2)c1/C=C1\N=C(c2ccccc2)c2ccccc21 ; Cc1ccc(C2=N/C(=C\c3c(-c4ccccc4)cc(-c4ccccc4)n3B(OCC(F)(F)C(F)(F)F)OCC(F)(F)C(F)(F)F)c3ccccc32)cc1 ; Cc1ccc(C2=N/C(=C\c3c(-c4ccccc4)cc(-c4ccccc4)n3B(OCC(F)(F)C(F)(F)F)OCC(F)(F)C(F)(F)F)c3ccccc32)c(C)c1 ; Cc1ccc(C2=N/C(=C\c3c(-c4ccc(C(C)(C)C)cc4)cc(-c4ccc(C(C)(C)C)cc4)n3B(OCC(F)(F)C(F)(F)F)OCC(F)(F)C(F)(F)F)c3ccccc32)c(C)c1]

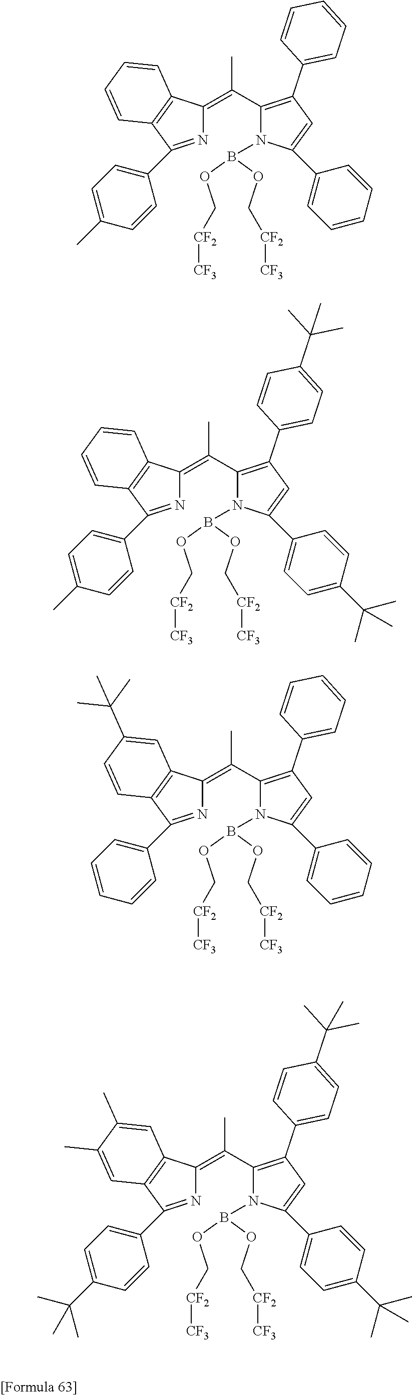

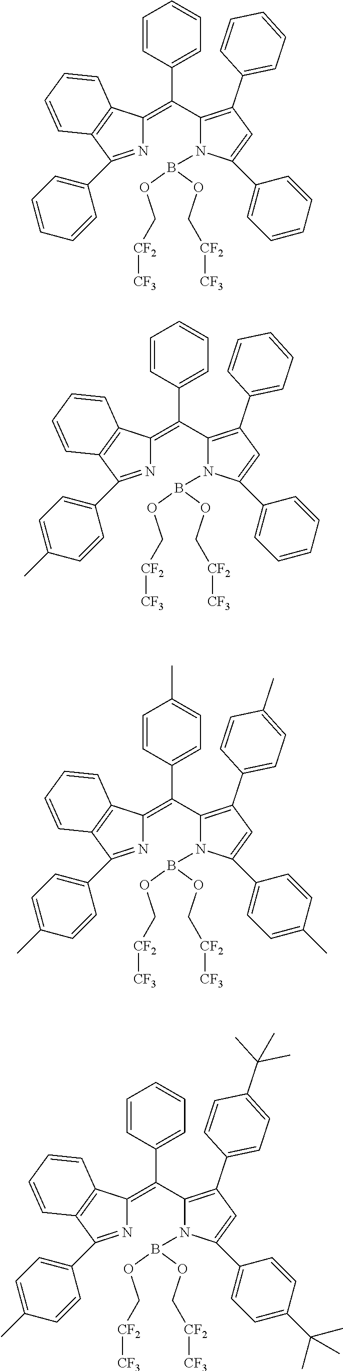

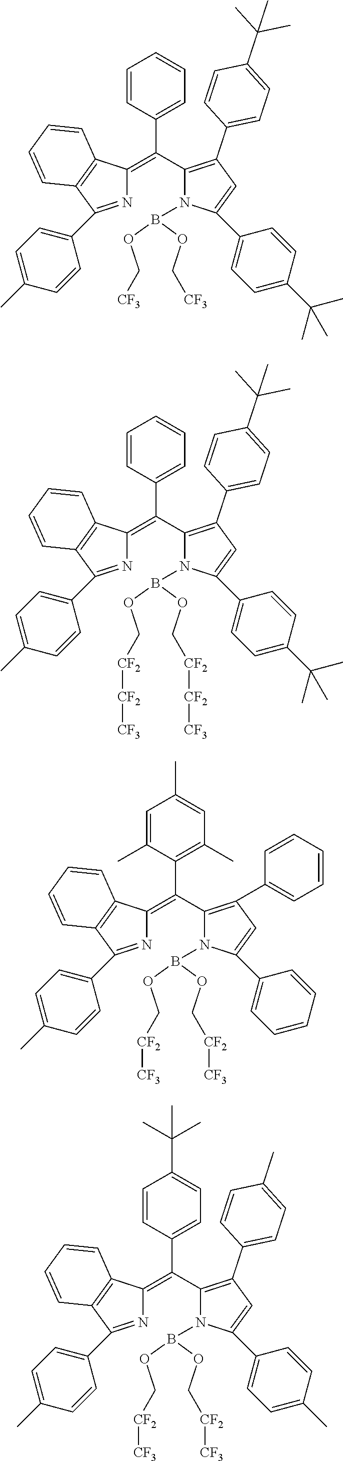

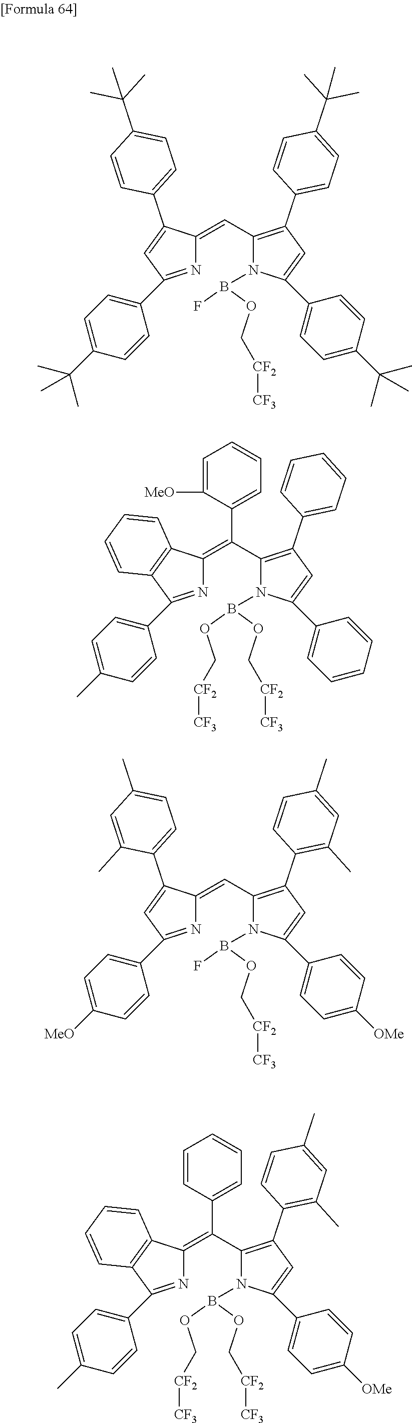

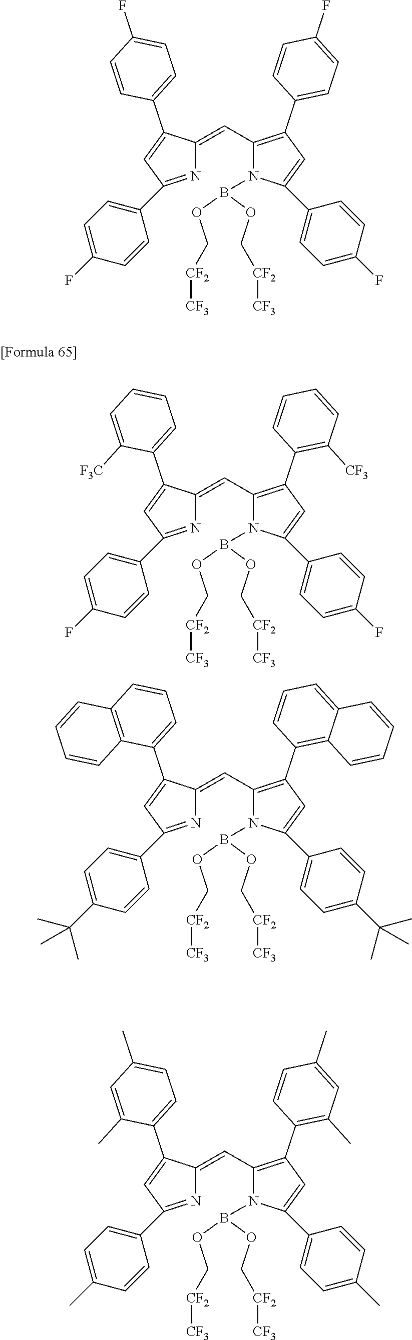

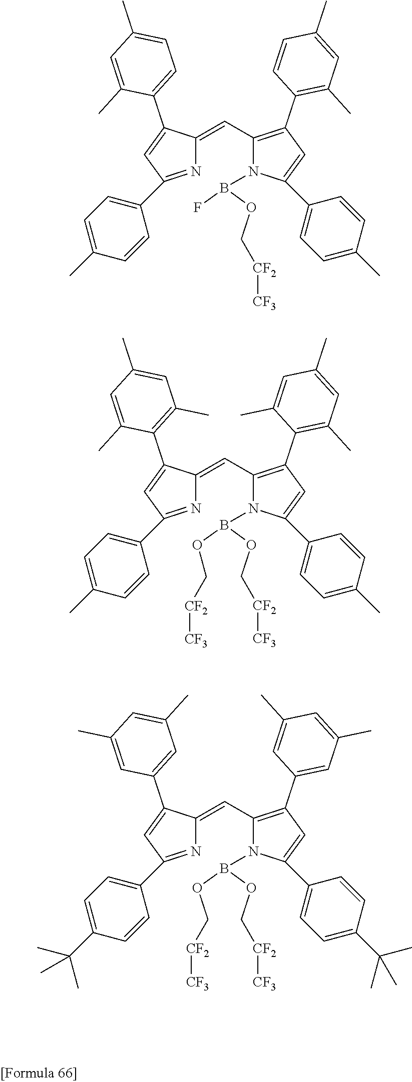

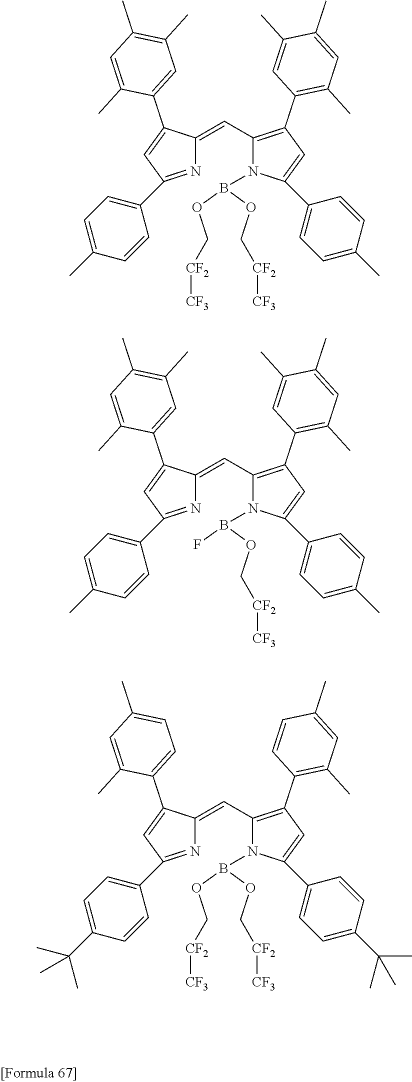

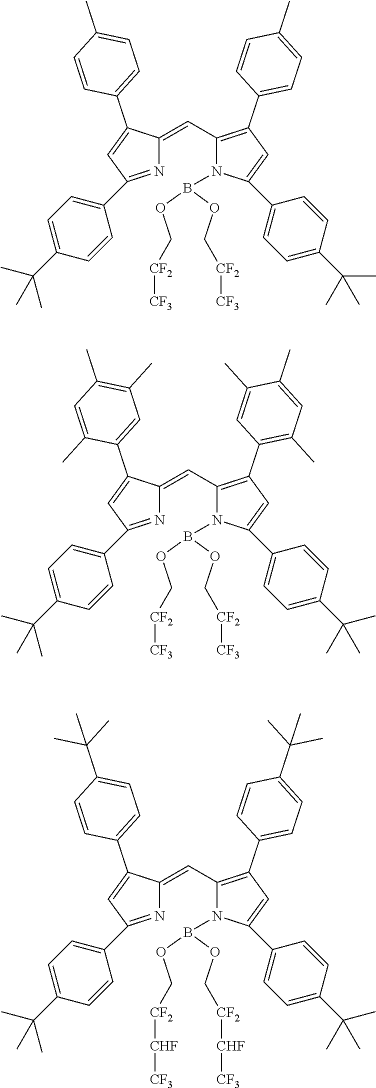

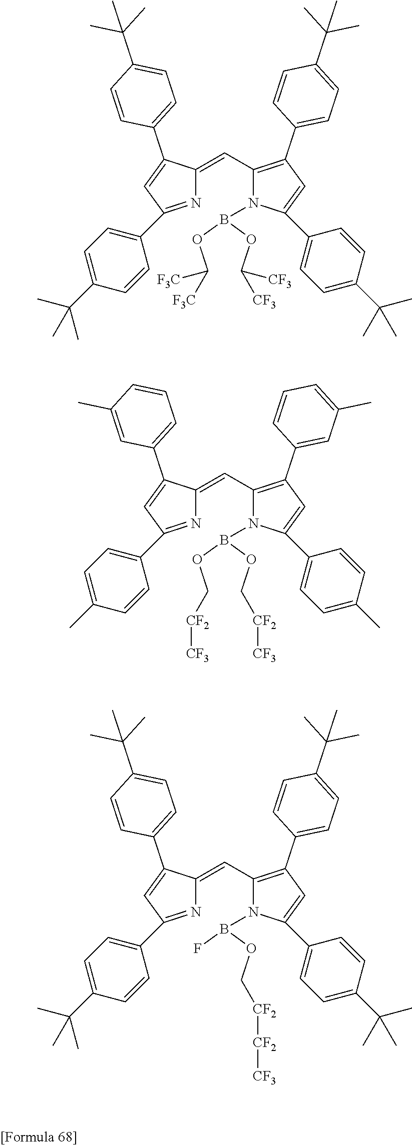

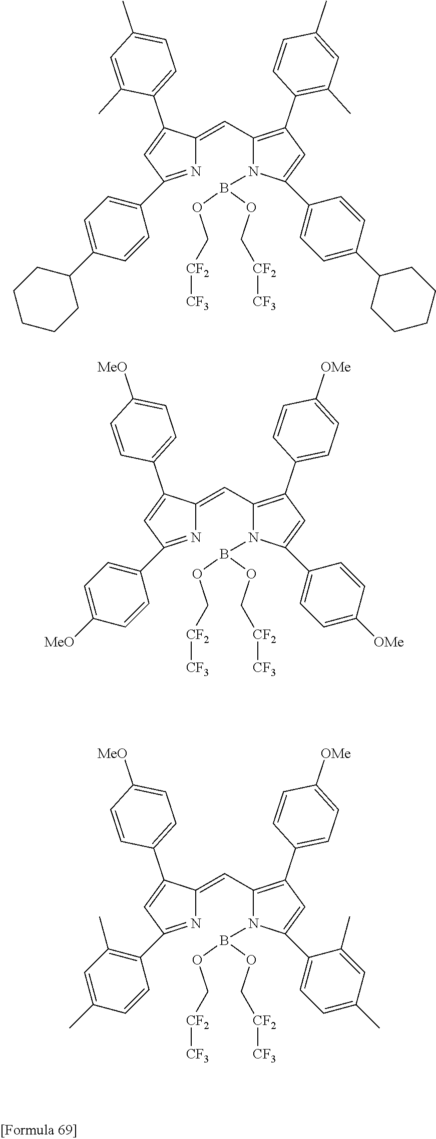

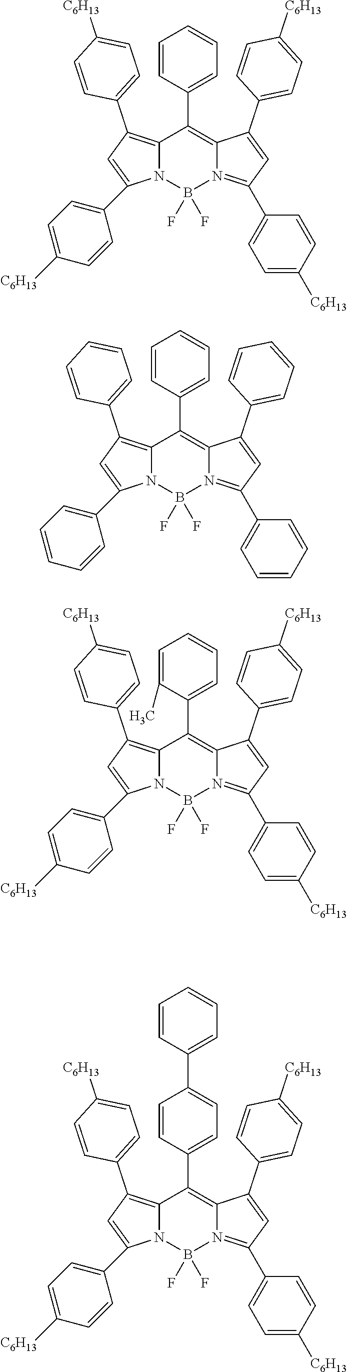

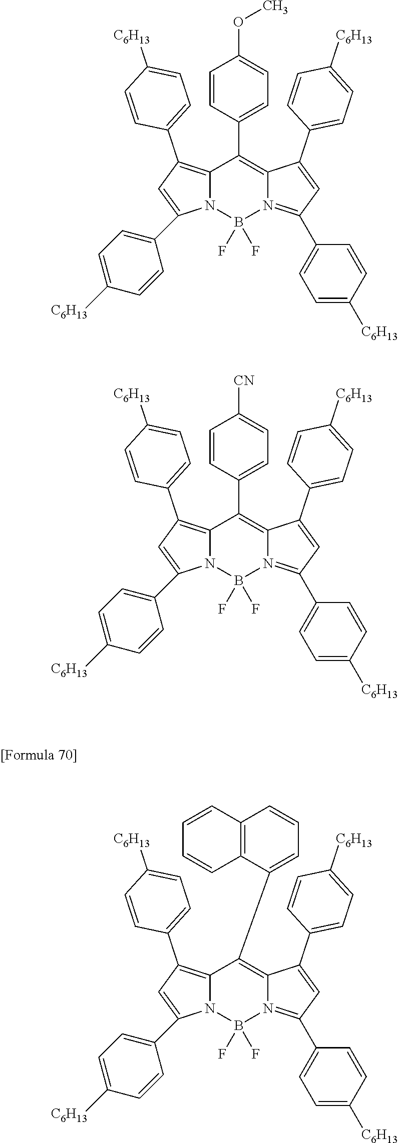

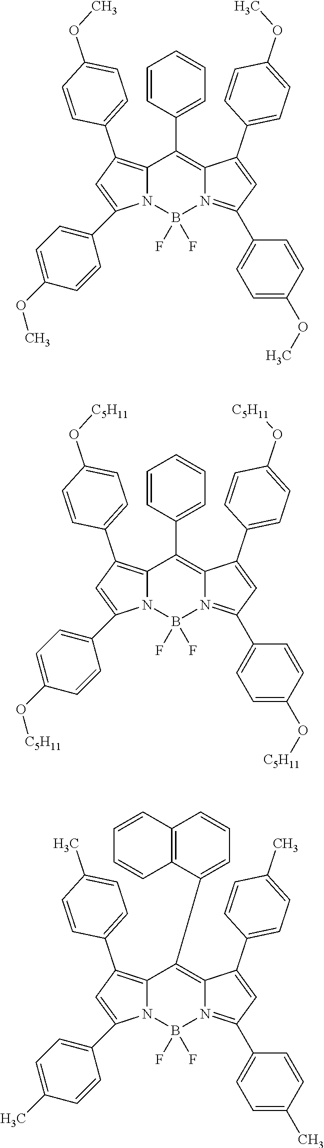

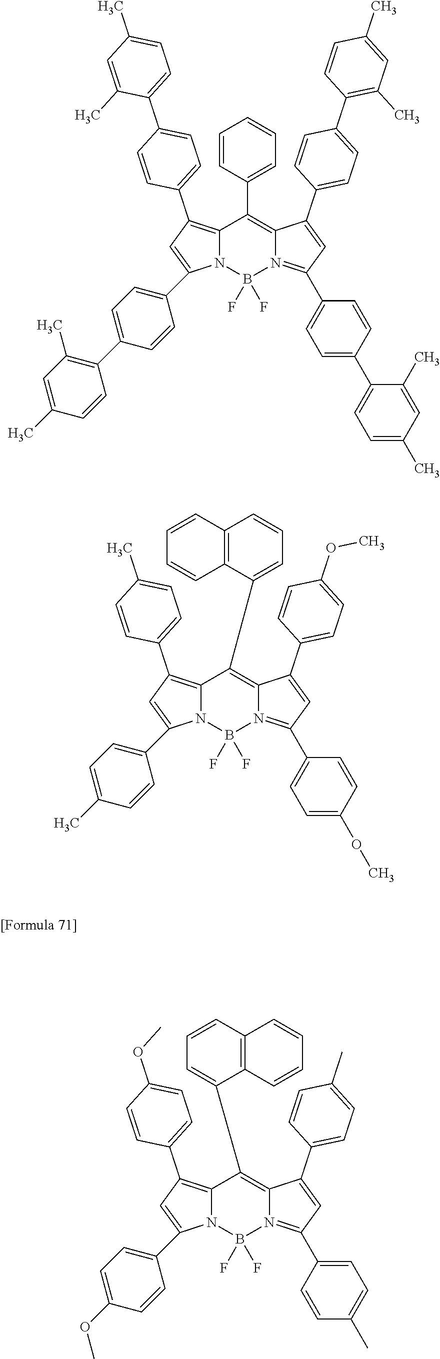

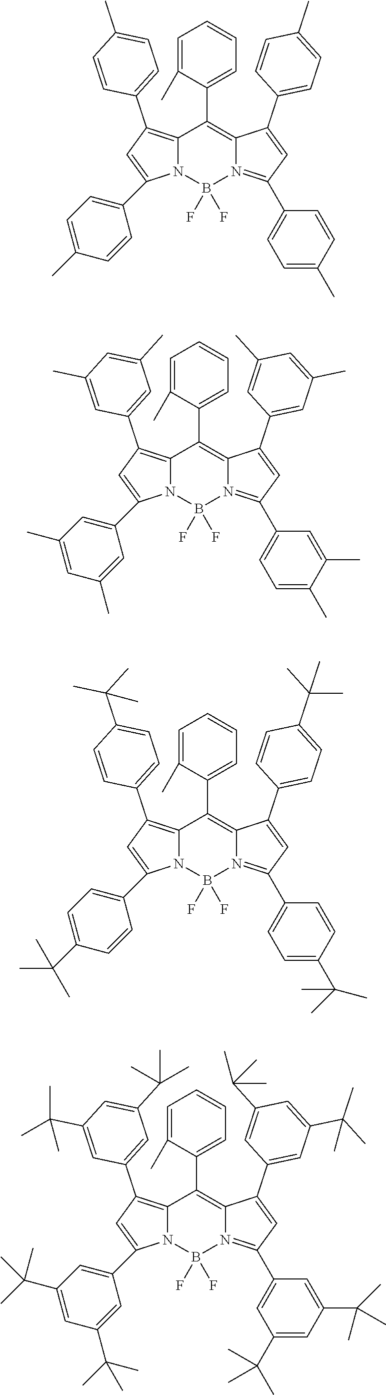

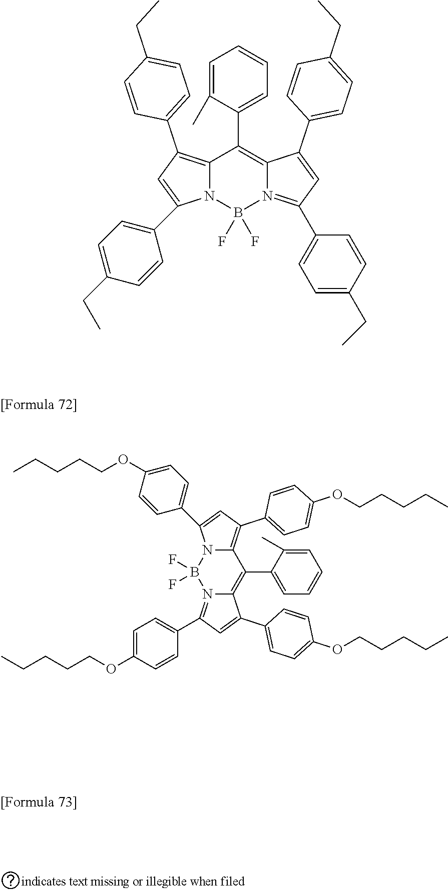

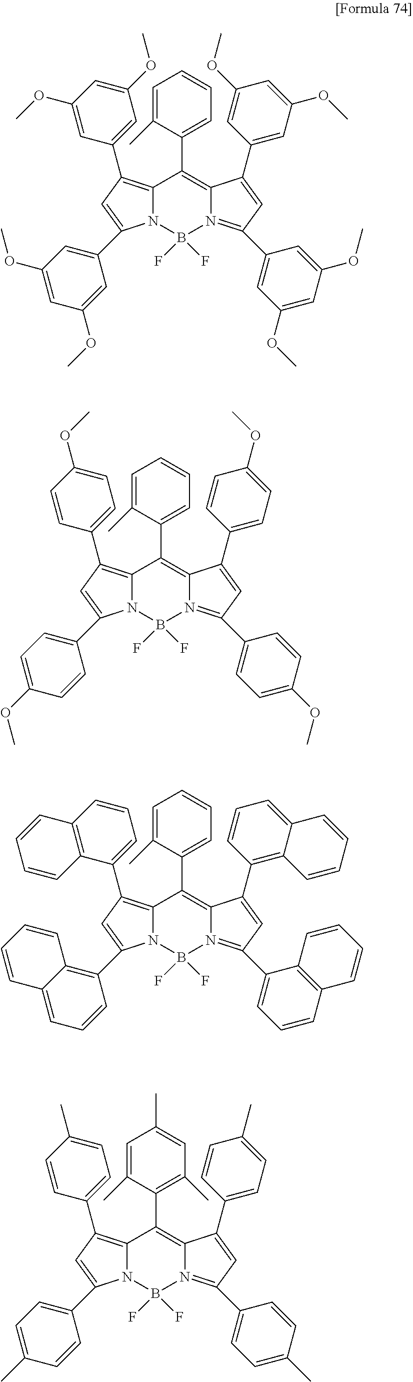

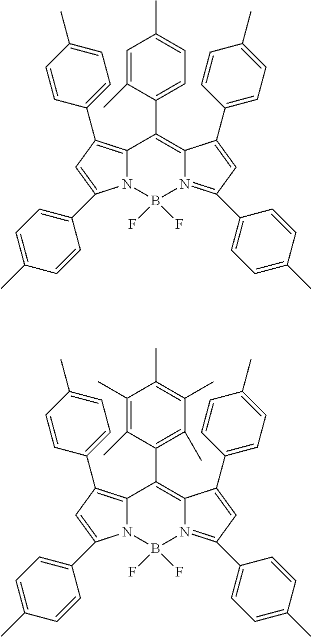

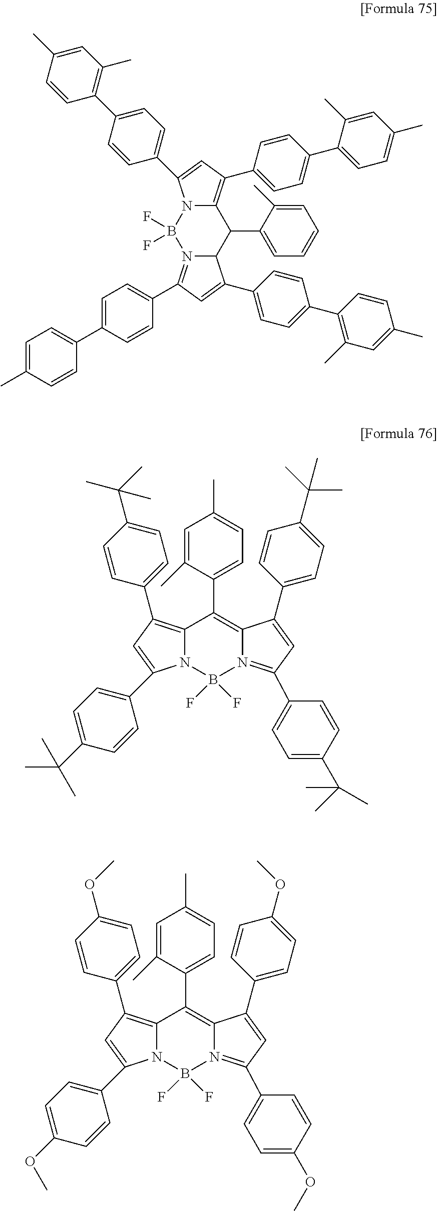

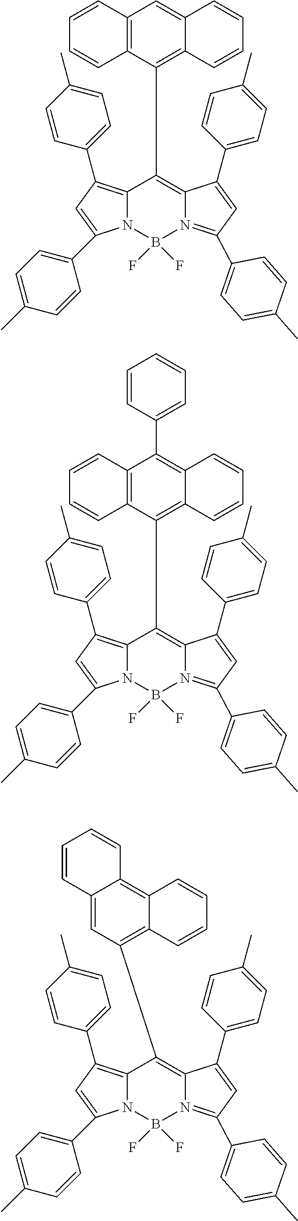

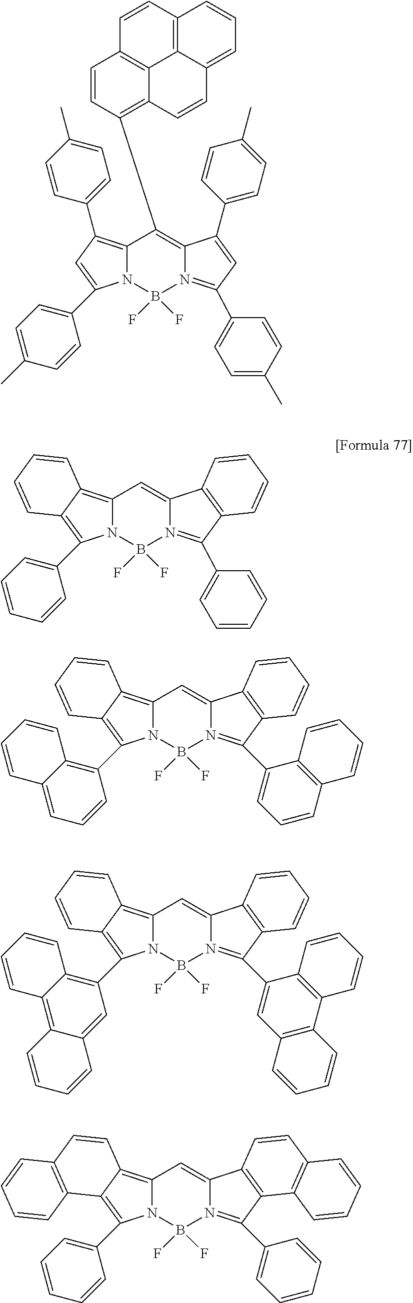

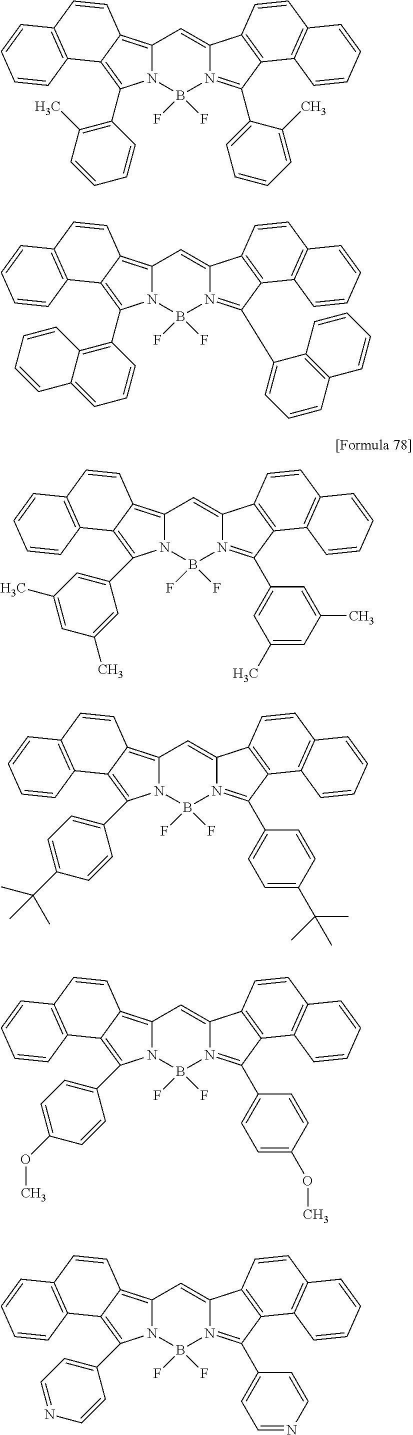

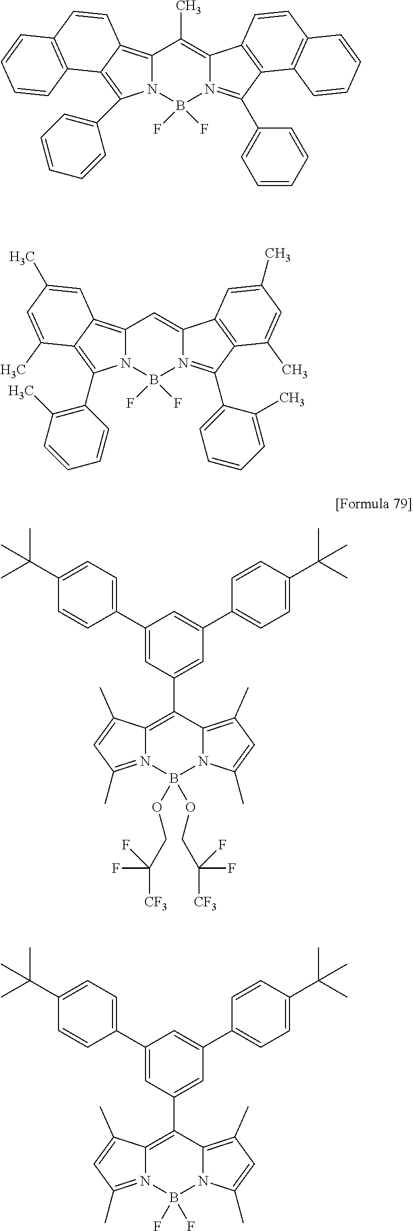

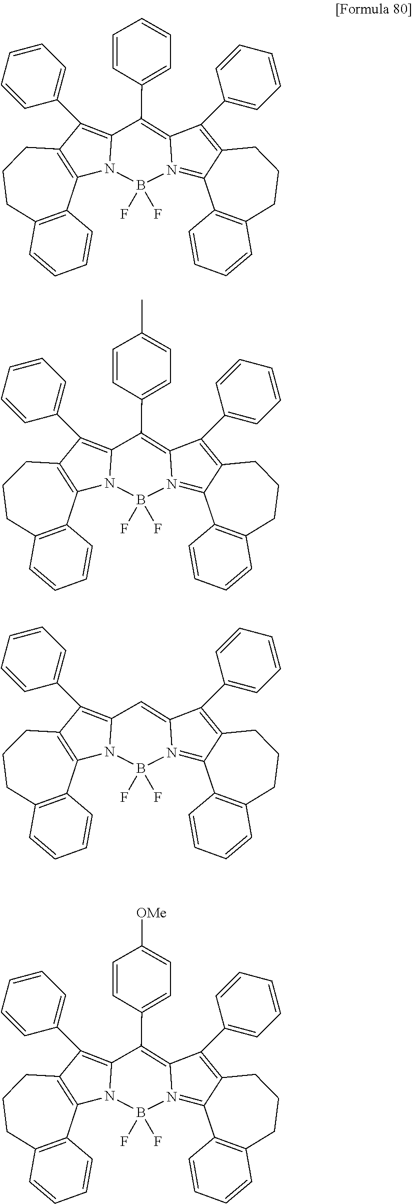

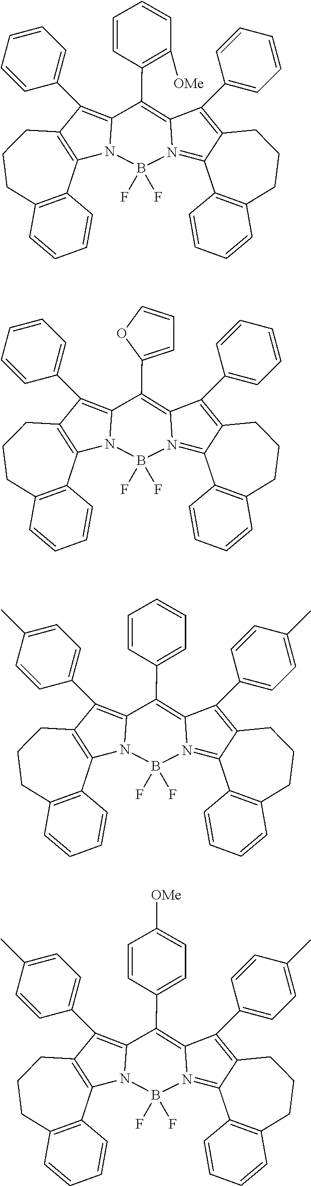

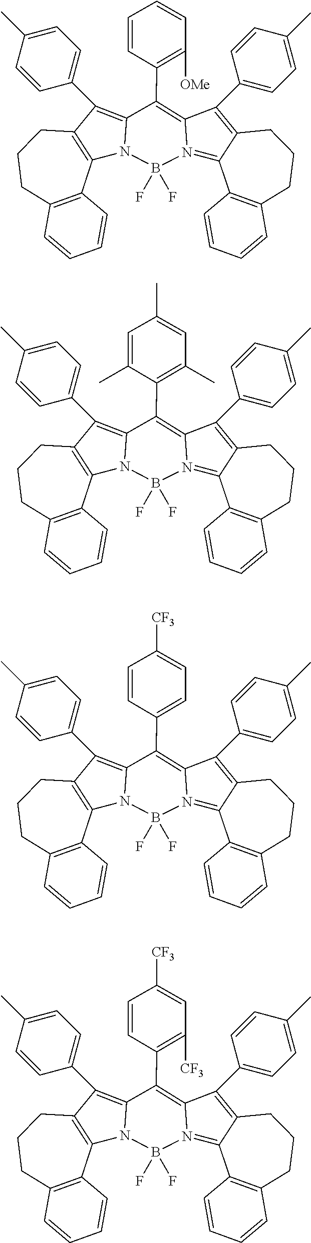

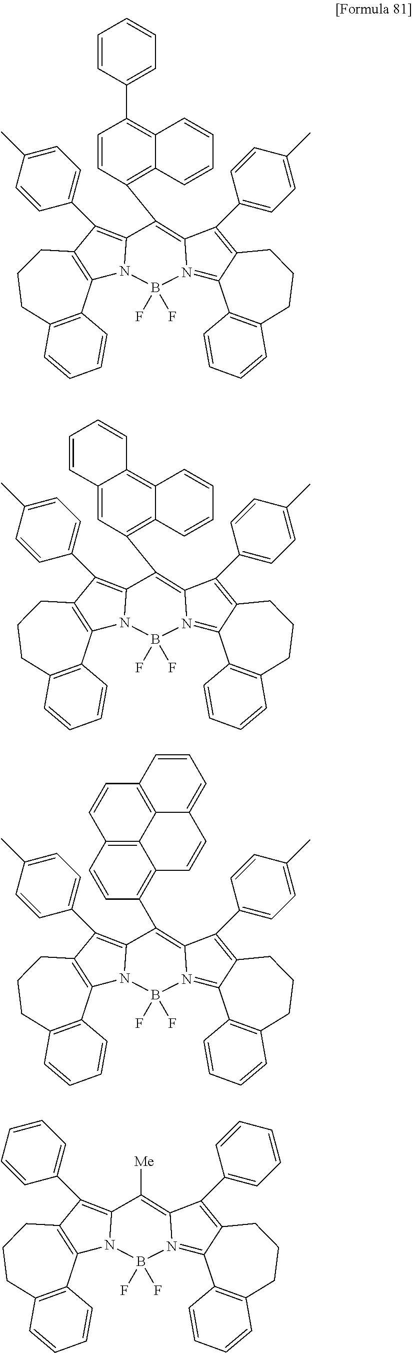

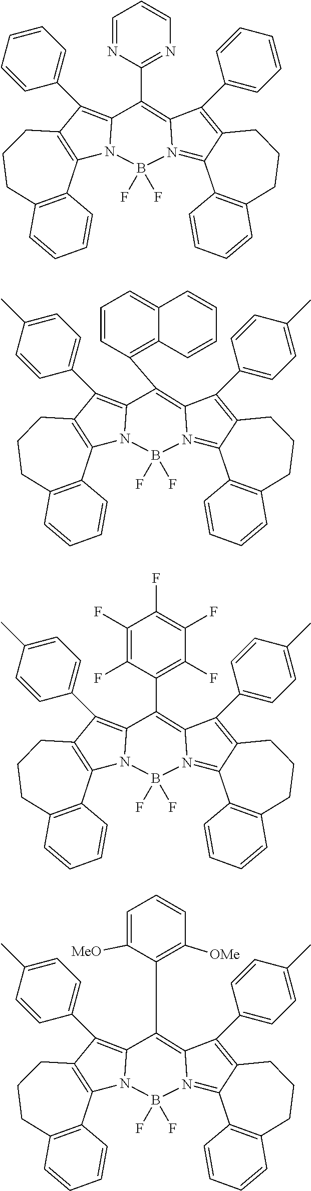

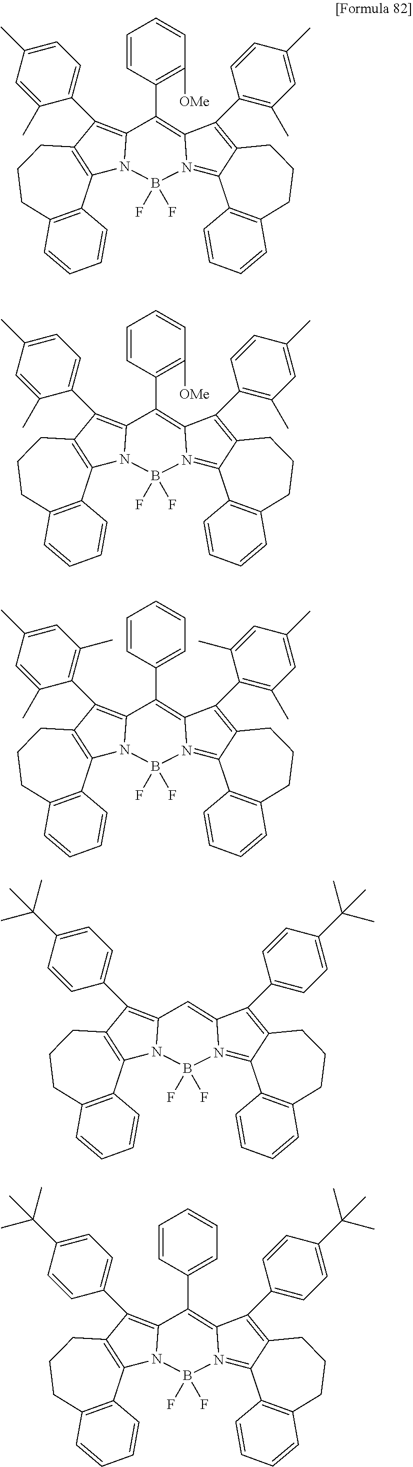

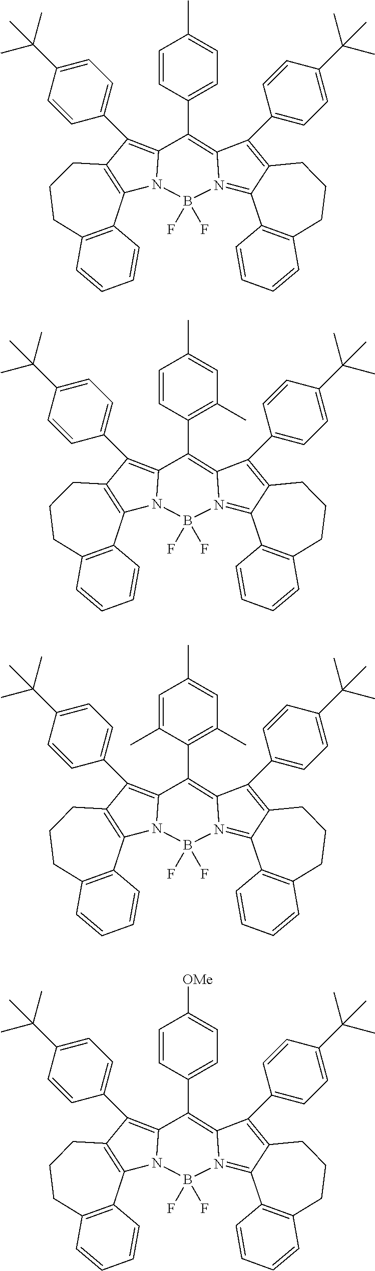

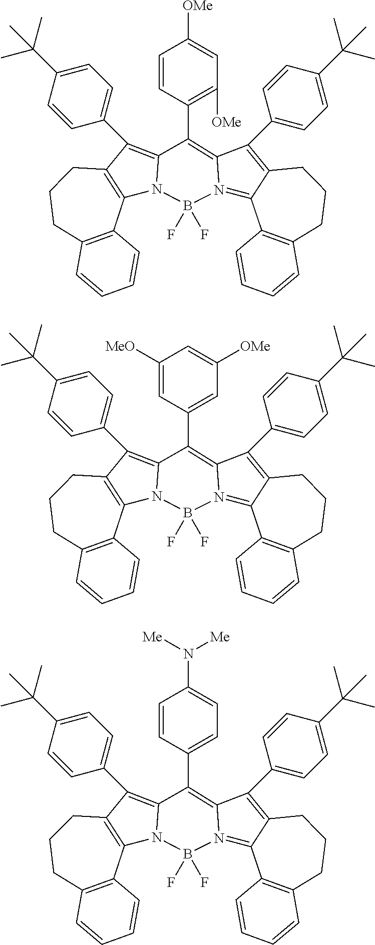

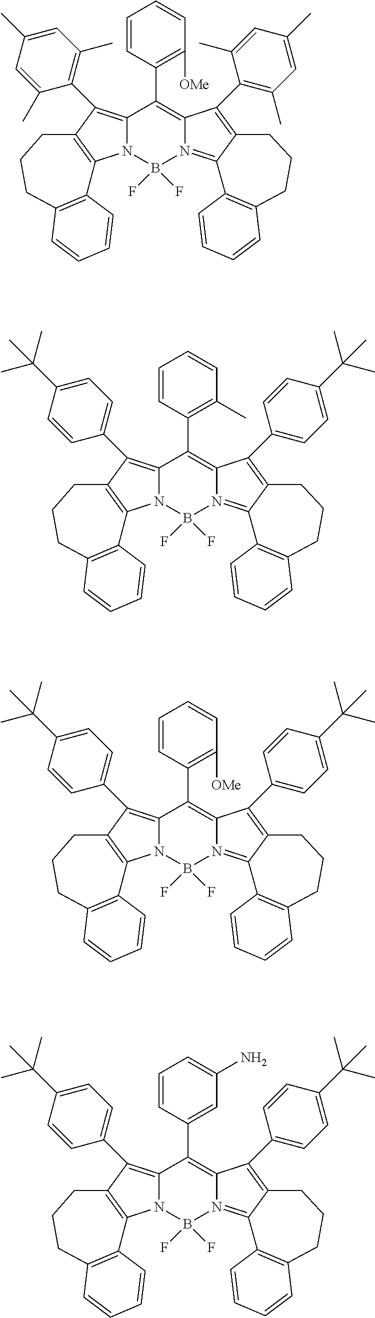

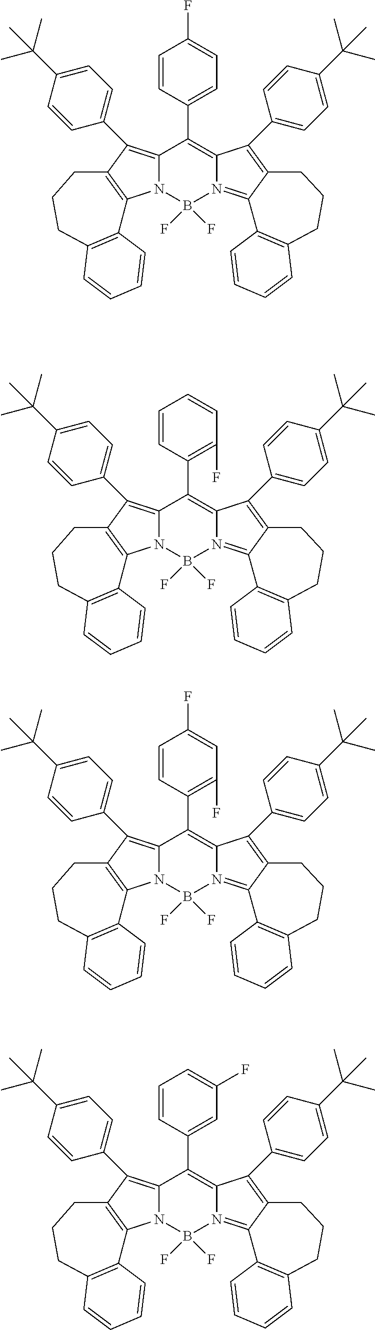

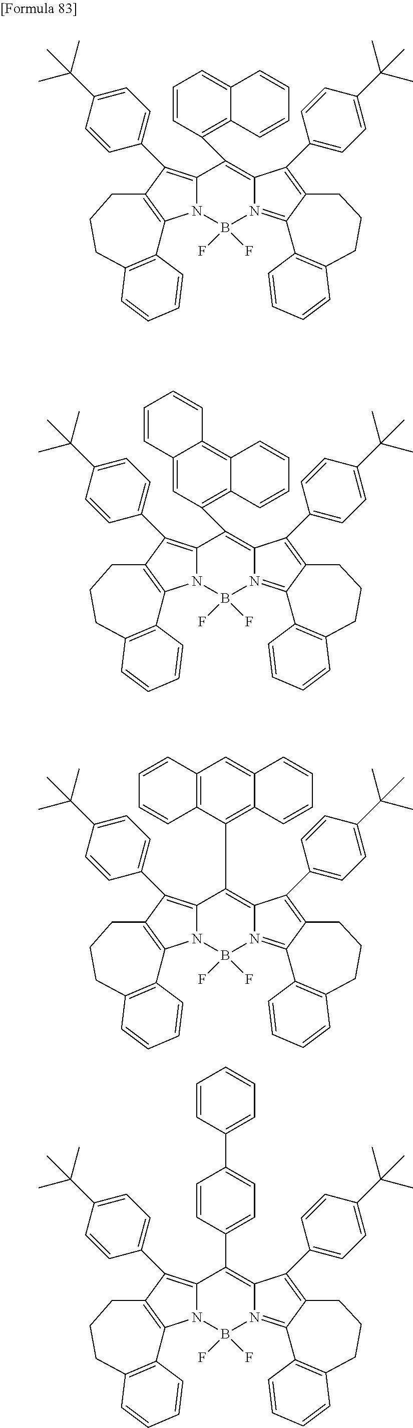

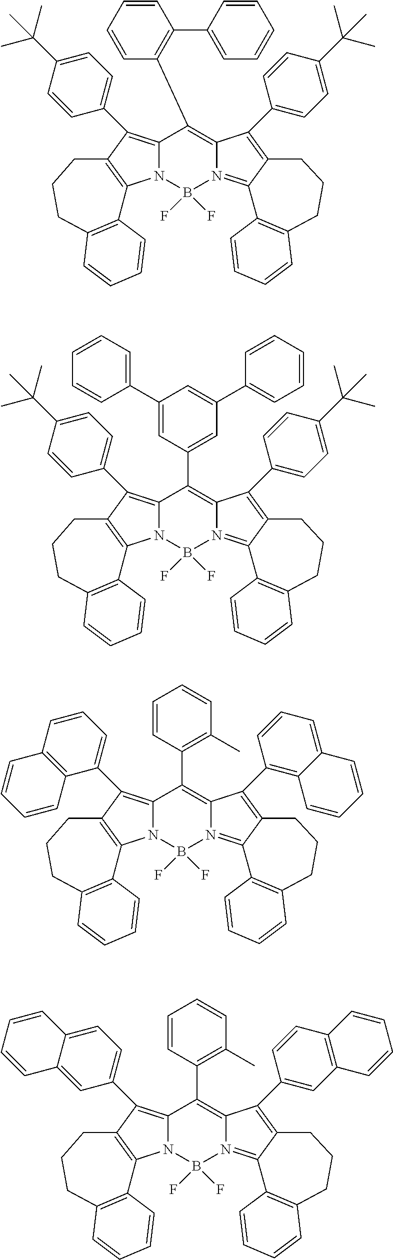

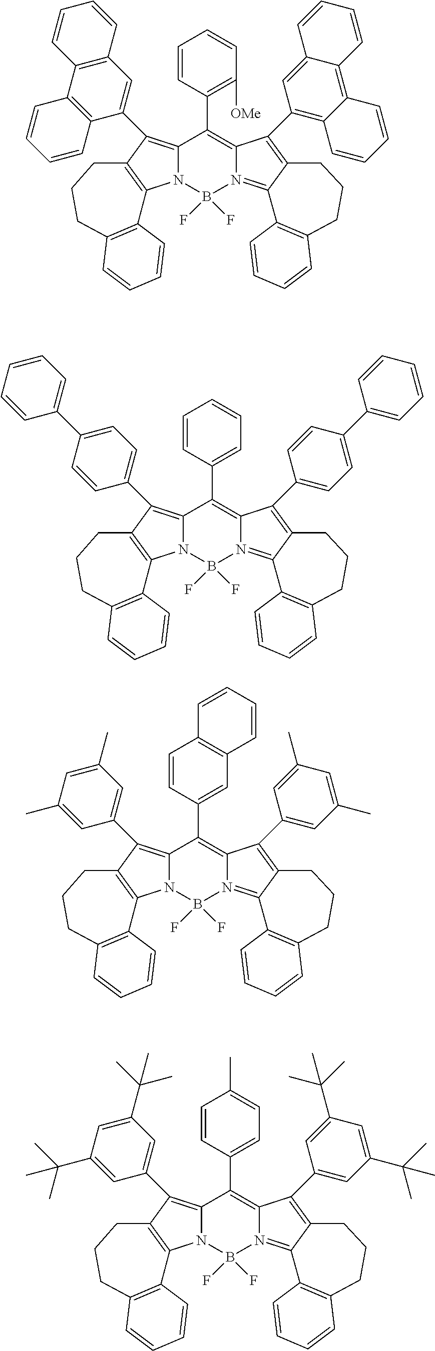

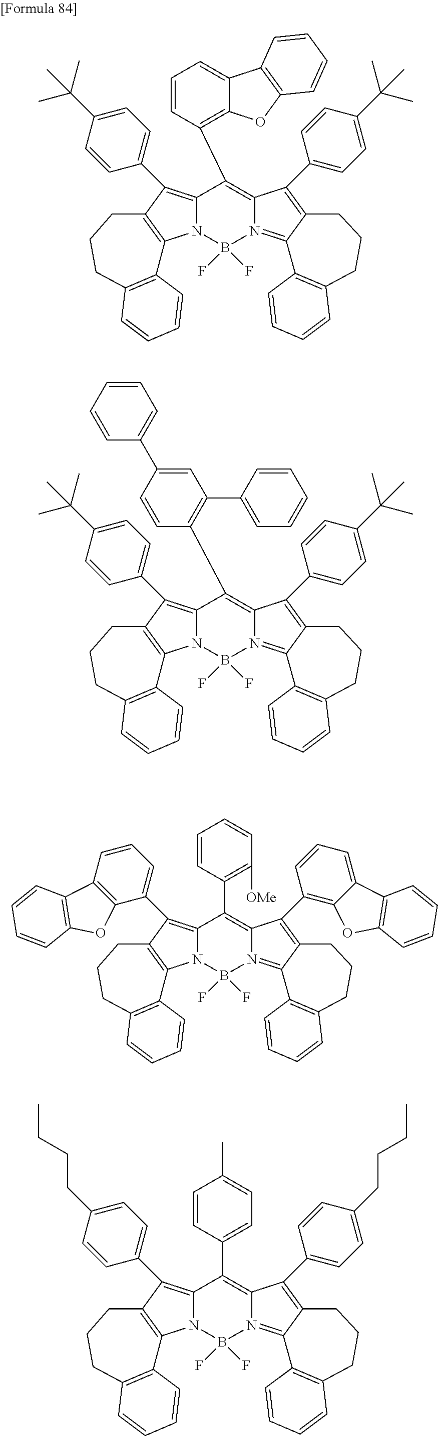

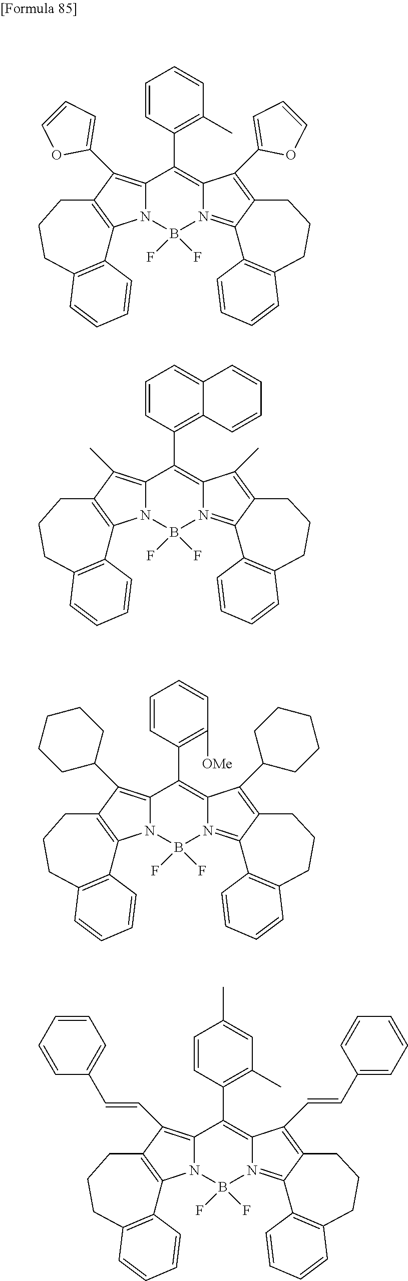

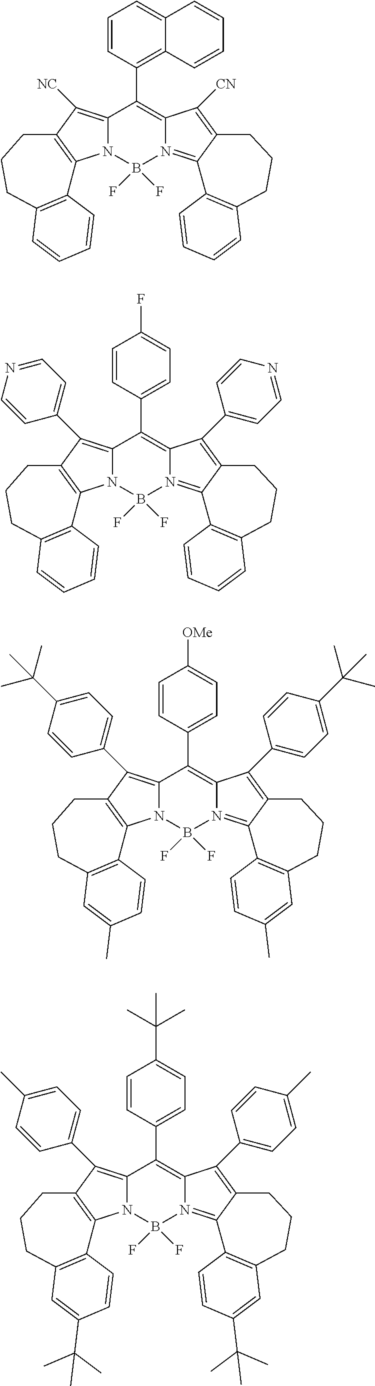

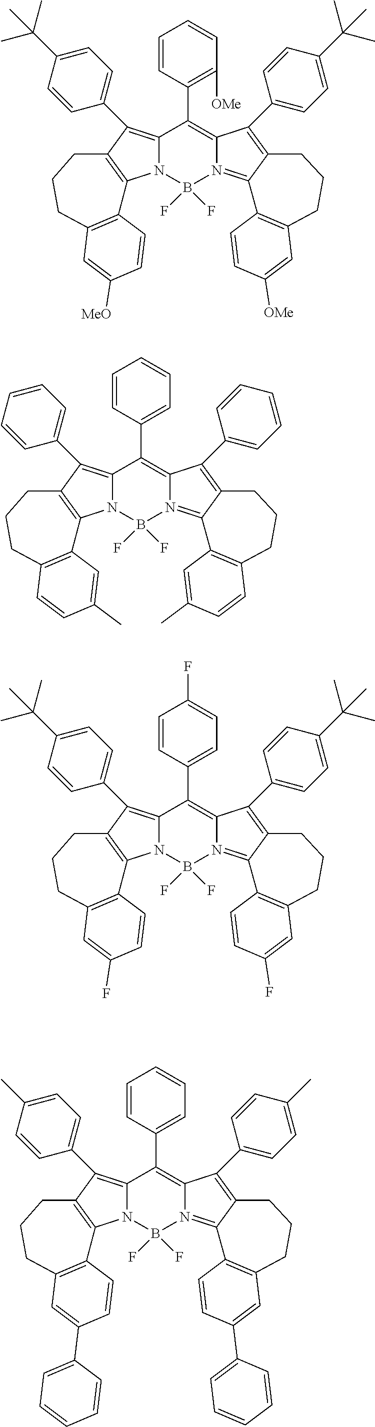

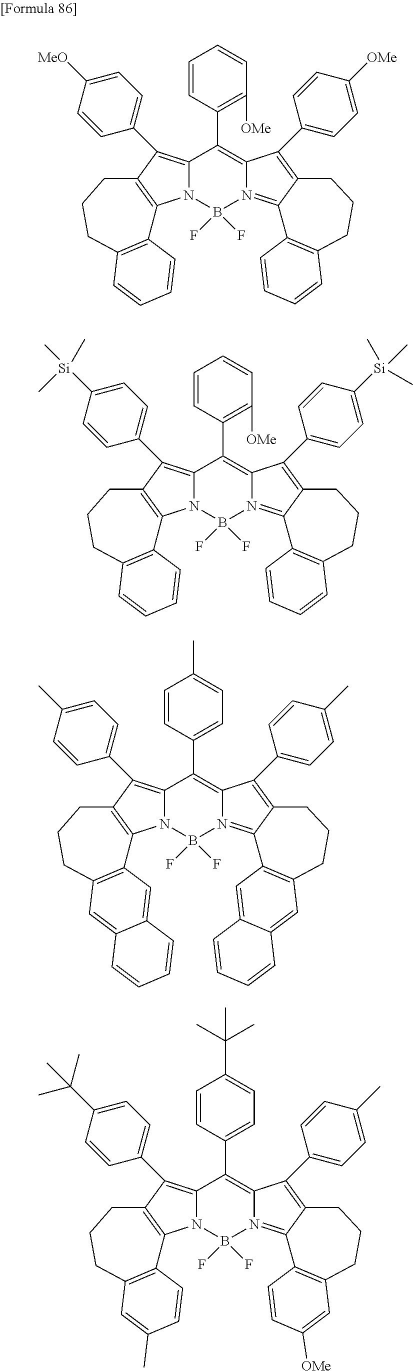

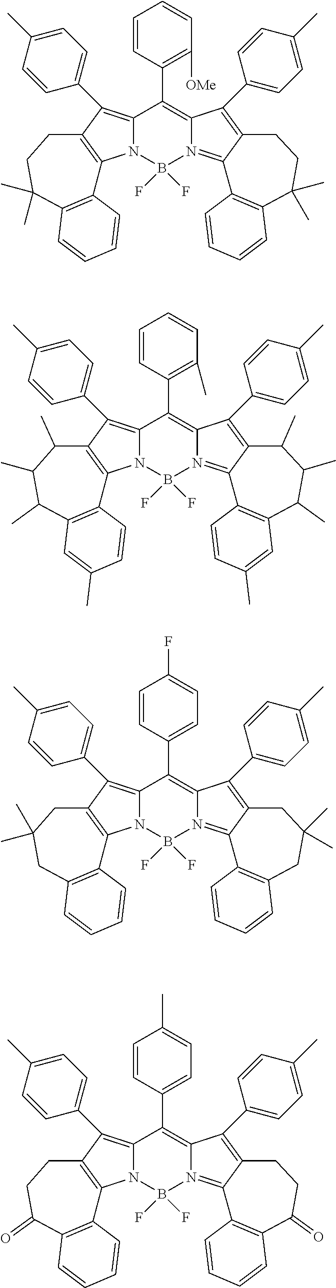

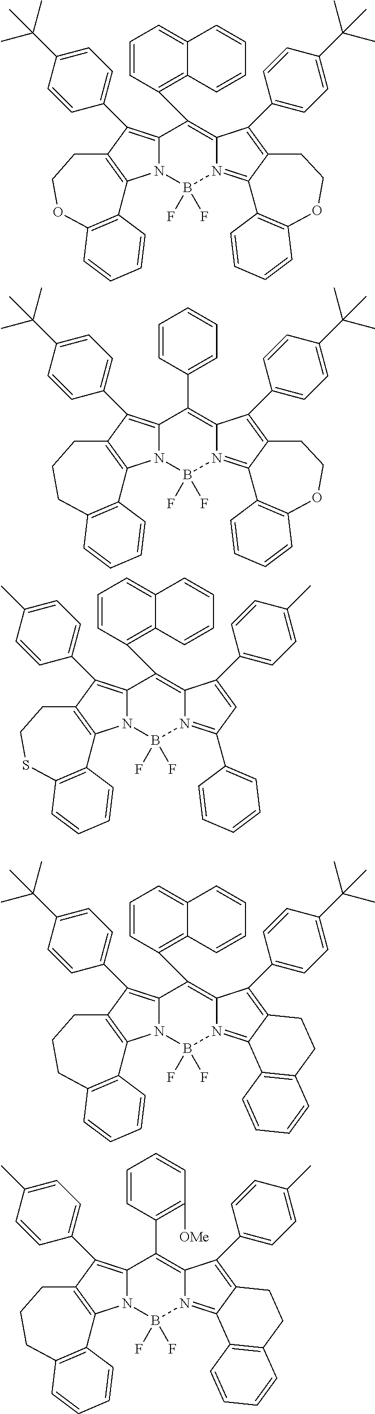

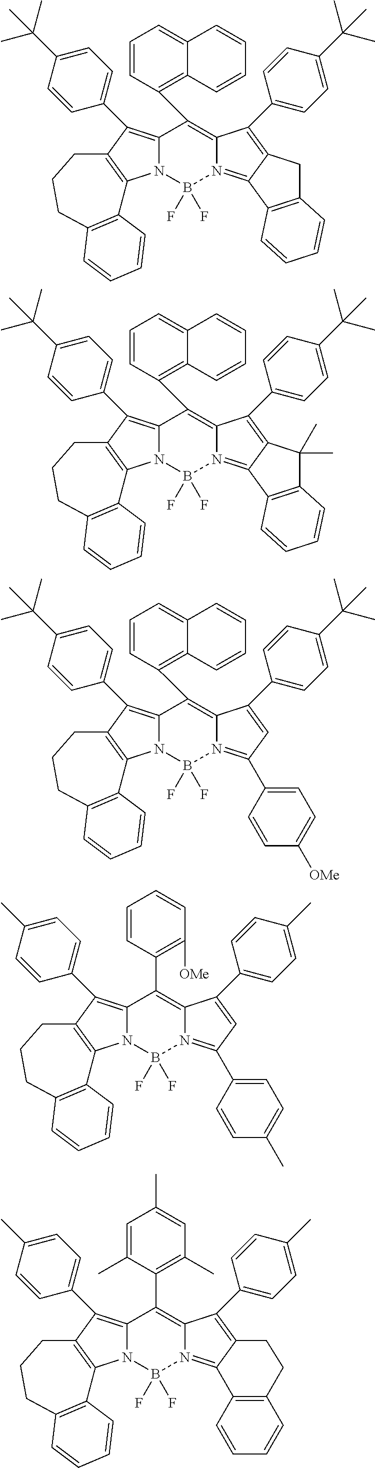

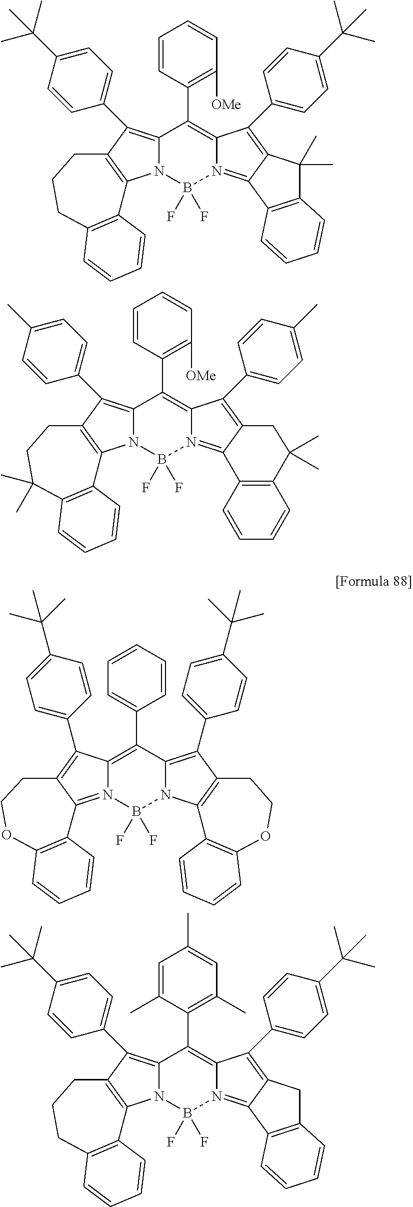

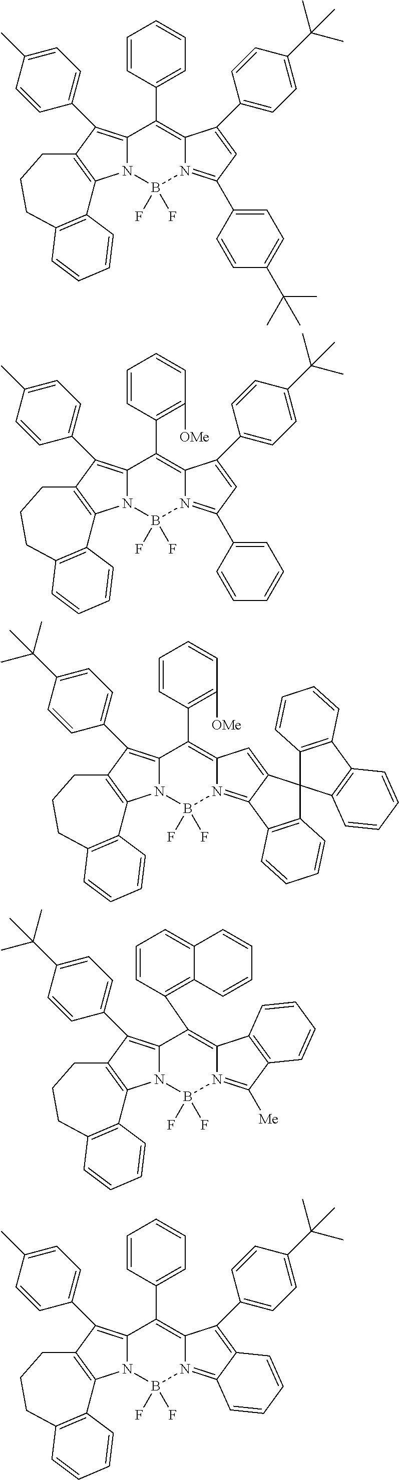

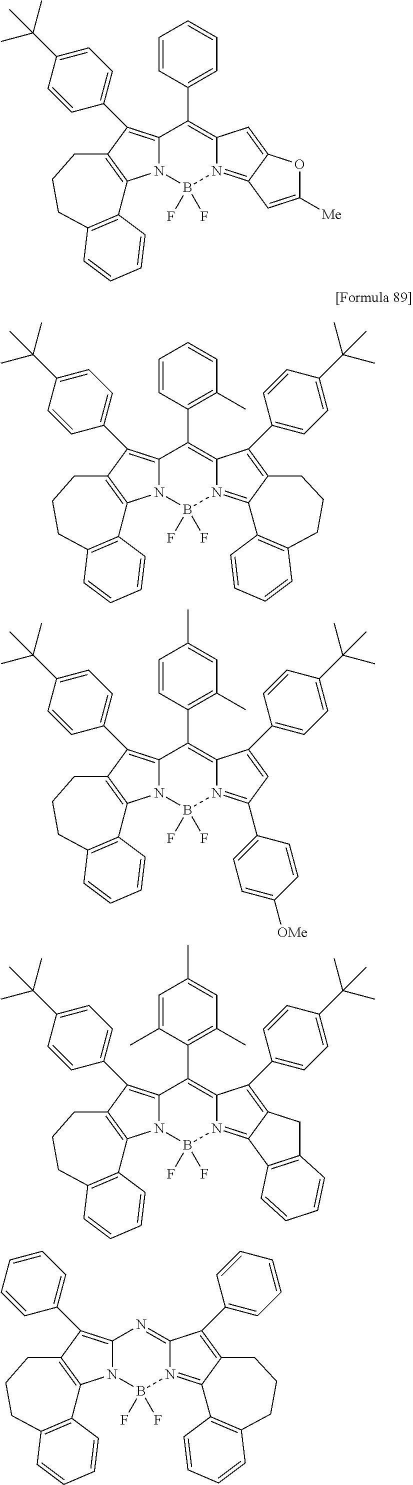

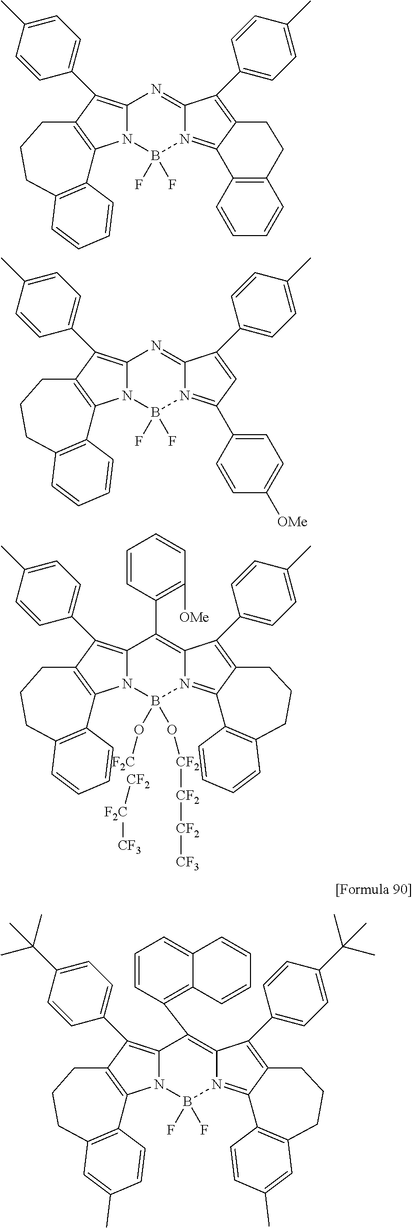

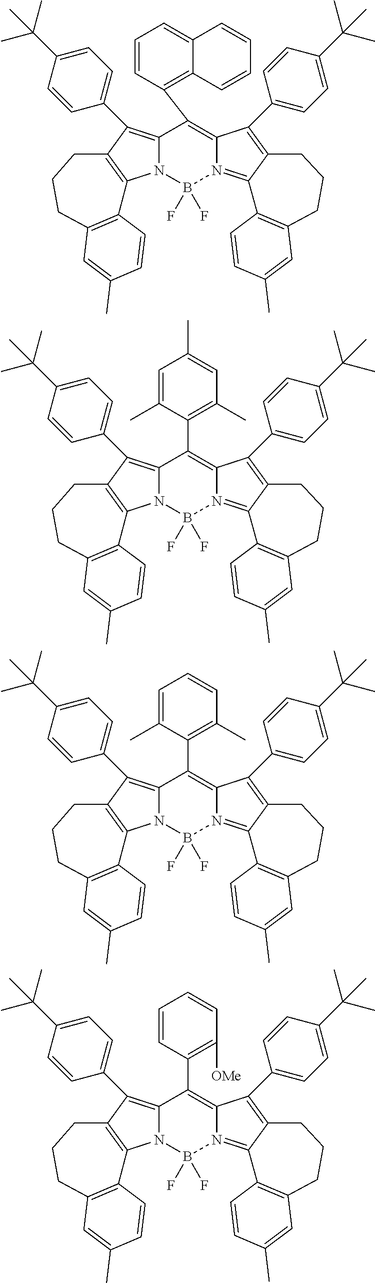

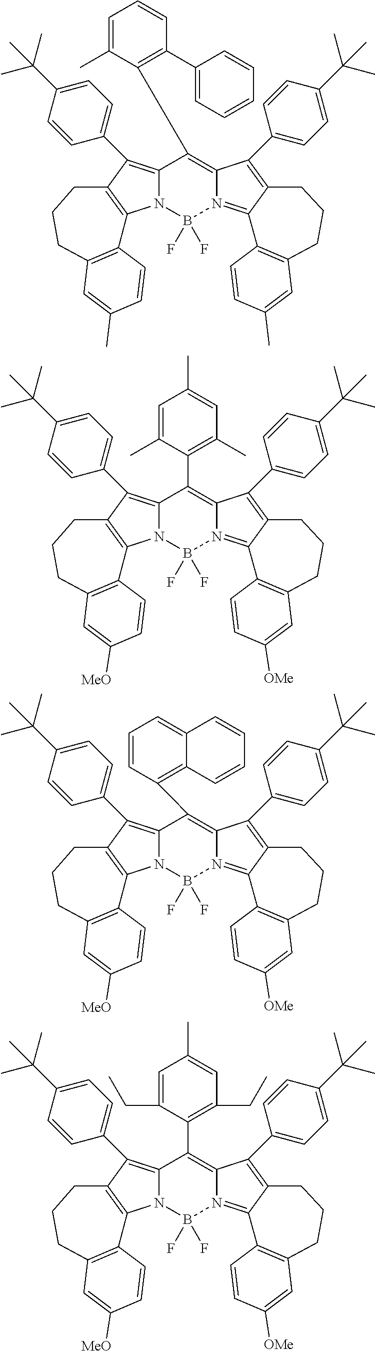

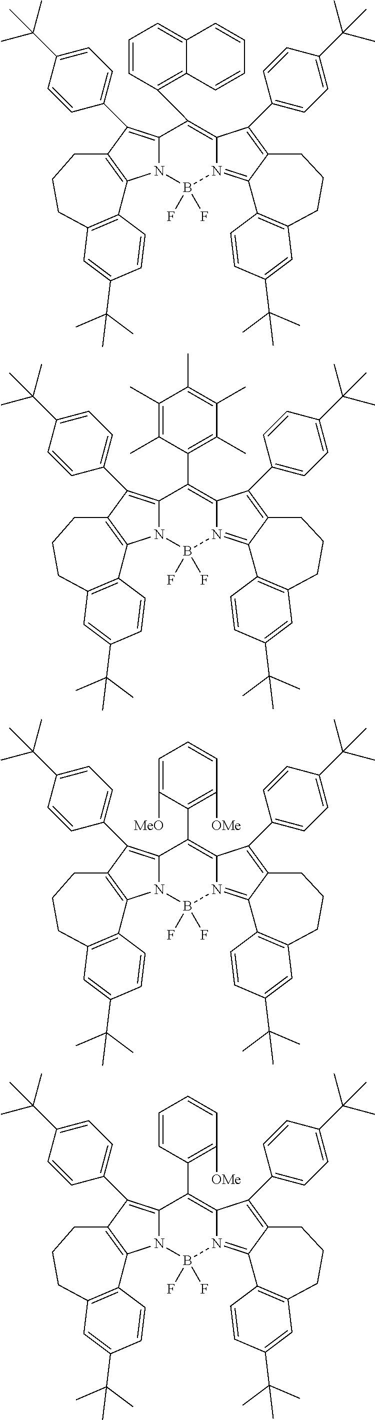

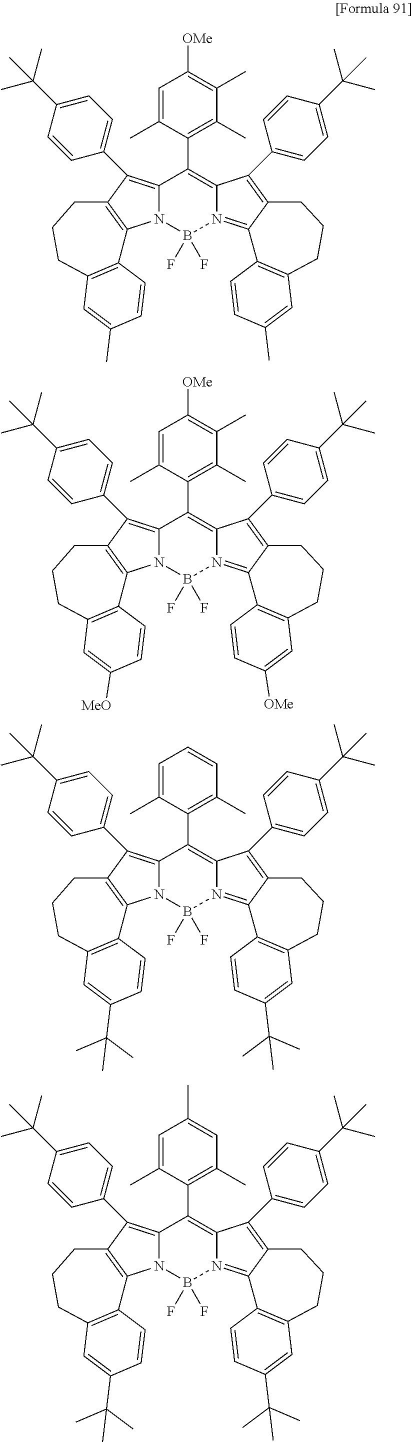

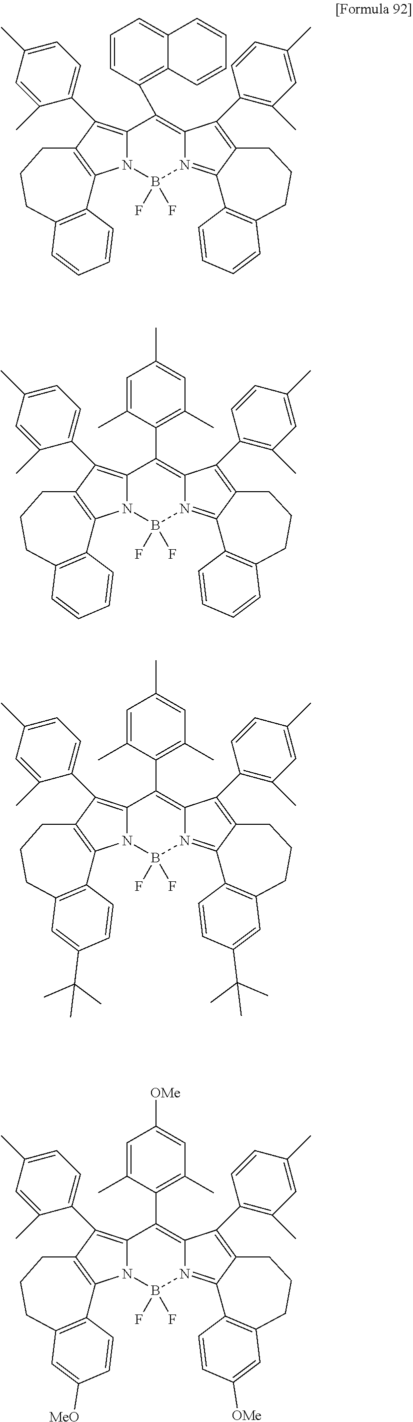

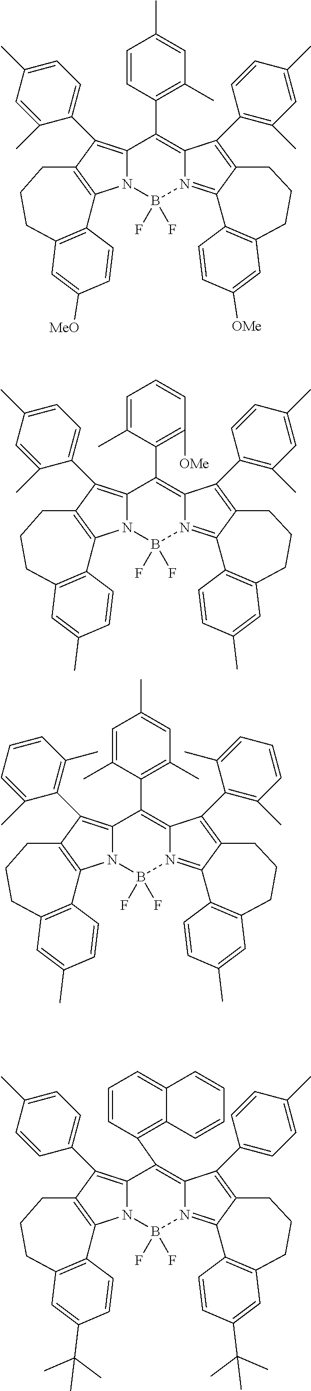

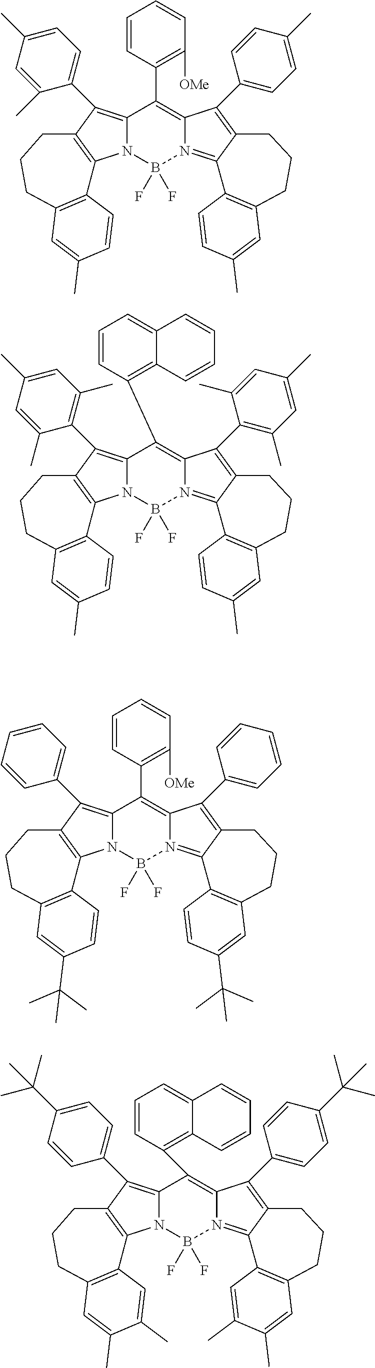

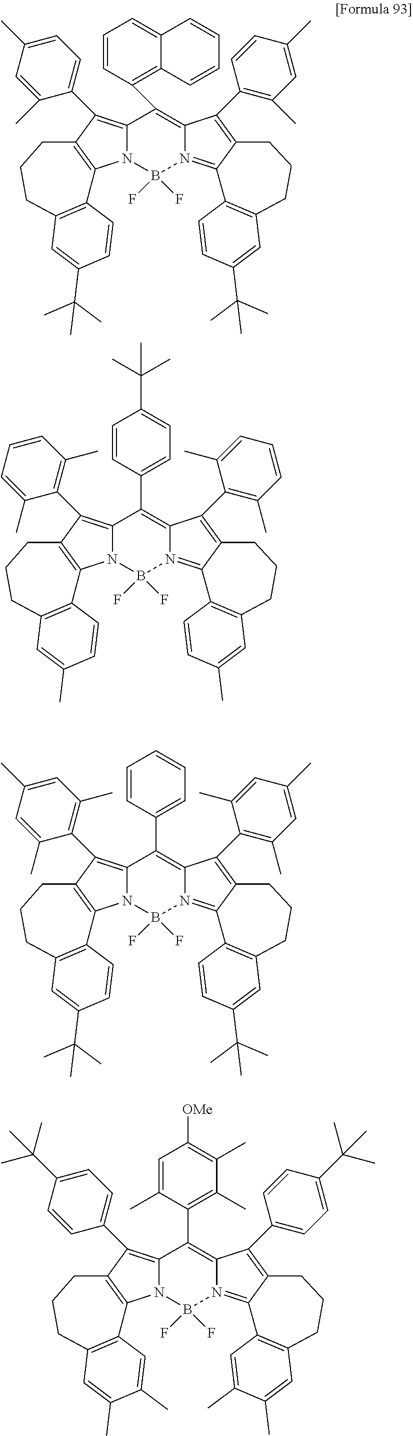

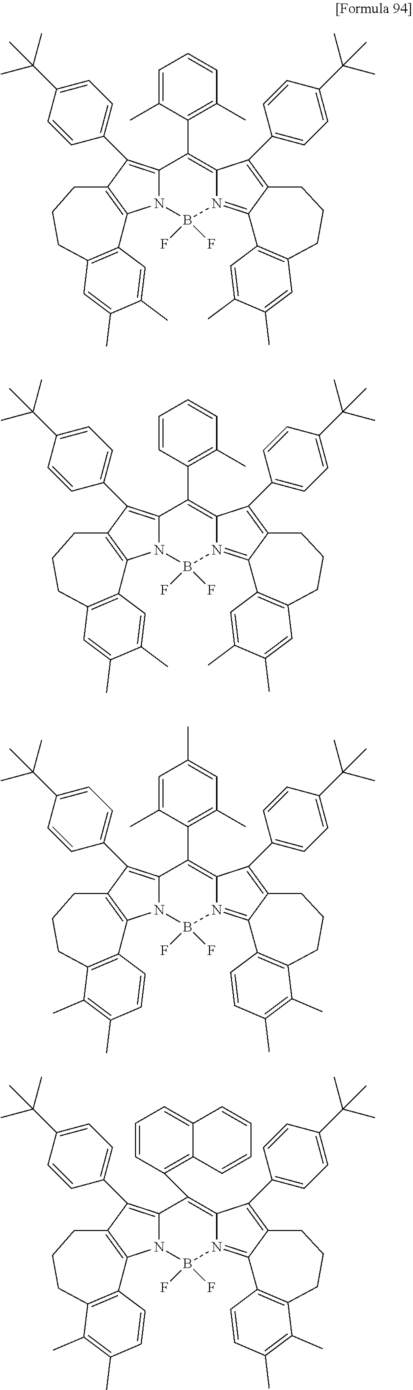

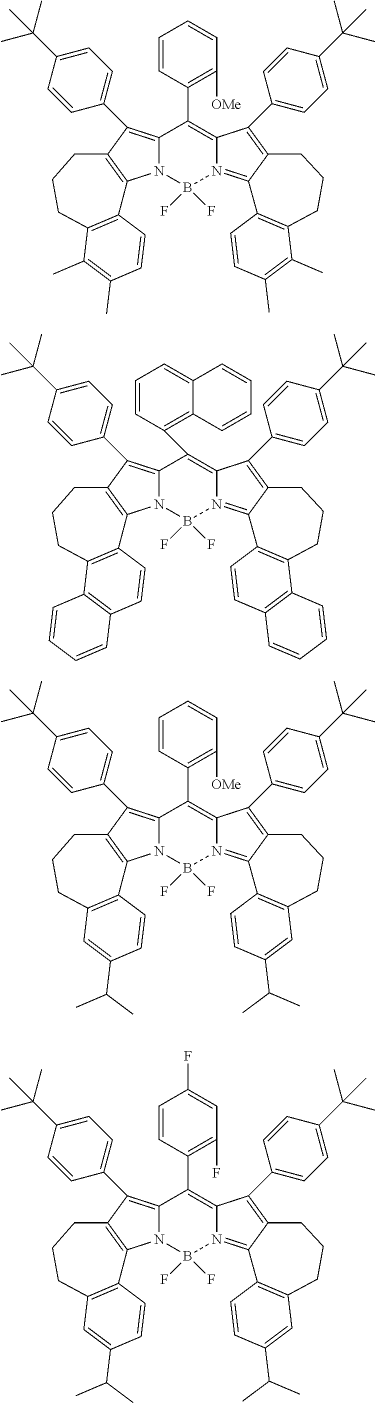

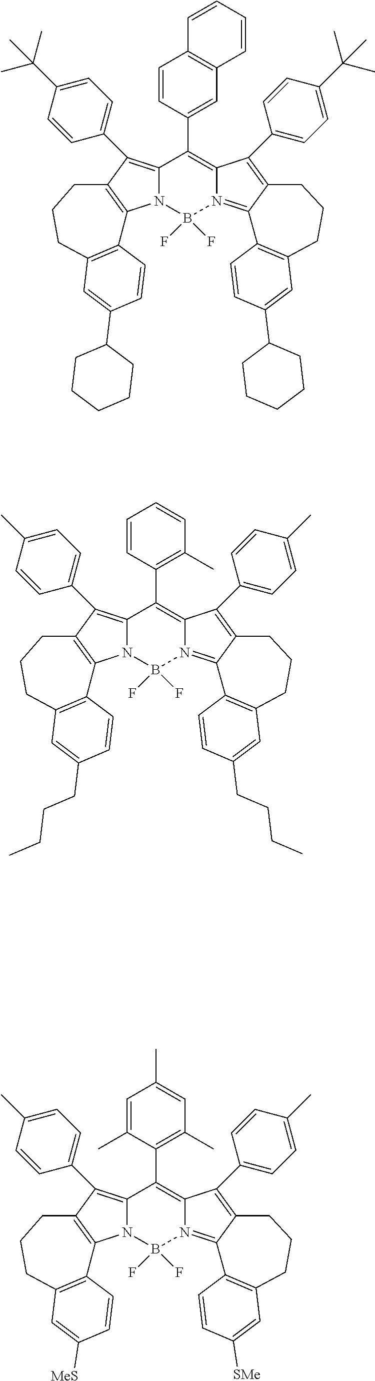

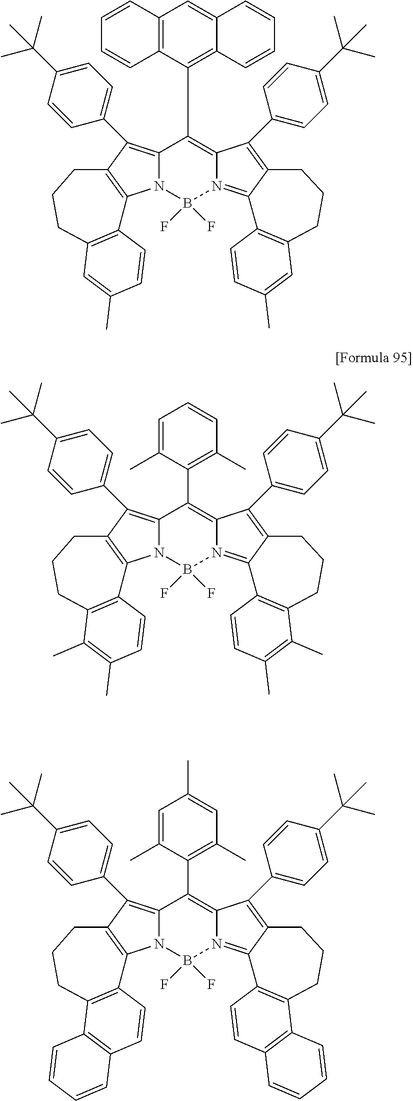

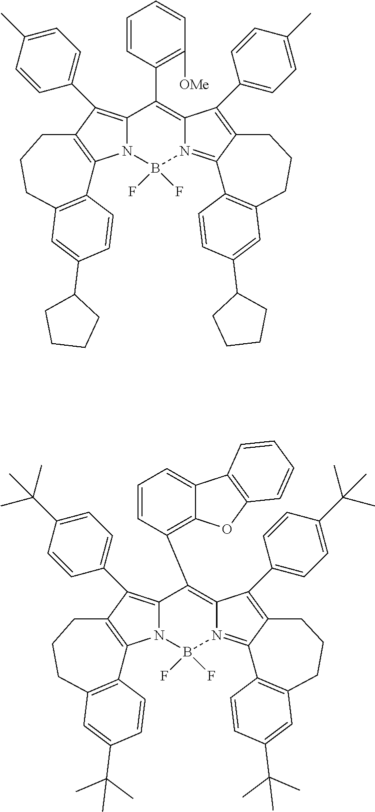

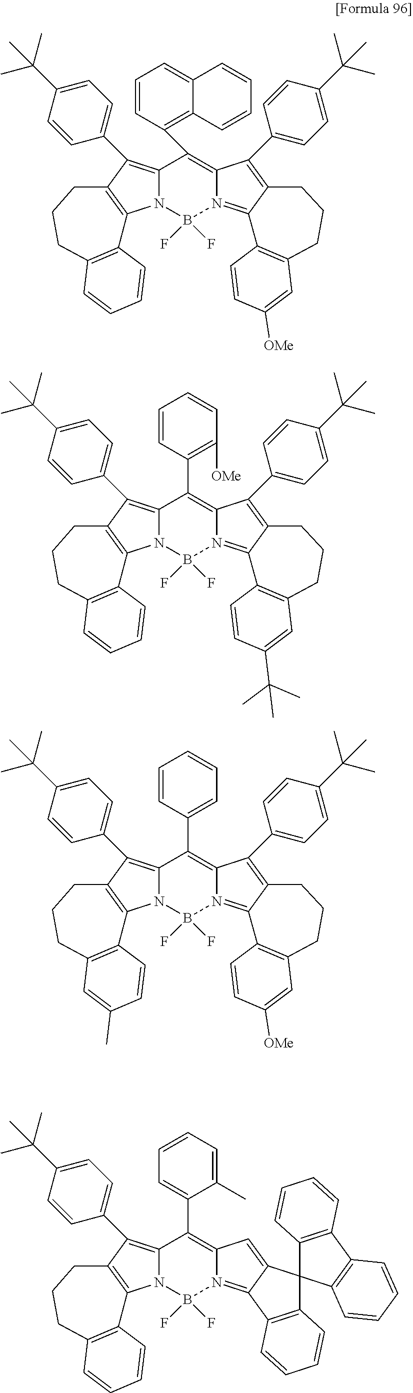

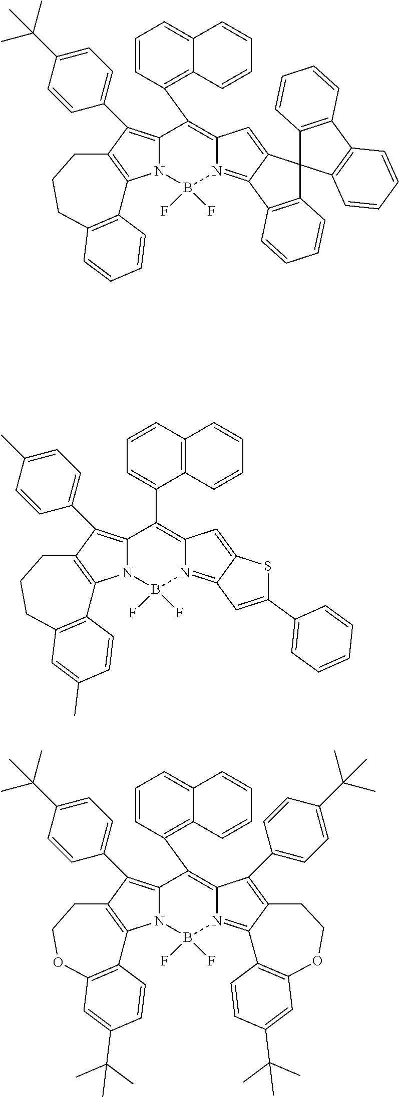

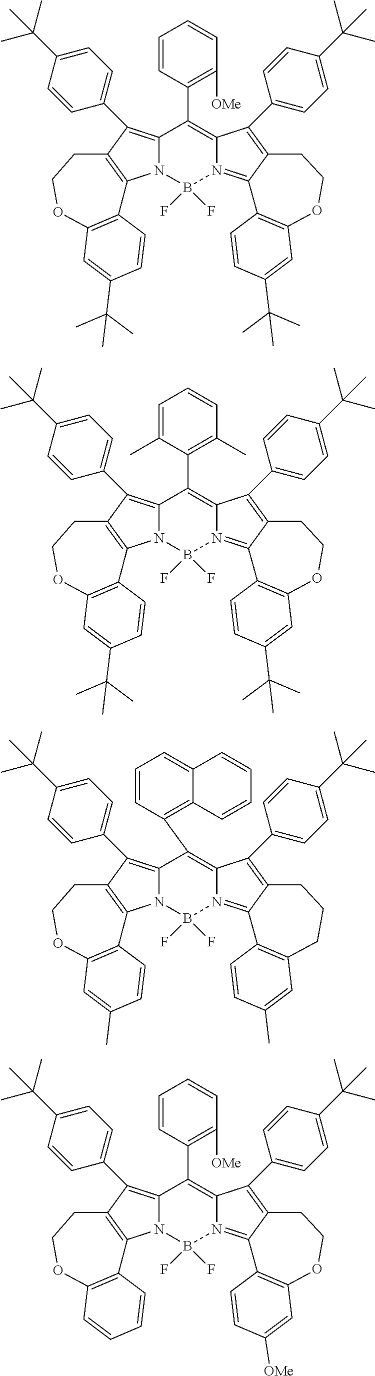

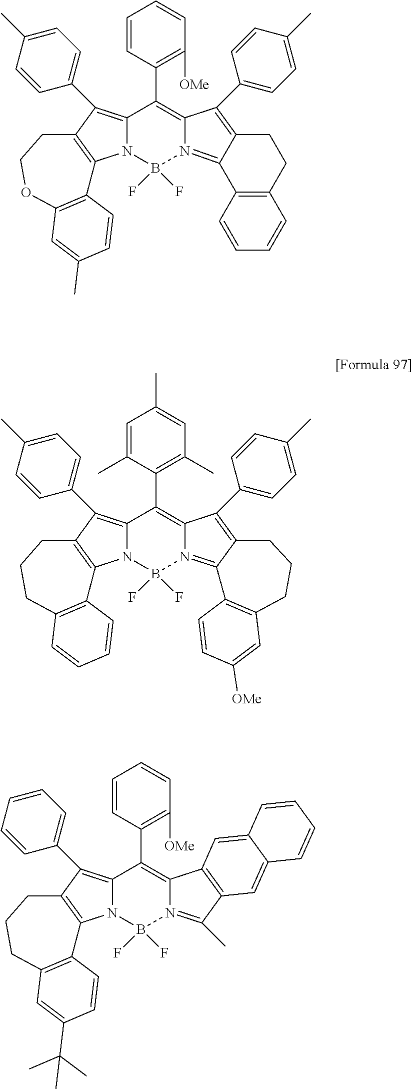

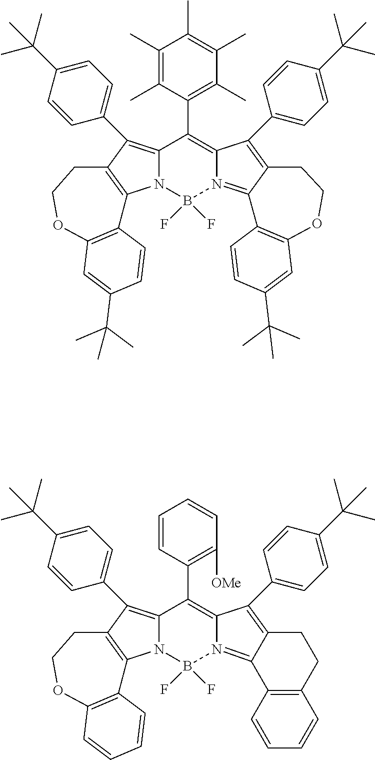

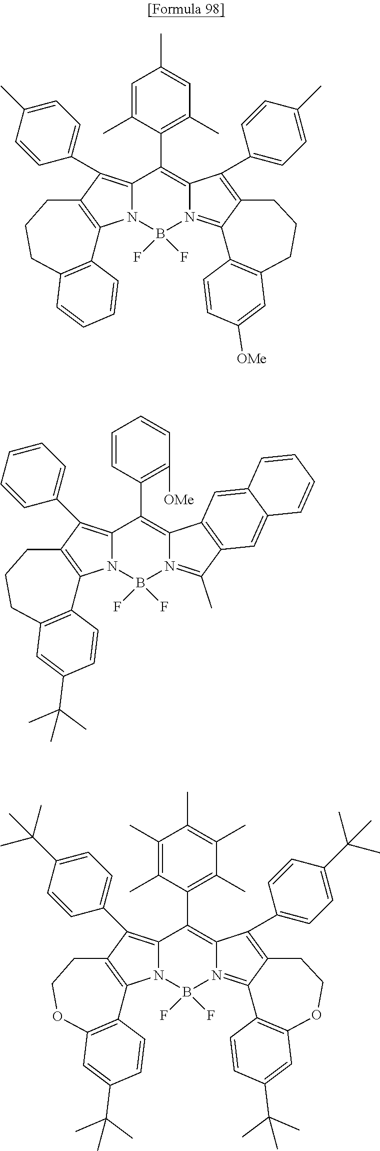

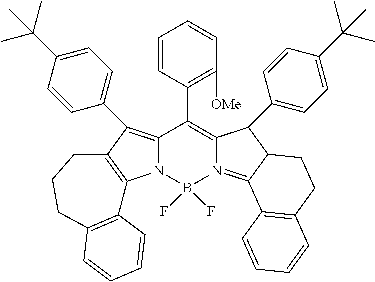

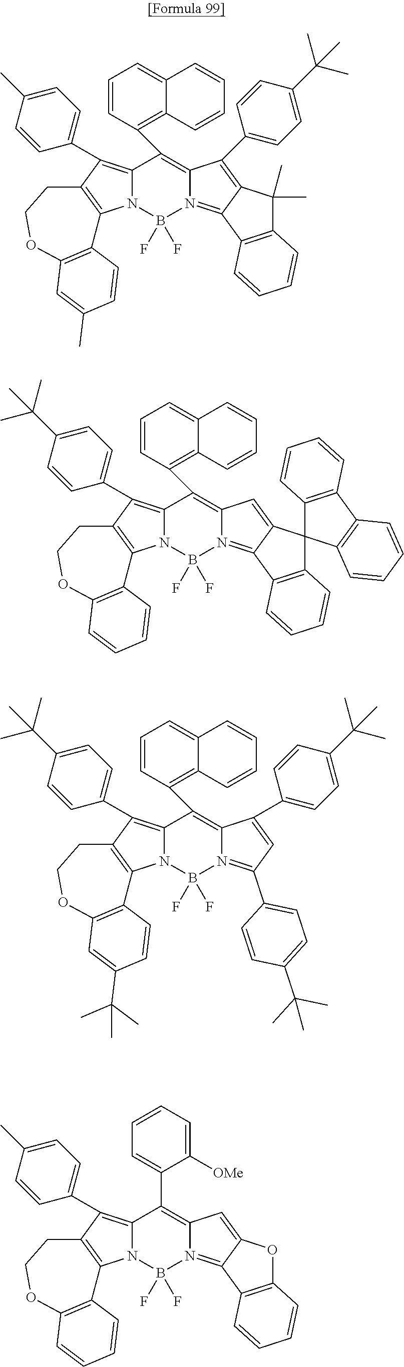

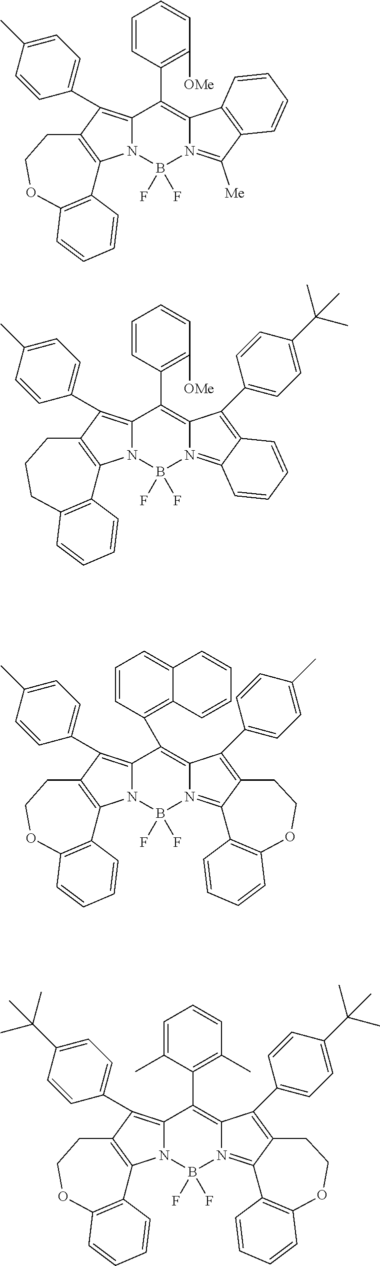

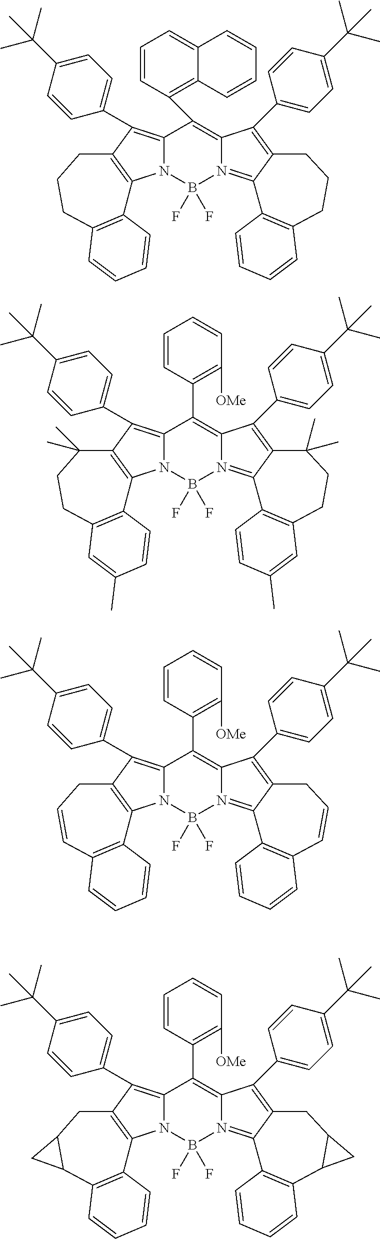

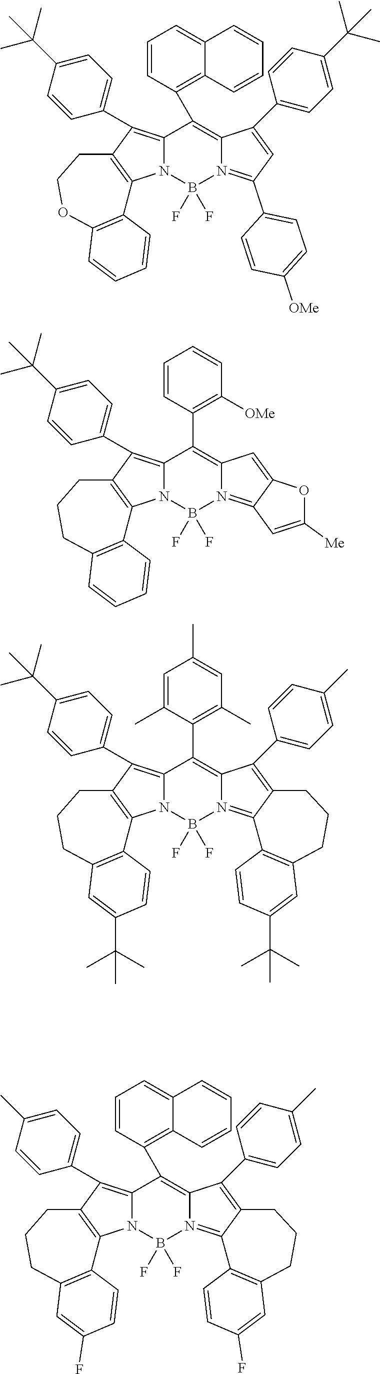

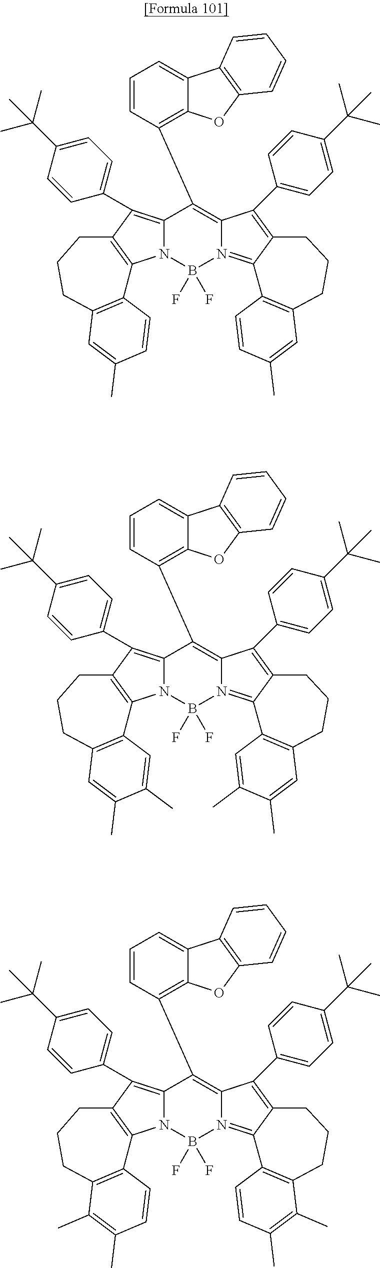

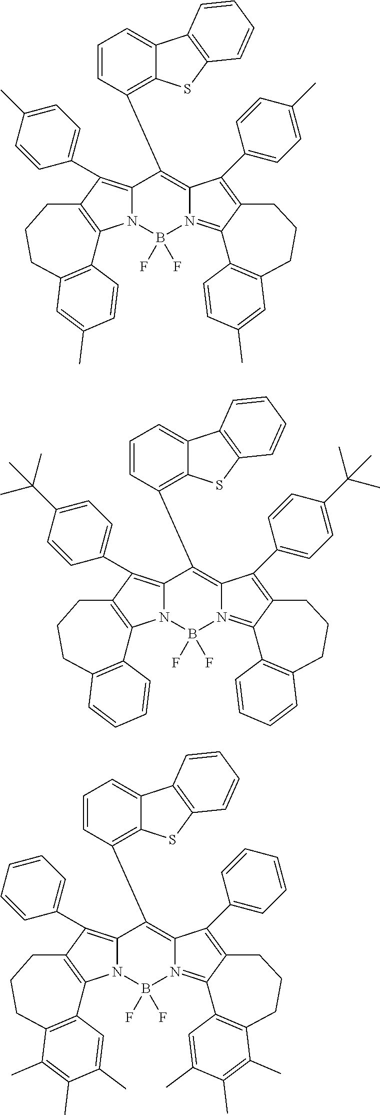

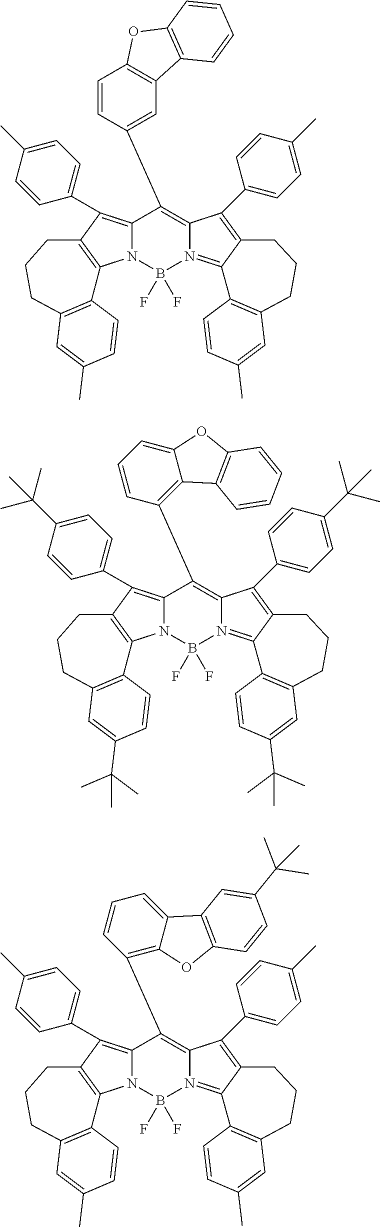

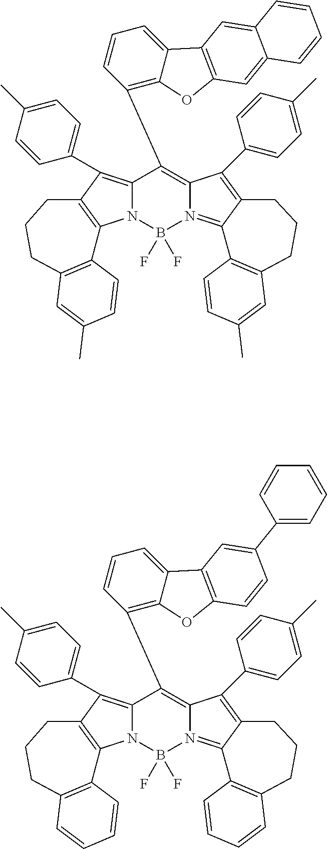

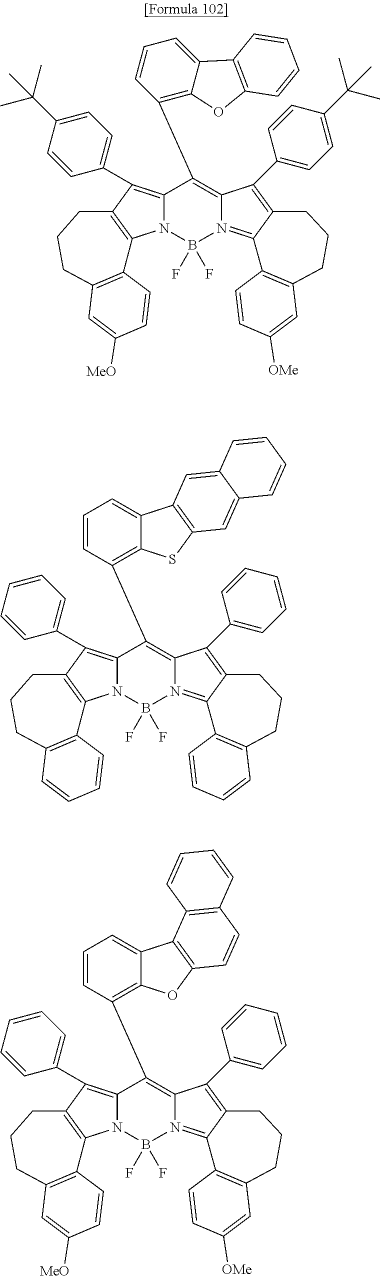

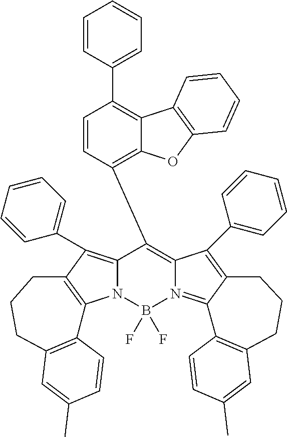

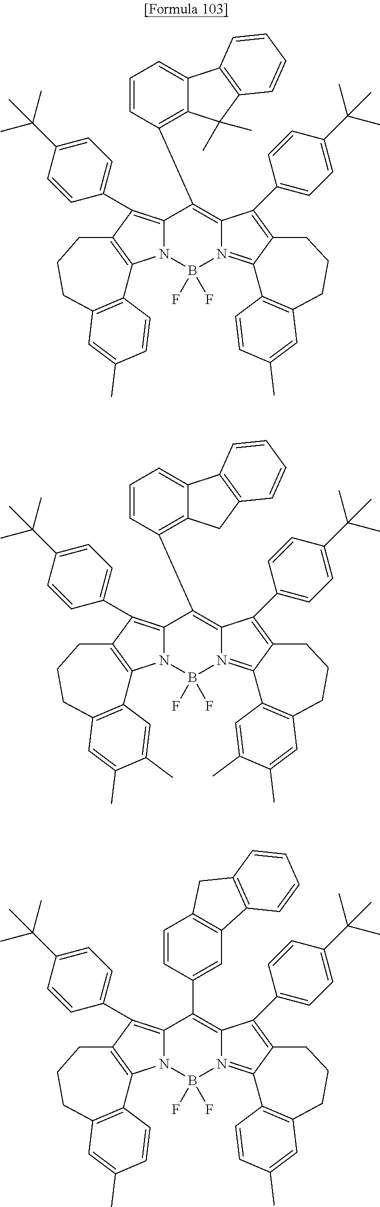

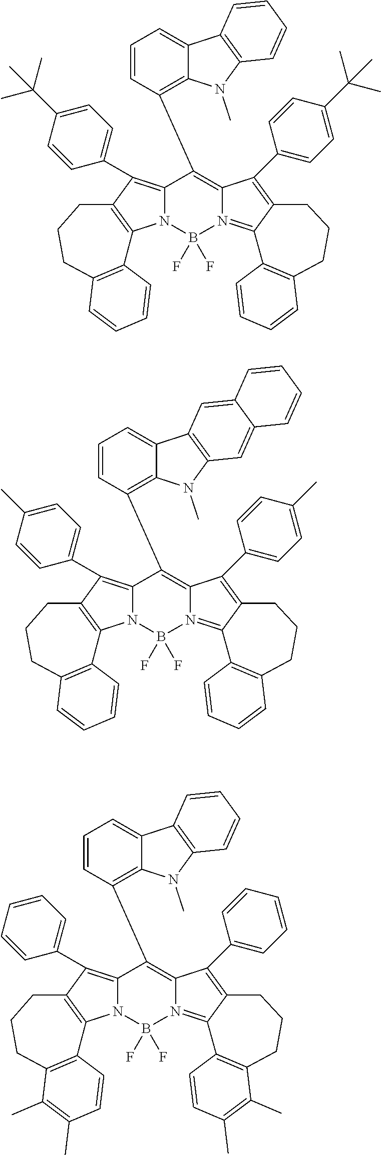

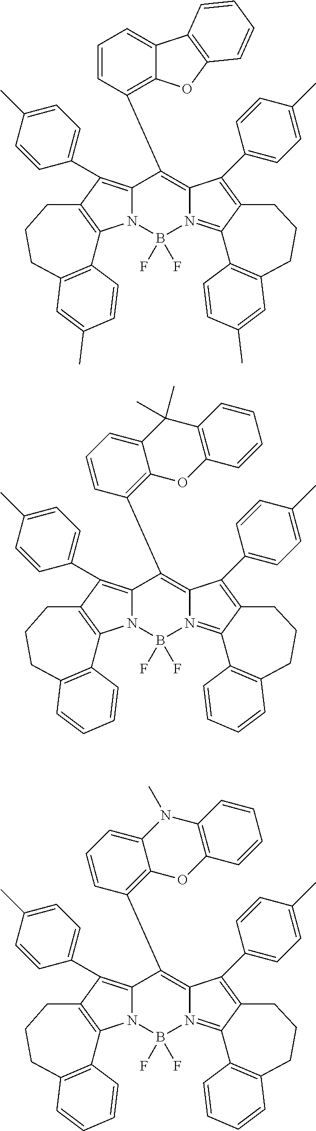

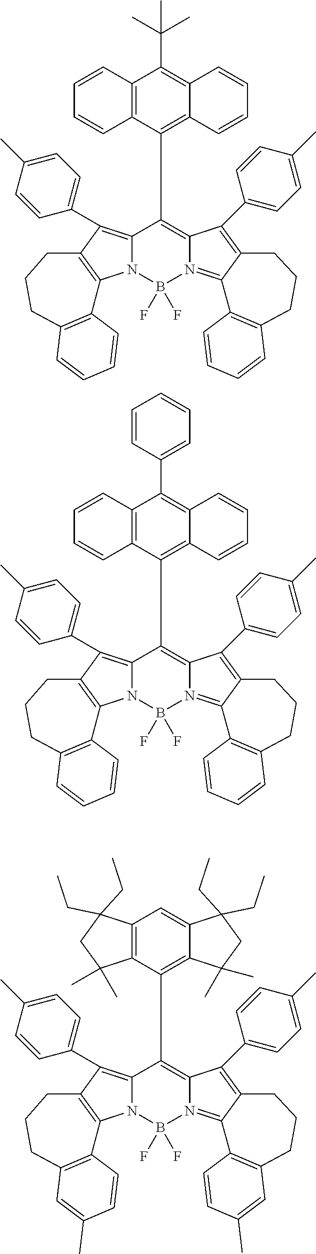

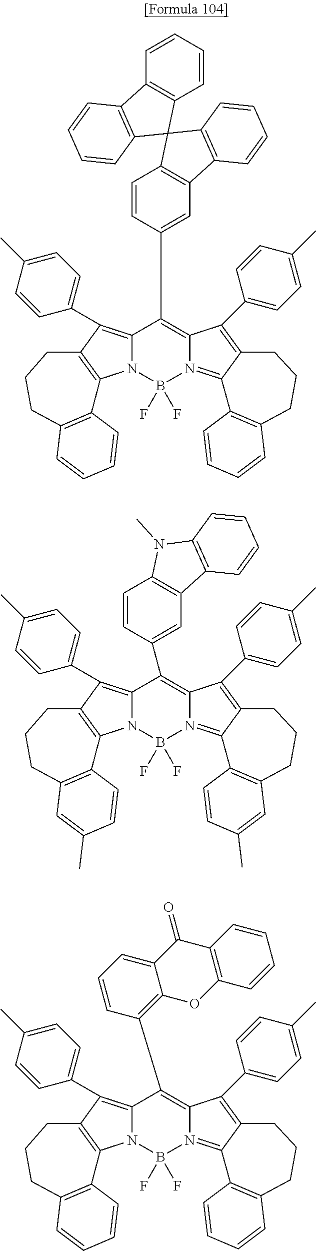

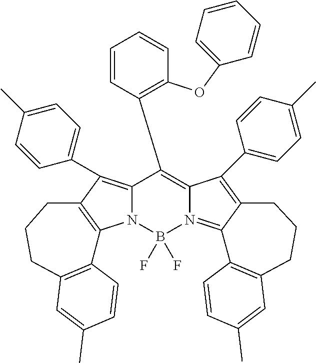

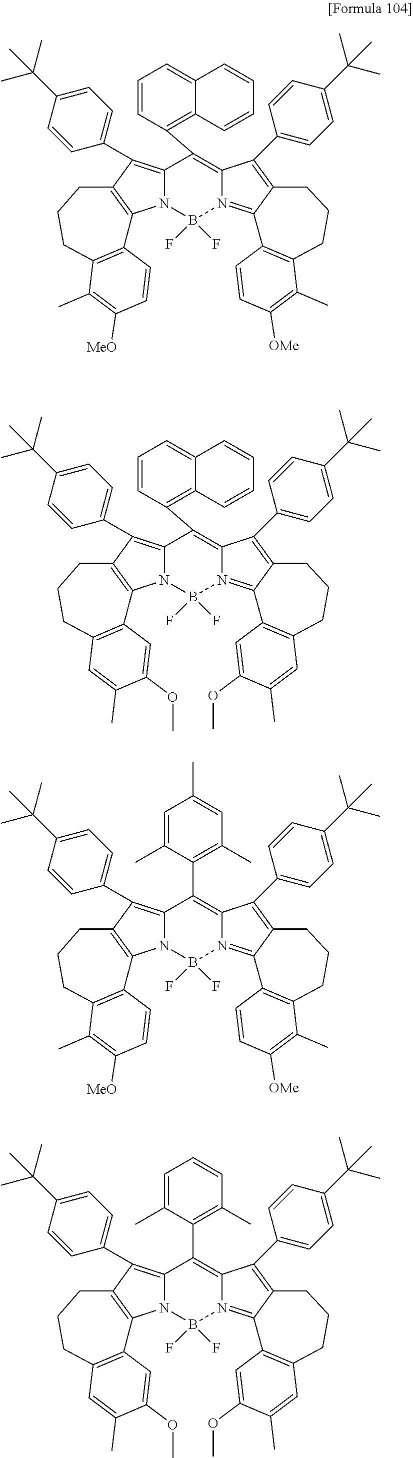

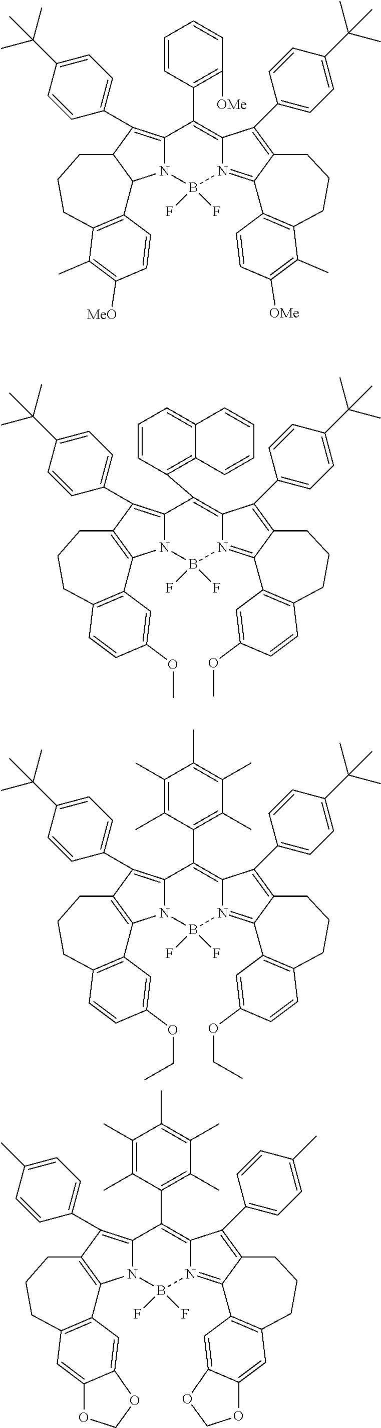

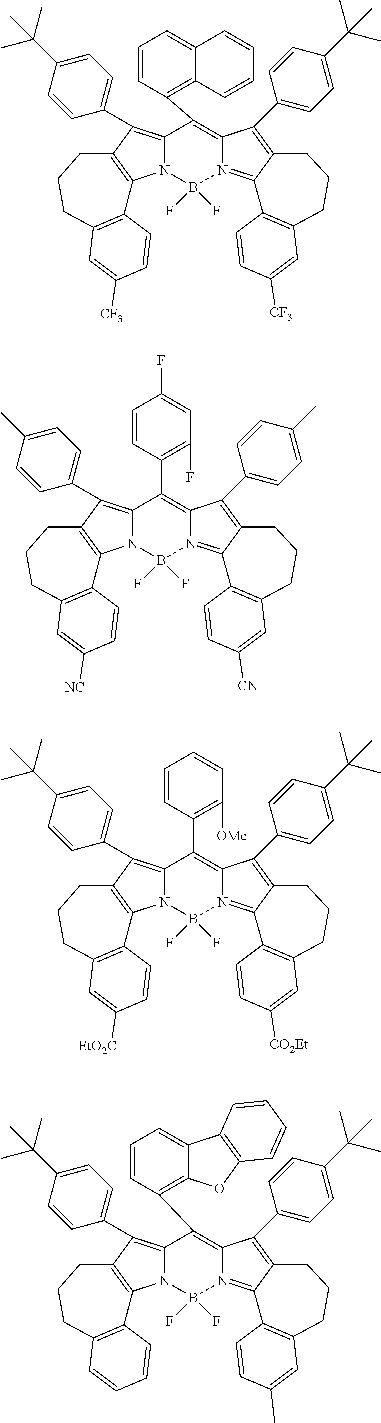

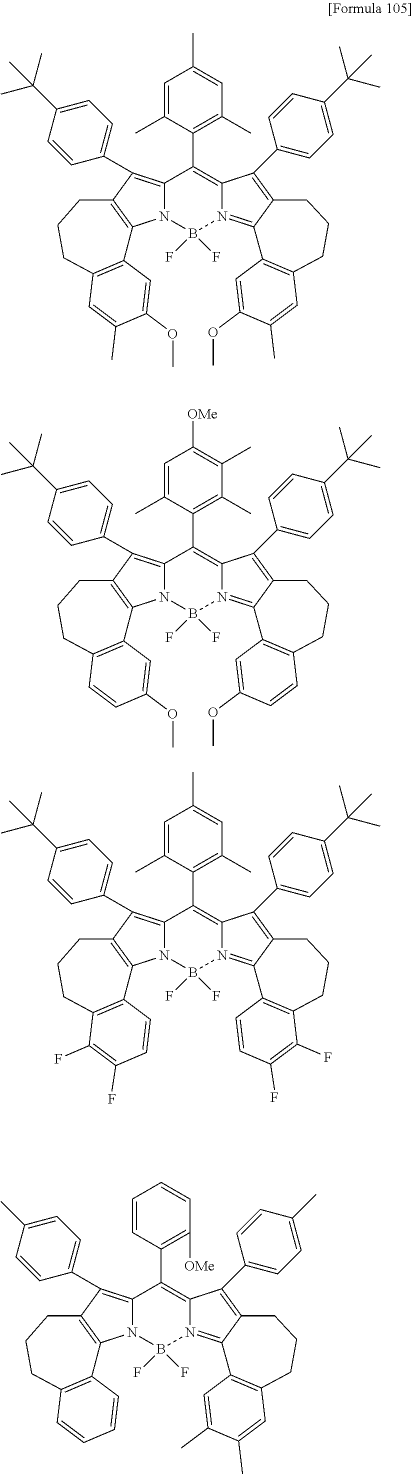

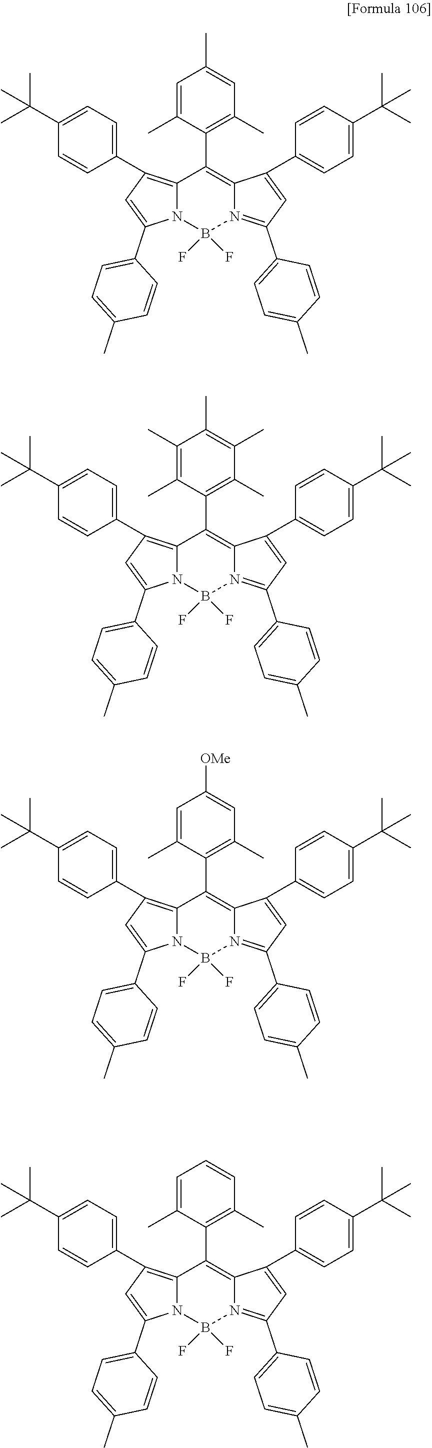

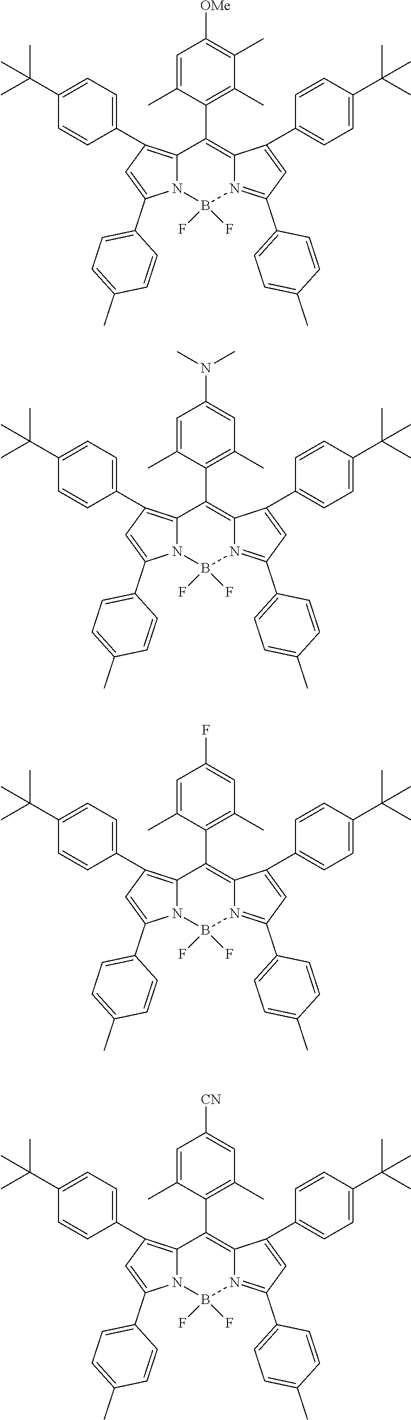

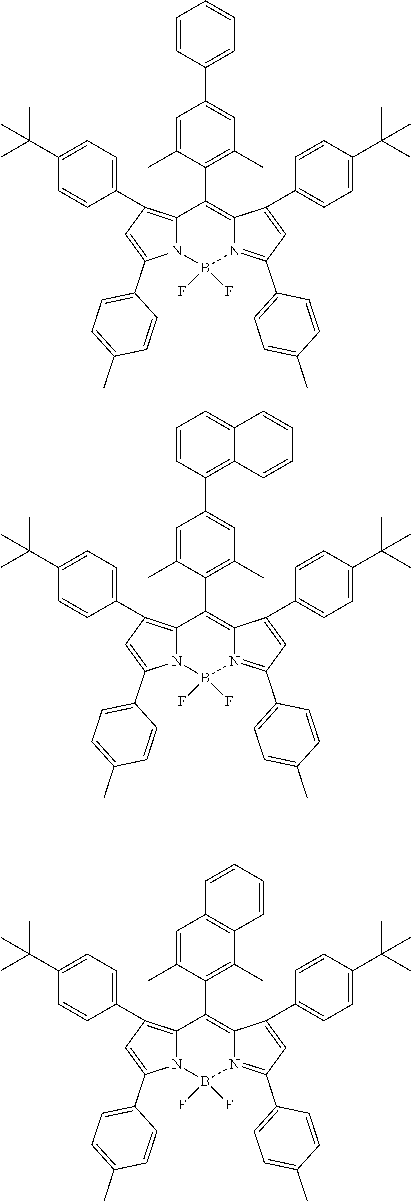

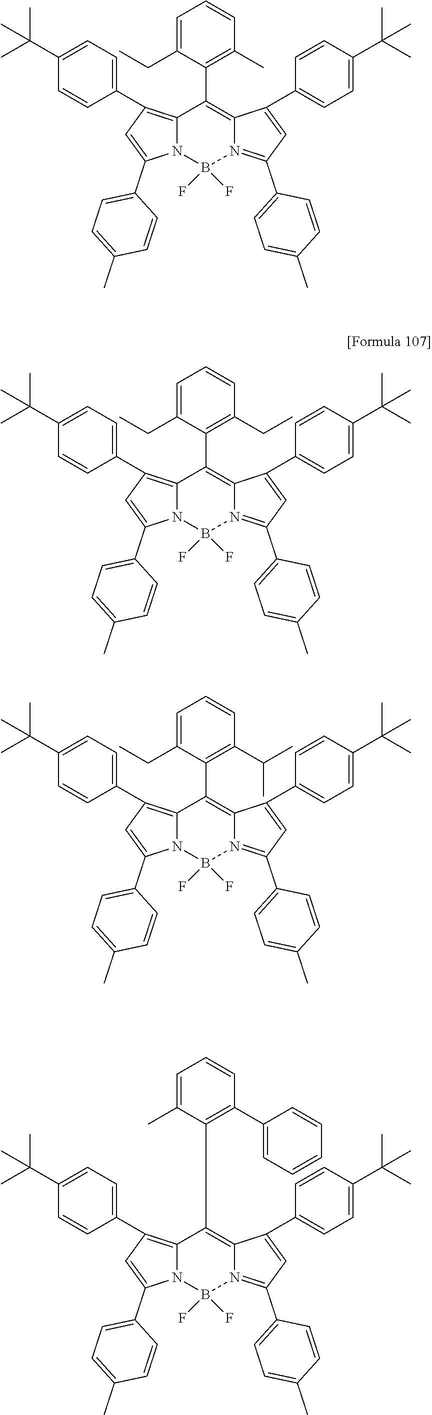

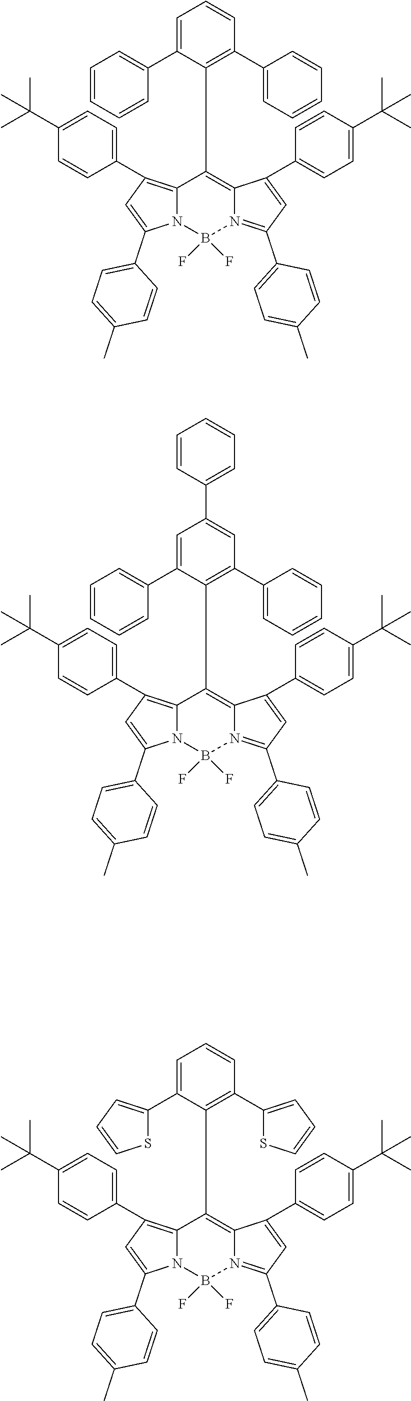

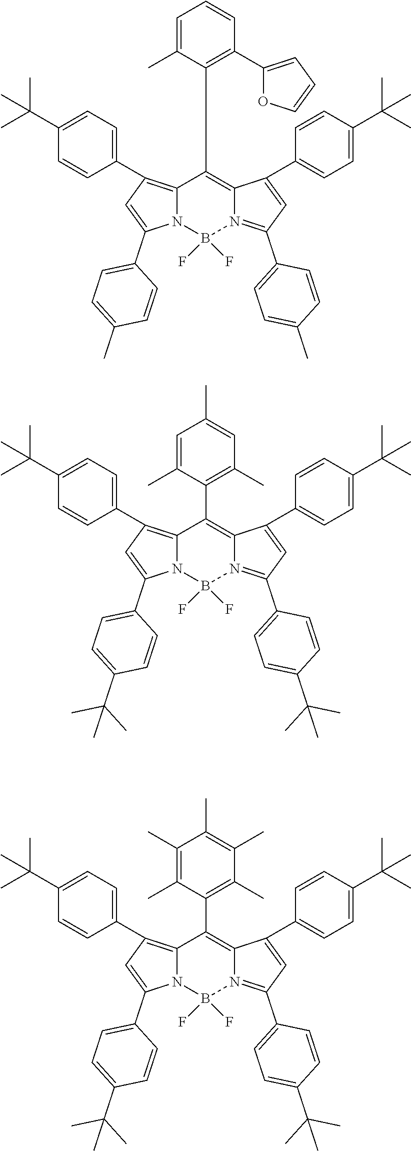

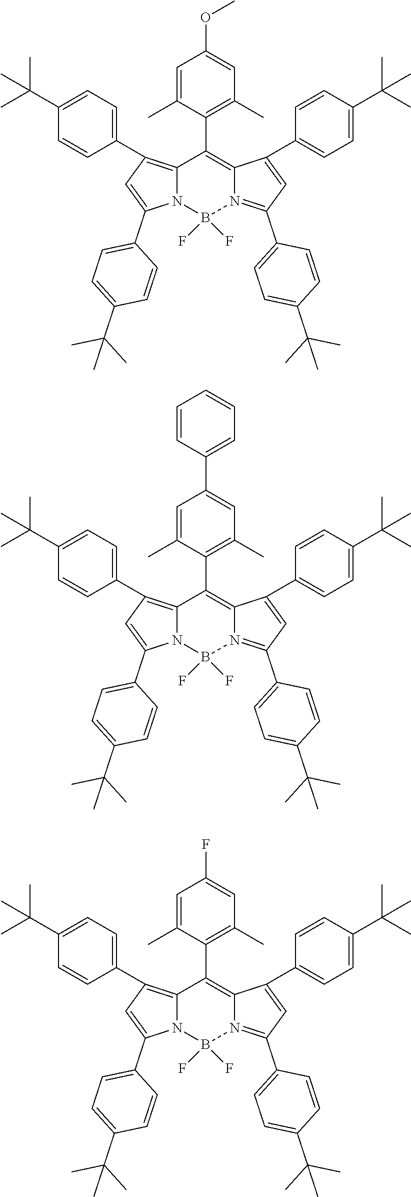

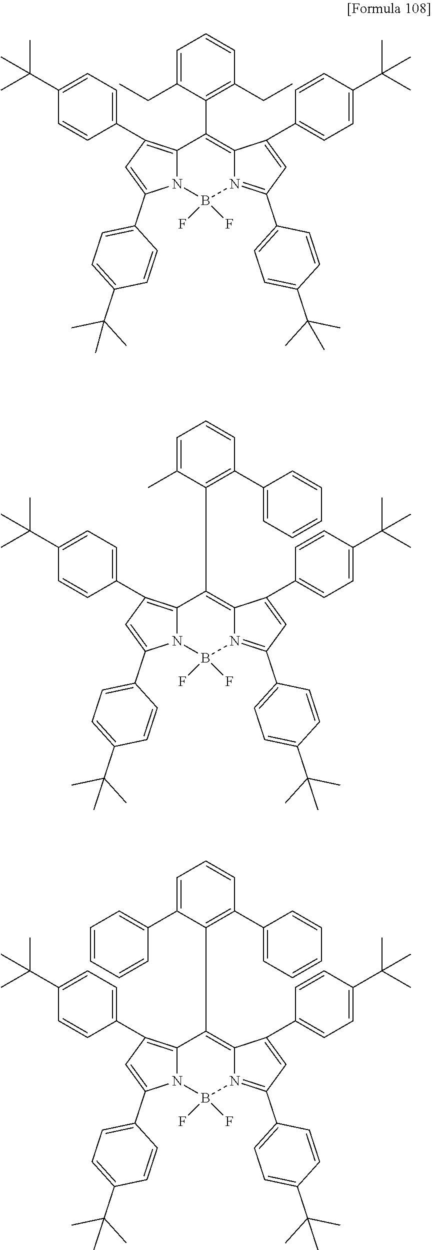

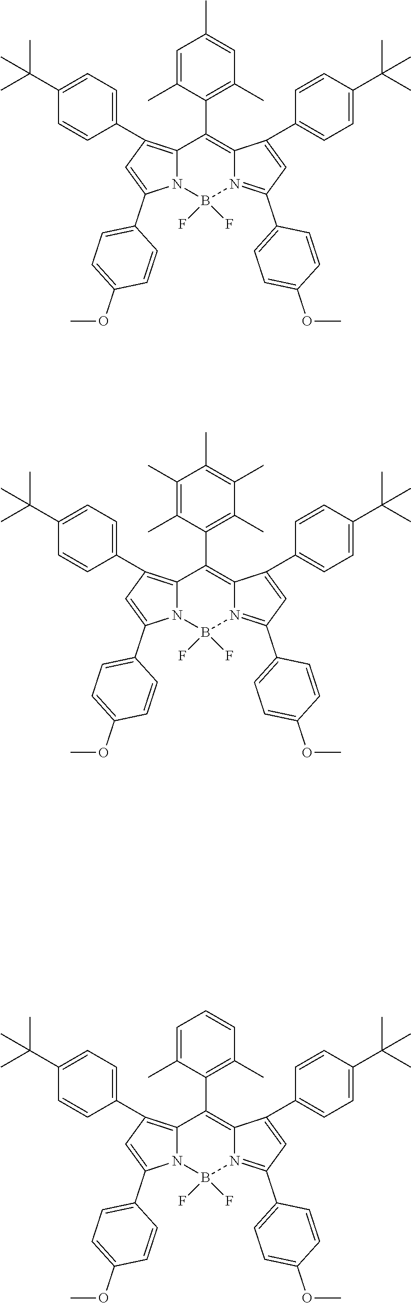

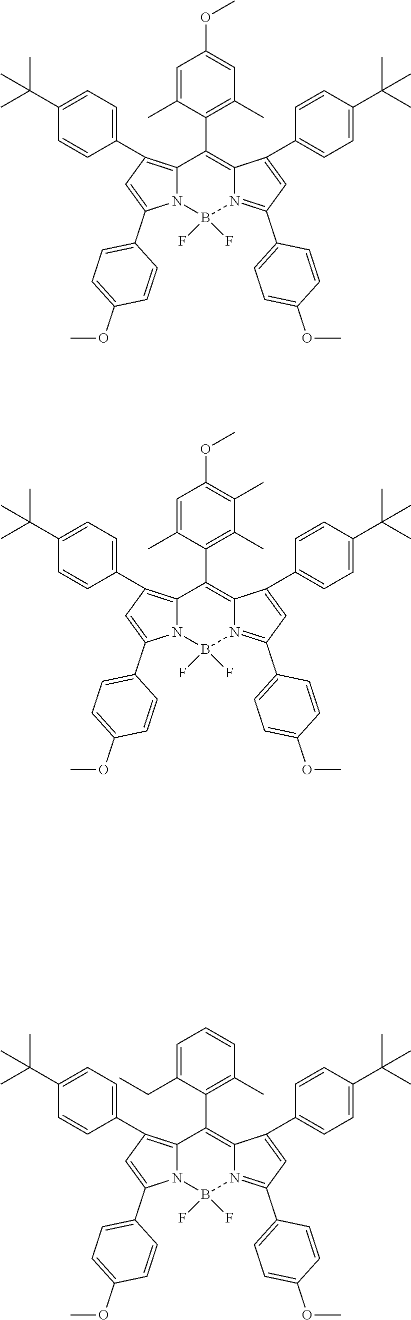

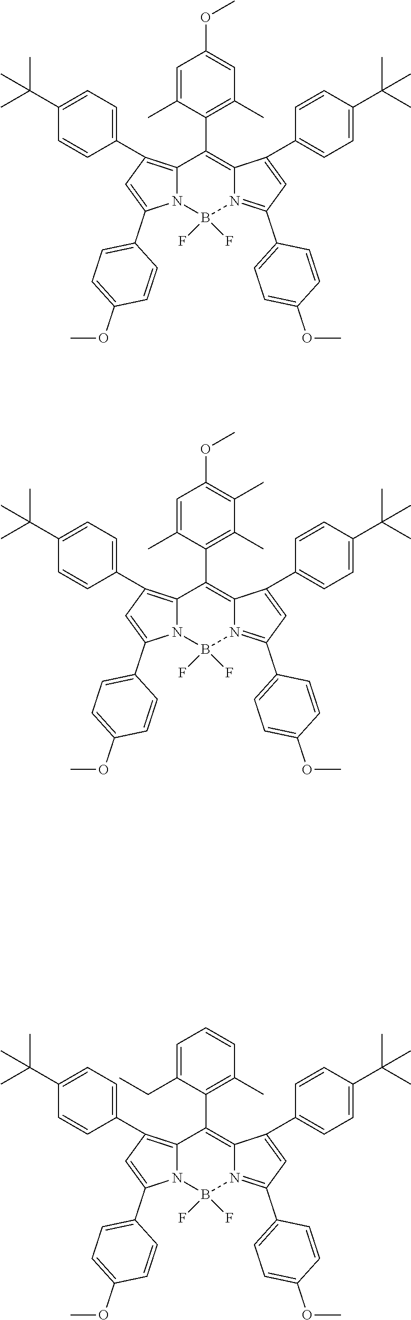

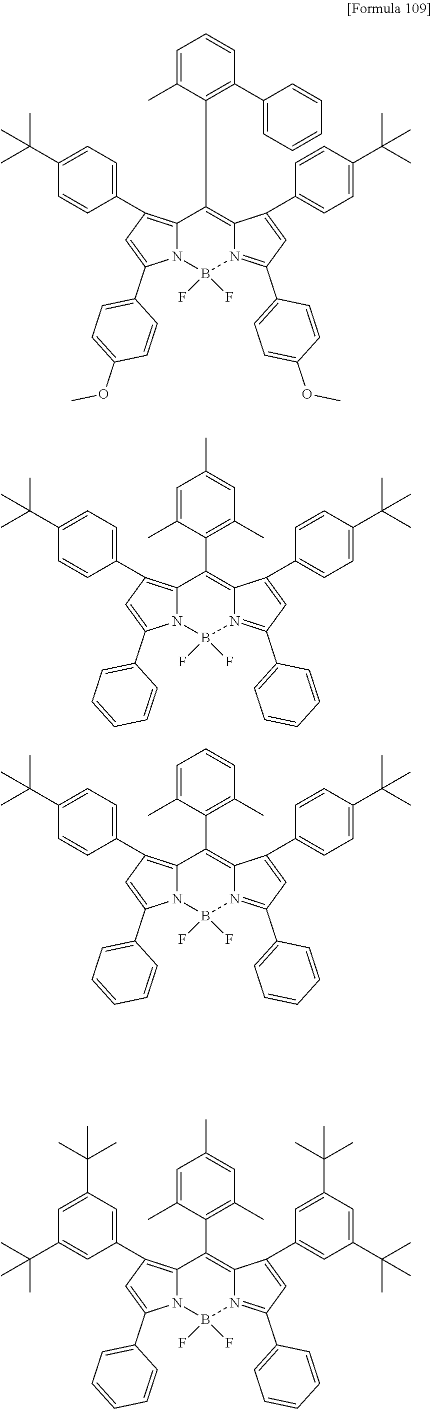

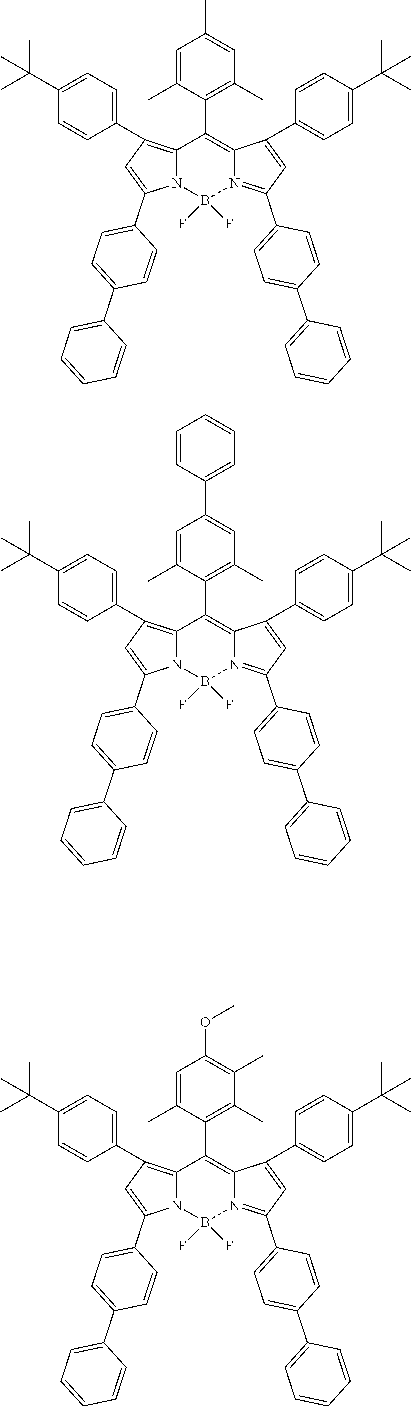

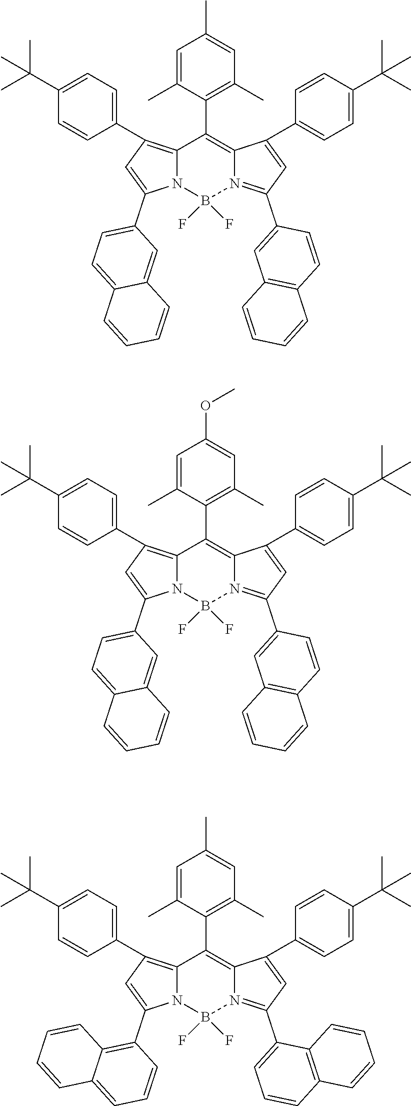

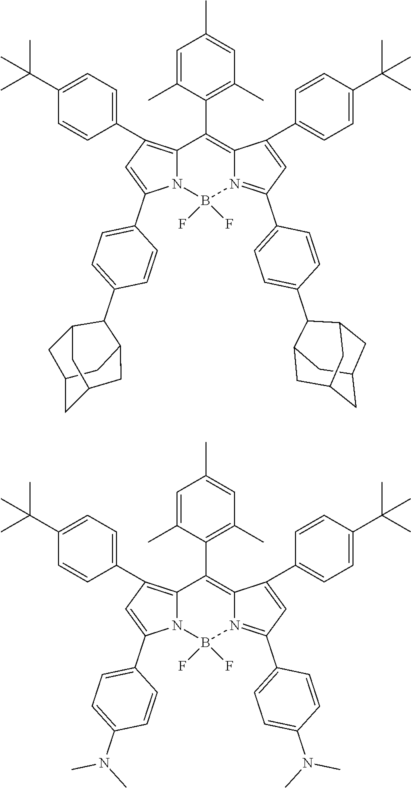

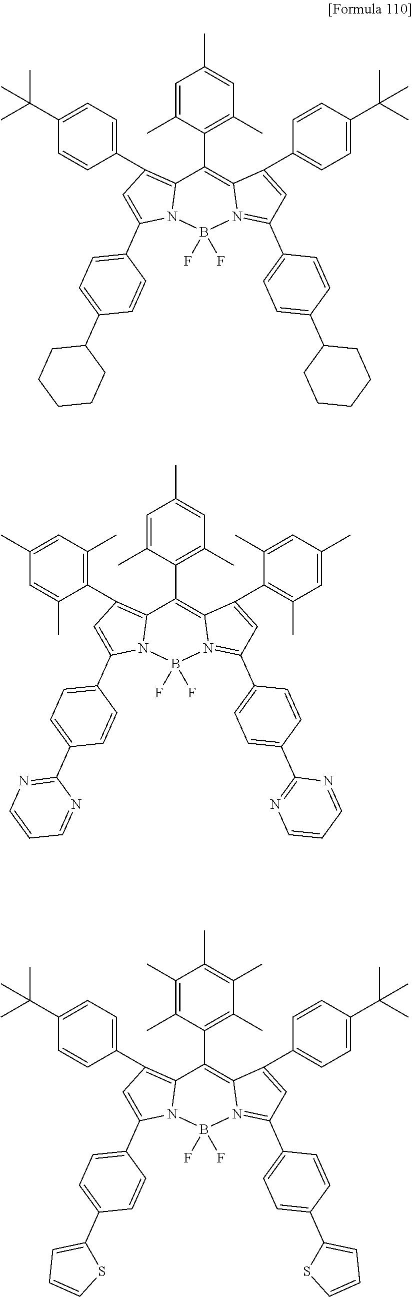

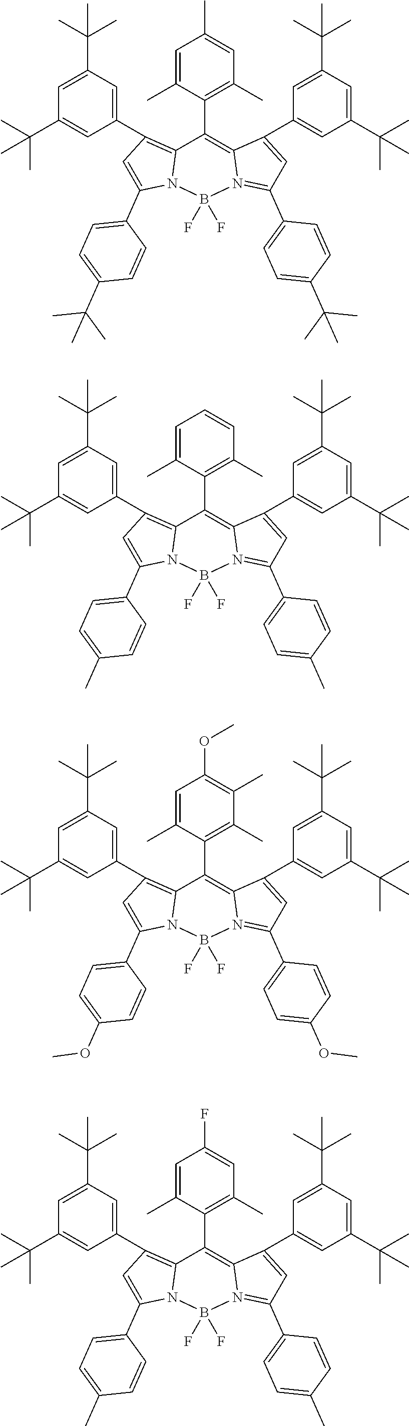

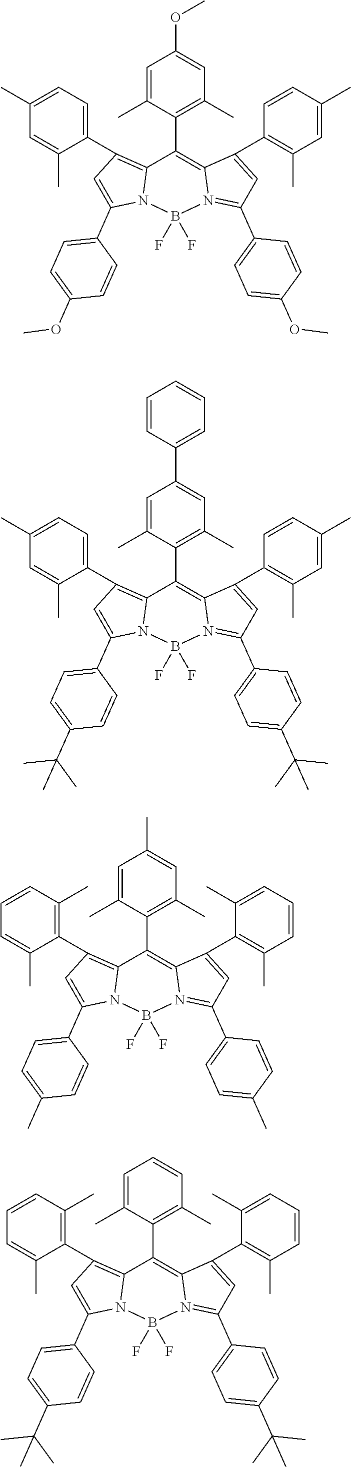

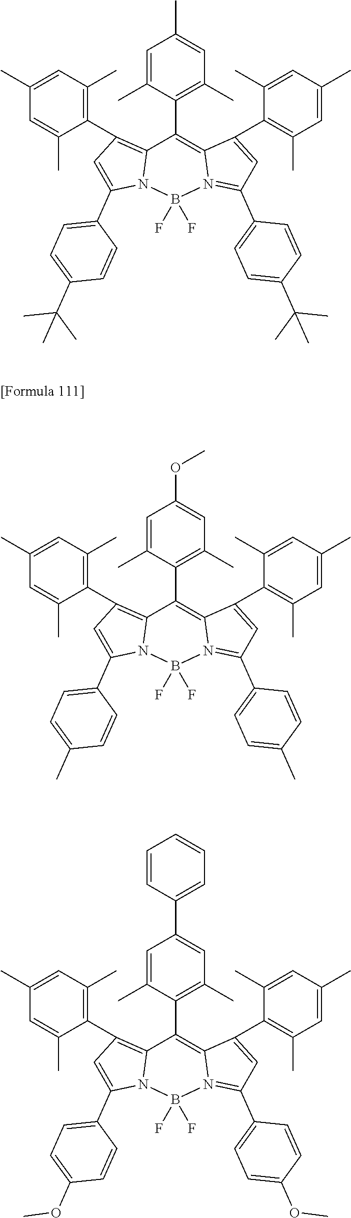

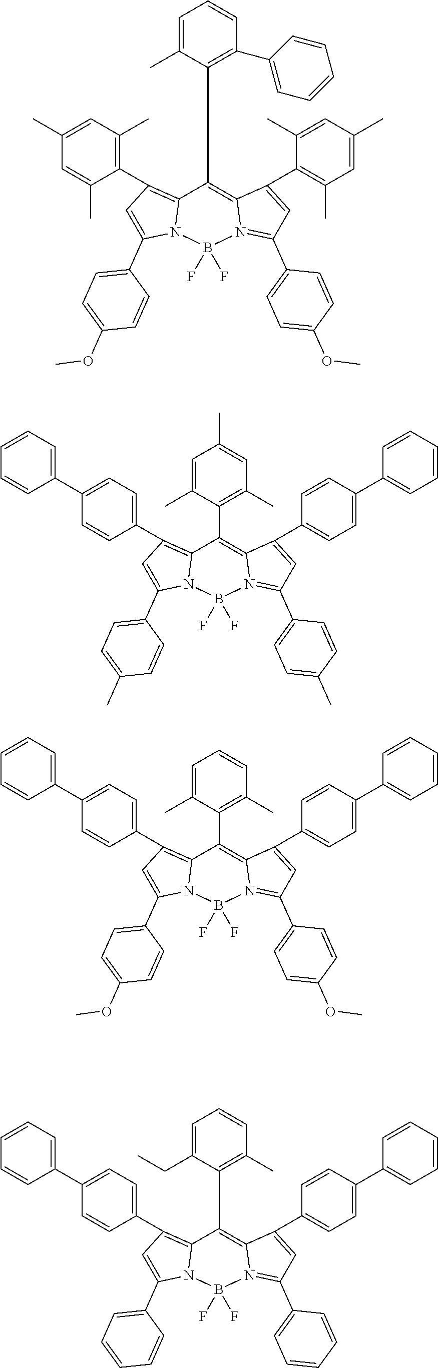

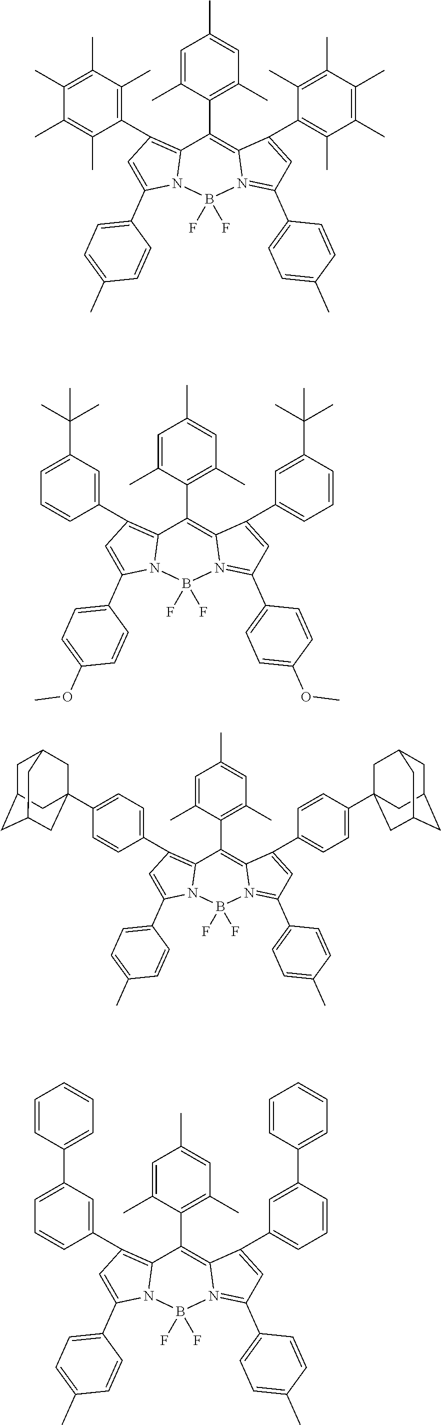

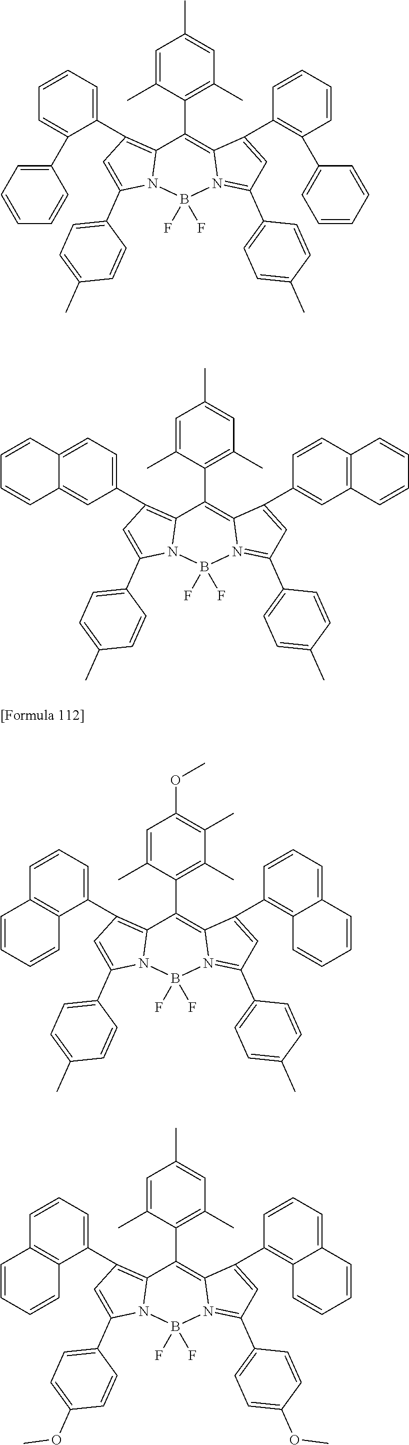

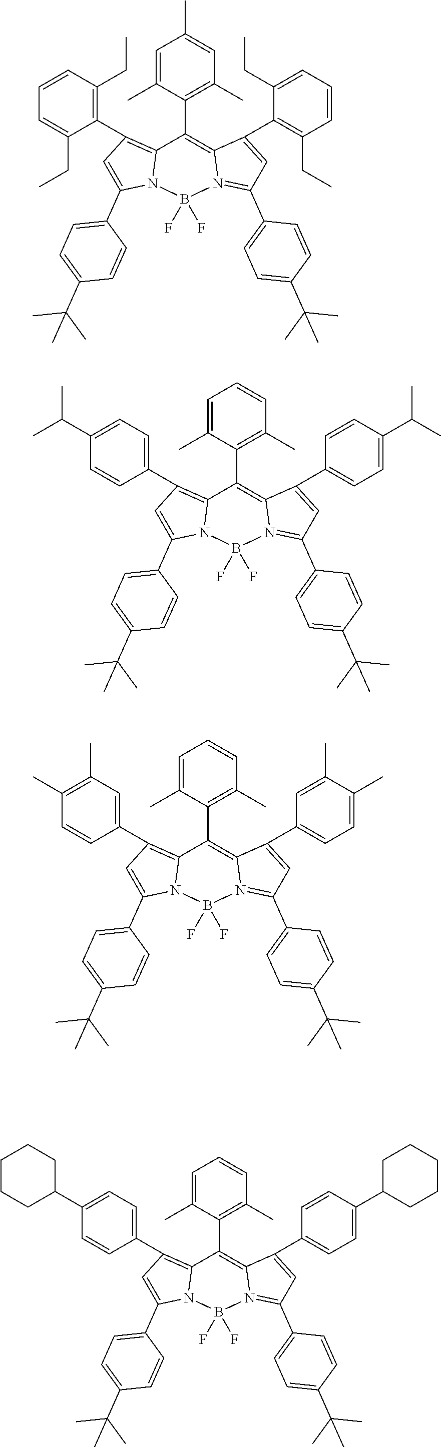

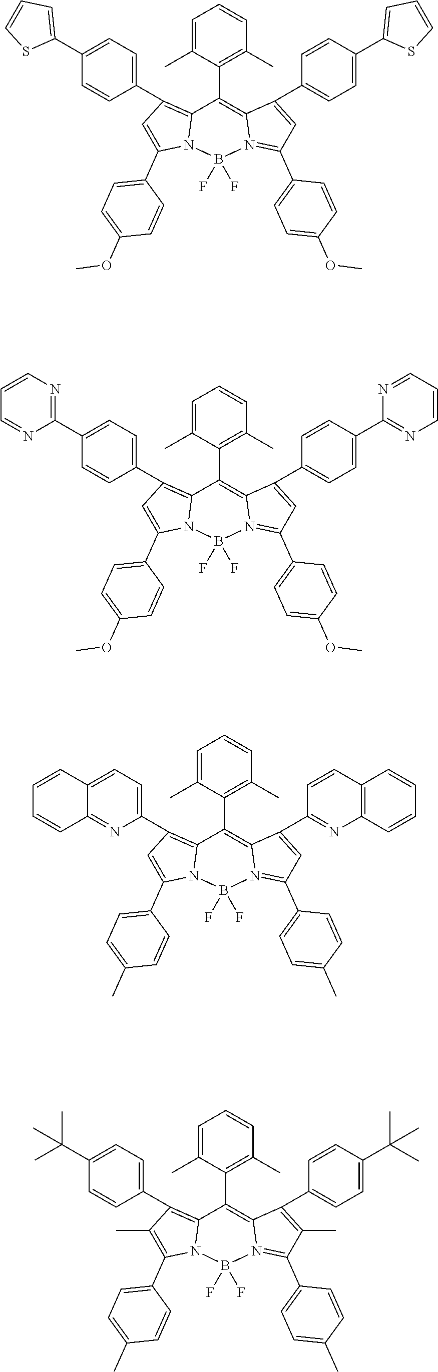

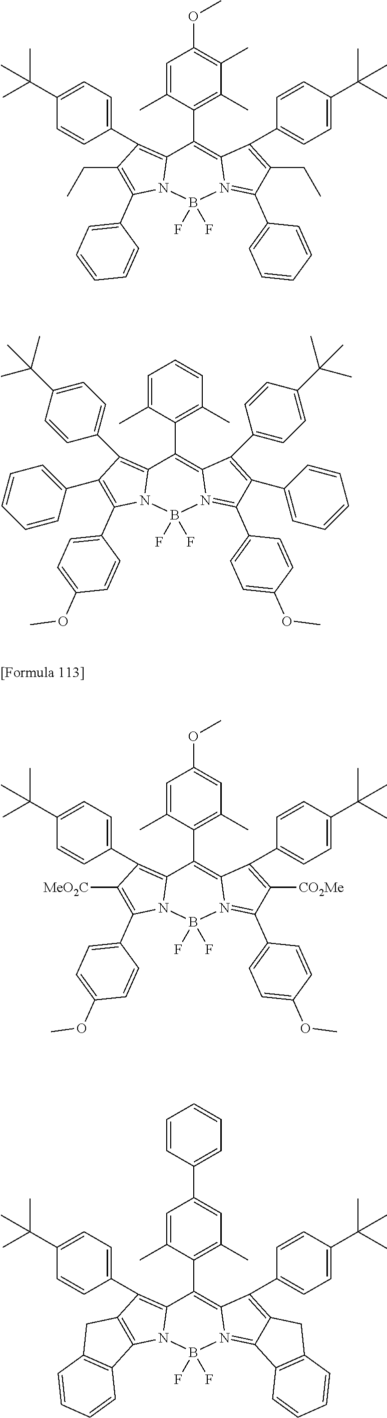

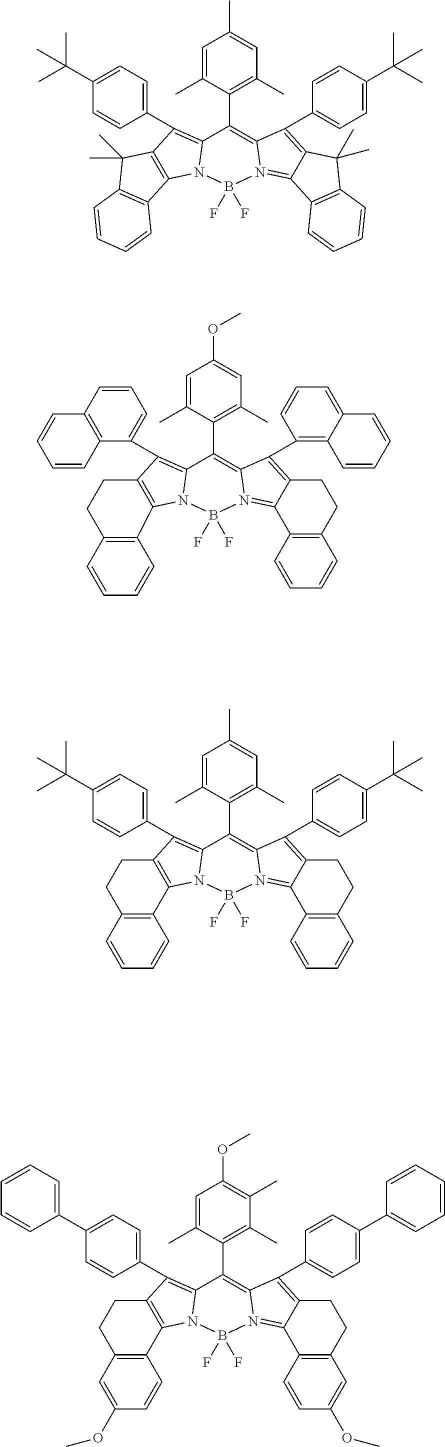

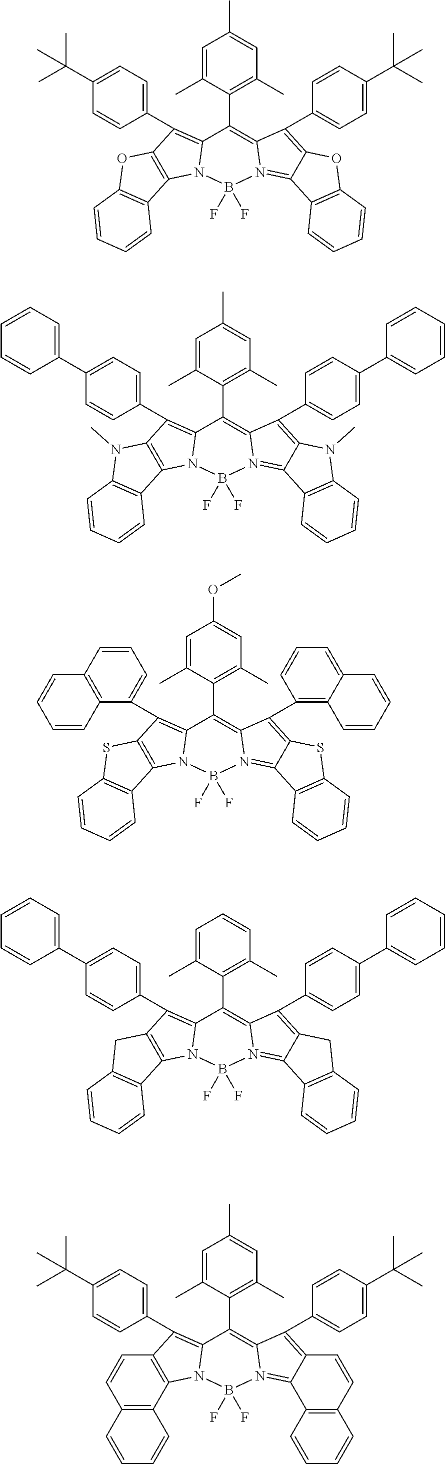

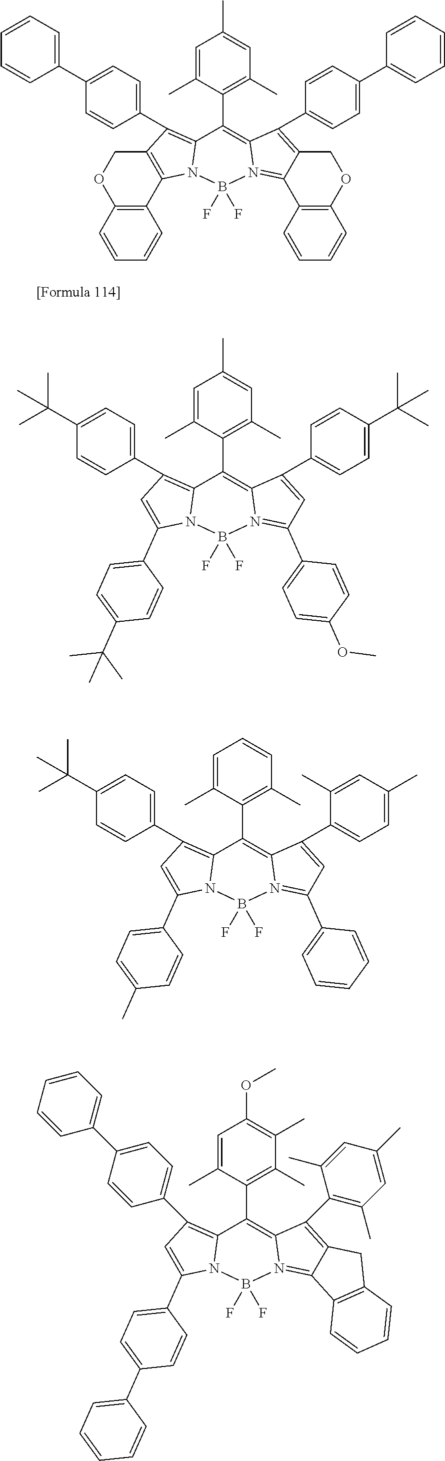

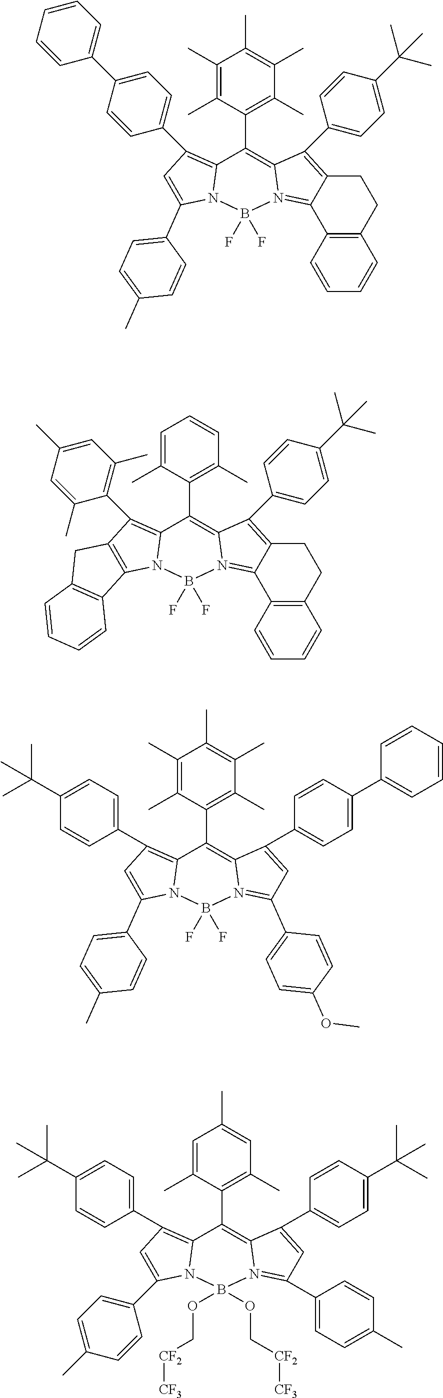

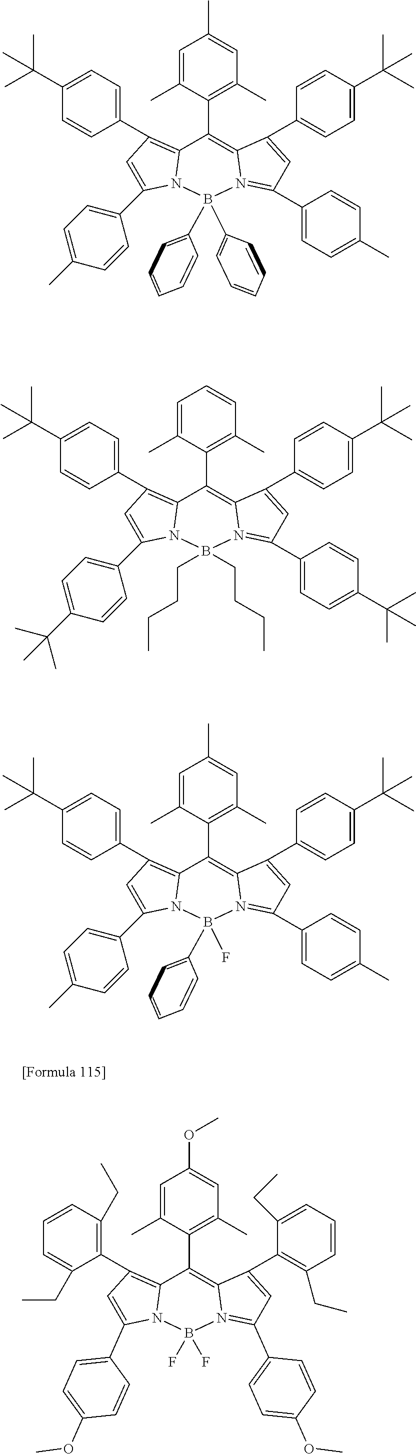

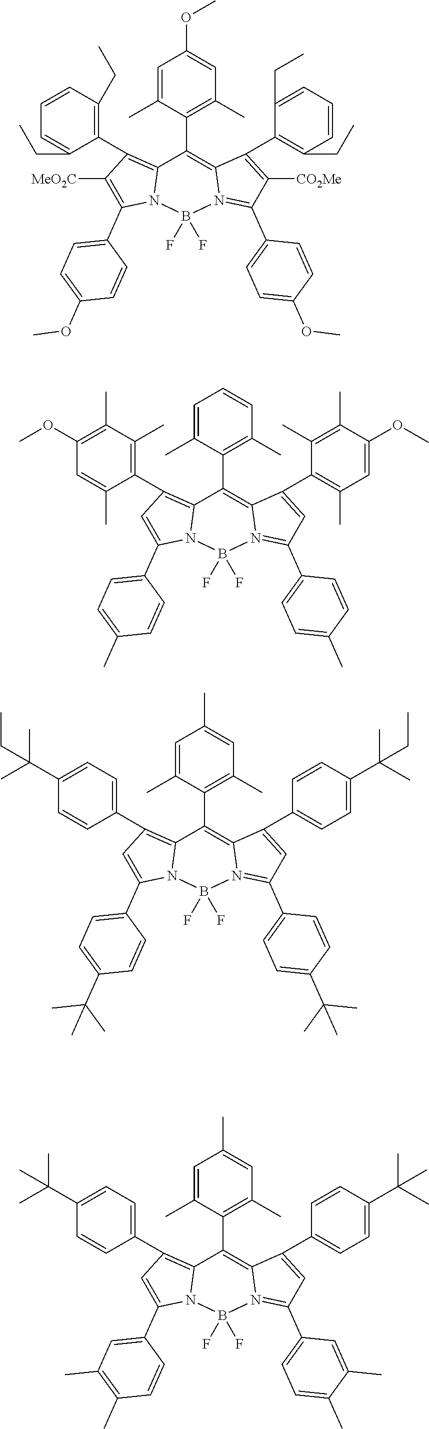

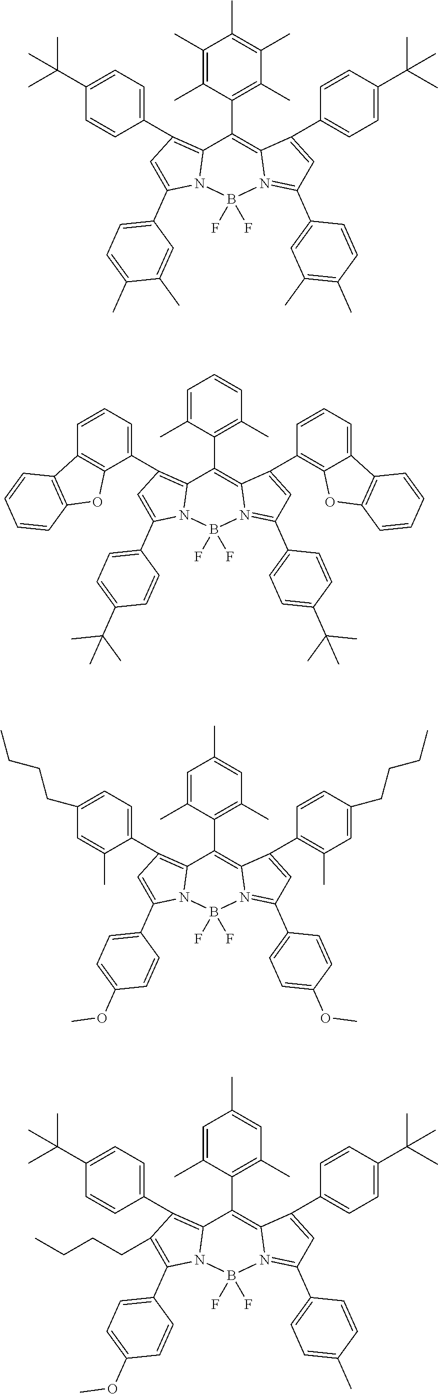

### Second Compound

The second compound is a delayed fluorescent compound.

In the exemplary embodiment, the second compound is a compound represented by a formula (2) below.

### Compound Represented by Formula (2)

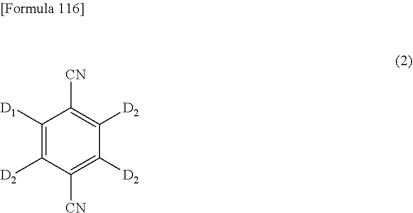

In the formula (2), D₁ is a group represented by a formula (2-1) below, D₂ is a group represented by a formula (2-2), and a plurality of D₂ are mutually the same group.

That “a plurality of D₂ are mutually the same group” means that all variables represented by the same symbol in the formula (2-2) are the same.

The variables in the formula (2-2) refer to R₁₆₁ to R₁₆₈. Specifically, in the formula (2), in the group represented by the formula (2-2) representing D₂, a plurality of R₁₆₁ are the same, a plurality of R₁₆₂ are the same, a plurality of R₁₆₃ are the same, a plurality of R₁₆₄ are the same, a plurality of R₁₆₅ are the same, a plurality of R₁₆₆ are the same, a plurality of R₁₆₇ are the same, and a plurality of R₁₆₈ are the same. Specifically, three D₂ in the formula (2) are mutually the same group also inclusive of a substituent.

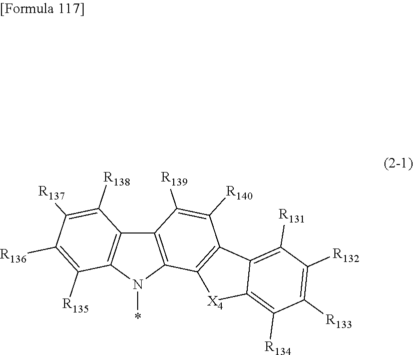

In the formula (2-1): X₄ is an oxygen atom or a sulfur atom, and R₁₃₁ to R₁₄₀ are each independently a hydrogen atom or a substituent;

R₁₃₁ to R₁₄₀ serving as the substituent are each independently a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkylsilyl group having 3 to 6 carbon atoms, a substituted or unsubstituted alkoxy group having 1 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and

* represents a bonding position to a benzene ring in the formula (2).

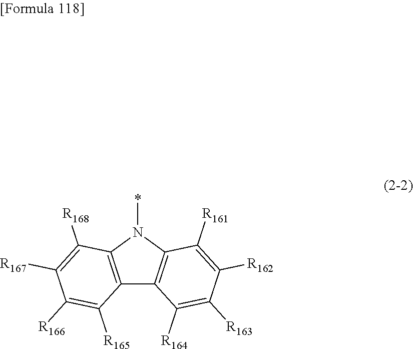

In the formula (2-2): R₁₆₁ to R₁₆₈ are each independently a hydrogen atom or a substituent;

R₁₆₁ to R₁₆₈ serving as the substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted alkylsilyl group having 3 to 6 carbon atoms, a substituted or unsubstituted alkoxy group having 1 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and

* each independently represents a bonding position to a benzene ring in the formula (2).

In the formula (2-1), X₄ is preferably a sulfur atom.

In the formula (2-1), X₄ is also preferably an oxygen atom.

In the second compound, the group represented by the formula (2-2) is preferably a group represented by one of formulae (2-20) to (2-26).

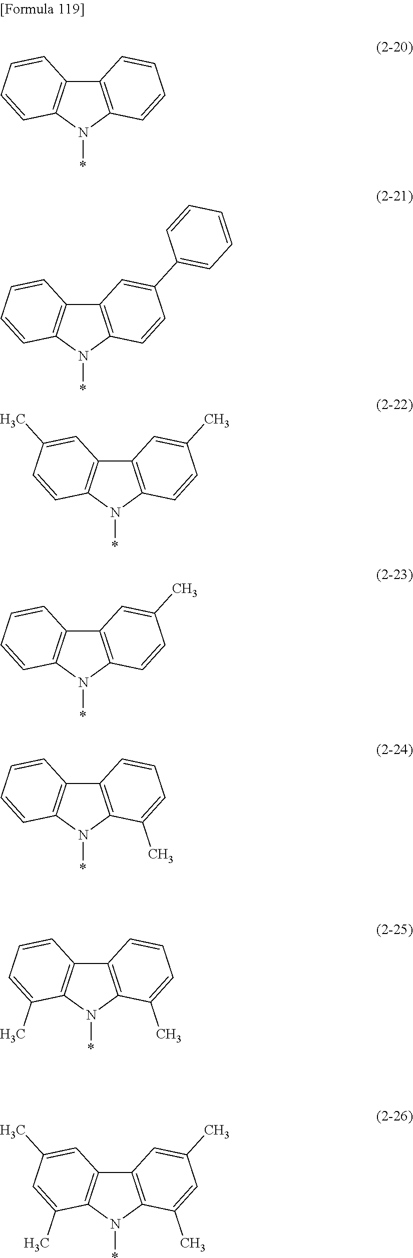

In the formulae (2-20) to (2-26), * each independently represents a bonding position to a benzene ring in the formula (2).

In the formula (2-2), R₁₆₁ to R₁₆₈ are preferably each independently a hydrogen atom, a substituted or unsubstituted aryl group having 6 to 14 ring carbon atoms, or a substituted or unsubstituted alkyl group having 1 to 6 carbon atoms, more preferably a hydrogen atom or a substituted or unsubstituted alkyl group having 1 to 6 carbon atoms.

In the formula (2-2), it is also preferable that at least one of R₁₆₁, R₁₆₃, R₁₆₆ or R₁₆₈ has a substituent, and the substituent is each independently a substituted or unsubstituted aryl group having 6 to 14 ring carbon atoms or a substituted or unsubstituted alkyl group having 1 to 6 carbon atoms, while R₁₆₂, R₁₆₄, R₁₆₅ and R₁₆₇ are each a hydrogen atom.

In the formulae (2-1) and (2-2), R₁₃₁ to R₁₄₀ and R₁₆₁ to R₁₆₈ serving as the substituent are preferably each independently a halogen atom, an unsubstituted aryl group having 6 to 14 ring carbon atoms, an unsubstituted heterocyclic group having 5 to 14 ring atoms, an unsubstituted alkyl group having 1 to 6 carbon atoms, an unsubstituted alkyl halide group having 1 to 6 carbon atoms, an unsubstituted alkylsilyl group having 3 to 6 carbon atoms, an unsubstituted alkoxy group having 1 to 6 carbon atoms, an unsubstituted aryloxy group having 6 to 14 ring carbon atoms, an unsubstituted alkylamino group having 2 to 12 carbon atoms, an unsubstituted alkylthio group having 1 to 6 carbon atoms, or an unsubstituted arylthio group having 6 to 14 ring carbon atoms.

In the formulae (2-1) and (2-2), R₁₃₁ to R₁₄₀ and R₁₆₁ to R₁₆₈ are preferably each independently a hydrogen atom, a substituted or unsubstituted aryl group having 6 to 14 ring carbon atoms, a substituted or unsubstituted heterocyclic group having 5 to 14 ring atoms, or a substituted or unsubstituted alkyl group having 1 to 6 carbon atoms, more preferably a hydrogen atom, a substituted or unsubstituted aryl group having 6 to 14 ring carbon atoms or a substituted or unsubstituted alkyl group having 1 to 6 carbon atoms, further preferably a hydrogen atom or a substituted or unsubstituted alkyl group having 1 to 6 carbon atoms.

In the formulae (2-1) and (2-2), it is more preferable that R₁₃₁ to R₁₄₀ and R₁₆₁ to R₁₆₈ serving as the substituent are each independently an unsubstituted aryl group having 6 to 14 ring carbon atoms or an unsubstituted alkyl group having 1 to 6 carbon atoms.

In the formula (2-1), it is also preferable that R₁₃₇ is a substituent and R₁₃₇ serving as the substituent is a substituted or unsubstituted aryl group having 6 to 14 ring carbon atoms or a substituted or unsubstituted alkyl group having 1 to 6 carbon atoms while R₁₃₁ to R₁₃₆ and R₁₃₈ to R₁₄₀ are each a hydrogen atom.

In the formulae (2-1) and (2-2), it is also preferable that R₁₃₁ to R₁₄₀ and R₁₆₁ to R₁₆₈ are each a hydrogen atom.

### Manufacturing Method of Second Compound

The second compound can be manufactured according to, for instance, a method described later in Examples. The second compound of the exemplary embodiment can be manufactured, for instance, by application of known substitution reactions and/or materials depending on a target compound according to reactions described later in Examples.

Specific examples of the second compound (the compound represented by the formula (2)) according to the exemplary embodiment are shown below. It should be noted that the second compound of the invention is not limited to the specific examples. Me represents a methyl group.

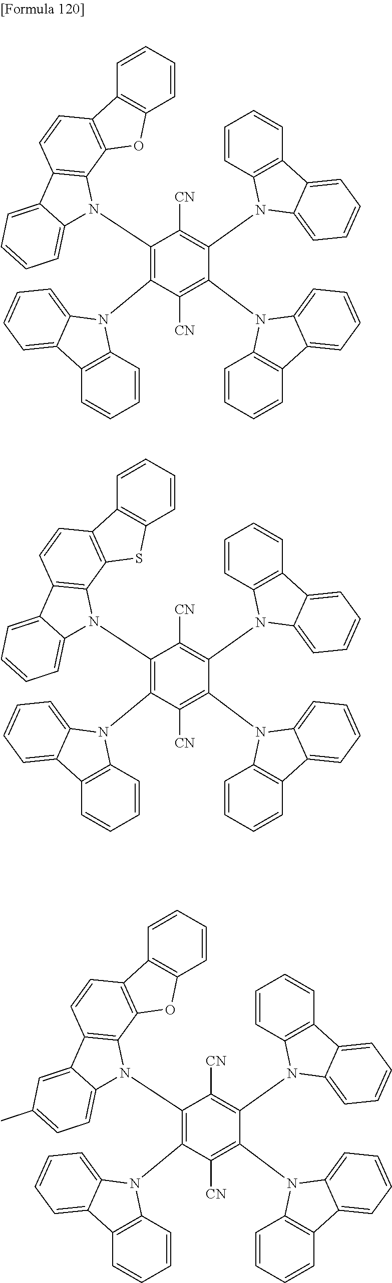 [SMILES: N#Cc1c(-n2c3ccccc3c3ccccc32)c(-n2c3ccccc3c3ccccc32)c(C#N)c(-n2c3ccccc3c3ccc4c5ccccc5oc4c32)c1-n1c2ccccc2c2ccccc21 ; N#Cc1c(-n2c3ccccc3c3ccccc32)c(-n2c3ccccc3c3ccccc32)c(C#N)c(-n2c3ccccc3c3ccc4c5ccccc5sc4c32)c1-n1c2ccccc2c2ccccc21 ; Cc1ccc2c(c1)c1ccc3c4ccccc4oc3c1n2-c1c(C#N)c(-n2c3ccccc3c3ccccc32)c(-n2c3ccccc3c3ccccc32)c(C#N)c1-n1c2ccccc2c2ccccc21]

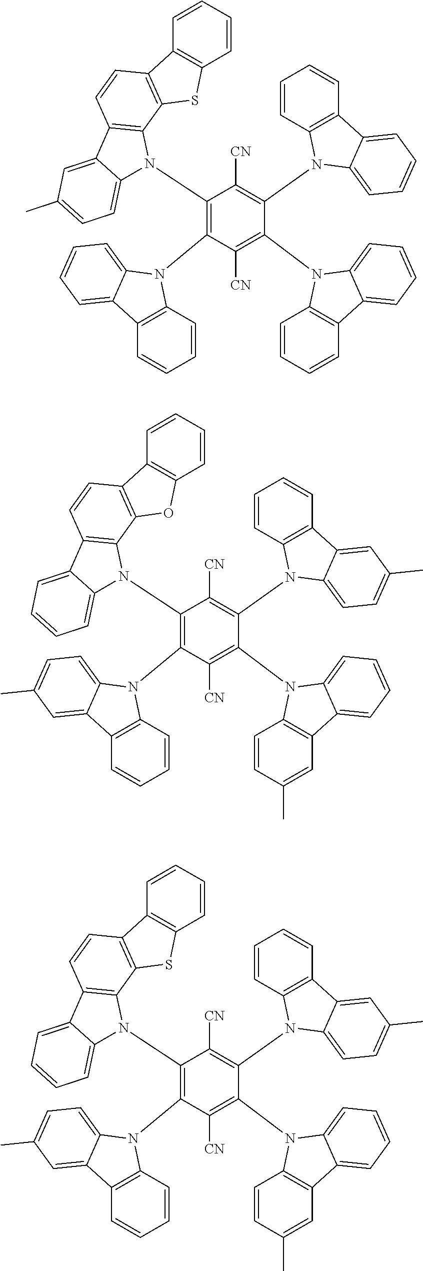

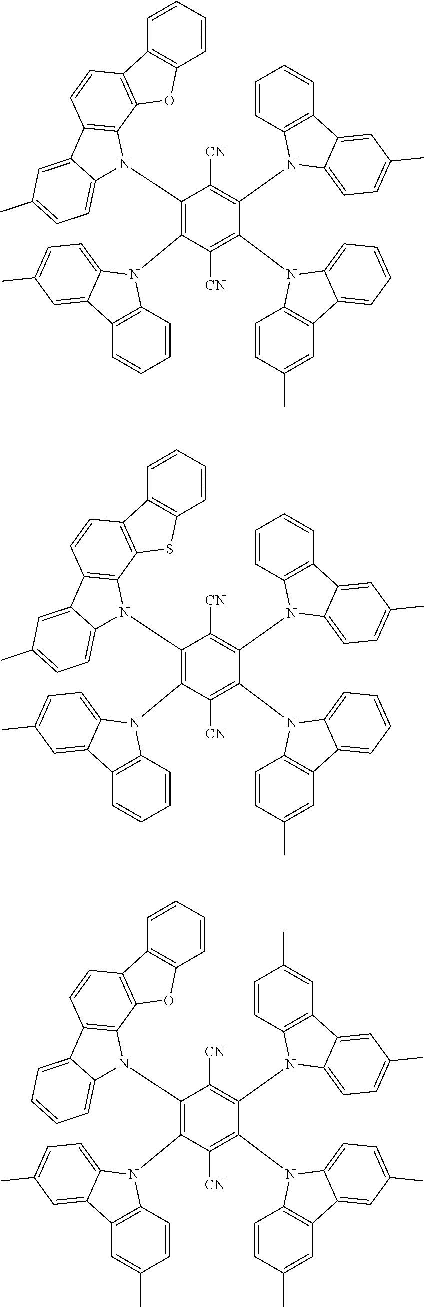

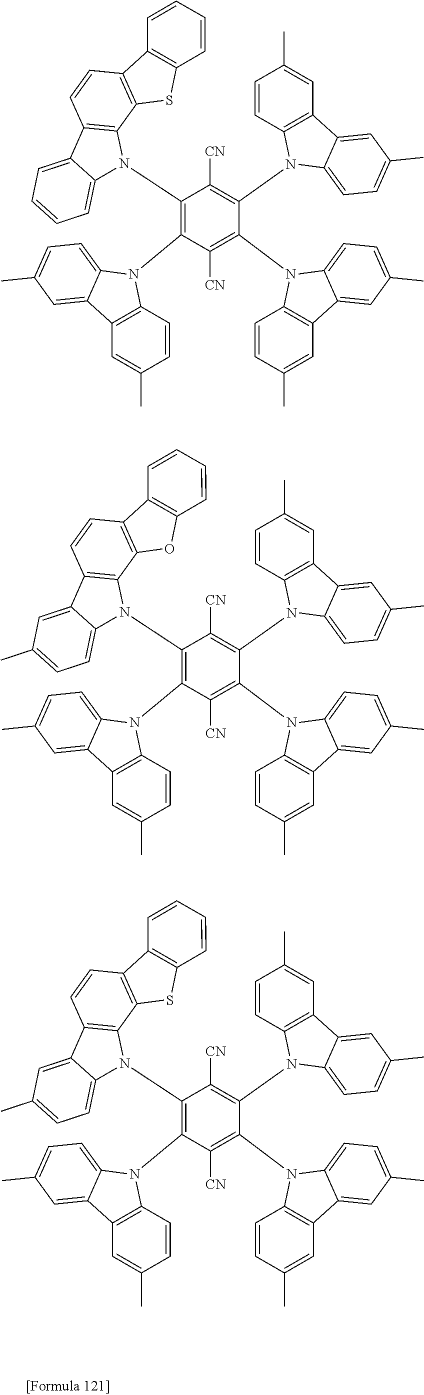

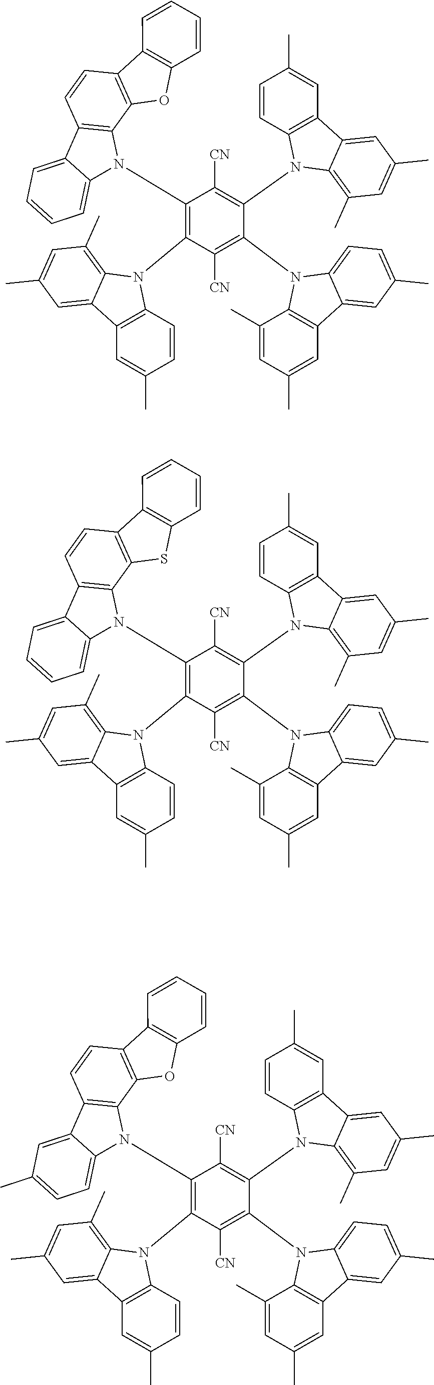

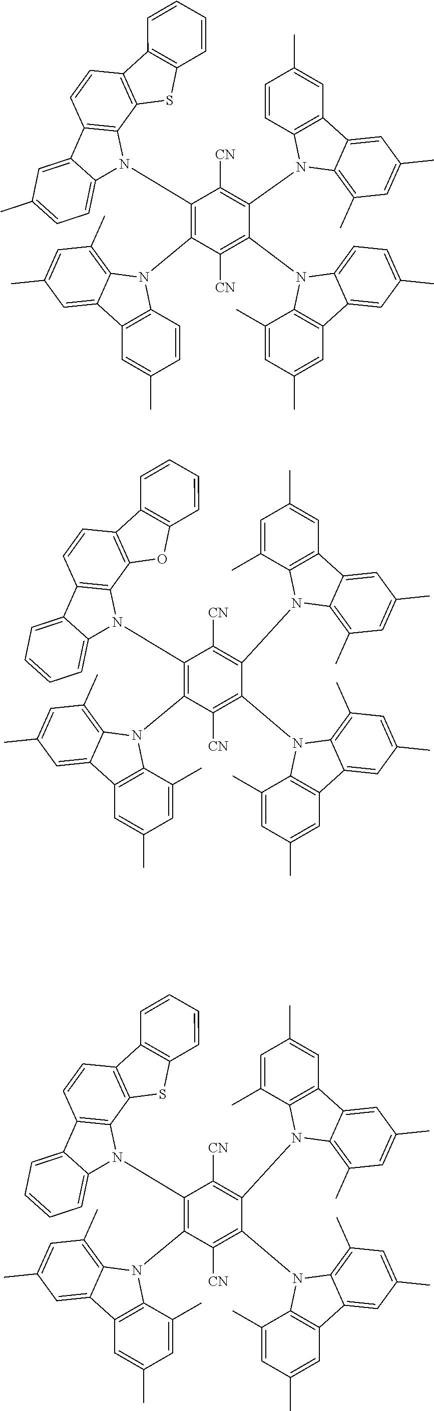

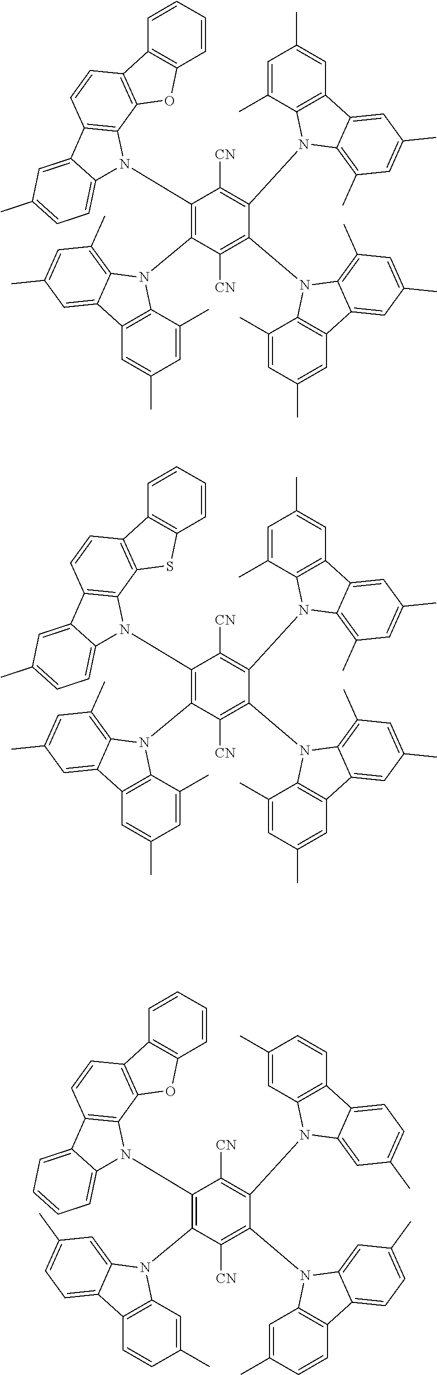

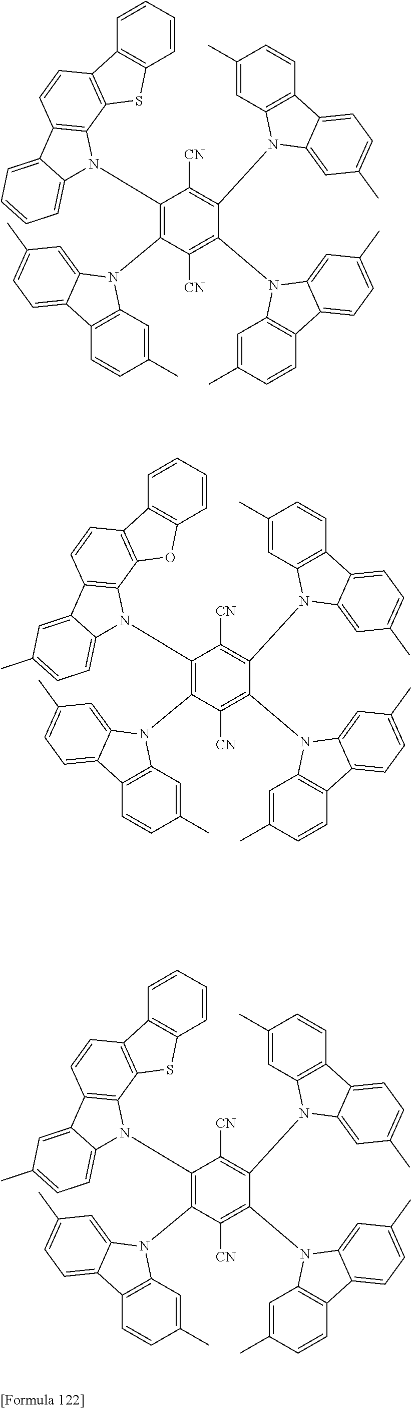

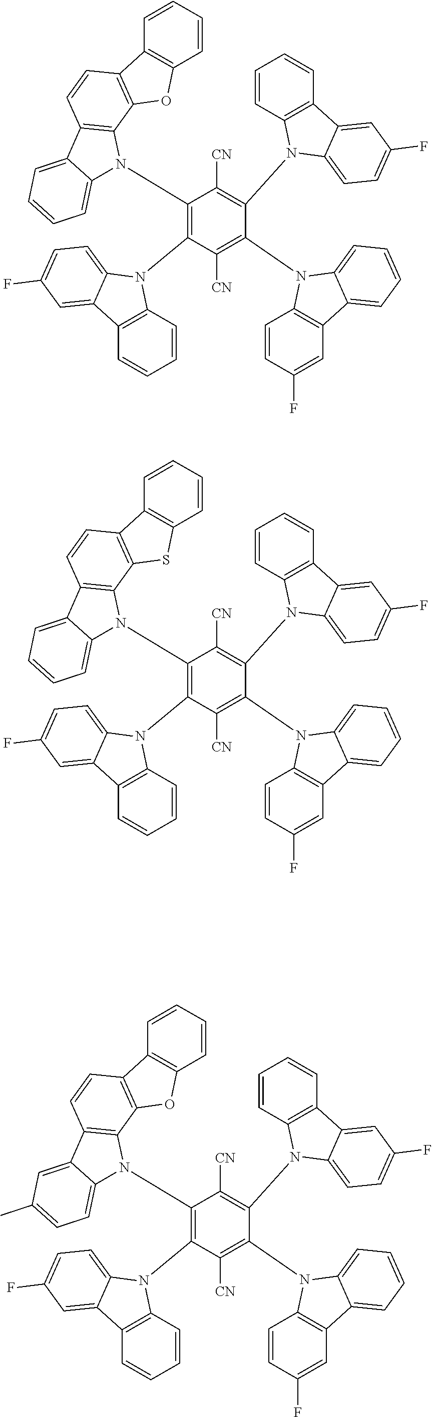 [SMILES: N#Cc1c(-n2c3ccccc3c3cc(F)ccc32)c(-n2c3ccccc3c3cc(F)ccc32)c(C#N)c(-n2c3ccccc3c3ccc4c5ccccc5oc4c32)c1-n1c2ccccc2c2cc(F)ccc21 ; N#Cc1c(-n2c3ccccc3c3cc(F)ccc32)c(-n2c3ccccc3c3cc(F)ccc32)c(C#N)c(-n2c3ccccc3c3ccc4c5ccccc5sc4c32)c1-n1c2ccccc2c2cc(F)ccc21 ; Cc1ccc2c(c1)c1ccc3c4ccccc4oc3c1n2-c1c(C#N)c(-n2c3ccccc3c3cc(F)ccc32)c(-n2c3ccccc3c3cc(F)ccc32)c(C#N)c1-n1c2ccccc2c2cc(F)ccc21]

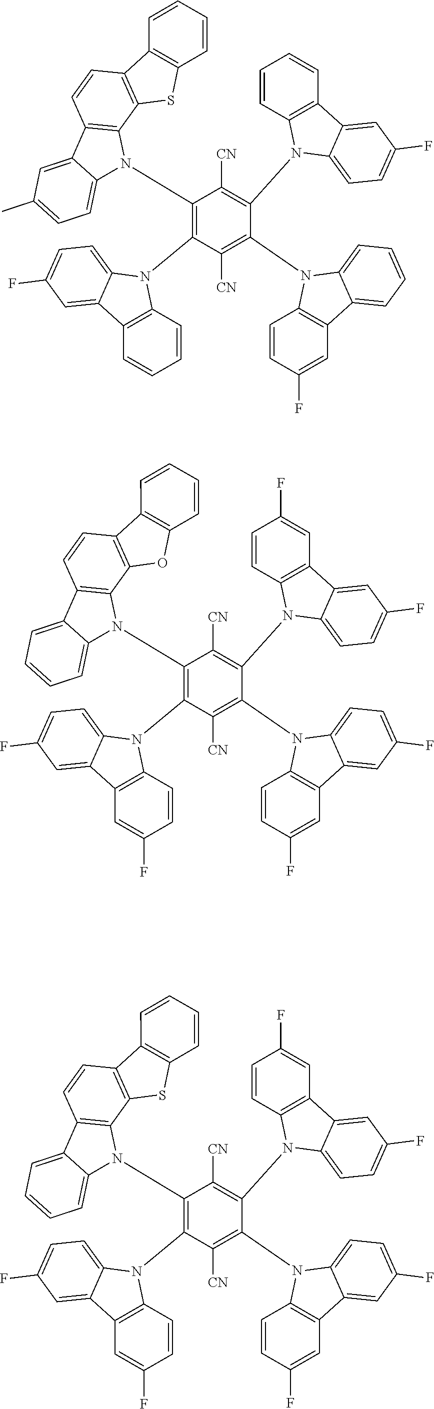 [SMILES: Cc1ccc2c(c1)c1ccc3c4ccccc4sc3c1n2-c1c(C#N)c(-n2c3ccccc3c3cc(F)ccc32)c(-n2c3ccccc3c3cc(F)ccc32)c(C#N)c1-n1c2ccccc2c2cc(F)ccc21 ; N#Cc1c(-n2c3ccc(F)cc3c3cc(F)ccc32)c(-n2c3ccc(F)cc3c3cc(F)ccc32)c(C#N)c(-n2c3ccccc3c3ccc4c5ccccc5oc4c32)c1-n1c2ccc(F)cc2c2cc(F)ccc21 ; N#Cc1c(-n2c3ccc(F)cc3c3cc(F)ccc32)c(-n2c3ccc(F)cc3c3cc(F)ccc32)c(C#N)c(-n2c3ccccc3c3ccc4c5ccccc5sc4c32)c1-n1c2ccc(F)cc2c2cc(F)ccc21]

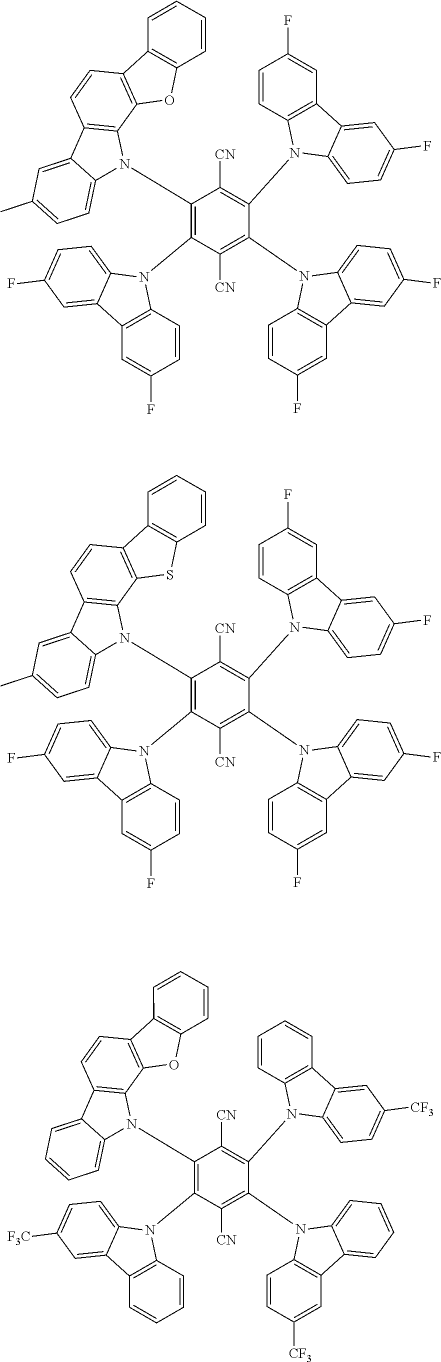 [SMILES: Cc1ccc2c(c1)c1ccc3c4ccccc4oc3c1n2-c1c(C#N)c(-n2c3ccc(F)cc3c3cc(F)ccc32)c(-n2c3ccc(F)cc3c3cc(F)ccc32)c(C#N)c1-n1c2ccc(F)cc2c2cc(F)ccc21 ; Cc1ccc2c(c1)c1ccc3c4ccccc4sc3c1n2-c1c(C#N)c(-n2c3ccc(F)cc3c3cc(F)ccc32)c(-n2c3ccc(F)cc3c3cc(F)ccc32)c(C#N)c1-n1c2ccc(F)cc2c2cc(F)ccc21 ; N#Cc1c(-n2c3ccccc3c3cc(C(F)(F)F)ccc32)c(-n2c3ccccc3c3cc(C(F)(F)F)ccc32)c(C#N)c(-n2c3ccccc3c3ccc4c5ccccc5oc4c32)c1-n1c2ccccc2c2cc(C(F)(F)F)ccc21]

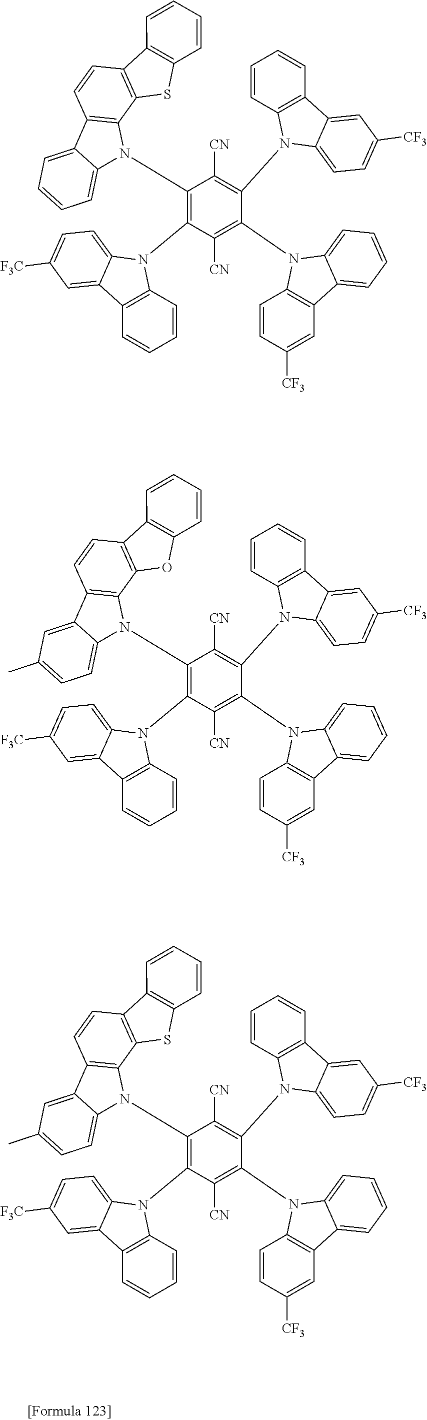 [SMILES: N#Cc1c(-n2c3ccccc3c3cc(C(F)(F)F)ccc32)c(-n2c3ccccc3c3cc(C(F)(F)F)ccc32)c(C#N)c(-n2c3ccccc3c3ccc4c5ccccc5sc4c32)c1-n1c2ccccc2c2cc(C(F)(F)F)ccc21 ; Cc1ccc2c(c1)c1ccc3c4ccccc4oc3c1n2-c1c(C#N)c(-n2c3ccccc3c3cc(C(F)(F)F)ccc32)c(-n2c3ccccc3c3cc(C(F)(F)F)ccc32)c(C#N)c1-n1c2ccccc2c2cc(C(F)(F)F)ccc21 ; Cc1ccc2c(c1)c1ccc3c4ccccc4sc3c1n2-c1c(C#N)c(-n2c3ccccc3c3cc(C(F)(F)F)ccc32)c(-n2c3ccccc3c3cc(C(F)(F)F)ccc32)c(C#N)c1-n1c2ccccc2c2cc(C(F)(F)F)ccc21]

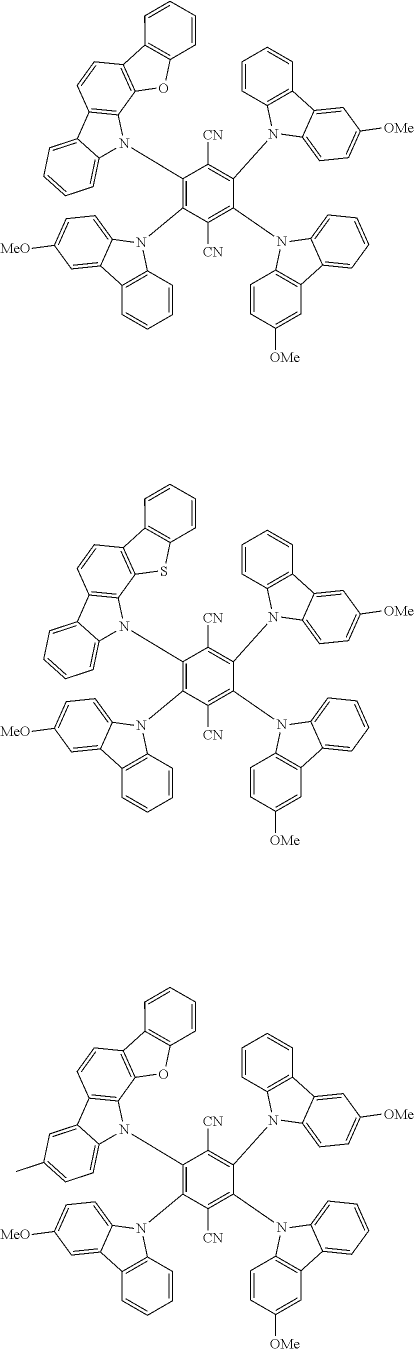 [SMILES: COc1ccc2c(c1)c1ccccc1n2-c1c(C#N)c(-n2c3ccccc3c3cc(OC)ccc32)c(-n2c3ccccc3c3ccc4c5ccccc5oc4c32)c(C#N)c1-n1c2ccccc2c2cc(OC)ccc21 ; COc1ccc2c(c1)c1ccccc1n2-c1c(C#N)c(-n2c3ccccc3c3cc(OC)ccc32)c(-n2c3ccccc3c3ccc4c5ccccc5sc4c32)c(C#N)c1-n1c2ccccc2c2cc(OC)ccc21 ; COc1ccc2c(c1)c1ccccc1n2-c1c(C#N)c(-n2c3ccccc3c3cc(OC)ccc32)c(-n2c3ccc(C)cc3c3ccc4c5ccccc5oc4c32)c(C#N)c1-n1c2ccccc2c2cc(OC)ccc21]

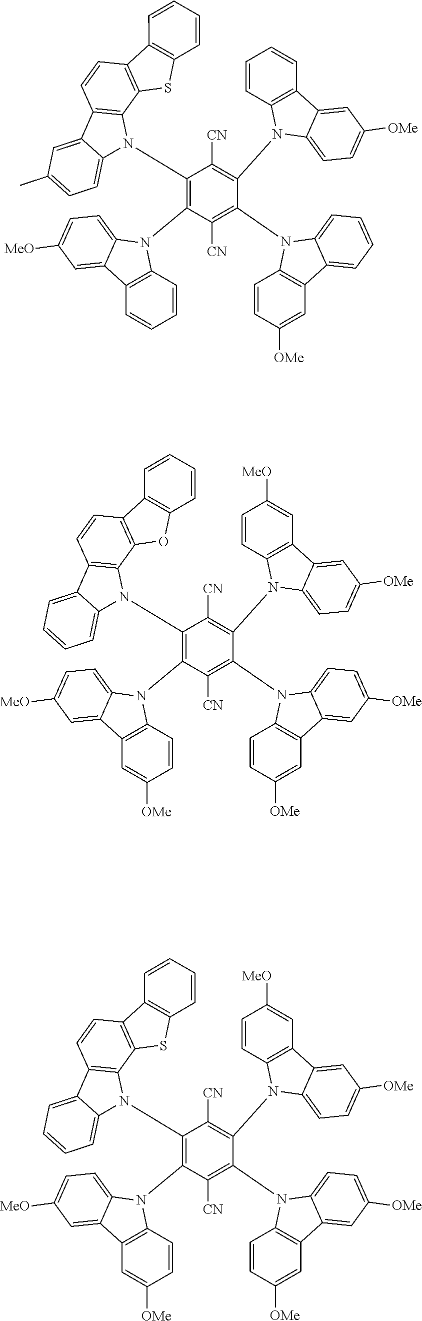 [SMILES: COc1ccc2c(c1)c1ccccc1n2-c1c(C#N)c(-n2c3ccccc3c3cc(OC)ccc32)c(-n2c3ccc(C)cc3c3ccc4c5ccccc5sc4c32)c(C#N)c1-n1c2ccccc2c2cc(OC)ccc21 ; COc1ccc2c(c1)c1cc(OC)ccc1n2-c1c(C#N)c(-n2c3ccc(OC)cc3c3cc(OC)ccc32)c(-n2c3ccccc3c3ccc4c5ccccc5oc4c32)c(C#N)c1-n1c2ccc(OC)cc2c2cc(OC)ccc21 ; COc1ccc2c(c1)c1cc(OC)ccc1n2-c1c(C#N)c(-n2c3ccc(OC)cc3c3cc(OC)ccc32)c(-n2c3ccccc3c3ccc4c5ccccc5sc4c32)c(C#N)c1-n1c2ccc(OC)cc2c2cc(OC)ccc21]

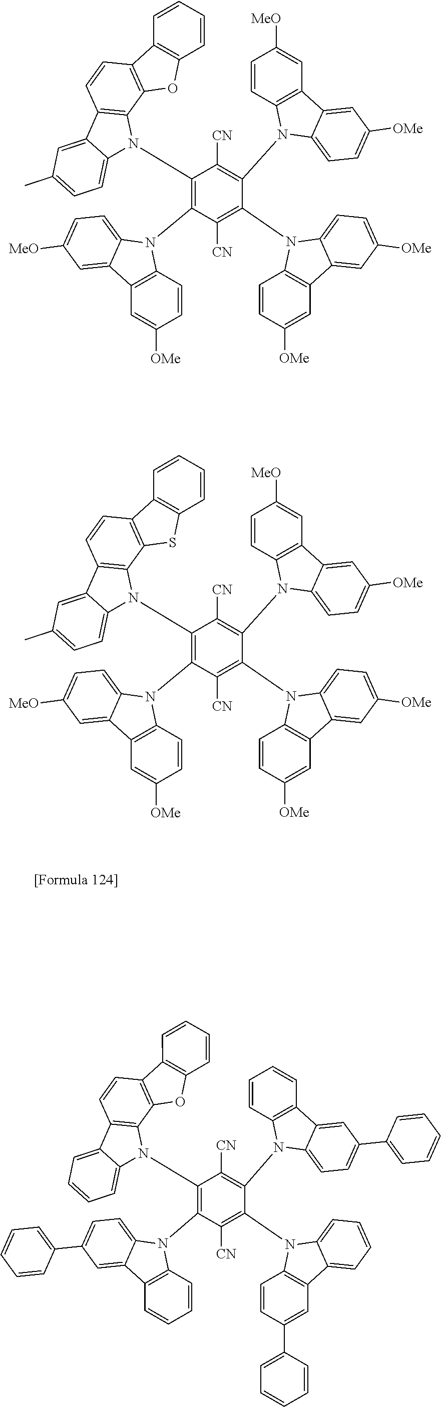 [SMILES: COc1ccc2c(c1)c1cc(OC)ccc1n2-c1c(C#N)c(-n2c3ccc(OC)cc3c3cc(OC)ccc32)c(-n2c3ccc(C)cc3c3ccc4c5ccccc5oc4c32)c(C#N)c1-n1c2ccc(OC)cc2c2cc(OC)ccc21 ; COc1ccc2c(c1)c1cc(OC)ccc1n2-c1c(C#N)c(-n2c3ccc(OC)cc3c3cc(OC)ccc32)c(-n2c3ccc(C)cc3c3ccc4c5ccccc5sc4c32)c(C#N)c1-n1c2ccc(OC)cc2c2cc(OC)ccc21 ; N#Cc1c(-n2c3ccccc3c3cc(-c4ccccc4)ccc32)c(-n2c3ccccc3c3cc(-c4ccccc4)ccc32)c(C#N)c(-n2c3ccccc3c3ccc4c5ccccc5oc4c32)c1-n1c2ccccc2c2cc(-c3ccccc3)ccc21]

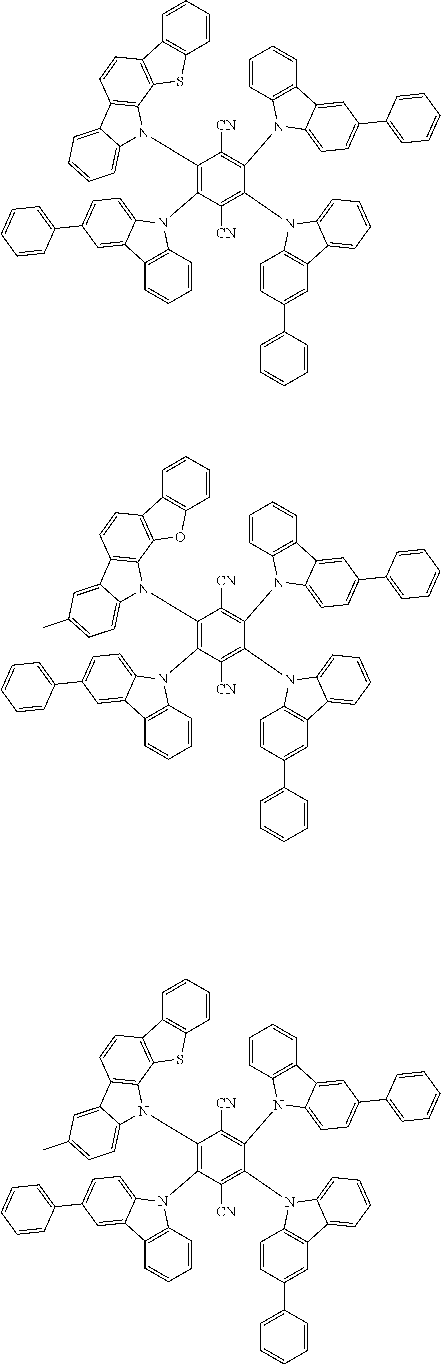 [SMILES: N#Cc1c(-n2c3ccccc3c3cc(-c4ccccc4)ccc32)c(-n2c3ccccc3c3cc(-c4ccccc4)ccc32)c(C#N)c(-n2c3ccccc3c3ccc4c5ccccc5sc4c32)c1-n1c2ccccc2c2cc(-c3ccccc3)ccc21 ; Cc1ccc2c(c1)c1ccc3c4ccccc4oc3c1n2-c1c(C#N)c(-n2c3ccccc3c3cc(-c4ccccc4)ccc32)c(-n2c3ccccc3c3cc(-c4ccccc4)ccc32)c(C#N)c1-n1c2ccccc2c2cc(-c3ccccc3)ccc21 ; Cc1ccc2c(c1)c1ccc3c4ccccc4sc3c1n2-c1c(C#N)c(-n2c3ccccc3c3cc(-c4ccccc4)ccc32)c(-n2c3ccccc3c3cc(-c4ccccc4)ccc32)c(C#N)c1-n1c2ccccc2c2cc(-c3ccccc3)ccc21]

### Delayed Fluorescence

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a mechanism of generating delayed fluorescence is explained in FIG. 10.38 in the document. The second compound of the exemplary embodiment is preferably a compound exhibiting thermally activated delayed fluorescence generated by such a mechanism.

In general, emission of delayed fluorescence can be confirmed by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

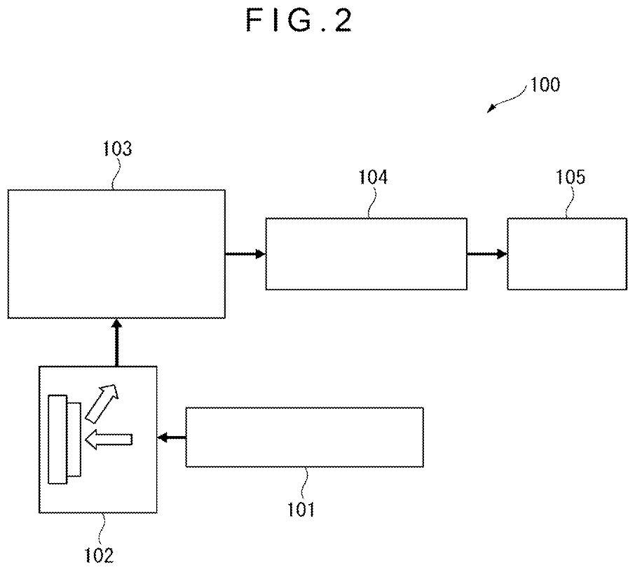

FIG. 2 shows a schematic diagram of an exemplary device for measuring the transient PL. An example of a method of measuring a transient PL using FIG. 2 and an example of behavior analysis of delayed fluorescence will be described.

A transient PL measuring device 100 in FIG. 2 includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. A device for measuring the transient PL is not limited to the device shown in FIG. 2.

The sample housed in the sample chamber 102 is obtained by forming a thin film, in which a matrix material is doped with a doping material at a concentration of 12 mass %, on the quartz substrate.

The thin film sample housed in the sample chamber 102 is irradiated with the pulse laser from the pulse laser 101 to excite the doping material. Emission is extracted in a direction of 90 degrees with respect to a radiation direction of the excited light. The extracted emission is divided by the spectrometer 103 to form a two-dimensional image in the streak camera 104. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was prepared as described above from a reference compound H1 as the matrix material and a reference compound D1 as the doping material and was measured in terms of the transient PL.

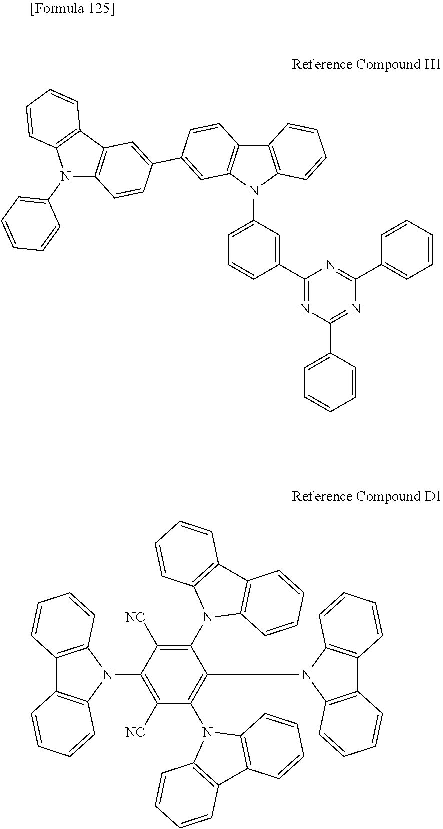 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

Herein, the decay curve was analyzed using the above-described thin film sample A and a thin film sample B. The thin film sample B was manufactured in the same manner as described above from a reference compound H2 as the matrix material and the reference compound D1 as the doping material.

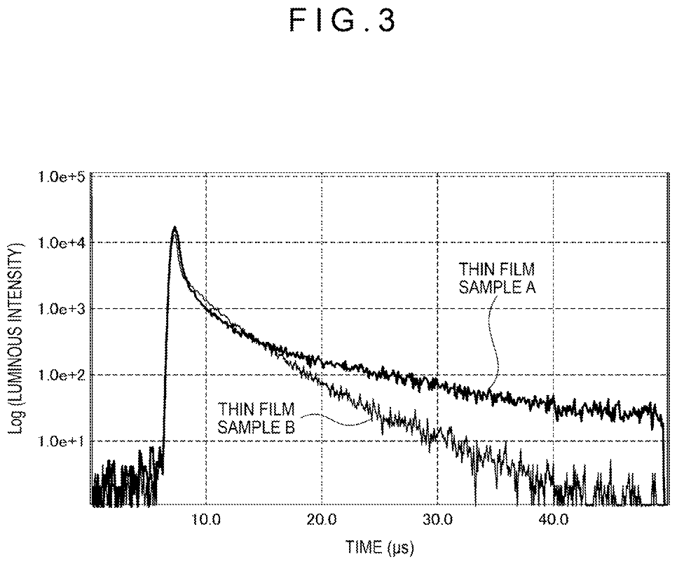

FIG. 3 shows a decay curve obtained from the measured transitional PL of the thin film sample A and the thin film sample B.

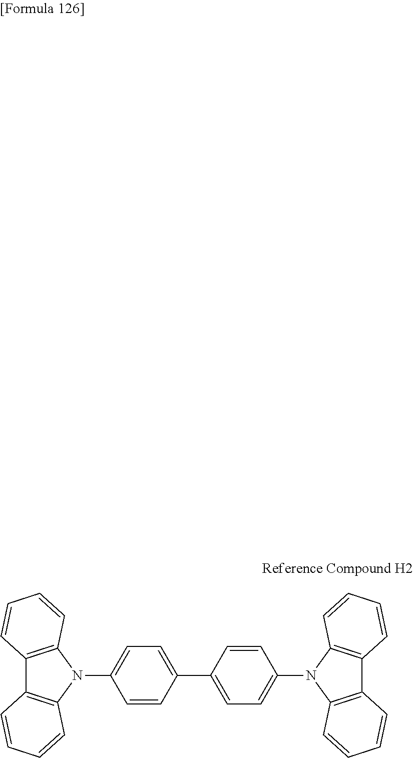 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by inverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

Herein, a sample manufactured according to a method shown below is used for measuring delayed fluorescence of the second compound. For instance, the second compound is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (manufactured by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969.

In the exemplary embodiment, provided that an amount of Prompt emission of the measurement target compound (the second compound) is denoted by X_{P} and an amount of Delay emission thereof is denoted by X_{D}, a value of X_{D}/X_{P} is preferably 0.05 or more.

The amounts of Prompt emission and Delay emission and a ratio of the amounts thereof in compounds other than the second compound herein are measured in the same manner as those of the second compound.

### Third Compound

The third compound may be a thermally activated delayed fluorescent compound or a compound exhibiting no thermally activated delayed fluorescence. However, the third compound is preferably a compound exhibiting no thermally activated delayed fluorescence.

In the exemplary embodiment, the third compound is a compound represented by a formula (3) below.

### Compound Represented by Formula (3)

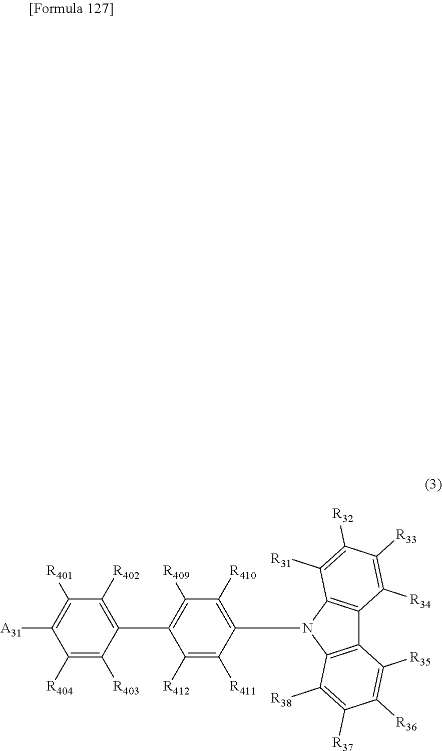

In the formula (3): A₃₁ is a group represented by a formula (31a), formula (31b), formula (31c), formula (31d), formula (31e) or formula (31f);

R₃₁ to R₃₈ are each independently a hydrogen atom or a substituent; R₄₀₁ to R₄₀₄ and R₄₀₉ to R₄₁₂ are each independently a hydrogen atom or a substituent;

R₃₁ to R₃₈ serving as the substituent and R₄₀₁ to R₄₀₄ and R₄₀₉ to R₄₁₂ serving as the substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms.

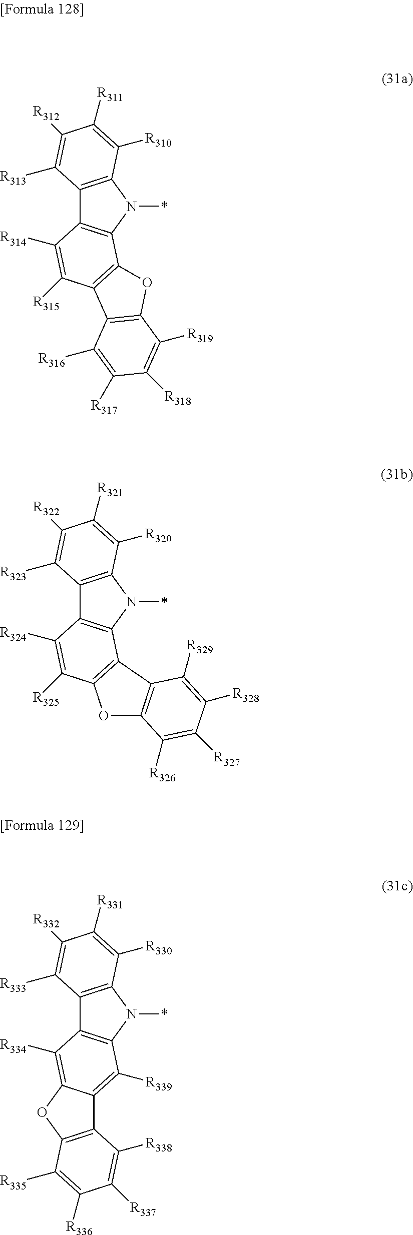

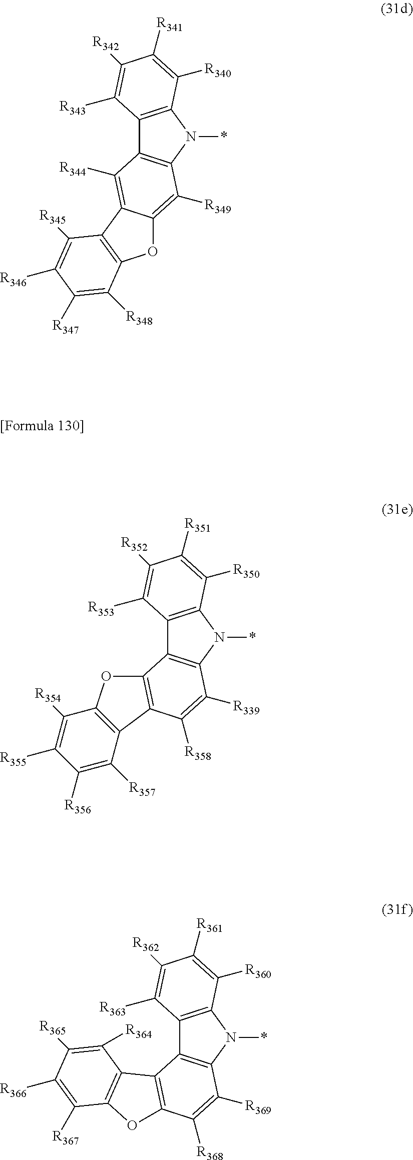

In the formula (31a), formula (31b), formula (31c), formula (31d), formula (31e) and formula (31f):

R₃₁₀ to R₃₁₉ are each independently a hydrogen atom or a substituent;

R₃₂₀ to R₃₂₉ are each independently a hydrogen atom or a substituent;

Ra₃₃₀ to R₃₃₉ are each independently a hydrogen atom or a substituent;

R₃₄₀ to R₃₄₉ are each independently a hydrogen atom or a substituent;

R₃₅₀ to R₃₅₉ are each independently a hydrogen atom or a substituent;

R₃₆₀ to R₃₆₉ are each independently a hydrogen atom or a substituent;

R₃₁₀ to R₃₁₉, Ra₃₂₀ to R₃₂₉, Ra₃₃₀ to R₃₃₉, Ra₃₄₀ to R₃₄₉, R₃₅₀ to R₃₄₅, and R₃₆₀ to R₃₆₉ serving as the substituent each independently represent the same as R₃₁ to R₃₈ serving as the substituent and R₄₀₁ to R₄₀₄ and R₄₀₉ to R₄₁₂ serving as the substituent in the formula (3); and * each independently represents a bonding position to a benzene ring having R₄₀₁ to R₄₀₄ in the formula (3).

### Manufacturing Method of Third Compound

The third compound (the compound represented by the formula (3)) can be manufactured according to, for instance, a method described later in Examples. The third compound of the exemplary embodiment can be manufactured, for instance, by application of known substitution reactions and/or materials depending on a target compound according to reactions described later in Examples.

### Specific Examples of Third Compound

Specific examples of the third compound (the compound represented by the formula (3)) according to the exemplary embodiment are shown below. It should be noted that the third compound of the invention is not limited to the specific examples.

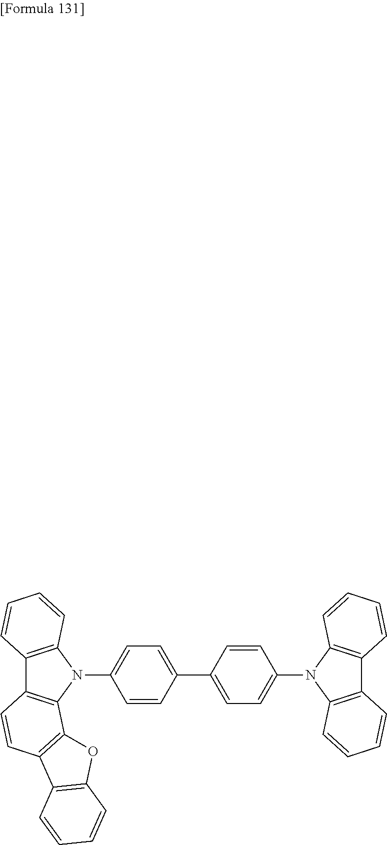 [SMILES: c1ccc2c(c1)oc1c2ccc2c3ccccc3n(-c3ccc(-c4ccc(-n5c6ccccc6c6ccccc65)cc4)cc3)c21]

Relationship between First Compound, Second Compound and Third Compound in Emitting Layer

In the organic EL device 1 of the exemplary embodiment, a singlet energy S₁(M1) of the first compound, a singlet energy S₁(M2) of the second compound, and a singlet energy S₁(M3) of the third compound in the emitting layer 5 satisfy a relationship of a numerical formula (Numerical Formula 1) below.

S₁(M3)>S₁(M2)>S₁(M1) (Numerical Formula 1)

In the emitting layer 5, an energy gap T_{77K}(M1) at 77K of the first compound, an energy gap T_{77K}(M2) at 77K of the second compound, and an energy gap T_{77K}(M3) at 77K of the third compound preferably satisfy a relationship represented by a numerical formula (Numerical Formula 2) below.

T_{77K}(M3)>T_{77K}(M2)>T_{77K}(M1) (Numerical Formula 2)

In the exemplary embodiment, a difference ΔST(M2) between a singlet energy S1(M2) of the second compound and the energy gap T_{77K}(M2) at 77K of the second compound preferably satisfies a relationship represented by one of numerical formulae (Numerical Formula 1A) to (Numerical Formula 1 D).

ΔST(M2)=S₁(M2)−T_{77K}(M2)<0.3 eV (Numerical Formula 1A)

ΔST(M2)=S₁(M2)−T_{77K}(M2)<0.2 eV (Numerical Formula 1B)

ΔST(M2)=S₁(M2)−T_{77K}(M2)<0.1 eV (Numerical Formula 1C)

ΔST(M2)=S₁(M2)−T_{77K}(M2)<0.01 eV (Numerical Formula 1D)

In the exemplary embodiment, a difference ΔST(M1) between a singlet energy S₁(M1) of the first compound and the energy gap T_{77K}(M1) at 77K of the first compound preferably satisfies a relationship represented by a numerical formula (Numerical Formula 1E).

ΔST(M1)=S₁(M1)−T_{77K}(M1)>0.3 eV (Numerical Formula 1E)

In the exemplary embodiment, a difference ΔST(M3) between a singlet energy S₁(M3) of the third compound and the energy gap T_{77K}(M1) at 77K of the third compound preferably satisfies a relationship represented by a numerical formula (Numerical Formula 1F).

ΔST(M3)=S₁(M3)−T_{77K}(M3)>0.3 eV (Numerical Formula 1F)

In the exemplary embodiment, an energy gap T_{77K}(M3) at 77K of the third compound is preferably 2.9 eV or more. With the energy gap T_{77K}(M3) of the third compound, it is believed that the triplet energy of the second compound (delayed fluorescent compound) can be efficiently trapped in the emitting layer.

### TADF Mechanism

In the organic EL device 1 of the exemplary embodiment, the second compound is preferably a compound having a small ΔST(M2), so that inverse intersystem crossing from the triplet energy level of the second compound to the singlet energy level thereof is easily caused by a heat energy given from the outside. An energy state conversion mechanism to perform spin exchange from the triplet state of electrically excited excitons within the organic EL device to the singlet state by inverse intersystem crossing is referred to as the TADF Mechanism.

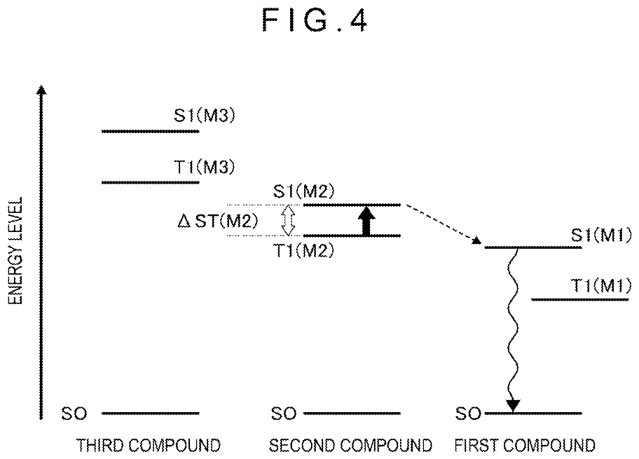

FIG. 4 shows an example of a relationship between energy levels of the first compound, the second compound, and the third compound in the emitting layer 5. In FIG. 4, S0 represents a ground state. S1(M1) represents the lowest singlet state of the first compound. T1(M1) represents the lowest triplet state of the first compound. S1(M2) represents the lowest singlet state of the second compound. T1(M2) represents the lowest triplet state of the second compound. S1(M3) represents the lowest singlet state of the third compound. T1(M3) represents the lowest triplet state of the third compound. A dashed arrow directed from S1(M2) to S1(M1) in FIG. 4 represents Förster energy transfer from the lowest singlet state of the second compound to the lowest singlet state of the first compound.

As shown in FIG. 4, when a compound having a small ΔST(M2) is used as the second compound, inverse intersystem crossing from the lowest triplet state T1(M2) to the lowest singlet state S1(M2) can be caused by a heat energy. Subsequently, Förster energy transfer from the lowest singlet state S1(M2) of the second compound to the first compound occurs to generate the lowest singlet state S1(M1). Consequently, fluorescence from the lowest singlet state S1(M1) of the first compound can be observed. It is inferred that the internal quantum efficiency can be theoretically raised up to 100% also by using delayed fluorescence by the TADF mechanism.

Relationship between Triplet Energy and Energy Gap at 77K

Here, a relationship between a triplet energy and an energy gap at 77K will be described. In the exemplary embodiment, the energy gap at 77K is different from a typical triplet energy in some aspects.

The triplet energy is measured as follows. First, a solution in which a compound (measurement target) is dissolved in an appropriate solvent is encapsulated in a quartz glass tube to prepare a sample. A phosphorescent spectrum (ordinate axis: phosphorescent luminous intensity, abscissa axis: wavelength) of the sample is measured at a low temperature (77K). A tangent is drawn to the rise of the phosphorescent spectrum close to the short-wavelength region. The triplet energy is calculated by a predetermined conversion equation based on a wavelength value at an intersection of the tangent and the abscissa axis.

Herein, the thermally activated delayed fluorescent compound used in the exemplary embodiment is preferably a compound having a small ΔST. When ΔST is small, intersystem crossing and inverse intersystem crossing are likely to occur even at a low temperature (77K), so that the singlet state and the triplet state coexist. As a result, the spectrum to be measured in the same manner as the above includes emission from both the singlet state and the triplet state. Although it is difficult to distinguish the emission from the singlet state from the emission from the triplet state, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is encapsulated in a quartz cell to provide a measurement sample. A phosphorescent spectrum (ordinate axis: phosphorescent luminous intensity, abscissa axis: wavelength) of the sample is measured at a low temperature (77K). A tangent is drawn to the rise of the phosphorescent spectrum close to the short-wavelength region. An energy amount is calculated by a conversion equation (F1) below based on a wavelength value λ_{edge} [nm] at an intersection of the tangent and the abscissa axis and is defined as an energy gap T_{77K} at 77K.

T_{77K}[eV]=1239.85/λ_{edge} Conversion Equation (F1):

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S₁

A method of measuring the singlet energy S₁ with use of a solution (occasionally referred to as a solution method) is exemplified by a method below.

A toluene solution of a measurement target compound at a concentration of 10 μmol/L is prepared and put in a quartz cell. An absorption spectrum (ordinate axis: absorption intensity, abscissa axis: wavelength) of the thus-obtained sample is measured at a normal temperature (300K). A tangent was drawn to the fall of the absorption spectrum close to the long-wavelength region, and a wavelength value λedge (nm) at an intersection of the tangent and the abscissa axis is assigned to a conversion equation (F2) below to calculate the singlet energy.

S₁ [eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the exemplary embodiment, a difference (S₁-T_{77K}) between the singlet energy S₁ and the energy gap T_{77K} at 77K is defined as ΔST.

When the organic EL device 1 of the exemplary embodiment emits light, it is preferable that a fluorescent compound mainly emits light in the emitting layer 5.

The organic EL device 1 of the exemplary embodiment preferably emits red light or green light.

When the organic EL device 1 of the exemplary embodiment emits green light, a main peak wavelength of the light emitted from the organic EL device 1 is preferably in a range from 500 nm to 560 nm.

When the organic EL device 1 of the exemplary embodiment emits red light, a main peak wavelength of the light emitted from the organic EL device 1 is preferably in a range from 600 nm to 660 nm.

When the organic EL device 1 of the exemplary embodiment emits blue light, a main peak wavelength of the light emitted from the organic EL device 1 is preferably in a range from 430 nm to 480 nm.

A main peak wavelength of the light emitted from the organic EL device 1 is measured as follows.

Voltage is applied on the organic EL device 1 such that a current density becomes 10 mA/cm², where spectral radiance spectrum is measured by a spectroradiometer CS-2000 (manufactured by Konica Minolta, Inc.).

A peak wavelength of an emission spectrum, at which the luminous intensity of the resultant spectral radiance spectrum is at the maximum, is measured and defined as the main peak wavelength (unit: nm).

### Film Thickness of Emitting Layer

A film thickness of the emitting layer 5 of the organic EL device 1 in the exemplary embodiment is preferably in a range from 5 nm to 50 nm, more preferably in a range from 7 nm to 50 nm, most preferably in a range from 10 nm to 50 nm. When the film thickness of the emitting layer is 5 nm or more, the formation of the emitting layer and the adjustment of the chromaticity are easy. When the film thickness of the emitting layer is 50 nm or less, an increase in the drive voltage is likely to be reducible.

### Content Ratios of Compounds in Emitting Layer

In the emitting layer 5 of the organic EL device 1 of the exemplary embodiment, the content ratio of the first compound is preferably in a range from 0.01 mass % to 10 mass %, more preferably in a range from 0.01 mass % to 5 mass %, further preferably in a range from 0.01 mass % to 1 mass %.

The content ratio of the second compound preferably ranges from 10 mass % to 80 mass %, more preferably from 10 mass % to 60 mass %, further preferably from 20 mass % to 60 mass %.

The content ratio of the third compound is preferably in a range from 10 mass % to 80 mass %.

An upper limit of the total of the respective content ratios of the first, second, and third compounds in the emitting layer 5 is 100 mass %. It is not excluded that the emitting layer 5 of the exemplary embodiment further contains a material(s) other than the first, second, and third compounds.

The emitting layer 5 may include a single type of the first compound or may include two or more types of the first compound. The emitting layer 5 may include a single type of the second compound or may include two or more types of the second compound. The emitting layer 5 may include a single type of the third compound or may include two or more types of the third compound.

According to the first exemplary embodiment, a high-performance organic EL device 1 is achievable. The organic EL device 1 according to the first exemplary embodiment is applicable to an electronic device such as a display device and a light-emitting device.

An arrangement of the organic EL device 1 will be further described below. It should be noted that the reference numerals will be occasionally omitted below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include indium oxide-tin oxide (ITO: Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

The elements belonging to the group 1 or 2 of the periodic table, which are a material having a small work function, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal are usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of materials for the cathode include elements belonging to the group 1 or 2 of the periodic table, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

When the hole transporting layer includes two or more layers, one of the layers with a larger energy gap is preferably provided closer to the emitting layer. An example of the material with a larger energy gap is HT-2 used in later-described Examples.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/(V·s) or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. The composite material exhibits excellent electron injecting performance and electron transporting performance since the electron donor generates electrons in the organic compound. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

### Second Exemplary Embodiment

### Electronic Device

An electronic device according to the exemplary embodiment is installed with the organic EL device according to the above exemplary embodiment. Examples of the electronic device include a display device and a light-emitting device. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited by the above-described exemplary embodiments but includes any modification and improvement as long as such modification and improvement are compatible with the invention.

For instance, the emitting layer is not limited to a single layer, but may be provided by laminating a plurality of emitting layers. When the organic EL device has the plurality of emitting layers, it is only required that at least one of the emitting layers satisfies the conditions described in the above exemplary embodiment. For instance, in some embodiments, the rest of the emitting layers is a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

It is preferable that a blocking layer is provided adjacent to at least one of a side of the emitting layer close to the anode or a side of the emitting layer close to the cathode. The blocking layer is preferably provided in contact with the emitting layer to block at least any of holes, electrons, excitons or combinations thereof.

Specifically, in the exemplary embodiment, an electron blocking layer as the first layer is provided adjacent to the side of the emitting layer close to the anode. Since the first layer contains the compound represented by the formula (A), the first layer serving as the electron blocking layer is considered to have a deeper ionization potential Ip (larger absolute value). As a result, electrons can be efficiently blocked.

Moreover, in the exemplary embodiment, a hole blocking layer as the second layer is provided adjacent to the side of the emitting layer close to the cathode. Since the second layer contains the compound represented by the formula (B), the second layer serving as the hole blocking layer is considered to have a shallower electron affinity level Af (smaller absolute value). As a result, holes can be efficiently blocked.

The emitting layer and the electron blocking layer are preferably bonded to each other. The emitting layer and the hole blocking layer are preferably bonded to each other.

Specific structure, shape and the like of the components in the invention may be designed in any manner as long as an object of the invention can be achieved.

Herein, numerical ranges represented by “x to y” represents a range whose lower limit is the value (x) recited before “to” and whose upper limit is the value (y) recited after “to.”

Rx and Ry are mutually bonded to form a ring, which means herein, for instance, that Rx and Ry contain a carbon atom, a nitrogen atom, an oxygen atom, a sulfur atom, a phosphorus atom or a silicon atom, the atom (a carbon atom, a nitrogen atom, an oxygen atom, a sulfur atom, a phosphorus atom or a silicon atom) contained in Rx and the atom (a carbon atom, a nitrogen atom, an oxygen atom, a sulfur atom, a phosphorus atom or a silicon atom) contained in Ry are mutually bonded through a single bond, a double bond, a triple bond or a divalent linking group to form a ring having 5 or more ring atoms (specifically, a heterocyclic ring or an aromatic hydrocarbon ring). x represents a number, a character or a combination of a number and a character. y represents a number, a character or a combination of a number and a character.

The divalent linking group is not limited. Examples of the divalent linking group include —O—, —CO—, —CO₂—, —S—, —SO—, —SO₂—, —NH—, —NRa—, and a group provided by a combination of two or more of these linking group.

Specific examples of the heterocyclic ring herein include, unless otherwise described, a cyclic structure (heterocyclic ring) obtained by removing a bond from a “heteroaryl group Sub₂” exemplarily shown in the later-described “Description of Each Substituent in Formula.” The heterocyclic ring may have a substituent.

Specific examples of the aromatic hydrocarbon ring herein include, unless otherwise described, a cyclic structure (aromatic hydrocarbon ring) obtained by removing a bond from a “aryl group Sub₁” exemplarily shown in the later-described “Description of Each Substituent in Formula.” The aromatic hydrocarbon ring may have a substituent.

Examples of Ra include a substituted or unsubstituted alkyl group Sub₃ having 1 to 30 carbon atoms, a substituted or unsubstituted aryl group Sub₁ having 6 to 30 ring carbon atoms, and a substituted or unsubstituted heteroaryl group Sub₂ having 5 to 30 ring atoms, which are exemplarily shown in the later-described “Description of Each Substituent in Formula.”

Rx and Ry are mutually bonded to form a ring, which means, for instance, that: an atom contained in Rx1 and an atom contained in Ry1 in a molecular structure represented by a formula (E1) below form a ring (cyclic structure) E represented by a formula (E2); an atom contained in Rx₁ and an atom contained in Ry₁ in a molecular structure represented by a formula (F1) below form a ring F represented by a formula (F2); an atom contained in Rx₁ and an atom contained in Ry₁ in a molecular structure represented by a formula (G1) below form a ring G represented by a formula (G2); an atom contained in Rx₁ and an atom contained in Ry₁ in a molecular structure represented by a formula (H1) below form a ring H represented by a formula (H2); and an atom contained in Rx₁ and an atom contained in Ry₁ in a molecular structure represented by a formula (I1) below form a ring I represented by a formula (I2).

In the formulae (E1) to (I1), * each independently represent a bonding position to another atom in a molecule. The two * in the formulae (E1), (F1), (G1), (H1) and (I1) correspond to two * in the formulae (E2), (F2), (G2), (H2) and (I2), respectively.

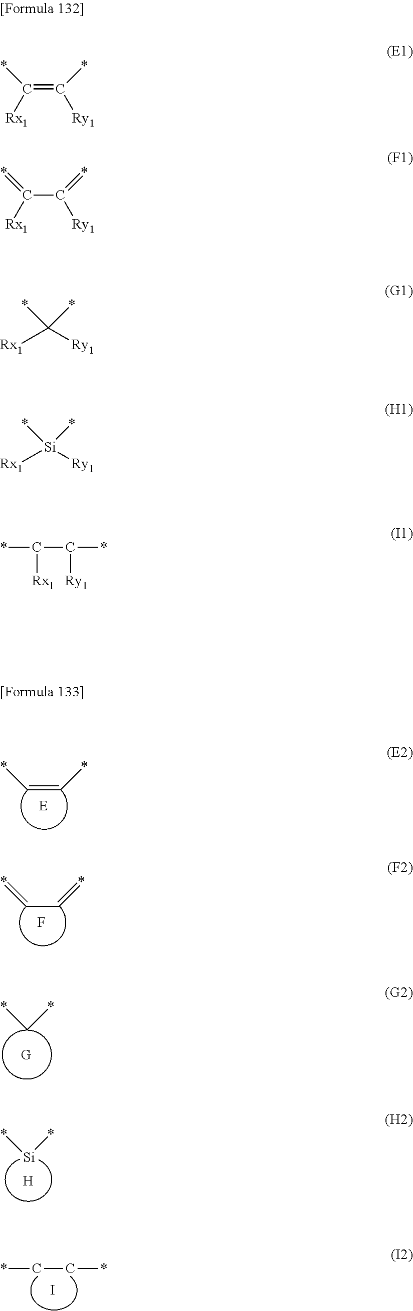

In the molecular structures represented by the formulae (E2) to (I2), E to I each represent a cyclic structure (the ring having 5 or more ring atoms). In the formulae (E2) to (I2), * each independently represent a bonding position to another atom in a molecule. The two * in the formula (E2) correspond to two * in the formula (E1). Similarly, two * in each of the formulae (F2) to (I2) correspond one-to-one to two * in in each of the formulae (F1) to (I1).

For instance, in the formula (E1), when Rx₁ and Ry₁ are mutually bonded to form the ring E in the formula (E2) and the ring E is an unsubstituted benzene ring, the molecular structure represented by the formula (E1) is a molecular structure represented by a formula (E3) below. Herein, two * in the formula (E3) each independently correspond to two * in the formula (E2) and the formula (E1).

For instance, in the formula (E1), when Rx₁ and Ry₁ are mutually bonded to form the ring E in the formula (E2) and the ring E is an unsubstituted pyrrole ring, the molecular structure represented by the formula (E1) is a molecular structure represented by a formula (E4) below. Herein, two * in the formula (E4) each independently correspond to two * in the formula (E2) and the formula (E1). In the formulae (E3) and (E4), * each independently represent a bonding position to another atom in a molecule.

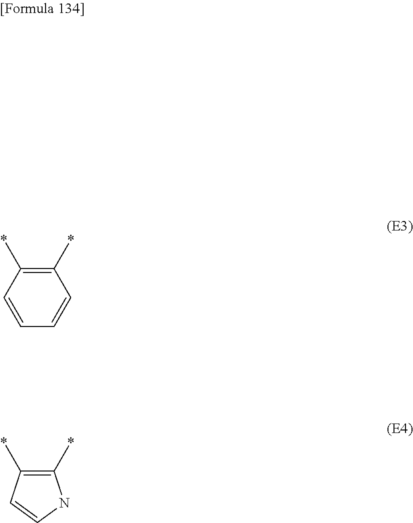 [SMILES: *c1ccccc1* ; *c1cc[nH]c1*]

Herein, the ring carbon atoms refer to the number of carbon atoms among atoms forming a ring of a compound (e.g., a monocyclic compound, fused-ring compound, bridged compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridinyl group has 5 ring carbon atoms, and a furanyl group has 4 ring carbon atoms. When a benzene ring and/or a naphthalene ring is substituted by a substituent (e.g., an alkyl group), the number of carbon atoms of the alkyl group is not counted in the number of the ring carbon atoms. When a fluorene ring is substituted by a substituent (e.g., a fluorene ring) (i.e., a spirofluorene ring is included), the number of carbon atoms of the fluorene ring as the substituent is not counted in the number of the ring carbon atoms of the fluorene ring.

Herein, the ring atoms refer to the number of atoms forming a ring of a compound (e.g., a monocyclic compound, fused-ring compound, bridged compound, carbon ring compound, and heterocyclic compound) in which the atoms are bonded to each other to form the ring (e.g., monocyclic ring, fused ring, ring assembly). Atom(s) not forming a ring and atom(s) included in a substituent when the ring is substituted by the substituent are not counted in the number of the ring atoms. Unless specifically described, the same applies to the “ring atoms” described later. For instance, a pyridine ring has six ring atoms, a quinazoline ring has ten ring atoms, and a furan ring has five ring atoms. A hydrogen atom(s) and/or an atom(s) of a substituent which are bonded to carbon atoms of a pyridine ring and/or quinazoline ring are not counted in the ring atoms. When a fluorene ring is substituted by a substituent (e.g., a fluorene ring) (i.e., a spirofluorene ring is included), the number of atoms of the fluorene ring as the substituent is not counted in the number of the ring atoms of the fluorene ring.

### Description of Each Substituent in Formulae Herein

The aryl group (occasionally referred to as an aromatic hydrocarbon group) herein is exemplified by an aryl group Sub₁. The aryl group Sub₁ is at least one group selected from the group consisting of a phenyl group, biphenyl group, terphenyl group, naphthyl group, anthryl group, phenanthryl group, fluorenyl group, pyrenyl group, chrysenyl group, fluoranthenyl group, benz[a]anthryl group, benzo[c]phenanthryl group, triphenylenyl group, benzo[k]fluoranthenyl group, benzo[g]chrysenyl group, benzo[b]triphenylenyl group, picenyl group, and perylenyl group.

Herein, the aryl group Sub₁ preferably has 6 to 30 ring carbon atoms, more preferably 6 to 20 ring carbon atoms, further preferably 6 to 14 ring carbon atoms, still further preferably 6 to 12 ring carbon atoms. Among the aryl group Sub₁, a phenyl group, biphenyl group, naphthyl group, phenanthryl group, terphenyl group and fluorenyl group are preferable. A carbon atom in a position 9 of each of 1-fluorenyl group, 2-fluorenyl group, 3-fluorenyl group and 4-fluorenyl group is preferably substituted by a substituted or unsubstituted alkyl group Sub₃ or a substituted or unsubstituted aryl group Sub₁ described later herein.

The heteroaryl group (occasionally referred to as a heterocyclic group, heteroaromatic cyclic group or aromatic heterocyclic group) herein is exemplified by a heterocyclic group Sub₂. The heterocyclic group Sub₂ is a group containing, as a hetero atom(s), at least one atom selected from the group consisting of nitrogen, sulfur, oxygen, silicon, selenium atom and germanium atom. The heterocyclic group Sub₂ preferably contains, as a hetero atom(s), at least one atom selected from the group consisting of nitrogen, sulfur and oxygen.

The heterocyclic group Sub₂ herein are, for instance, at least one group selected from the group consisting of a pyridyl group, pyrimidinyl group, pyrazinyl group, pyridazinyl group, triazinyl group, quinolyl group, isoquinolinyl group, naphthyridinyl group, phthalazinyl group, quinoxalinyl group, quinazolinyl group, phenanthridinyl group, acridinyl group, phenanthrolinyl group, pyrrolyl group, imidazolyl group, pyrazolyl group, triazolyl group, tetrazolyl group, indolyl group, benzimidazolyl group, indazolyl group, imidazopyridinyl group, benzotriazolyl group, carbazolyl group, furyl group, thienyl group, oxazolyl group, thiazolyl group, isoxazolyl group, isothiazolyl group, oxadiazolyl group, thiadiazolyl group, benzofuranyl group, benzothienyl group, benzoxazolyl group, benzothiazolyl group, benzisoxazolyl group, benzisothiazolyl group, benzoxadiazolyl group, benzothiadiazolyl group, dibenzofuranyl group, dibenzothienyl group, piperidinyl group, pyrrolidinyl group, piperazinyl group, morpholyl group, phenazinyl group, phenothiazinyl group, and phenoxazinyl group.

Herein, the heterocyclic group Sub₂ preferably has 5 to 30 ring atoms, more preferably 5 to 20 ring atoms, further preferably 5 to 14 ring atoms. Among the above heterocyclic group Sub₂, a 1-dibenzofuranyl group, 2-dibenzofuranyl group, 3-dibenzofuranyl group, 4-dibenzofuranyl group, 1-dibenzothienyl group, 2-dibenzothienyl group, 3-dibenzothienyl group, 4-dibenzothienyl group, 1-carbazolyl group, 2-carbazolyl group, 3-carbazolyl group, 4-carbazolyl group, and 9-carbazolyl group are further more preferable. A nitrogen atom in position 9 of 1-carbazolyl group, 2-carbazolyl group, 3-carbazolyl group and 4-carbazolyl group is preferably substituted by the substituted or unsubstituted aryl group Sub₁ or the substituted or unsubstituted heterocyclic group Sub₂ described herein.

Herein, the heterocyclic group Sub₂ may be a group derived from any one of partial structures represented by formulae (XY-1) to (XY-18) below.

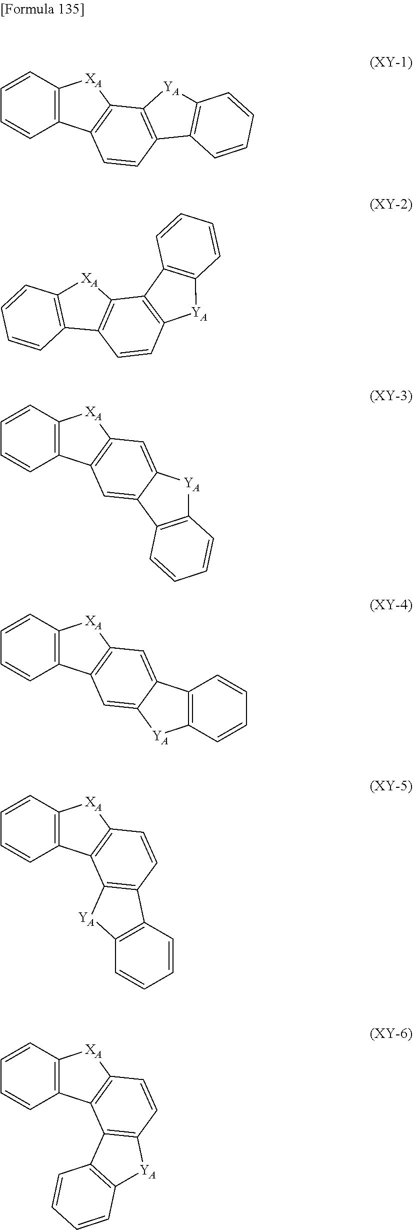

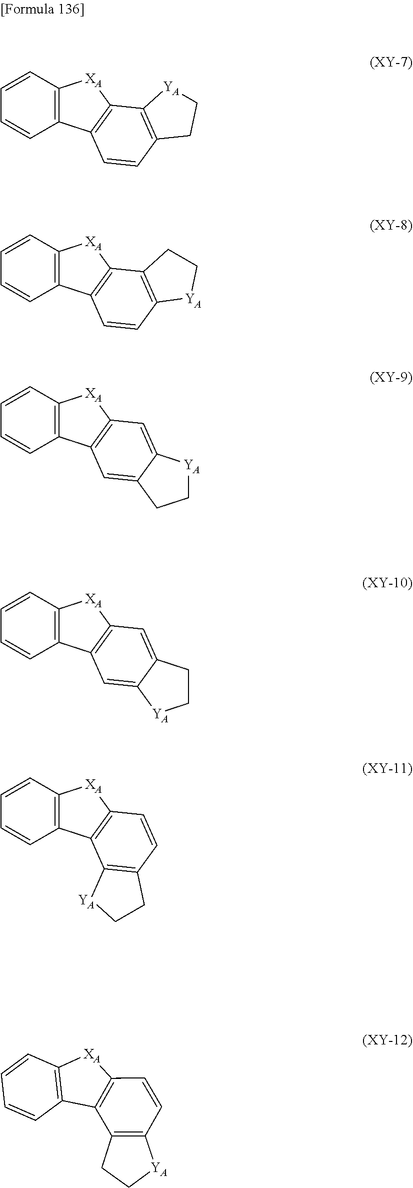

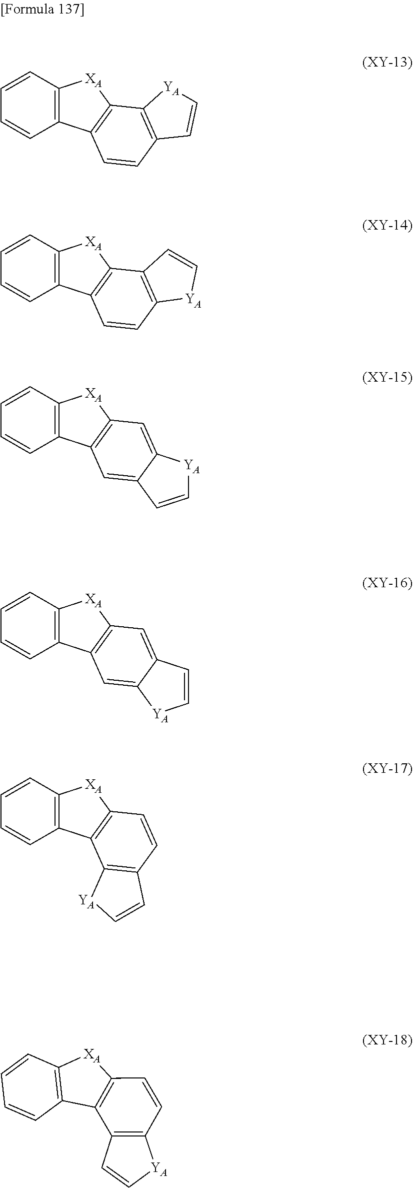

In the formulae (XY-1) to (XY-18), X_{A} and Y_{A} each independently represent a hetero atom, and preferably represent an oxygen atom, sulfur atom, selenium atom, silicon atom or germanium atom. Each of the partial structures represented by the respective formulae (XY-1) to (XY-18) has a bond at any position to provide a heterocyclic group. The heterocyclic group may be substituted.

Herein, the heterocyclic group Sub₂ may be a group represented by one of formulae (XY-19) to (XY-22) below. Moreover, the position of the bond may be changed as needed.

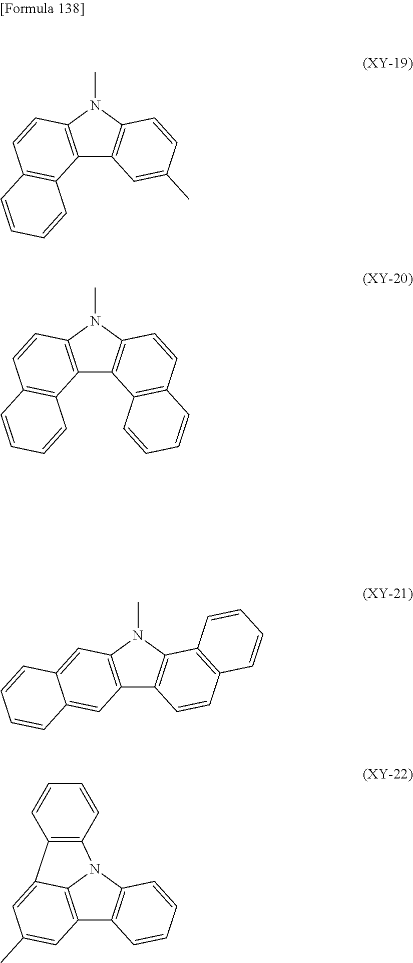

The alkyl group herein may be any one of a linear alkyl group, branched alkyl group and cyclic alkyl group.

The alkyl group herein is exemplified by an alkyl group Sub₃.

The linear alkyl group herein is exemplified by a linear alkyl group Sub₃₁.

The branched alkyl group herein is exemplified by a branched alkyl group Sub₃₂.

The cyclic alkyl group herein is exemplified by a cyclic alkyl group Sub₃₃.

For instance, the alkyl group Sub₃ is at least one group selected from the group consisting of the linear alkyl group Sub₃₁, branched alkyl group Sub₃₂, and cyclic alkyl group Sub₃₃.

The linear alkyl group Sub₃₁ or branched alkyl group Sub₃₂ is exemplified by at least one group selected from the group consisting of a methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, amyl group, isoamyl group, 1-methylpentyl group, 2-methylpentyl group, 1-pentylhexyl group, 1-butylpentyl group, 1-heptyloctyl group, and 3-methylpentyl group.

Herein, the linear alkyl group Sub₃₁ or branched alkyl group Sub₃₂ preferably has 1 to 30 carbon atoms, more preferably 1 to 20 carbon atoms, further preferably 1 to 10 carbon atoms, still further preferably 1 to 6 carbon atoms. The linear alkyl group Sub₃₁ or branched alkyl group Sub₃₂ is still further preferably a methyl group, ethyl group, propyl group, isopropyl group, n-butyl group, s-butyl group, isobutyl group, t-butyl group, n-pentyl group, n-hexyl group, amyl group, isoamyl group and neopentyl group.

Herein, the cyclic alkyl group Sub₃₃ is exemplified by a cycloalkyl group Sub₃₃₁.

The cycloalkyl group Sub₃₃₁ herein is exemplified by at least one group selected from the group consisting of a cyclopropyl group, cyclobutyl group, cyclopentyl group, cyclohexyl group, 4-metylcyclohexyl group, adamantyl group and norbomyl group. The cycloalkyl group Sub₃₃₁ preferably has 3 to 30 ring carbon atoms, more preferably 3 to 20 ring carbon atoms, further preferably 3 to 10 ring carbon atoms, still further preferably 5 to 8 ring carbon atoms. Among the cycloalkyl group Sub₃₃₁, a cyclopentyl group and a cyclohexyl group are still further preferable.

Herein, an alkyl halide group is exemplified by an alkyl halide group Sub₄. The alkyl halide group Sub₄ is provided by substituting the alkyl group Sub₃ with at least one halogen atom, preferably at least one fluorine atom.

Herein, the alkyl halide group Sub₄ is exemplified by at least one group selected from the group consisting of a fluoromethyl group, difluoromethyl group, trifluoromethyl group, fluoroethyl group, trifluoromethylmethyl group, trifluoroethyl group, and pentafluoroethyl group.

Herein, a substituted silyl group is exemplified by a substituted silyl group Sub₅. The substituted silyl group Sub₅ is exemplified by at least one group selected from the group consisting of an alkylsilyl group Sub₅₁ and an arylsilyl group Sub₅₂.

Herein, the alkylsilyl group Sub₅₁₁ is exemplified by a trialkylsilyl group Sub₅₁₁ having the above-described alkyl group Sub₃.

The trialkylsilyl group Sub₅₁₁ is exemplified by at least one group selected from the group consisting of a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyl dimethylsilyl group, propyldimethylsilyl group, and triisopropylsilyl group. Three alkyl groups Suba in the trialkylsilyl group Sub₅₁₁ may be mutually the same or different.

Herein, the arylsilyl group Sub₅₂ is exemplified by at least one group selected from the group consisting of a dialkylarylsilyl group Sub₅₂₁, alkyldiarylsilyl group Sub₅₂₂ and triarylsilyl group Sub₅₂₃.

The dialkylarylsilyl group Sub₅₂₁ is exemplified by a dialkylarylsilyl group including two alkyl groups Suba and one aryl group Sub₁. The dialkylarylsilyl group Sub₅₂₁ preferably has 8 to 30 carbon atoms.

The alkyldiarylsilyl group Sub₅₂₂ is exemplified by an alkyldiarylsilyl group including one alkyl group Suba and two aryl groups Sub₁. The alkyldiarylsilyl group Sub₅₂₂ preferably has 13 to 30 carbon atoms.

The triarylsilyl group Sub₅₂₃ is exemplified by a triarylsilyl group including three aryl groups Sub₁. The triarylsilyl group Sub₅₂₃ preferably has 18 to 30 carbon atoms.

Herein, a substituted or unsubstituted alkyl sulfonyl group is exemplified by an alkyl sulfonyl group Sub₆. The alkyl sulfonyl group Sub₆ is represented by —SO₂Rw. R_{w} in —SO₂R_{w} represents a substituted or unsubstituted alkyl group Sub₃ described above.

Herein, an aralkyl group (occasionally referred to as an arylalkyl group) is exemplified by an aralkyl group Sub₇. An aryl group in the aralkyl group Sub₇ includes, for instance, at least one of the above-described aryl group Sub₁ or the above-described heteroaryl group Sub₂.

The aralkyl group Sub₇ herein is preferably a group having the aryl group Sub₁ and is represented by —Z₃-Z₄. Z₃ is exemplified by an alkylene group corresponding to the above alkyl group Sub₃. Z₄ is exemplified by the above aryl group Sub₁. In this aralkyl group Sub₇, an aryl moiety has 6 to 30 carbon atoms (preferably 6 to 20 carbon atoms, more preferably 6 to 12 carbon atoms) and an alkyl moiety has 1 to 30 carbon atoms (preferably 1 to 20 carbon atoms, more preferably 1 to 10 carbon atoms, further preferably 1 to 6 carbon atoms). The aralkyl group Sub₇ is exemplified by at least one group selected from the group consisting of a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

The alkoxy group herein is exemplified by an alkoxy group Sub₈. The alkoxy group Sub₈ is represented by —OZ₁. Z₁ is exemplified by the above alkyl group Sub₃. The alkoxy group Sub₈ is exemplified by at least one group selected from the group consisting of a methoxy group, ethoxy group, propoxy group, butoxy group, pentyloxy group and hexyloxy group. The alkoxy group Sub₈ preferably has 1 to 30 carbon atoms, more preferably 1 to 20 carbon atoms.

Herein, an alkoxy halide group is exemplified by an alkoxy halide group Sub₉. The alkoxy halide group Sub₉ is provided by substituting the alkoxy group Sub₈ with at least one halogen atom, preferably at least one fluorine atom.

Herein, an aryloxy group (occasionally referred to as an arylalkoxy group) is exemplified by an arylalkoxy group Sub₁₀. An aryl group in the arylalkoxy group Sub₁₀ includes at least one of the aryl group Sub₁ or the heteroaryl group Sub₂.

The arylalkoxy group Sub₁₀ herein is represented by —OZ₂. Z₂ is exemplified by the aryl group Sub₁ or the heteroaryl group Sub₂. The arylalkoxy group Sub₁₀ preferably has 6 to 30 ring carbon atoms, more preferably 6 to 20 ring carbon atoms. The arylalkoxy group Sub₁₀ is exemplified by a phenoxy group.

Herein, a substituted amino group is exemplified by a substituted amino group Sub₁₁. The substituted amino group Sub₁₁ is exemplified by at least one group selected from the group consisting of an arylamino group Sub₁₁₁ and an alkylamino group Sub₁₁₂.

The arylamino group Sub₁₁₁ is represented by —NHR_{V1} or —N(R_{V1})2. R_{V1} is exemplified by the aryl group Sub₁. Two R_{V1} in —N(R_{V1})2 are mutually the same or different.

The alkylamino group Sub₁₁₂ is represented by —NHR_{V2} or —N(R_{V2})2. R_{V2} is exemplified by the alkyl group Sub₃. Two R_{V2} in —N(R_{V2})2 are mutually the same or different.

Herein, the alkenyl group is exemplified by an alkenyl group Sub₁₂. The alkenyl group Sub₁₂, which is linear or branched, is exemplified by at least one group selected from the group consisting of a vinyl group, propenyl group, butenyl group, oleyl group, eicosapentaenyl group, docosahexaenyl group, styryl group, 2,2-diphenylvinyl group, 1,2,2-triphenylvinyl group, and 2-phenyl-2-propenyl group.

The alkynyl group herein is exemplified by an alkynyl group Sub₁₃. The alkynyl group Sub₁₃ may be linear or branched and is at least one group selected from the group consisting of an ethynyl group, a propynyl group and a 2-phenylethynyl group.

The alkylthio group herein is exemplified by an alkylthio group Sub₁₄.

The alkylthio group Sub₁₄ is represented by —SR_{V3}. R_{V3} is exemplified by the alkyl group Sub₃. The alkylthio group Sub₁₄ preferably has 1 to 30 carbon atoms, more preferably 1 to 20 carbon atoms.

The arylthio group herein is exemplified by an arylthio group Sub₁₅.

The arylthio group Sub₁₅ is represented by —SR_{V4}. R_{V4} is exemplified by the aryl group Sub₁. The arylthio group Sub₁₅ preferably has 6 to 30 ring carbon atoms, more preferably 6 to 20 ring carbon atoms.

Herein, examples of the halogen atom include a fluorine atom, a chlorine atom, a bromine atom and an iodine atom, among which a fluorine atom is preferable.

A substituted phosphino group herein is exemplified by a substituted phosphino group Sub₁₆. The substituted phosphino group Sub₁₆ is exemplified by a phenyl phosphanyl group.

An arylcarbonyl group herein is exemplified by an arylcarbonyl group Sub₁₇. The arylcarbonyl group Sub₁₇ is represented by —COY′. Y′ is exemplified by the aryl group Sub₁. Herein, the arylcarbonyl group Sub₁₇ is exemplified by at least one group selected from the group consisting of a phenyl carbonyl group, diphenyl carbonyl group, naphthyl carbonyl group, and triphenyl carbonyl group.

An acyl group herein is exemplified by an acyl group Sub₁₈. The acyl group Sub₁₈ is represented by —COR′. R′ is exemplified by the alkyl group Sub₃. The acyl group Sub₁₈ herein is exemplified by at least one group selected from the group consisting of an acetyl group and a propionyl group.

A substituted phosphoryl group herein is exemplified by a substituted phosphoryl group Sub₁₉ such as an aryl phosphoryl group and alkyl phosphoryl group. The substituted phosphoryl group Sub₁₉ is represented by a formula (P) below.

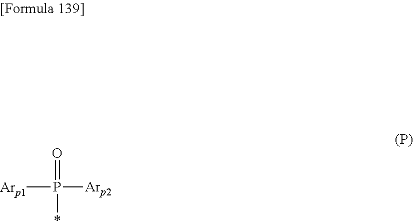

In the formula (P), Ar_{P1} and Ar_{P2} are any one substituent selected from the group consisting of the above alkyl group Sub₃ and the above aryl group Sub₁.

An ester group herein is exemplified by an ester group Sub₂₀. The ester group Sub₂₀ is exemplified by at least one group selected from the group consisting of an alkyl ester group and an aryl ester group.

An alkyl ester group herein is exemplified by an alkyl ester group Sub₂₀₁. The alkyl ester group Sub₂₀₁ is represented by —C(═O)OR^{E}. R^{E} is exemplified by a substituted or unsubstituted alkyl group Sub₃ described above.

An aryl ester group herein is exemplified by an aryl ester group Sub₂₀₂. The aryl ester group Sub₂₀₂ is represented by —C(═O)OR^{Ar}. R^{Ar} is exemplified by a substituted or unsubstituted aryl group Sub₁ described above.

A siloxanyl group herein is exemplified by a siloxanyl group Sub₂₁. The siloxanyl group Sub₂₁ is a silicon compound group through an ether bond. The siloxanyl group Sub₂₁ is exemplified by a trimethylsiloxanyl group.

A carbamoyl group herein is represented by —CONH₂.

A substituted carbamoyl group herein is exemplified by a carbamoyl group Sub₂₂. The carbamoyl group Sub₂₂ is represented by —CONH—Ar^{C} or —CONH—R^{C}. Ar^{C} is exemplified by at least one group selected from the group consisting of the above-described aryl group Sub₁ (preferably 6 to 10 ring carbon atoms) and the above-described heteroaryl group Sub₂ (preferably 5 to 14 ring atoms). Ar^{C} may be a group formed by bonding the aryl group Sub₁ and the heteroaryl group Sub₂.

R^{C} is exemplified by a substituted or unsubstituted alkyl group Sub₃ described above (preferably having 1 to 6 carbon atoms).

Herein, “carbon atoms forming a ring (ring carbon atoms)” mean carbon atoms forming a saturated ring, unsaturated ring, or aromatic ring. “Atoms forming a ring (ring atoms)” mean carbon atoms and hetero atoms forming a hetero ring including a saturated ring, unsaturated ring, or aromatic ring.

Herein, a hydrogen atom includes isotope having different numbers of neutrons, specifically, protium, deuterium and tritium.

Hereinafter, an alkyl group Sub₃ means at least one group of a linear alkyl group Sub₃₁, a branched alkyl group Sub₃₂, or a cyclic alkyl group Sub₃₃ described in “Description of Each Substituent.”

Similarly, a substituted silyl group Sub₅ means at least one group of an alkylsilyl group Sub₅₁ or an arylsilyl group Sub₅₂.

Similarly, a substituted amino group Sub₁₁ means at least one group of an arylamino group Sub₁₁₁ or an alkylamino group Sub₁₁₂.

Herein, a substituent for a “substituted or unsubstituted” group is exemplified by a substituent R_{F1}. The substituent R_{F1} is at least one group selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group.

Herein, the substituent R_{F1} for a “substituted or unsubstituted” group may be a diaryl boron group (Ar_{B1}Ar_{B2}B—). Ar_{B1} and Ar_{B2} are exemplified by the above-described aryl group Sub₁. Ar_{B1} and Ar_{B2} in Ar_{B1}Ar_{B2}B— are the same or different.

Specific examples and preferable examples of the substituent R_{F1} are the same as those of the substituents described in “Description of Each Substituent” (e.g., an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, and carbamoyl group Sub₂₂).

The substituent R_{F1} for a “substituted or unsubstituted” group may be further substituted by at least one group (hereinafter, also referred to as a substituent R_{F2}) selected from the group consisting of an aryl group Sub₁, heteroaryl group Sub₂, alkyl group Suba, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group. Moreover, a plurality of substituents R_{F2} may be bonded to each other to form a ring.

“Unsubstituted” for a “substituted or unsubstituted” group means that a group is not substituted by the above-described substituent R_{F1} but bonded with a hydrogen atom.

Herein, “XX to YY carbon atoms” in the description of “substituted or unsubstituted ZZ group having XX to YY carbon atoms” represent carbon atoms of an unsubstituted ZZ group and do not include carbon atoms of the substituent R_{F1} Of the substituted ZZ group.

Herein, “XX to YY atoms” in the description of “substituted or unsubstituted ZZ group having XX to YY atoms” represent atoms of an unsubstituted ZZ group and do not include atoms of the substituent R_{F1} of the substituted ZZ group.

The same description as the above applies to “substituted or unsubstituted” in compounds or partial structures thereof described herein.

Herein, when the substituents are bonded to each other to form a ring, the ring is structured to be a saturated ring, an unsaturated ring, an aromatic hydrocarbon ring or a hetero ring.

Herein, examples of the aromatic hydrocarbon group in the linking group include a divalent or multivalent group obtained by eliminating one or more atoms from the above monovalent aryl group Sub₁.

Herein, examples of the heterocyclic group in the linking group include a divalent or multivalent group obtained by eliminating one or more atoms from the above monovalent heteroaryl group Sub₂.

### EXAMPLES

Example(s) of the invention will be described below. However, the invention is not limited to Example(s).

### Compounds

The compound represented by the formula (A) and the compound represented by the formula (B) used for manufacturing the organic EL device in Examples are shown below.

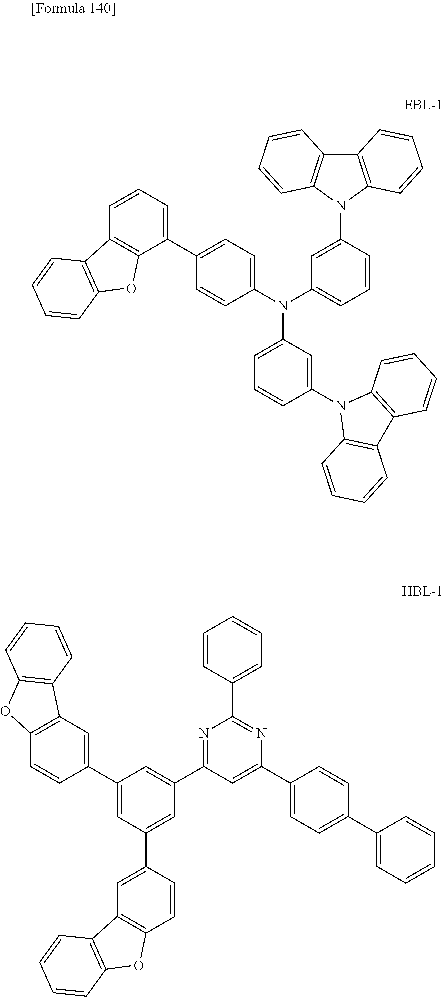 [SMILES: c1cc(N(c2ccc(-c3cccc4c3oc3ccccc34)cc2)c2cccc(-n3c4ccccc4c4ccccc43)c2)cc(-n2c3ccccc3c3ccccc32)c1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

The compound represented by the formula (1) and the compound represented by the formula (2) used for manufacturing the organic EL device in Example 1 are shown below.

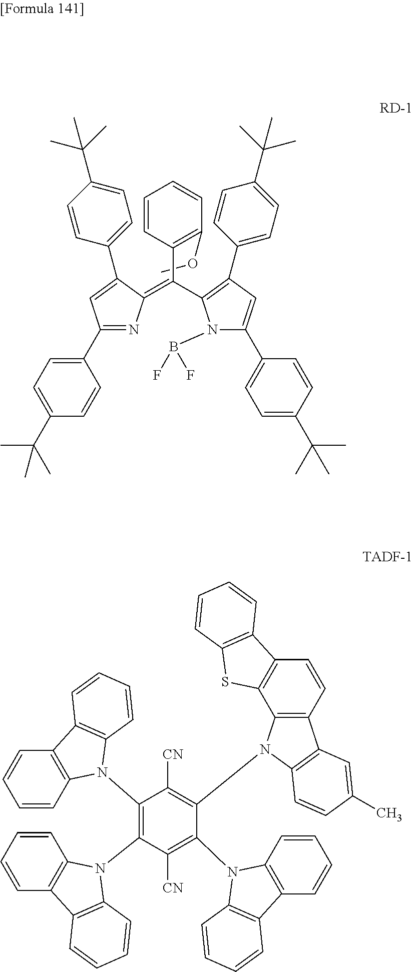 [SMILES: COc1ccccc1/C(=C1/N=C(c2ccc(C(C)(C)C)cc2)C=C1c1ccc(C(C)(C)C)cc1)c1c(-c2ccc(C(C)(C)C)cc2)cc(-c2ccc(C(C)(C)C)cc2)n1B(F)F ; Cc1ccc2c(c1)c1ccc3c4ccccc4sc3c1n2-c1c(C#N)c(-n2c3ccccc3c3ccccc32)c(-n2c3ccccc3c3ccccc32)c(C#N)c1-n1c2ccccc2c2ccccc21]

The compound represented by the formula (3) used for manufacturing the organic EL device in Examples are shown below.

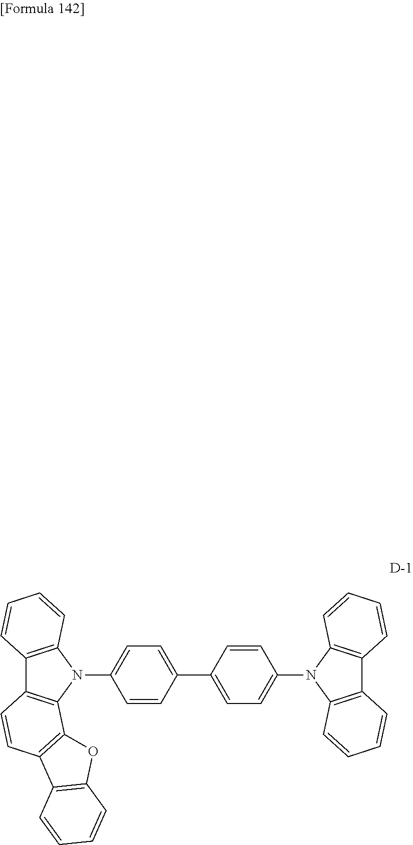 [SMILES: c1ccc2c(c1)oc1c2ccc2c3ccccc3n(-c3ccc(-c4ccc(-n5c6ccccc6c6ccccc65)cc4)cc3)c21]

Structures of other compounds used for manufacturing the organic EL device in Examples are shown below.

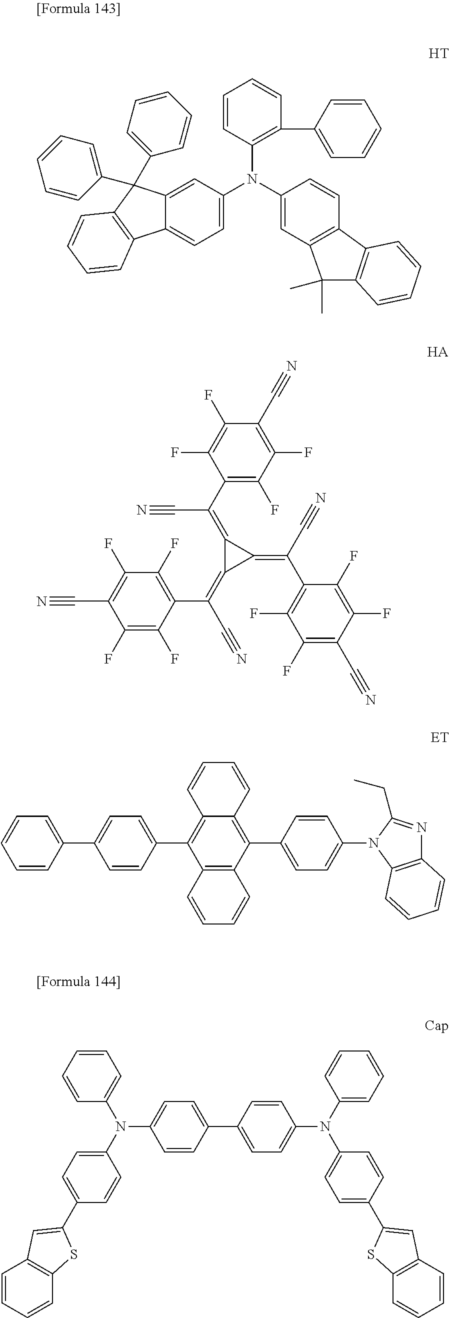 [SMILES: CC1(C)c2ccccc2-c2ccc(N(c3ccc4c(c3)C(c3ccccc3)(c3ccccc3)c3ccccc3-4)c3ccccc3-c3ccccc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CCc1nc2ccccc2n1-c1ccc(-c2c3ccccc3c(-c3ccc(-c4ccccc4)cc3)c3ccccc23)cc1 ; c1ccc(N(c2ccc(-c3ccc(N(c4ccccc4)c4ccc(-c5cc6ccccc6s5)cc4)cc3)cc2)c2ccc(-c3cc4ccccc4s3)cc2)cc1]

### Preparation of Organic EL Device

The organic EL devices were prepared and evaluated as follows.

### Example 1

### Manufacture of Bottom Emission Type Organic EL Device

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for one minute. A film of ITO was 130 nm thick.

After the glass substrate having the transparent electrode line was cleaned, the glass substrate was mounted on a substrate holder of a vacuum evaporation apparatus. Firstly, a compound HT and a compound HA were co-deposited on a surface of the glass substrate where the transparent electrode line was provided in a manner to cover the transparent electrode, thereby forming a 10-nm-thick hole injecting layer. The concentrations of the compound HT and the compound HA in the hole injecting layer were 97 mass % and 3 mass %, respectively.

Next, a compound HT was vapor-deposited on a hole injecting layer to form a 200-nm-thick hole transporting layer.

Next, a compound EBL-1 was vapor-deposited on the hole transporting layer to form a 10-nm-thick electron blocking layer as the first layer.

Next, a fluorescent compound RD-1 (the first compound), a delayed fluorescent compound TADF-1 (the second compound), and a compound D-1 (the third compound) were co-deposited on the electron blocking layer to form a 25-nm-thick emitting layer. The concentrations of the compound RD-1, the compound TADF-1, and the compound D-1 in the emitting layer were 1 mass %, 25 mass %, and 74 mass %, respectively.

Next, a compound HBL-1 was vapor-deposited on the emitting layer to form a 10-nm-thick hole blocking layer as the second layer.

Next, the compound ET was vapor-deposited on the hole blocking layer to form a 30-nm-thick electron transporting layer.

Next, lithium fluoride (LiF) was vapor-deposited on the electron transporting layer to form a 1-nm-thick electron injecting electrode (cathode).

Subsequently, metal aluminum (Al) was vapor-deposited on the electron injectable electrode to form an 80-nm-thick metal Al cathode.

A device arrangement of the organic EL device in Example 1 is schematically shown as follows.

ITO(130)/HT:HA(10.97%:3%)/HT(200)EBL-1(10)/D-1:TADF-1:RD-1(25.74%:25%:1%)/HBL-1(10)/ET(30)/LiF(1)/Al(80)

Numerals in parentheses represent a film thickness (unit: nm).

The numerals (97%:3%) represented by percentage in the same parentheses indicate a ratio (mass %) between the compound HT and the compound HA in the hole injecting layer, and the numerals (74%:25%:1%) represented by percentage in the same parentheses indicate a ratio (mass %) between the third compound, the second compound, and the first compound in the emitting layer. Similar notations apply to the description below.

Table 1 shows structures of the electron blocking layer, the emitting layer, and the hole blocking layer of the organic EL device manufactured in Example 1.

TABLE 1

| | | | | | | Hole Blocking |
|---|---|---|---|---|---|---|
| | Electron Blocking Layer | | | | | Layer |
| | (First Layer) | | Emitting Layer | | | (Second Layer) |
| | Compound of | Ip | First | Second | Third | Compound of |
| | Formula (A) | [eV] | Compound | Compound | Compound | Formula (B) |
| Example 1 | EBL-1 | 5.86 | RD-1 | TADF-1 | D-1 | HBL-1 |

### Evaluation

An organic EL device manufactured in Example 1 was driven. As a result, the organic EL device in Example 1 emitted red light.

### Evaluation of Compounds

Physical properties of compounds described in Tables 1 and 2 were measured according to the following methods.

### Ionization Potential Ip

Ionization potential Ip of a compound EBL-1 was measured according to the following method.

The ionization potential Ip was measured under atmosphere using a photoelectron spectroscope (“AC-3” manufactured by RIKEN KEIKI Co., Ltd.). Specifically, the measurement target material was irradiated with light and the amount of electrons generated by charge separation was measured to measure the ionization potential.

### Delayed Fluorescence of Compound TADF-1

Delayed fluorescence properties were checked by measuring transient photoluminescence (PL) using a device shown in FIG. 2. The compound TADF-1 was dissolved in toluene to prepare a dilute solution with an absorbance of 0.05 or less at the excitation wavelength to eliminate the contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the above sample solution was measured with a spectrofluorometer FP-8600 (manufactured by JASCO Corporation), and the fluorescence spectrum of a 9,10-diphenylanthracene ethanol solution was measured under the same conditions. Using the fluorescence area intensities of both spectra, the total fluorescence quantum yield is calculated by an equation (1) in Morris et al. J. Phys. Chem. 80 (1976) 969.

Prompt emission was observed immediately when the excited state was achieved by exciting the compound TADF-1 with a pulse beam (i.e., a beam emitted from a pulse laser) having a wavelength to be absorbed by the compound TADF-1, and Delay emission was observed not immediately when the excited state was achieved but after the excited state was achieved. The delayed fluorescence in Examples means that an amount of Delay Emission is 5% or more with respect to an amount of Prompt Emission. Specifically, provided that the amount of Prompt emission is denoted by X_{P} and the amount of Delay emission is denoted by X_{D}, the delayed fluorescence means that a value of X_{D}/X_{P} is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

It was confirmed that the amount of Delay Emission was 5% or more with respect to the amount of Prompt Emission in the compound TADF-1.

Specifically, it was found that a value of X_{D}/X_{P} was 0.05 or more in the compound TADF-1.

### Singlet Energy S₁

A singlet energy S₁ of each of the compound RD-1, the compound TADF-1, and the compound D-1 was measured according to the above-described solution method.

### Energy Gap T77K at 77K

An energy gap T_{77K} at 77K of the compound TADF-1 was measured. ΔST was checked from the measurement results of the energy gap T_{77K} and the value of the singlet energy S₁ described above. The compound TADF-1 was measured according to the measurement method of the energy gap T_{77K} described in the above “Relationship between Triplet Energy and Energy Gap at 77K.”

### Main Peak Wavelength λ of Compound

A main peak wavelength λ of the compound RD-1 was measured according to the following method.

A toluene solution of a measurement target compound at a concentration of 5 μmol/L was prepared and put in a quartz cell. An emission spectrum (ordinate axis: luminous intensity, abscissa axis: wavelength) of the thus-obtained sample was measured at a normal temperature (300K). In Examples, the emission spectrum was measured using a spectrophotometer manufactured by Hitachi, Ltd. (device name: F-7000). It should be noted that the machine for measuring the emission spectrum is not limited to the machine used herein. A peak wavelength of the emission spectrum exhibiting the maximum luminous intensity was defined as a main peak wavelength λ.

TABLE 2

| | | S₁ | ΔST | λ |
|---|---|---|---|---|
| | | [eV] | [eV] | [nm] |
| First Compound | RD-1 | 2.02 | — | 609 |
| Second Compound | TADF-1 | 2.32 | <0.01 | — |
| Third Compound | D-1 | 3.42 | — | — |

### Explanation of Table 2

“-” represents no measurement.

“<0.01” represents ΔST of less than 0.01 eV.

### Example 2

### Manufacture of Bottom Emission Type Organic EL Device

An organic EL device in Example 2 was manufactured in the same manner as in Example 1 except that the compound RD-1 in the emitting layer was replaced by a compound RD-2 below.

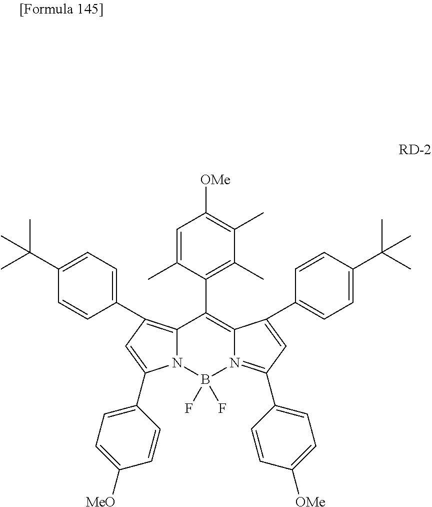

A device arrangement of the organic EL device in Example 2 is roughly shown as follows.

ITO(130)/HT:HA(10.97%:3%)/HT(200)/EBL-1(10)/D-1:TADF-1:RD-2(25.74%:25%:1%)/HBL-1(10)/ET(30)/LiF(1)/Al(80)

### Example 3

### Manufacture of Bottom Emission Type Organic EL Device

An organic EL device in Example 3 was manufactured in the same manner as in Example 1 except that the compound RD-1 in the emitting layer was replaced by a compound RD-3 below.

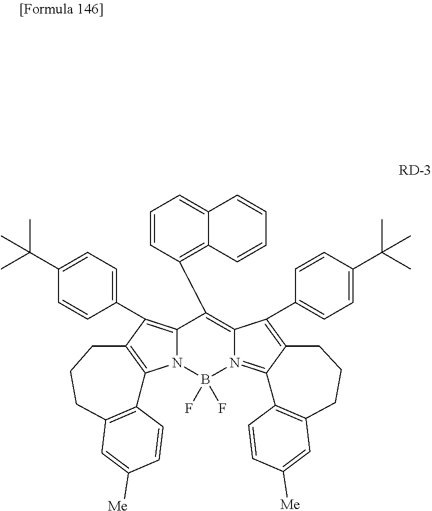

A device arrangement of the organic EL device in Example 3 is roughly shown as follows.

ITO(130)/HT:HA(10.97%:3%)/HT(200)EBL-1(10)/D-1:TADF-1:RD-3(25.74%:25%:1%)/HBL-1(10)/ET(30)/LiF(1)/Al(80)

### Evaluation of Organic EL Devices

The organic EL devices in Examples 2 and 3 were evaluated as follows. Measurement results are shown in Table 3.

### Drive Voltage

A voltage (unit: V) was measured when current was applied between the anode and the cathode such that a current density was 10 mA/cm².

### External Quantum Efficiency EQE

Voltage was applied on each of the organic EL devices such that a current density was 10 mA/cm², where spectral radiance spectra were measured by a spectroradiometer CS-2000 (manufactured by Konica Minolta, Inc.). The external quantum efficiency EQE (unit: %) was calculated based on the obtained spectral-radiance spectra, assuming that the spectra was provided under a Lambertian radiation.

### Main Peak Wavelength λp and Full Width at Half Maximum FWHM When Device is Driven

Voltage was applied on each of the organic EL devices such that a current density of the organic EL device was 10 mA/cm², where spectral radiance spectrum was measured by a spectroradiometer CS-2000 (manufactured by Konica Minolta, Inc.). A main peak wavelength λp (unit: nm) and a full width at half maximum FWHM (unit: nm) were acquired from the obtained spectral radiance spectrum.

### CIE1931 Chromaticity

Voltage was applied on each of the organic EL devices manufactured such that a current density was 10 mA/cm², where coordinates (x, y) of CIE1931 chromaticity were measured by a spectroradiometer CS-1000 (manufactured by Konica Minolta, Inc.).

### Lifetime LT95

Voltage was applied on the manufactured organic EL devices so that a current density was 50 mA/cm², where a time (LT95 (unit: hr)) elapsed before luminance was reduced to 95% of the initial luminance was measured.

TABLE 3

| | First | Voltage | EQE | λp | Full Width at Half | | LT95 |
|---|---|---|---|---|---|---|---|
| | Compound | [V] | [%] | [nm] | Maximum [nm] | CIE | [hr] |
| Example 2 | RD-2 | 4.4 | 16.5 | 632 | 42 | (0.68, 0.32) | 101 |
| Example 3 | RD-3 | 4.3 | 16.7 | 616 | 37 | (0.66, 0.34) | 88 |

### Example 4

### Manufacture of Top Emission Type Organic EL Device

An APC (Ag—Pd—Cu) layer (reflective layer) having a film thickness of 100 nm, which was silver alloy layer, and an indium zinc oxide (IZO: registered trademark) layer having a thickness of 10 nm were sequentially formed by sputtering on a glass substrate.

Subsequently, this conductive material layer was patterned by etching using a resist pattern as a mask using a normal lithography technique to form an anode. The substrate formed with this lower electrode was ultrasonic-cleaned in isopropyl alcohol for five minutes, and then UV-ozone-cleaned for 30 minutes.

Subsequently, the compound HT and the compound HA were co-deposited by using vacuum deposition to form a 10-nm-thick hole injecting layer. The concentrations of the compound HT and the compound HA in the hole injecting layer were 97 mass % and 3 mass %, respectively.

Next, the compound HT was vapor-deposited on the hole injecting layer to form a 180-nm-thick hole transporting layer (HT).

Next, a compound EBL-1 was vapor-deposited on the hole transporting layer to form a 10-nm-thick electron blocking layer as the first layer.

Next, a fluorescent compound RD-2 (the first compound), a delayed fluorescent compound TADF-1 (the second compound), and a compound D-1 (the third compound) were co-deposited on the electron blocking layer to form a 25-nm-thick emitting layer. The concentrations of the compound RD-2, the compound TADF-1, and the compound D-1 in the emitting layer were 1 mass %, 25 mass %, and 74 mass %, respectively.

Next, the compound HBL-1 was vapor-deposited on the emitting layer to form a 15-nm-thick hole blocking layer as the second layer.

Next, the compound ET was vapor-deposited on the hole blocking layer to form a 45-nm-thick electron transporting layer.

Next, lithium fluoride (LiF) was vapor-deposited on the electron transporting layer to form a 1-nm-thick electron injecting electrode (cathode).

Subsequently, Mg and Ag were vapor-deposited at a film thickness ratio of 15:85 on the electron injectable electrode to form a 15-nm-thick cathode formed of semi-transparent MgAg alloy. Cap was used to form a film on the cathode by vacuum deposition to form a 65-nm capping layer.

A device arrangement of the organic EL device in Example 4 is roughly shown as follows.

APC(100)/IZO(10)/HT:HA(10.97%:3%)/HT(180)/EBL-1(10)/D-1:TADF-1:RD-2(25.74%:25%:1%)/HBL-1(15)/ET(45)/LiF(1)/MgAg(15)/Cap(65)

### Examples 5 to 8

### Manufacture of Top Emission Type Organic EL Device

Organic EL devices in Examples 5 to 8 were manufactured in the same manner as in Example 4 except that a film thickness of the hole transporting layer (HT) was changed as shown in Table 4 and the compound RD-2 in the emitting layer was replaced by the compound RD-3.

### Evaluation of Organic EL Devices

The organic EL devices in Examples 5 to 8 were evaluated in the same manner as in Examples 2 and 3 in terms of the drive voltage, the main peak wavelength λp and the full width at half maximum FWHM when the device was driven, CIE1931 chromaticity, and the lifetime LT95. Moreover, luminance-current efficiency (L/J) was measured according to the following methods.

### Luminance-Current Efficiency (L/J)

Voltage was applied on each of the organic EL devices manufactured so that the current density was 10 mA/cm², where luminance L (unit: cd/m²) was measured using a spectroradiometer (manufactured by Konica Minolta, Inc., product name: CS-2000).

A luminance-current efficiency (unit: cd/A) was calculated based on the obtained luminance.

Measurement results are shown in Table 4.

TABLE 4

| | HT Thickness | First | Voltage | L/J | λp | Full Width at Half | | LT95 |
|---|---|---|---|---|---|---|---|---|
| | [nm] | Compound | [V] | [cd/A] | [nm] | Maximum [nm] | CIE | [hr] |
| Example 4 | 180 | RD-2 | 4.5 | 37 | 628 | 35 | (0.68, 0.32) | 101 |
| Example 5 | 175 | RD-3 | 4.3 | 57 | 614 | 32 | (0.66, 0.34) | 88 |
| Example 6 | 180 | RD-3 | 4.4 | 58 | 616 | 32 | (0.67, 0.33) | 91 |
| Example 7 | 185 | RD-3 | 4.4 | 53 | 619 | 33 | (0.68, 0.33) | 88 |
| Example 8 | 190 | RD-3 | 4.4 | 46 | 621 | 35 | (0.68, 0.32) | 92 |

### Synthesis Example 1: Synthesis of Compound D-1

### (1-1) Synthesis of Compound D-1

A synthesis scheme of the compound D-1 is shown below.

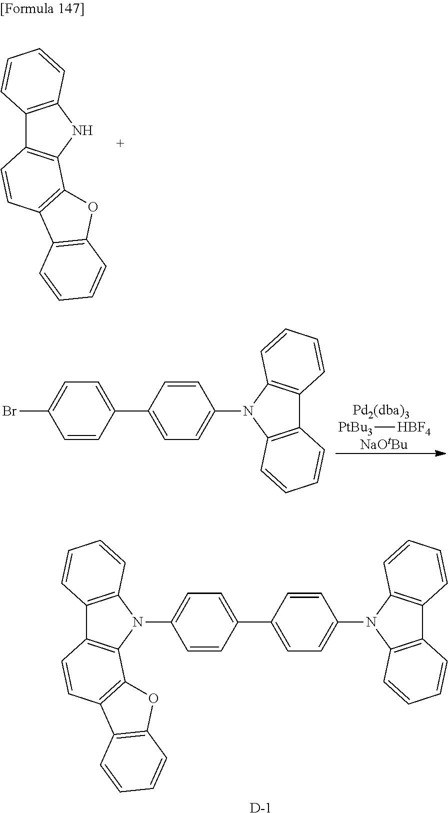

Under nitrogen atmosphere, xylene (675 mL) was added into a mixture of 12H-benzofuro[2,3-a]carbazole (26.6 g, 103 mmol), 9-(4′-bromo-[1,1′-biphenyl]-4-yl)-9H-carbazole (41.2 g, 103 mmol), tris(dibenzylideneacetone)dipalladium (1.90 g, 2.07 mmol), tri-tert-butylphosphonium tetrafluoroborate (1.20 mg, 4.14 mmol), and sodium tert-butoxide (11.9 g, 124 mmol), and stirred at 130 degrees C. for eight hours. After the reaction, a solid was collected by filtration. The solid collected by filtration was recrystallized with toluene to obtain the compound D-1 (51.5 g, a yield of 87%). The obtained compound was identified as the compound D-1 by analysis according to LC-MS (Liquid chromatography mass spectrometry).

### Synthesis Example 2: Synthesis of Compound TADF-1

### (2-1) Synthesis of Intermediate A1 and Intermediate A2

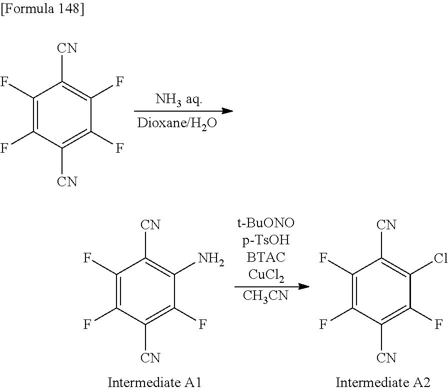

Under nitrogen atmosphere, into a 2000-mL three-necked flask, tetrafluoroterephthalonitrile (25 g, 125 mmol), 1,4-dioxane (625 mL) and water (400 mL) were put.

Next, 30 mass % ammonia water (13 mL) was put into the mixture and heated at 80 degrees C. for ten hours with stirring and then returned to the room temperature (25 degrees C.). A solvent was distilled away from the mixture using an evaporator. The obtained solid was purified by silica-gel column chromatography to obtain a white solid (24 g). The obtained solid was identified as an intermediate A1 (a yield of 98%) by GC-MS (Gas Chromatograph Mass Spectrometry).

Under nitrogen atmosphere, into a 2000-mL three-necked flask, the intermediate A1 (24 g, 122 mmol), p-toluenesulfonic acid (p-TsOH) (25 g, 146 mmol), benzyltrimethylammonium chloride (BTAC) (45.3 g. 244 mmol), copper chloride (11) (0.16 g, 1.22 mmol) and acetonitrile (400 mL) were put. Next, tert-Butyl nitrite (t-BuONO) (15 g, 146 mmol) was put into the mixture and stirred at 25 degrees C. for six hours. A solvent was distilled away from the mixture using an evaporator. The obtained solid was purified by silica-gel column chromatography to obtain a white solid (17 g). The obtained solid was identified as an intermediate A2 (a yield of 65%) by GC-MS.

### (2-2) Synthesis of Intermediate A3

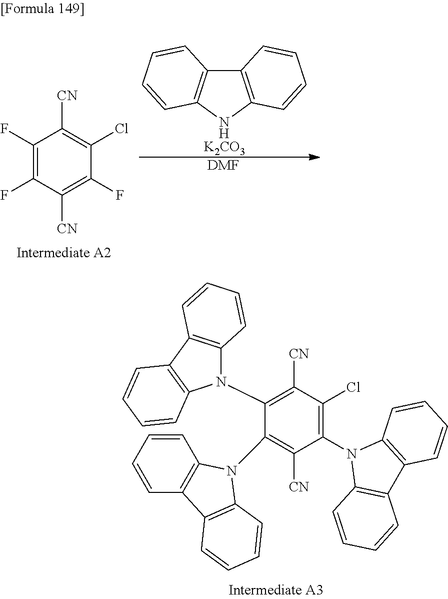

Under nitrogen atmosphere, into a 1000-mL three-necked flask, the intermediate A2 (10 g, 46 mmol), carbazole (23 g, 138 mmol), potassium carbonate (19 g, 138 mmol) and DMF (450 mL) were put and stirred at 0 degrees C. for 24 hours. 300 mL of a saturated aqueous solution of ammonium chloride was added to the reaction mixture. The deposited solid was purified by silica-gel column chromatography to obtain a yellow solid (26 g). The obtained solid was identified as an intermediate A3 (a yield of 85%) by analysis of ASAP-MS (Atmospheric Pressure Solid Analysis Probe Mass Spectrometry).

### (2-3) Synthesis of Intermediate C2 and Intermediate D2

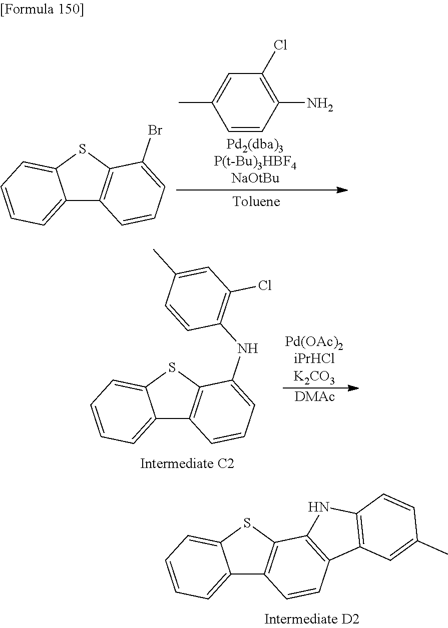

Under nitrogen atmosphere, to a 1-L three-necked flask, 4-bromodibenzothiophene (26.0 g, 100 mmol), 2-chloro-4-methylaniline (17 g, 120 mmol), tris(dibenzylideneacetone)dipalladium(0) (Pd₂dba₃) (0.9 g, 1 mmol), tri-tert-butylphosphonium tetrafluoroborate (P(t-Bu)₃HBF₄) (2.3 g, 8 mmol), sodium tert-butoxide (NaOtBu) (11.5 g, 120 mmol) and toluene (350 mL) were added, heated at 60 degrees C. for seven hours with stirring, and subsequently cooled to the room temperature (25 degrees C.). The reaction solution was purified by silica-gel column chromatography to obtain a white solid (26 g). The obtained solid was identified as an intermediate C2 (a yield of 80%) by GC-MS.

Under nitrogen atmosphere, to a 1-L three-necked flask, intermediate C2(26.0 g, 80 mmol), 1,3-bis(2,6-diisopropylphenyl)imidazolium chloride (IPrHCl) (1.4 g, 3.2 mmol), acetic acid palladium(II) (Pd(OAc)2) (0.36 g, 1.6 mmol), potassium carbonate (22.0 g, 160 mmol) and N,N-dimethylacetamide (DMAc) (400 mL) were added, stirred at 130 degrees C. for seven hours, and subsequently cooled to the room temperature (25 degrees C.). The reaction solution was purified by silica-gel column chromatography to obtain a white solid (21 g). The obtained solid was identified as an intermediate D2 (a yield of 91%) by GC-MS.

### (2-4) Synthesis of Compound TADF-1

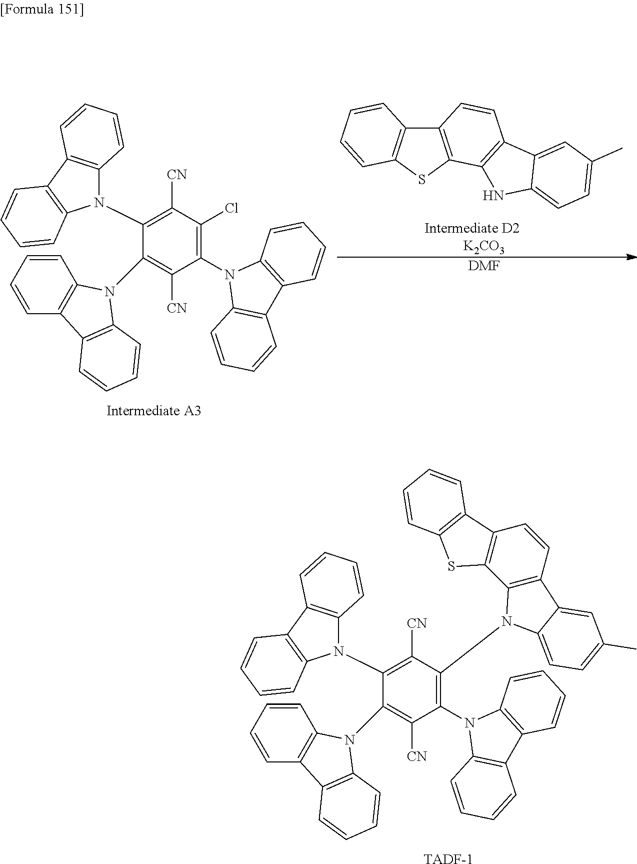

Under nitrogen atmosphere, into a 100-mL three-necked flask, an intermediate A3 (2 g, 3.0 mmol), the intermediate D2 (1.0 g, 3.6 mmol), potassium carbonate (0.6 g, 4.5 mmol) and DMF 30 mL were put and stirred at 70 degrees C. for eight hours. 50 mL of a saturated aqueous solution of ammonium chloride was added to the reaction mixture. The deposited solid was purified by silica-gel column chromatography to obtain a red solid (1.8 g). The obtained solid was identified as the compound TADF-1 (a yield of 66%) by ASAP-MS.

### Synthesis Example 3: Synthesis of Compound RD-2

A pyrrole compound (2-1) (2.5 g) and 4-methoxy-2,3,6-trimethylbenzaldehyde (0.73 g) were dissolved in dichloromethane (50 ml), to which 10 drops of trifluoroacetate were added, and, under nitrogen stream, stirred at 25 degrees C. for 24 hours. After water was added to the reaction mixture, an organic layer was separated from the reaction mixture, washed with saturated saline solution (50 ml), subsequently, added with magnesium sulfate, and filtered. A solvent was removed from the filtrate with an evaporator to obtain a pyrromethane compound (2-2) (residue).

The obtained pyrromethane compound (2-2) was dissolved in 1,2-dichloroethane (50 mL), to which 2,3-dichloro-5,6-dicyano-p-benzoquinone (DDQ) (0.9 g) was added. Under nitrogen stream, the obtained mixture was stirred at the room temperature (25 degrees C.) for two hours. Formation of a compound (2-3) was confirmed by LC-MS analysis. Subsequently, diisopropylethylamine (5.4 mL) and boron trifluoride-diethyl ether complex (3.9 mL) were added to the mixture and stirred at 80 degrees C. for one hour. After the reaction solution was cooled to the room temperature, water (50 mL) was put into the reaction solution, and the reaction solution was subjected to extraction with ethyl acetate (50 mL). An organic layer was washed with water (50 mL), subsequently added with magnesium sulfate, and filtered. A solvent was removed from the filtrate with an evaporator. Subsequently, the residue was purified by silica-gel column chromatography (heptane/toluene=1/2, volume ratio). The purified substance was further condensed, to which methanol (50 mL) was added. The obtained mixture was heated at 60 degrees C. for 10 minutes with stirring, and then left to cool. The deposited solid was filtered and dried in a vacuum to obtain magenta powder (1.7 g). The obtained powder was analyzed by LC-MS and the magenta powder was confirmed to be the compound RD-2 that was a pyrromethene metal complex.

Compound RD-2: MS (m/z) molecular weight; 817

The compound RD-2 was sublimated and purified at 270 degrees C. under pressure of 1×10⁻³ Pa using an oil diffusion pump. A solid adhering on a wall of a glass pipe was collected and purity of the solid by LC-MS analysis was confirmed to be 99%.

Luminescence properties of the compound RD-2 in a solution are shown below.

A singlet energy S₁ of the compound RD-2 was measured according to the above-described solution method. A main peak wavelength λ of the compound RD-2 was measured according to the same method as the main peak wavelength λ of the compound RD-1 described above.

Main peak wavelength λ of the compound RD-2: 622 nm

Singlet energy S₁ of the compound RD-2: 1.99 eV

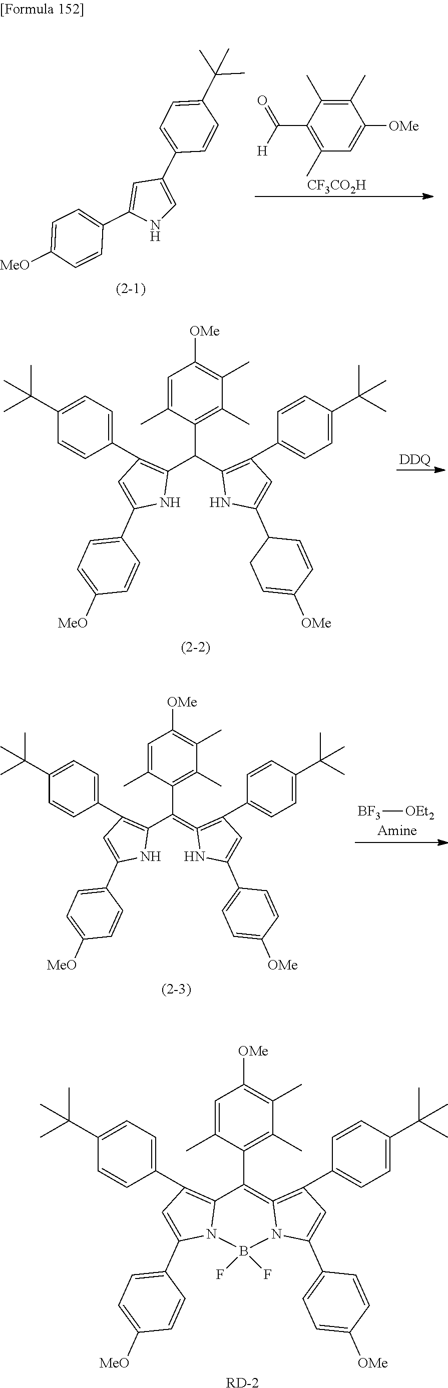

### Synthesis Example 4: Synthesis of Compound RD-3

A mixture solution of a pyrrole compound (3-1) (2.0 g), 1-naphtoyl chloride (1.2 g), and o-xylene (60 mL) was heated at 130 degrees C. for five hours with stirring under nitrogen stream. The mixture solution was cooled to the room temperature (25 degrees C.), subsequently, added with methanol to deposit a solid. The solid was filtered and dried in a vacuum to obtain a compound (3-2) (2.5 g).

Next, a mixture solution of the compound (3-2) (2.5 g), the pyrrole compound (3-1) (1.7 g), trifluoromethanesulfonic anhydride (2.9 g), and toluene (100 mL) was heated at 110 degrees C. for six hours with stirring under nitrogen stream. After the mixture solution was cooled to the room temperature, water (100 mL) was put into the mixture solution, and the mixture solution was subjected to extraction with ethyl acetate (100 mL). An organic layer was washed with water (50 mL), subsequently added with magnesium sulfate, and filtered. A solvent was removed from the filtrate with an evaporator to obtain a pyrromethene body (3-3) (residue).

Subsequently, diisopropylethylamine (5.4 mL) and a boron trifluoride-diethyl ether complex (3.9 mL) were added to a mixture solution of the obtained pyrromethene body (3-3) and toluene (100 mL) under nitrogen stream and stirred at 80 degrees C. for one hour. Subsequently, water (100 mL) was put into the mixture solution, and the mixture solution was subjected to extraction with ethyl acetate (100 mL). An organic layer was washed with water (50 mL), subsequently added with magnesium sulfate, and filtered. A solvent was removed from the filtrate with an evaporator. Subsequently, the residue was purified by silica-gel column chromatography (heptane/toluene=1/2, volume ratio). The purified substance was further condensed, to which methanol (100 mL) was added. The obtained mixture was heated at 60 degrees C. for 10 minutes with stirring, and then left to cool. The deposited solid was filtered and dried in a vacuum to obtain magenta powder (2.1 g). The obtained powder was analyzed by LC-MS and the magenta powder was confirmed to be the compound RD-3 that was a pyrromethene metal complex.

Compound RD-3: MS (m/z) 842[M+H]⁺

The compound RD-3 was sublimated and purified at 290 degrees C. under pressure of 1×10⁻³ Pa using an oil diffusion pump. A solid adhering on a wall of a glass pipe was collected and purity of the solid by LC-MS analysis was confirmed to be 99%.

Luminescence properties of the compound D-3 in a solution are shown below.

A singlet energy S₁ of the compound RD-3 was measured according to the above-described solution method. A main peak wavelength λ of the compound RD-3 was measured according to the same method as the main peak wavelength λ of the compound RD-1 described above.

Main peak wavelength λ of the compound RD-3: 613 nm

Singlet energy S₁ of the compound RD-3: 2.01 eV

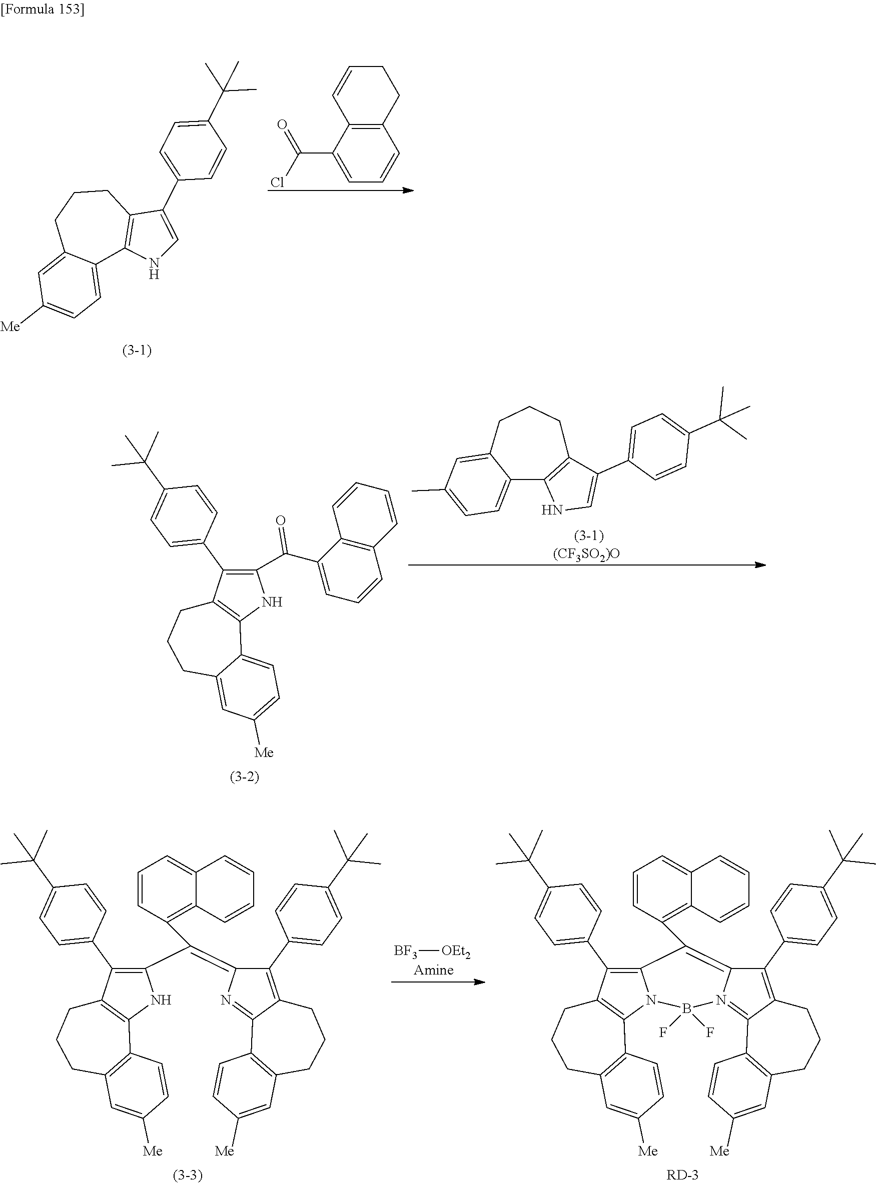

### EXPLANATION OF CODES

1 . . . organic EL device, 2 . . . substrate, 3 . . . anode, 4 . . . cathode, 5 . . . emitting layer, 6 . . . first layer, 7 . . . second layer, 10 . . . organic layer.

1: An organic electroluminescence device, comprising:
an anode;
a cathode;
an emitting layer provided between the anode and the cathode;
a first layer provided between the anode and the emitting layer and adjacent to the emitting layer; and
a second layer provided between the cathode and the emitting layer and adjacent to the emitting layer, wherein
the emitting layer comprises a first compound, a second compound, and a third compound,
the first layer comprises a compound represented by a formula (A) below,
the second layer comprises a compound represented by a formula (B) below,
the first compound is a fluorescent compound and is represented by a formula (1) below,
the second compound is a delayed fluorescent compound and is represented by a formula (2) below,
the third compound is represented by a formula (3) below, and
a singlet energy S₁(M1) of the first compound, a singlet energy S₁(M2) of the second compound, and a singlet energy S₁(M3) of the third compound satisfy a relationship of a numerical formula (Numerical Formula 1) below, 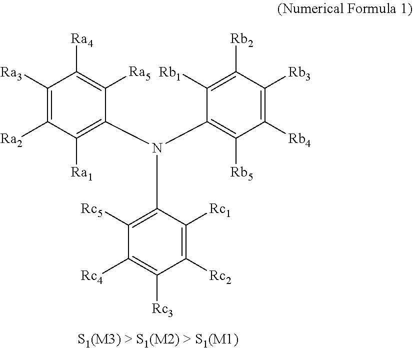
wherein:
Ra₁ to Ra₅, Rb₁ to Rb₅, and Rc₃ to Rc₅ are each independently a hydrogen atom or a substituent; Ra₁ to Ra₅, Rb₁ to Rb₅, and Rc₃ to Rc₅ serving as the substituent are each independently a halogen atom, a cyano group, a substituted or unsubstituted aryl group having 6 to 30 ring carbon atoms, or a substituted or unsubstituted heteroaryl group having 5 to 30 ring atoms;
Rc₁ is a hydrogen atom or a substituent, or a pair of Rc₁ and Rc₂ are mutually bonded to form a ring, and Rc₁ serving as the substituent is a substituted or unsubstituted aryl group having 6 to 12 ring carbon atoms;
Rc₂ is a hydrogen atom or a substituent, or a pair of Rc₁ and Rc₂ are mutually bonded to form a ring;
when a pair of Rc₁ and Rc₂ are mutually bonded to form a ring, the ring at least comprises a five-membered ring, the five-membered ring comprising at least one of a carbon atom, an oxygen atom, a sulfur atom, or a nitrogen atom;
Rc₁ and Rc₂ are not hydrogen atoms at the same time;
Rc₂ serving as the substituent is a substituted or unsubstituted aryl group having 6 to 30 ring carbon atoms, a substituted or unsubstituted heteroaryl group having 5 to 30 ring atoms, a substituted or unsubstituted aryloxy group having 6 to 30 ring carbon atoms, a substituted or unsubstituted arylthio group having 6 to 30 ring carbon atoms, or a substituted or unsubstituted amino group, 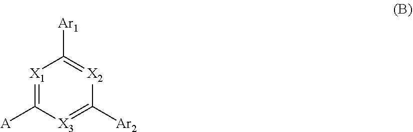
wherein:
X₁ to X₃ are each independently a nitrogen atom or CR₁, at least one of X₁ to X₃ is a nitrogen atom;
R₁ is a hydrogen atom or a substituent;
R₁ serving as the substituent is each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms;
Ar₁ and Ar₂ are each independently represented by a formula (1B) below, or are a substituted or unsubstituted aryl group having 6 to 30 ring carbon atoms, or a substituted or unsubstituted heteroaryl group having 5 to 30 ring atoms; and
A is represented by a formula (1B) below, 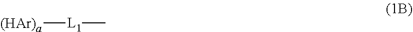
wherein:
HAr is represented by a formula (2B) below;
a is 1, 2, 3, 4 or 5;
when a is 1, L₁ is a single bond or a divalent linking group;
when a is 2, 3, 4 or 5, L₁ is a trivalent to hexavalent linking group;
a plurality of HAr are mutually the same or different;
the linking group is a group derived from a substituted or unsubstituted aryl group having 6 to 30 ring carbon atoms, a group derived from a substituted or unsubstituted heteroaryl group having 5 to 30 ring atoms, a group derived from a group formed by mutually bonding two groups selected from the group consisting of a substituted or unsubstituted aryl group having 6 to 30 ring carbon atoms and a substituted or unsubstituted heteroaryl group having 5 to 30 ring atoms, or a group derived from a group formed by mutually bonding three groups selected from the group consisting of a substituted or unsubstituted aryl group having 6 to 30 ring carbon atoms and a substituted or unsubstituted heteroaryl group having 5 to 30 ring atoms; and
the mutually bonded groups are the same or different, 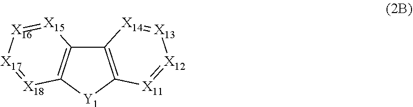
wherein:
X₁₁ to X₁₈ are each independently a nitrogen atom, CR₁₃, or a carbon atom bonded to L₁;
a plurality of R₁₃ are mutually the same or different;
Y₁ is an oxygen atom, a sulfur atom, NR₁₈, SiR₁₁R₁₂, CR₁₄R₁₅, a nitrogen atom bonded to L₁, a silicon atom bonded to each of R₁₆ and L₁, or a carbon atom bonded to each of R₁₇ and L₁;
among carbon atoms in X₁₁ to X₁₈, R₁₁ to R₁₂, and R₁₄ to R₁₅ as well as a nitrogen atom, a silicon atom, and a carbon atom in Y₁, one atom is bonded to L₁;
R₁₁ and R₁₂ are mutually the same or different, R₁₄ and R₁₅ are mutually the same or different;
R₁₁₈ to R₁₈ are each independently a hydrogen atom or a substituent, or at least one pair of a pair of adjacent ones of R₁₃, a pair of R₁₁ and R₁₂, or a pair of R₁₄ and R₁₅ are bonded to each other to form a ring; and
R₁₁ to R₁₈ serving as the substituent are each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms, 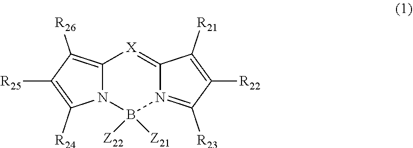
wherein:
X is a nitrogen atom, or a carbon atom bonded to Y;
Y is a hydrogen atom or a substituent;
R₂₁ to R₂₆ are each independently a hydrogen atom or a substituent, or at least one of a pair of R₂₁ and R₂₂, a pair of R₂₂ and R₂₃, a pair of R₂₄ and R₂₅, or a pair of R₂₅ and R₂₆ are mutually bonded to form a ring;
Y and R₂₁ to R₂₆ serving as the substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;
Z₂₁ and Z₂₂ are each independently a substituent, or are mutually bonded to form a ring; and
Z₂₁ and Z₂₂ serving as the substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms, 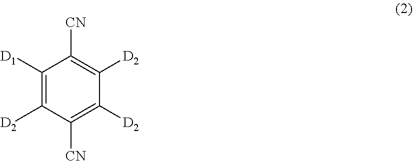
wherein: D₁ is a group represented by a formula (2-1) below, D₂ is a group represented by a formula (2-2), and a plurality of D₂ are mutually the same group, 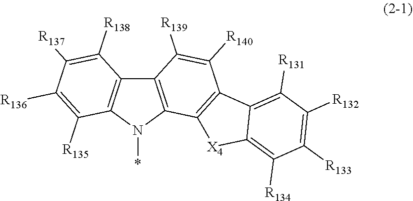
wherein:
X₄ is an oxygen atom or a sulfur atom, and R₁₃₁ to R₁₄₀ are each independently a hydrogen atom or a substituent;
R₁₃₁ to R₁₄₀ serving as the substituent are each independently a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkylsilyl group having 3 to 6 carbon atoms, a substituted or unsubstituted alkoxy group having 1 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and
* represents a bonding position to a benzene ring in the formula (2), 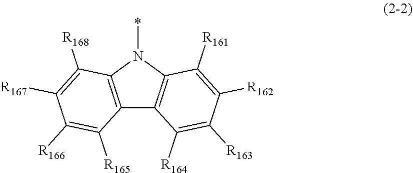
wherein:
R₁₆₁ to R₁₆₈ are each independently a hydrogen atom or a substituent;
R₁₆₁ to R₁₆₈ serving as the substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted alkylsilyl group having 3 to 6 carbon atoms, a substituted or unsubstituted alkoxy group having 1 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and
* each independently represents a bonding position to a benzene ring in the formula (2), 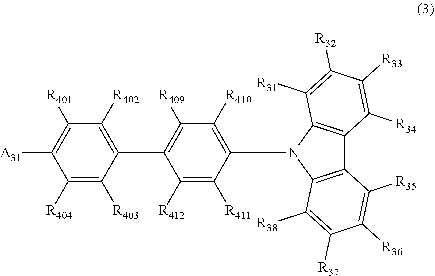
wherein:
A₃₁ is a group represented by a formula (31a), formula (31b), formula (31c), formula (31d), formula (31e) or formula (31f);
R₃₁ to R₃₈ are each independently a hydrogen atom or a substituent; R₄₀₁ to R₄₀₄ and R₄₀₉ to R₄₁₂ are each independently a hydrogen atom or a substituent; and
R₃₁ to R₃₈ serving as the substituent and R₄₀₁ to R₄₀₄ and R₄₀₉ to R₄₁₂ serving as the substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms, 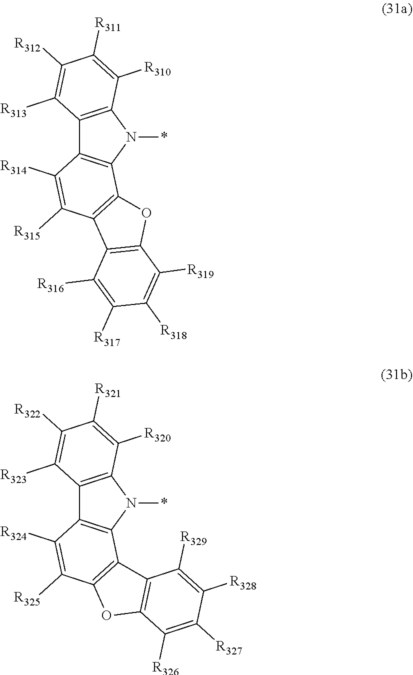 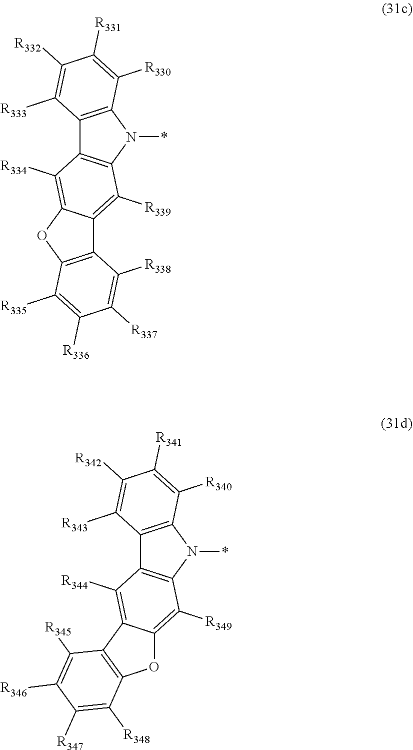 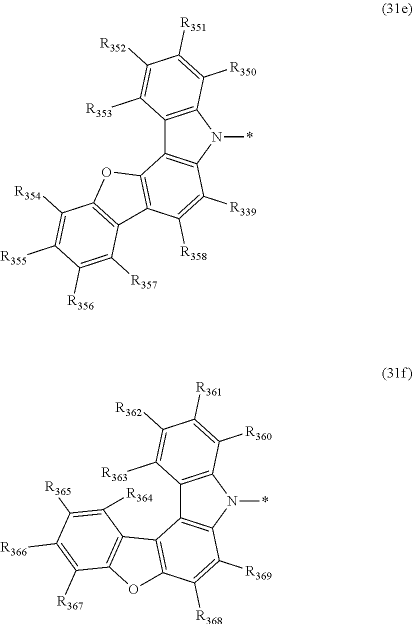
in the formula (31a), formula (31b), formula (31c), formula (31d), formula (31e) and formula (31 f):
R₃₁₀ to R₃₁₉ are each independently a hydrogen atom or a substituent;
R₃₂₀ to R₃₂₉ are each independently a hydrogen atom or a substituent;
R₃₃₀ to R₃₃₉ are each independently a hydrogen atom or a substituent;
R₃₄₀ to R₃₄₉ are each independently a hydrogen atom or a substituent;
R₃₅₀ to R₃₅₉ are each independently a hydrogen atom or a substituent;
R₃₆₀ to R₃₆₉ are each independently a hydrogen atom or a substituent;
R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ serving as the substituent each independently represent the same as R₃₁ to R₃₈ serving as the substituent and R₄₀₁ to R₄₀₄ and R₄₀₉ to R₄₁₂ serving as the substituent in the formula (3); and
* each independently represents a bonding position to a benzene ring having R₄₀₁ to R₄₀₄ in the formula (3).

2: The organic electroluminescence device according to claim 1, wherein
Rc₁ is a hydrogen atom or a substituent, and
Rc₂ is a hydrogen atom or a substituent.

3: The organic electroluminescence device according to claim 1, wherein
the compound represented by the formula (A) is represented by a formula (1X) below, 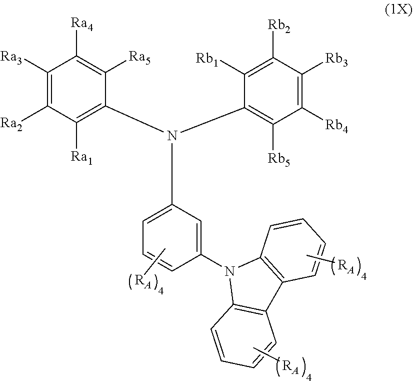
where:
Ra₁ to Ra₅ and Rb₁ to Rb₃ each represent the same as Ra₁ to Ra₅ and Rb₁ to Rb₅ in the formula (A);
R_{A} is a hydrogen atom or a substituent;
R_{A} serving as the substituent is each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms; and
when a plurality of R_{A} are present, the plurality of R_{A} are mutually the same or different.

4: The organic electroluminescence device according to claim 1, wherein
Ra₁ to Ra₅ and Rb₁ to Rb₅ are each independently a hydrogen atom or a substituted or unsubstituted heteroaryl group having 5 to 30 ring atoms.

5: The organic electroluminescence device according to claim 1, wherein
one of Ra₁ to Ra₁ is a substituent and Ra₁ to Ra₁ not being the substituent are hydrogen atoms,
one of Rb₁ to Rb₅ is a substituent and Rb₁ to Rb₅ not being the substituent are hydrogen atoms, and
Rc₃ to Rc₅ are hydrogen atoms.

6: The organic electroluminescence device according to claim 1, wherein
the compound represented by the formula (A) has an ionization potential Ip of 5.78 eV or more.

7: The organic electroluminescence device according to claim 1, wherein
in the formula (B), two or three of X₁ to X₃ are nitrogen atoms.

8: The organic electroluminescence device according to claim 1, wherein
L₁ serving as the linking group is a trivalent to hexavalent residue derived from a substituted or unsubstituted aryl group having 6 to 30 ring carbon atoms.

9: The organic electroluminescence device according to claim 1, wherein
in the formula (1B), a is 2 and L₁ is a linking group,
L₁ as the linking group is a trivalent residue derived from a substituted or unsubstituted aryl group having 6 to 30 ring carbon atoms or
a trivalent residue derived from a substituted or unsubstituted heteroaryl group having 5 to 30 ring atoms.

10: The organic electroluminescence device according to claim 1, wherein
in the formula (2B), Y₁ is an oxygen atom or a sulfur atom.

11: The organic electroluminescence device according to claim 1, wherein
in the formula (2B), Y₁ is an oxygen atom or a sulfur atom, and
one of X₁₁ to X₁₈ is a carbon atom bonded to L₁ and the rest of X₁₁ to X₁₈ are each CR₁₃.

12: The organic electroluminescence device according to claim 1, wherein
in the formula (2B), X₁₃ or X₁₆ is a carbon atom bonded to L₁.

13: The organic electroluminescence device according to claim 1, wherein
in the formulae (2-1) and (2-2), R₁₃₁ to R₁₄₀ and R₁₆₁ to R₁₆₈ are each independently a hydrogen atom, a substituted or unsubstituted aryl group having 6 to 14 ring carbon atoms, a substituted or unsubstituted heterocyclic group having 5 to 14 ring atoms, or a substituted or unsubstituted alkyl group having 1 to 6 carbon atoms.

14: The organic electroluminescence device according to claim 1, wherein
in the formulae (2-1) and (2-2), R₁₃₁ to R₁₄₀ and R₁₆₁ to R₁₆₈ are each independently a hydrogen atom, a substituted or unsubstituted aryl group having 6 to 14 ring carbon atoms, or a substituted or unsubstituted alkyl group having 1 to 6 carbon atoms.

15: The organic electroluminescence device according to claim 1, wherein
in the formulae (2-1) and (2-2), R₁₃₁ to R₁₄₀ and R₁₆₁ to R₁₆₈ are each independently a hydrogen atom, or a substituted or unsubstituted alkyl group having 1 to 6 carbon atoms.

16: The organic electroluminescence device according to claim 1, wherein
the compound represented by the formula (1) is a compound represented by a formula (n) below, 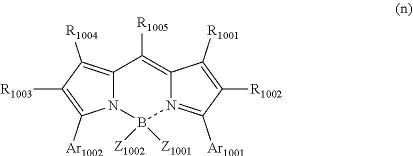
wherein:
Ar₁₀₀₁ and Ar₁₀₀₂ are each independently selected from the group consisting of a substituted or unsubstituted aryl group having 6 to 30 ring carbon atoms, and a substituted or unsubstituted heteroaryl group having 5 to 30 ring atoms;
R₁₀₀₁ to R₁₀₀₅ are each independently a hydrogen atom or a substituent, or at least one pair of a pair of R₁₀₀₁ and R₁₀₀₂, a pair of R₁₀₀₂ and Ar₁₀₀₁, a pair of Ar₁₀₀₂ and R₁₀₀₃, or a pair of R₁₀₀₃ and R₁₀₀₄ are bonded to each other to form a ring, and
R₁₀₀₁ to R₁₀₀₅ serving as the substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group; and
Z₁₀₀₁ and Z₁₀₀₂ are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

17: The organic electroluminescence device according to claim 16, wherein
the compound represented by the formula (n) is a compound represented by a formula (n+1A) or a formula (n+1B) below, 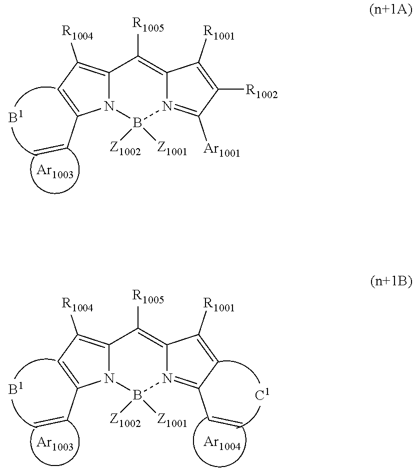
in the formula (n+1A), R₁₀₀₁, R₁₀₀₂, R₁₀₀₄, R₁₀₀₅, Ar₁₀₀₁, Z₁₀₀₁ and Z₁₀₀₂ each independently represent the same as R₁₀₀₁, R₁₀₀₂, R₁₀₀₄, R₁₀₀₅, Ar₁₀₀₁, Z₁₀₀₁ and Z₁₀₀₂ in the formula (n),
in the formula (n+1B), R₁₀₀₁, R₁₀₀₄, R₁₀₀₅, Z₁₀₀₁ and Z₁₀₀₂ each independently represent the same as R₁₀₀₁, R₁₀₀₄, R₁₀₀₅, Z₁₀₀₁ and Z₁₀₀₂ in the formula (n),
Ar₁₀₀₃ and Ar₁₀₀₄ are each independently selected from the group consisting of a substituted or unsubstituted aromatic hydrocarbon ring having 6 to 30 ring carbon atoms, and a substituted or unsubstituted aromatic heterocyclic ring having 5 to 30 ring atoms,
B¹ is a bridging structure in which three or more atoms are bonded in series, the atoms being selected from the group consisting of a substituted or unsubstituted carbon atom, a substituted or unsubstituted silicon atom, a substituted or unsubstituted nitrogen atom, a substituted or unsubstituted phosphorus atom, an oxygen atom, and a sulfur atom,
C¹ is abridging structure in which one or more atoms are bonded in series, the atoms being selected from the group consisting of a substituted or unsubstituted carbon atom, a substituted or unsubstituted silicon atom, a substituted or unsubstituted nitrogen atom, a substituted or unsubstituted phosphorus atom, an oxygen atom, and a sulfur atom, and
when B¹ is a trimethylene group, R₁₀₀₄ is neither a hydrogen atom nor a halogen atom.

18: The organic electroluminescence device according to claim 17, wherein
B¹ is a bridging structure represented by a formula (n+2A) or a formula (n+2B) below, 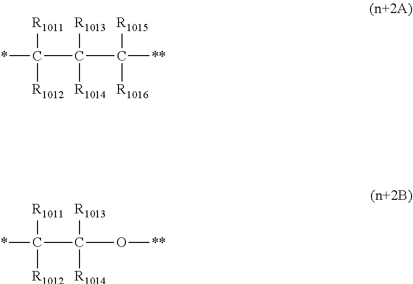
in the formula (n+2A), R₁₀₁₁ to R₁₀₁₆ are each independently a hydrogen atom or a substituent, or at least one pair of pairs of adjacent two or more of R₁₀₁₁ to R₁₀₁₆ are bonded to each other to form a ring,
in the formula (n+2B), R₁₀₁₁ to R₁₀₁₄ are each independently a hydrogen atom or a substituent, or at least one pair of pairs of adjacent two or more of R₁₀₁₁ to R₁₀₁₄ are bonded to each other to form a ring,
R₁₀₁₁ to R₁₀₁₆ serving as the substituent are each independently a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted heteroaryl group having 5 to 30 ring atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted alkynyl group having 2 to 30 carbon atoms, a halogen atom, a carboxy group, a substituted or unsubstituted amino group, a nitro group, a cyano group, a substituted or unsubstituted silyl group, a hydroxy group, an ester group, a siloxanyl group, or a carbamoyl group, and
* represents a bonding moiety to a pyrrole ring, and ** represents a bonding moiety to Ar₁₀₀₃ in the formula (n+1A) and the formula (n+1B).

19: The organic electroluminescence device according to claim 16, wherein
R₁₀₀₅ is a group represented by a formula (n+3), 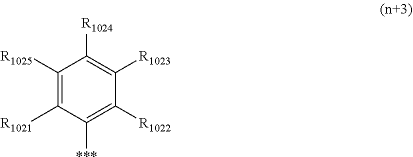
wherein:
R₁₀₂₁ and R₁₀₂₂ are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, and a substituted or unsubstituted heteroaryl group having 5 to 30 ring atoms;
R₁₀₂₃ to R₁₀₂₅ are each independently a hydrogen atom or a substituent, or at least one pair of a pair of R₁₀₂₃ and R₁₀₂₄, or a pair of R₁₀₂₄ and R₁₀₂₅ are bonded to each other to form a ring;
R₁₀₂₃ to R₁₀₂₅ serving as the substituent are each independently a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted heteroaryl group having 5 to 30 ring atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsulfonyl group having 1 to 30 carbon atoms, a substituted or unsubstituted arylcarbonyl group having 6 to 30 ring carbon atoms, a substituted or unsubstituted acyl group having 1 to 30 carbon atoms, a halogen atom, a carboxy group, a substituted or unsubstituted amino group, a nitro group, a cyano group, a substituted or unsubstituted silyl group, a hydroxy group, an ester group, a siloxanyl group, or a carbamoyl group; and
*** represents a bonding position to a carbon atom bonded to R₁₀₀₅ in the formula (n).

20: The organic electroluminescence device according to claim 1, wherein
a substituent for a substituted or unsubstituted group is a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted heteroaryl group having 5 to 30 ring atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted alkynyl group having 2 to 30 carbon atoms, a substituted or unsubstituted aralkyl group having 7 to 30 carbon atoms, a substituted or unsubstituted alkylsulfonyl group having 1 to 30 carbon atoms, a substituted or unsubstituted arylcarbonyl group having 6 to 30 ring carbon atoms, a substituted or unsubstituted acyl group having 1 to 30 carbon atoms, a halogen atom, a carboxy group, a substituted or unsubstituted amino group, a nitro group, a cyano group, a substituted or unsubstituted silyl group, a substituted phosphoryl group, a hydroxy group, a substituted phosphino group, an ester group, a siloxanyl group, or a carbamoyl group.

21: The organic electroluminescence device according to claim 1, wherein
the emitting layer does not comprise a metal complex.

22: An electronic device, comprising the organic electroluminescence device according to claim 1.
